(19) United States
(12) Patent Application Publication
(10) Pub. No.: US 2023/0174488 A1
(43) Pub. Date: Jun. 8, 2023

(54) SARS-COV-2 INHIBITORS HAVING COVALENT MODIFICATIONS FOR TREATING CORONAVIRUS INFECTIONS

(71) Applicant: Insilico Medicine IP Limited, Hong Kong (HK)

(72) Inventors: Aleksandrs Zavoronkovs, Pak Shek Kok (HK); Yan A. Ivanenkov, Moscow (RU); Bogdan Zagribelnyy, Pak Shek Kok (HK)

(21) Appl. No.: 18/049,757

(22) Filed: Oct. 26, 2022

Related U.S. Application Data
(63) Continuation of application No. PCT/CN2021/091093, filed on Apr. 29, 2021.
(60) Provisional application No. 63/017,878, filed on Apr. 30, 2020.
(60) Provisional application No. 63/059,095, filed on Jul. 30, 2020.
(60) Provisional application No. 63/112,087, filed on Nov. 10, 2020.

(51) Int. Cl. C07D 213/56; A61P 31/14; C07D 405/12; C07D 401/12; C07D 491/107; C07D 413/12; C07D 413/14; C07C 237/22; C07D 471/04; C07D 239/26; C07D 487/04; C07D 237/08; C07D 233/32; C07D 217/16; C07D 403/12; C07D 417/12; C07D 417/14; C07D 491/048; C07D 409/12

(52) U.S. Cl. CPC C07D 213/56; A61P 31/14; C07C 237/22; C07D 217/16; C07D 233/32; C07D 237/08; C07D 239/26; C07D 401/12; C07D 403/12; C07D 405/12; C07D 409/12; C07D 413/12; C07D 413/14; C07D 417/12; C07D 417/14; C07D 471/04; C07D 487/04; C07D 491/048; C07D 491/107

(57) ABSTRACT

Provided herein are compounds, pharmaceutical compositions and methods for treating a SARS-CoV-2 infection.

### CROSS-REFERENCE TO RELATED APPLICATIONS

This application is a continuation of International Application No. PCT/CN2021/091093, filed on Apr. 29, 2021, which claims the benefit of U.S. Provisional Pat. Application No. 63/017,878, filed on Apr. 30, 2020, U.S. Provisional Pat. Application No. 63/059,095, filed on Jul. 30, 2020, and U.S. Provisional Pat. Application No. 63/112,087, filed on Nov. 10, 2020, all of which are incorporated herein by reference in their entirety.

### FIELD OF THE DISCLOSURE

The present disclosure relates to compounds and/or materials for use as potential SARS-CoV-2 inhibitors.

### BACKGROUND OF THE DISCLOSURE

SARS-CoV2 (also known as 2019-nCoV or COVID-19) first appeared in 2019. Symptoms linked with the disease include fever, myalgia, cough, dyspnea and fatigue (Huang et al., 2020). Currently, there is no treatment available for SARS-CoV-2. Nevertheless, treatments with well-known drugs such as chloroquine or investigational drugs such as remdesivir are suggested for this disease (Colson et al., 2020; Wang et al., 2020). A cocktail of human immunodeficiency virus (HIV) drugs, lopinavir/ritonavir is also being investigated as a therapy for SARS-CoV2 as they exhibited anti-coronavirus effect in vitro (Que et al., 2003; Chu et al., 2004; Chan et al., 2015; Li and De Clercq, 2020).

SARS-CoV2 is a beta-coronavirus and is member of the family Coronaviridae, which comprises the largest positive-sense, single-stranded RNA viruses. (Cui et al., 2019). The virus contains four non-structural proteins: papain-like (PL^{pro}) and 3-chymotrypsin-like (3CL^{pro}) proteases, RNA polymerase and helicase (Zumla et al., 2016). Both proteases (PL^{pro} and 3CL^{pro}) are involved with transcription and replication of the virus. Amongst the four types, the 3CL^{pro} is considered to be mainly involved in the replication of the virus (de Wit et al., 2016). 3CLpro hydrolyses the viral polyproteins pp1a and pp1ab to produce functional proteins during coronavirus replication. A study reported that the cysteine protease 3CL^{pro} of SARS-CoV2 showed 96% sequence similarity with that of SARS-CoV (Xu et al., 2020). Because of its highly conserved sequence and essential functional properties, 3CL^{pro} has been validated as a potential target for the development of drugs to treat SARS-CoV-2.

Because viable treatments remain elusive, there is a need for a compound and/or method for inhibiting SARS-CoV-2 and for a treatment for a subject infected with the SARS-CoV-2.

### BRIEF SUMMARY

In one aspect, provided herein is a compound comprising of Formula (X), or a pharmaceutically acceptable salt or solvate thereof:

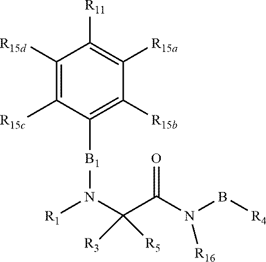

wherein,
B₁ and B are each independently a bond, C₁-C₄ alkylene, C₁-C₄ heteroalkylene, or C₃-C₆ cyclene linker, wherein the alkylene, heteroalkylene or cyclene is optionally substituted;
R₁ is halo acetyl, glyoxyl, heterocyclo acyl, cyanide acetyl, vinylsulfonyl, vinylsulfinyl, or acrylo acyl;
R₃ is an optionally substituted heteroaryl;
R₄ is an C₁-C₆ alkyl, aryl, heteroaryl, cycloalkyl or heterocycloalkyl, each of which is optionally substituted;
R₅ is H, C₁-C₆ alkyl, or C₁-C₃ haloalkyl
R₁₁ is amino, halogen, —CN, —OH, C₁-C₆ alkyl, C₁-C₆ haloalkyl, C₁-C₆ alkoxy, cycloalkyl, heterocycloalkyl, aryl, or heteroaryl, wherein each of the alkyl, alkoxy, aryl, cycloalkyl, heterocycloalkyl, or heteroaryl is optionally substituted;
R₁₅ₐ, R_{15b}, R_{15c}, and R_{15d} are each independently H, amino, halogen, —CN, —OH, —OCF₃, C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl is optionally substituted with one, two, or three R²⁰;
wherein optionally, R₁₅ₐ and R₁₁, taken in combination with the carbon atom to which they attach, form a 5-6 membered substituted or unsubstituted ring; or
wherein optionally, R₁₅ₐ and R_{15b}, taken in combination with the carbon atom to which they attach, form a 5-6 membered substituted or unsubstituted ring;
R₁₆ is H, C₁-C₆ alkyl, or C₁-C₃ haloalkyl; and
R₂₀ is oxo, halogen, —CN, —NH₂, —NH(C₁—₆ alkyl), —N(C₁—₆ alkyl)₂, —OH, —CO₂H, —CO₂—C₁—₆ alkyl, —C(═O)NH₂, —C(═O)NH(C₁—₆ alkyl), —C(═O)N(C₁—₆ alkyl)₂, —S(═O)₂NH₂, —S(═O)₂NH(C₁—₆ alkyl), —S(═O)₂N(C₁—₆ alkyl)₂, C₁-C₆ alkyl, C₁-C₆ haloalkyl, C₃₋₈ cycloalkyl, C₁-C₆ heteroalkyl, C₁-C₆ alkoxy, C₁₋₆ fluoroalkoxy, C₂₋₇ heterocycloalkyl, aryl, heteroaryl, aryloxy, alkylthio, arylthio, alkylsulfoxide, arylsulfoxide, cycloalkylsulfone, alkylsulfone, and arylsulfone.

In some embodiments, the compound has a structure of Formula (XA), or a pharmaceutically acceptable salt or solvate thereof:

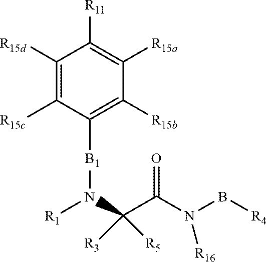

In another aspect, described herein is a compound has a structure of Formula (XB), or a pharmaceutically acceptable salt or solvate thereof:

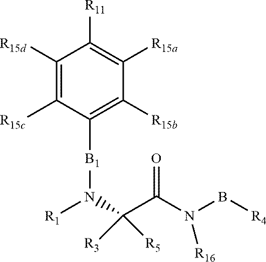

In another aspect, described herein is a compound having the structure of Formula (XI), or a pharmaceutically acceptable salt or solvate thereof:

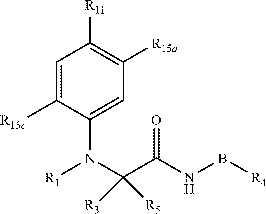

wherein,
B is a bond, C₁-C₄ alkylene, or C₃-C₆ cyclene linker;
R₁ is halo acetyl, glyoxyl, heterocyclo acyl or acrylo acyl;
R₃ is a heteroaryl optionally substituted with one, two, or three R₁₈;
R₄ is an aryl, heteroaryl, cycloalkyl or heterocycloalkyl, each of which is optionally substituted with one, two, three, or four R¹⁹;
R₅ is H, C₁-C₆ alkyl, or C₁-C₃ haloalkyl;
R₁₁ is amino, halogen, —CN, —OH, —OCF₃, C₁-C₆ alkyl, C₁-C₆ haloalkyl, C₁-C₆ alkoxy, cycloalkyl, heterocycloalkyl, aryl, or heteroaryl, wherein each of the alkyl, aryl, cycloalkyl, heterocycloalkyl, or heteroaryl is optionally substituted with one, two, or three R₁₇;
R₁₅ₐ and R_{15c} are each independently H, amino, halogen, —CN, —OH, —OCF₃, C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl is optionally substituted with one, two, or three R²⁰;
each R₁₇, R₁₈ , R₁₉, and R₂₀ is independently selected from oxo, halogen, —CN, —NH₂, —NH(C₁—₆ alkyl), -N(C₁₋₆ alkyl)₂, —OH, —CO₂H, —CO₂—C₁—₆ alkyl, —C(═O)NH₂, —C(═O)NH(C₁—₆ alkyl), —C(═O)N(C₁—₆ alkyl)₂, —S(═O)₂NH₂, —S(═O)₂NH(C₁—₆ alkyl), —S(═O)₂N(C₁—₆ alkyl)₂, C₁-C₆ alkyl, C₁-C₆ haloalkyl, C₃₋₈ cycloalkyl, C₁-C₆ heteroalkyl, C₁-C₆ alkoxy, C₁₋₆ fluoroalkoxy, C₂-₇ heterocycloalkyl, aryl, heteroaryl, aryloxy, alkylthio, arylthio, alkylsulfoxide, arylsulfoxide, cycloalkylsulfone, alkylsulfone, and arylsulfone.

In another aspect, described herein is a compound having a structure of Formula (XI): or a pharmaceutically acceptable salt or solvate thereof:

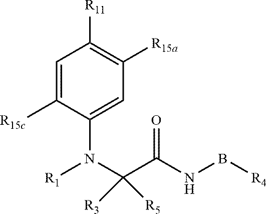

wherein,
B is a bond, C₁-C₄ alkylene, or C₃-C₆ cyclene linker;
R₁ is halo acetyl, glyoxyl, heterocyclo acyl or acrylo acyl;
R₃ is a heteroaryl optionally substituted with one, two, or three R₁₈;
R₄ is a substituted cycloalkyl or an optionally substituted heterocycloalkyl, wherein when substituted the each of which is substituted with one, two, three, or four R¹⁹;
R₅ is H, C₁-C₆ alkyl, or C₁-C₃ haloalkyl;
R₁₁ is amino, halogen, —CN, —OH, —OCF₃, C₁-C₆ alkyl, C₁-C₆ haloalkyl, C₁-C₆ alkoxy, cycloalkyl, heterocycloalkyl, aryl, or heteroaryl, wherein each of the alkyl, aryl, cycloalkyl, heterocycloalkyl, or heteroaryl is optionally substituted with one, two, or three R₁₇;
R₁₅ₐ and R_{15c} are each independently H, amino, halogen, —CN, —OH, —OCF₃, C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl is optionally substituted with one, two, or three R²⁰;
each R₁₇, R₁₈ , R₁₉, and R₂₀ is independently selected from oxo, halogen, —CN, —NH₂, —NH(C₁—₆ alkyl), —N(C_{1—6} alkyl)₂, —OH, —CO₂H, —CO₂—C₁—₆ alkyl, —C(═O)NH₂, —C(═O)NH(C₁—₆ alkyl), —C(═O)N(C_{1—6} alkyl)₂, —S(═O)₂NH₂, —S(═O)₂NH(C₁—₆ alkyl), —S(═O)₂N(C_{1—6} alkyl)₂, C₁-C₆ alkyl, C₁-C₆ haloalkyl, C₃₋₈ cycloalkyl, C₁-C₆ heteroalkyl, C₁-C₆ alkoxy, C₁₋₆ fluoroalkoxy, C₂-₇ heterocycloalkyl, aryl, heteroaryl, aryloxy, alkylthio, arylthio, alkylsulfoxide, arylsulfoxide, alkylsulfone, and arylsulfone.

In some embodiments, the compound has the structure of Formula (XI), or a pharmaceutically acceptable salt or solvate thereof:

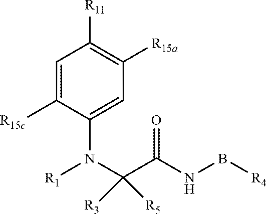

wherein,
B is a bond, C₁-C₄ alkylene, or C₃-C₆ cyclene linker;
R₁ is halo acetyl, glyoxyl, heterocyclo acyl or acrylo acyl;
R₃ is a heteroaryl optionally substituted with one, two, or three R₁₈;
R₄ is an aryl, heteroaryl, cycloalkyl or heterocycloalkyl, each of which is optionally substituted with one, two, three, or four R¹⁹;
R₅ is H, C₁-C₆ alkyl, or C₁-C₃ haloalkyl;
R₁₁ is a cycloalkyl, heterocycloalkyl, aryl, or heteroaryl, wherein each of the cycloalkyl, heterocycloalkyl, aryl, or heteroaryl is optionally substituted with one, two, or three R₁₇;
R₁₅ₐ and R_{15c} are each independently H, amino, halogen, —CN, —OH, —OCF₃, C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl is optionally substituted with one, two, or three R²⁰;
each R₁₇, R₁₈ , R₁₉, and R₂₀ is independently selected from oxo, halogen, —CN, —NH₂, —NH(C₁—₆ alkyl), —N(C_{1—6} alkyl)₂, —OH, —CO₂H, —CO₂—C₁—₆ alkyl, —C(═O)NH₂, —C(═O)NH(C₁—₆ alkyl), —C(═O)N(C_{1—6} alkyl)₂, —S(═O)₂NH₂, —S(═O)₂NH(C₁—₆ alkyl), —S(═O)₂N(C_{1—6} alkyl)₂, C₁-C₆ alkyl, C₁-C₆ haloalkyl, C₃₋₈ cycloalkyl, C₁-C₆ heteroalkyl, C₁-C₆ alkoxy, C₁₋₆ fluoroalkoxy, C₂-₇ heterocycloalkyl, aryl, heteroaryl, aryloxy, alkylthio, arylthio, alkylsulfoxide, arylsulfoxide, alkylsulfone, and arylsulfone.

In some embodiments, the compound has the structure of Formula (XIA), or a pharmaceutically acceptable salt or solvate thereof:

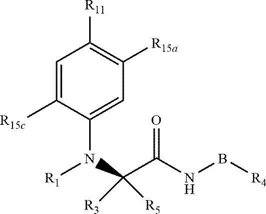

In some embodiments, B or B₁ is independently C₁-C₄ alkylene, or C₃-C₆ cyclene linker. In some embodiments, B or B₁ is independently an C2 or C3 alkylene linker. In some embodiments, B and B₁ is bond. In some embodiments, R₃ is a 6-membered heteroaryl containing 1 to 3 N atoms. In some embodiments, 6-membered heteroaryl is pyridine, pyrimidine, pyrazine, or pyridazine.

In some embodiments, the compound has the structure of Formula (XII), or a pharmaceutically acceptable salt or solvate thereof:

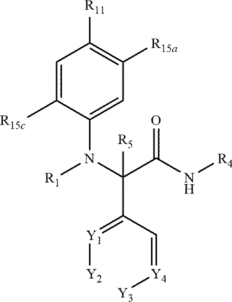

wherein, Y₁, Y₂, Y₃ and Y₄ are each independently CH or N, provided that at least one of Y₁, Y₂, Y₃, or Y₄ is CH.

In some embodiments, Y₂ is N; and Y₁, Y₃ and Y₄ are each CH. In some embodiments, Y₂ and Y₄ are each N; and Y₁ and Y₃ are CH. In some embodiments, Y₁ and Y₄ are N; and Y₂ and Y₃ are CH. In some embodiments, Y₂ and Y₃ are N; and Y₁ and Y₄ are CH. In some embodiments, R₅ is C₁-C₆ alkyl. In some embodiments, R₅ is H.

In some embodiments, the compound has the structure of Formula (XIIA), or a pharmaceutically acceptable salt or solvate thereof:

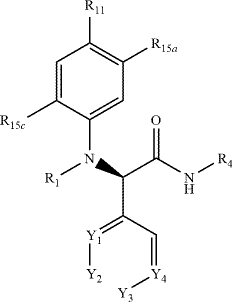

In some embodiments, the compound has the structure of Formula (XIIB), or a pharmaceutically acceptable salt or solvate thereof:

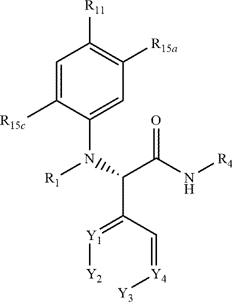

In some embodiments, the compound has a stereochemical purity of at least 80%.

In some embodiments, R₁₅ₐ is H; and R_{15b} is amino, halogen, —CN, —OH, —OCF₃, C₁-C₆ alkyl, C₂-C₆ alkenyl, C₂-C₆ alkynyl, C₁-C₆ haloalkyl, or C₁-C₆ alkoxy. In some embodiments, R₁₅ₐ is amino, halogen, —CN, —OH, —OCF₃, C₁-C₆ alkyl, C₂-C₆ alkenyl, C₂-C₆ alkynyl, C₁-C₆ haloalkyl, or C₁-C₆ alkoxy; and R_{15b} is H. In some embodiments, R₁₅ₐ and R_{15c} are each H. In some embodiments, R₁₁ is heteroaryl, optionally substituted with one, two, or three R₁₇. In some embodiments, he heteroaryl is a 5-membered heteroaryl. In some embodiments, the heteroaryl is furan, thiophene, oxazole, thiazole, isoxazole, triazole, oxadiazole, or thiadiazole. In some embodiments, R₁₁ is an unsubstituted heteroaryl. In some embodiments, R₄ is heterocycloalkyl optionally substituted with one, two, or three R₁₉. In some embodiments, R₄ is cycloalkyl, optionally substituted with one two or three R₁₉. In some embodiments, cycloalkyl is a cyclobutyl, cyclopentyl, cyclohexyl or spiro[3,3]heptanyl. In some embodiments, each R₁₉ is independently halogen, oxo, —CN, —NH₂, —NH(C₁—₆ alkyl), —N(C₁—₆ alkyl)₂, —OH, —CO₂H, —CO₂—C₁—₆ alkyl, —C(═O)NH₂, —C(═O)NH(C₁—₆ alkyl), —C(═O)N(C₁—₆ alkyl)₂, —S(═O)₂NH₂, —S(═O)₂NH(C₁—₆ alkyl), —S(═O)₂N(C₁—₆ alkyl)₂, C₁-C₆ alkyl, C₁-C₆ haloalkyl, C₃₋₈ cycloalkyl, C₁-C₆ heteroalkyl, C₁-C₆ alkoxy, C₁₋₆ fluoroalkoxy, C₂-₇ heterocycloalkyl, aryl, heteroaryl, aryloxy, alkylthio, arylthio, alkylsulfoxide, arylsulfoxide, alkylsulfone, and arylsulfone. In some embodiments, each R₁₉ is independently halogen. In some embodiments, R₁ is halo acetyl, heterocyclo acyl or acrylo acyl. In some embodiments, the halo acetyl is mono substituted halo acetyl or di substituted halo acetyl.

In another aspect, described herein is a compound comprising a structure of one of Formula A, derivative thereof, prodrug thereof, salt thereof, or stereoisomer thereof, or having any chirality at any chiral center, or tautomer, polymorph, solvate, or combination thereof:

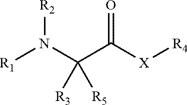

wherein:
R₁ is an electrophilic moiety;
R₂, R₃, R₄, and R₅ are subsituents other then hydrogen; and
X is a heteroatom.

In another aspect described herein is a compound comprising a structure of one of Formula A, derivative thereof, prodrug thereof, salt thereof, or stereoisomer thereof, or having any chirality at any chiral center, or tautomer, polymorph, solvate, or combination thereof:

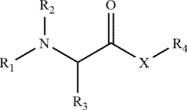

wherein,
R₁ is an electrophilic moiety;
R₂, R₃ and R₄ are substituents other than hydrogen; and
X is a heteroatom.

In some embodiments, R₁ is an electrophilic moiety that is capable of forming a covalent bond with the cysteine residue at position 145 of SARS-CoV-2 main protease; R₂ is an optionally substituted C₃-C₁₂ alkyl, alkenyl, cycloalkyl, cycloalkenyl, heterocycle (heterocyclic), aryl, or heteroaryl; R₃ is an optionally substituted C₃-C₁₂ alkyl, alkenyl, cycloalkyl, cycloalkenyl, heterocycle (heterocyclic), aryl, or heteroaryl; X is NH, O, S, or bond; and R₄ is an optionally substituted C₃-C₁₂ alkyl, alkenyl, cycloalkyl, cycloalkenyl, heterocycle (heterocyclic), aryl, or heteroaryl.

In some embodiments, he electrophilic moiety that can be used for covalent modifications for R₁, which may be optionally branched, can be based on: (a) Michael acceptor (α, β-unsaturated carbonyls and sulfonyls) patterns (for example, acryloyl, vinyl sulfonyl); (b) α-halogeno acyls (for example α-chloroacetyl); (c) α,β-epoxy acyls; (d) glyoxyl; (e) β,γ-diketoacyls; (f) 3,4-dioxoalkyl3,4-dioxoalkyls; (g) 2,3-dioxoalkyls; and (h) α-ketoacyls (for example pyruvyl).

In another aspect, provided herein is a compound comprising a structure of one of Formula (I), Formula (II), Formula (III), or Formula (IV), derivative thereof, prodrug thereof, salt thereof, or stereoisomer thereof, or having any chirality at any chiral center, or tautomer, polymorph, solvate, or combination thereof:

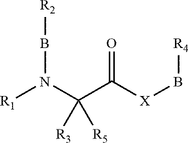

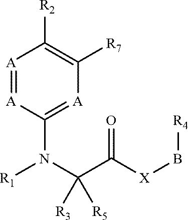

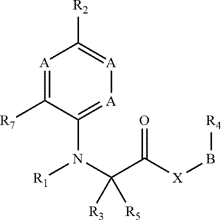

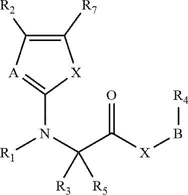

wherein:
R₁, R₂, R₃, R₄, R₅, or R₇ are a chemical moiety;
X is NH, O, S, CH₂, or a bond;
each A is individually CH or N; and
B is a bond or a linker.

In some embodiments, R₅ and/or R₆ is independently selected from H, CH₃, C₂H₅, or CF₃.

In some embodiments, Hal is a halogen, such as F, Cl, Br, or I.

In some embodiments, R₂, R₃, R₄, R₇, and/or R₈ are each independently selected from H, CH₃, CF₃, CHF₂, CH₂F, C₂H₅, Hal, —CN, or an optionally substituted moiety selected from C₃-C₁₂ alkyl, C₃-C₁₂ alkenyl, cycloalkyl, cycloalkenyl, heterocycloalkyl, heterocycloalkenyl, aryl, heteroaryl, fused heterocycle, fused aryl, fused heterocycle-aryl, spirocycle, or combinations thereof.

In some embodiments, the compound, or a pharmaceutically acceptable salt or solvate thereof is selected from Table 1.

In another aspect, provided herein is a pharmaceutical composition comprising a compound described herein and a pharmaceutically acceptable carrier or excipient.

In another aspect, provided herein is a method of treating or preventing a SARS-CoV-2 infection in a patient in need thereof, comprising administering to the patient a compound described herein, or a pharmaceutical compound described herein. In some embodiments, the compound or the pharmaceutical composition is administered to the patient until the infection is reduced or eliminated. In some embodiments, the method comprises treating one or more symptoms of SARS-CoV-2 in the patient in need thereof.

In another aspect, provided herein is an in vivo method of inhibiting a protease of SARS-CoV-2, comprising contacting the protease with a compound as described herein. In some embodiments, the compound bind to a cysteine residue of the protease. In some embodiments, the compound binds reversibly or irreversibly to the cysteine residue. In some embodiments, the protease is 3CL-protease. In some embodiments, the cysteine is cysteine 145 of 3CL-protease.

Other objects, features and advantages of the combinations and methods described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings below.

FIG. 1 shows the schematic for covalent 3CL-protease inhibitors for treating viral infections.

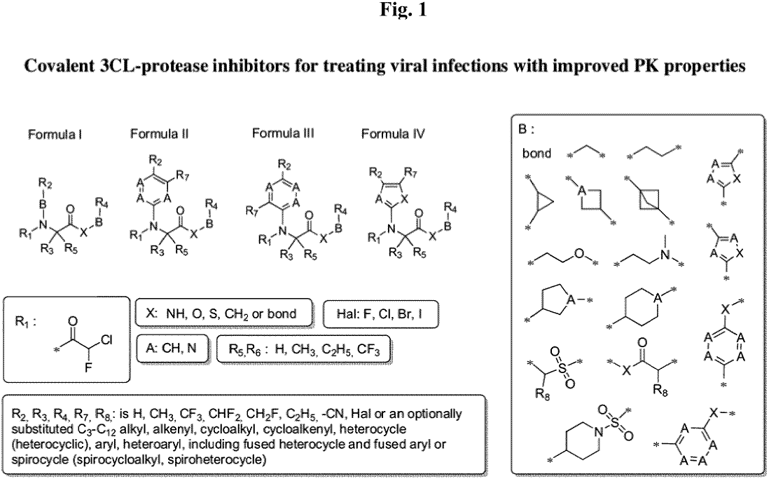

FIG. 2 shows PK profile for INSCoV-614(1B) when administered orally, SQ, and IV.

FIG. 3 shows the PK profile for INSCoV-614A(2A) when administered orally, SQ, and IV.

FIG. 4 shows the X-Ray structure of INSCoV-601I(1) in complex with SARS-CoV-2 M^{pro} (resolution 1.88 Angstroms).

### DETAILED DESCRIPTION

In the following detailed description, reference is made to the accompanying drawings, which form a part thereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

The present disclosure includes compounds and/or materials for use as SARS-CoV-2 inhibitors and for treating a subject infected with SARS-CoV-2. These compounds include the chemical structures associated with compound identifiers INSCoV (e.g., INSCoV-number), and derivatives thereof which are provided herein. The compounds have various chemical structures that have been identified as inhibiting SARS-CoV-2.

### Compounds

In an aspect, provided herein is a compound comprising a structure of one of Formula A*, derivative thereof, prodrug thereof, salt thereof, or stereoisomer thereof, or having any chirality at any chiral center, or tautomer, polymorph, solvate, or combination thereof:

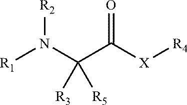

wherein:
R₁ is an electrophilic moiety;
R₂, R₃, R₄, and R₅ are subsituents other then hydrogen; and
X is a hetero atom.

In an aspect, provided herein is a compound comprising a structure of one of Formula A, derivative thereof, prodrug thereof, salt thereof, or stereoisomer thereof, or having any chirality at any chiral center, or tautomer, polymorph, solvate, or combination thereof:

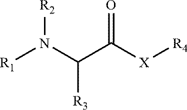

wherein:
R₁ is an electrophilic moiety;
R₂, R₃, and R₄ are subsituents other then hydrogen; and
X is a hetero atom.

In some embodiments, the variables are defined as follows:
R₁ is an electrophilic moiety that is capable of forming a covalent bond with the cysteine residue at position 145 of SARS-CoV-2 main protease;
R₂ is an optionally substituted C₃-C₁₂ alkyl, alkenyl, cycloalkyl, cycloalkenyl, heterocycle (heterocyclic), aryl, or heteroaryl;
R₃ is an optionally substituted C₃-C₁₂ alkyl, alkenyl, cycloalkyl, cycloalkenyl, heterocycle (heterocyclic), aryl, or heteroaryl;
X is CH₂, NH, O, S, or bond; and
R₄ is an optionally substituted C₃-C₁₂ alkyl, alkenyl, cycloalkyl, cycloalkenyl, heterocycle (heterocyclic), aryl, or heteroaryl.

In some embodiments, R₁ is An electrophilic moiety that can be used for covalent modifications. In some embodiments, R₁ is:
(a) Michael acceptor (α, β-unsaturated carbonyls and sulfonyls) patterns (for example, acryloyl, vinyl sulfonyl),
(b) α-halogeno acyls (for example α-chloroacetyl),
(c) α, β-epoxy acyls,
(d) glyoxyl,
(e) β,γ-diketoacyls,
(f) 3,4-dioxoalkyls,
(g) 2,3-dioxoalkyls, and
(h) α-ketoacyls (for example pyruvyl).

In some embodiments, the covalent modification is with a Michael acceptor (α,β-unsaturated carbonyls and sulfonyls) patterns (for example, acryloyl, vinyl sulfonyl). In some embodiments, the covalent modification is with a α-halogeno acyl (for example a-chloroacetyl). In some embodiments, the covalent modification is with an α, β-epoxy acyls. In some embodiments, the covalent modification is with a glyoxyl. In some embodiments, the covalent modification is with a β,γ-diketoacyl. In some embodiments, the covalent modification is with a 3,4-dioxoalkyl. In some embodiments, the covalent modification is with a 2,3-dioxoalkyl. In some embodiments, the covalent modification is with an α-ketoacyl (for example pyruvyl).

In some embodiments, the compound has the structure of in Formula (I), Formula (II), Formula (III), or Formula (IV); or derivative thereof, prodrug thereof, salt thereof, or stereoisomer thereof, or having any chirality at any chiral center, or tautomer, polymorph, solvate, or combination thereof:

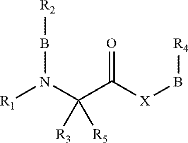

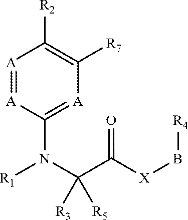

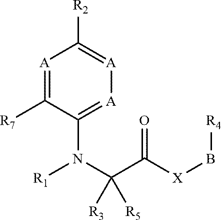

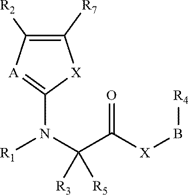

wherein:
R₁, R₂, R₃, R₄, R₅, or R₇ are independently a chemical moiety;
X is NH, O, S, CH₂, or a bond;
each A is independently CH or N; and
B is a bond or a linker.

In some embodiments, the compound has the structure of Formula (I), or a pharmaceutically acceptable salt or solvate thereof.

In some embodiments, the compound has the structure of Formula (II), or a pharmaceutically acceptable salt or solvate thereof.

In some embodiments, the compound has the structure of Formula (III), or a pharmaceutically acceptable salt or solvate thereof.

In some embodiments, the compound has the structure of Formula (IV), or a salt or solvate therof.

In some embodiments, each A is independently each A is independently CH or N. In some embodiments, each A is independently CH. In some embodiments, each A is independently N.

In some embodiments, X is selected from NH, O, S, CH₂, or a bond. In some embodiments, X is NH. In some embodiments, X is O. In some embodiments, X is S. In some embodiments, X is CH₂. In some embodiments, X is a bond.

In one aspect, provided herein is a compound comprising of Formula (IX), or a pharmaceutically acceptable salt or solvate thereof:

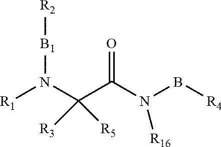

wherein,
B₁ and B are each independently a bond, C₁-C₄ alkylene, C₁-C₄ heteroalkylene, or C₃-C₆ cyclene linker, wherein the alkylene, heteroalkylene or cyclene is optionally substituted;
R₁ is

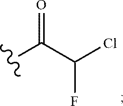

R₂ is an optionally substituted heteroaryl, optionally substituted aryl, optionally substituted cycloalkyl, or optionally substituted heterocycloalkyl;
R₃ is an optionally substituted heteroaryl, optionally substituted aryl, optionally substituted cycloalkyl, or optionally substituted heterocycloalkyl;
R₄ is an C₁-C₆ alkyl, aryl, heteroaryl, cycloalkyl or heterocycloalkyl, each of which is optionally substituted;
R₅ is H, C₁-C₆ alkyl, or C₁-C₃ haloalkyl, wherein the alkyl or haloalkyl is optionally substituted;
R₁₁ is amino, halogen, —CN, —OH, C₁-C₆ alkyl, C₁-C₆ haloalkyl, C₁-C₆ alkoxy, cycloalkyl, heterocycloalkyl, aryl, or heteroaryl, wherein each of the alkyl, alkoxy, aryl, cycloalkyl, heterocycloalkyl, or heteroaryl is optionally substituted;
and R₁₆ is H, C₁-C₆ alkyl, or C₁-C₃ haloalkyl, wherein the alkyl or haloalkyl is optionally substituted.

In some embodiments, R₂ is an optionally substituted cycloalkyl or heterocycloalkyl. In some embodiments, R₂ is an optionally substituted spiro-cycloalkyl or spiro-heterocycloalkyl.

In some embodiments, R₂ is an optionally substituted aryl. In some embodiments, R₂ is an optionally substituted phenyl. In some embodiments, R₂ is an optionally substituted heteroaryl. In some embodiments, R₂ is an optionally substituted 5-membered heteroaryl. In some embodiments, R₂ is an optionally substituted 6-membered heteroaryl. In some embodiments, R₂ is

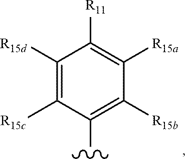

wherein R₁₁, R₁₅ₐ, R_{15b}, R_{15c}, and R_{15d} have their meanings assigned below. In some embodiments, R₁₅ₐ, R_{15b}, R_{15c}, and R_{15d} are each independently H, amino, halogen, —CN, —OH, heteroalkyl, alkyl, alkenyl, alkynyl, haloalkyl, alkoxy, cycloalkyl, heterocycloalkyl, aryl or heteroaryl, wherein the heteroalkyl, alkyl, alkenyl, alkoxy, cycloalkyl, heterocycloalkyl, aryl or heteroaryl is optionally substituted. In some embodiments, R₁₁ is amino, halogen, —CN, —OH, C₁-C₆ alkyl, C₁-C₆ haloalkyl, C₁-C₆ alkoxy, cycloalkyl, heterocycloalkyl, aryl, or heteroaryl, wherein each of the alkyl, alkoxy, aryl, cycloalkyl, heterocycloalkyl, or heteroaryl is optionally substituted.

In one aspect, provided herein is a compound comprising of Formula (X), or a pharmaceutically acceptable salt or solvate thereof:

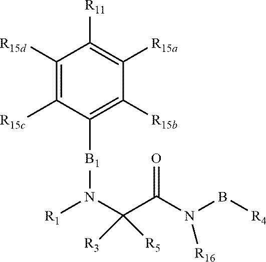

wherein,
B₁ and B are each independently a bond, C₁-C₄ alkylene, C₁-C₄ heteroalkylene, or C₃-C₆ cyclene linker, wherein the alkylene, heteroalkylene or cyclene is optionally substituted;
R₁ is an electrophilic moiety;
R₃ is an optionally substituted heteroaryl;
R₄ is an C₁-C₆ alkyl, aryl, heteroaryl, cycloalkyl or heterocycloalkyl, each of which is optionally substituted;
R₅ is H, C₁-C₆ alkyl, or C₁-C₃ haloalkyl, wherein the alkyl or haloalkyl is optionally substituted;
R₁₁ is amino, halogen, —CN, —OH, C₁-C₆ alkyl, C₁-C₆ haloalkyl, C₁-C₆ alkoxy, cycloalkyl, heterocycloalkyl, aryl, or heteroaryl, wherein each of the alkyl, alkoxy, aryl, cycloalkyl, heterocycloalkyl, or heteroaryl is optionally substituted;
R₁₅ₐ, R_{15b}, R_{15c}, and R_{15d} are each independently H, amino, halogen, —CN, —OH, heteroalkyl, alkyl, alkenyl, alkynyl, haloalkyl, or alkoxy, wherein the heteroalkyl, alkyl, alkenyl, or alkynyl is optionally substituted;
wherein optionally, R₁₅ₐ and R₁₁, taken in combination with the carbon atom to which they attach, form a 5-6 membered substituted or unsubstituted ring; or
wherein optionally, R₁₅ₐ and R_{15b}, taken in combination with the carbon atom to which they attach, form a 5-6 membered substituted or unsubstituted ring;
and R₁₆ is H, C₁-C₆ alkyl, or C₁-C₃ haloalkyl, wherein the alkyl or haloalkyl is optionally substituted.

In some embodiments, R₁₅ₐ, R_{15b}, R_{15c}, and R_{15d} are each independently H, amino, halogen, —CN, —OH, —OCF₃, C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl is optionally substituted.

In some embodiments, R₁₅ₐ, R_{15b}, R_{15c}, and R_{15d} are each independently H, amino, halogen, —CN, —OH, —OCF₃, C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl is optionally substituted with one, two, or three R²⁰ wherein R₂₀ is oxo, halogen, —CN, —NH₂, —NH(C₁—₆ alkyl), —N(C₁—₆ alkyl)₂, —OH, —CO₂H, —CO₂—C₁—₆ alkyl, —C(═O)NH₂, —C(═O)NH(C₁—₆ alkyl), —C(═O)N(C₁—₆ alkyl)₂, —S(═O)₂NH₂, —S(═O)₂NH(C₁—₆ alkyl), —S(═O)₂N(C₁—₆ alkyl)₂, C₁-C₆ alkyl, C₁-C₆ haloalkyl, C₃₋₈ cycloalkyl, C₁-C₆ heteroalkyl, C₁-C₆ alkoxy, C₁₋₆ fluoroalkoxy, C₂-₇ heterocycloalkyl, aryl, heteroaryl, aryloxy, alkylthio, arylthio, alkylsulfoxide, arylsulfoxide, cycloalkylsulfone, alkylsulfone, and arylsulfone.

In some embodiments, R₁ is configured to interact with 3CL-protease. In some embodiments, R₁ is an acyl group such as halo acetyl, glyoxyl, heterocyclo acyl, cyanide acetyl, or acrylo acyl. In some embodiments, R₁ is a sulfonyl or sulfinyl group such as vinylsulfonyl or vinylsulfinyl.

In another aspect, provided herein is a compound having the structure of Formula (X), or a pharmaceutically acceptable salt or solvate thereof:

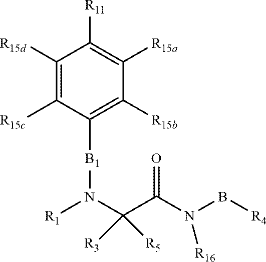

wherein,
B₁ and B are each independently a bond, C₁-C₄ alkylene, C₁-C₄ heteroalkylene, or C₃-C₆ cyclene linker, wherein the alkylene, heteroalkylene or cyclene is optionally substituted;
R₁ is halo acetyl, glyoxyl, heterocyclo acyl, cyanide acetyl, vinylsulfonyl, vinylsulfinyl, or acrylo acyl;
R₃ is an optionally substituted heteroaryl;
R₄ is an C₁-C₆ alkyl, aryl, heteroaryl, cycloalkyl or heterocycloalkyl, each of which is optionally substituted;
Rs is H, C₁-C₆ alkyl, or C₁-C₃ haloalkyl;
R₁₁ is amino, halogen, —CN, —OH, C₁-C₆ alkyl, C₁-C₆ haloalkyl, C₁-C₆ alkoxy, cycloalkyl, heterocycloalkyl, aryl, or heteroaryl, wherein each of the alkyl, alkoxy, aryl, cycloalkyl, heterocycloalkyl, or heteroaryl is optionally substituted;
R₁₅ₐ, R_{15b}, R_{15c}, and R_{15d} are each independently H, amino, halogen, —CN, —OH, —OCF₃, C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl is optionally substituted with one, two, or three R²⁰;
wherein optionally, R₁₅ₐ and R₁₁, taken in combination with the carbon atom to which they attach, form a 5-6 membered substituted or unsubstituted ring; or
wherein optionally, R₁₅ₐ and R_{15b}, taken in combination with the carbon atom to which they attach, form a 5-6 membered substituted or unsubstituted ring;
R₁₆ is H, C₁-C₆ alkyl, or C₁-C₃ haloalkyl; and
R₂₀ is oxo, halogen, —CN, —NH₂, —NH(C₁—₆ alkyl), —N(C₁—₆ alkyl)₂, —OH, —CO₂H, —CO₂—C₁—₆ alkyl, —C(═O)NH₂, —C(═O)NH(C₁—₆ alkyl), —C(═O)N(C₁—₆ alkyl)₂, —S(═O)₂NH₂, —S(═O)₂NH(C₁—₆ alkyl), —S(═O)₂N(C₁—₆ alkyl)₂, C₁-C₆ alkyl, C₁-C₆ haloalkyl, C₃₋₈ cycloalkyl, C₁-C₆ heteroalkyl, C₁-C₆ alkoxy, C₁₋₆ fluoroalkoxy, C₂-₇ heterocycloalkyl, aryl, heteroaryl, aryloxy, alkylthio, arylthio, alkylsulfoxide, arylsulfoxide, cycloalkylsulfone, alkylsulfone, and arylsulfone.

In some embodiments, the compound has the structure of Formula (XA), or a pharmaceutically acceptable salt or solvate thereof:

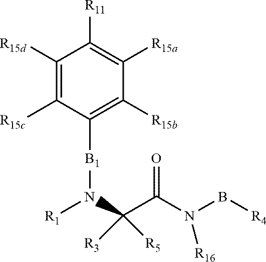

In some embodiments, the compound has the structure of Formula (XB), or a pharmaceutically acceptable salt or solvate thereof:

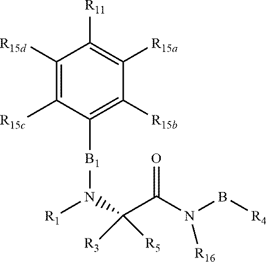

In some embodiments, compound has the structure of Formula (XI), or a pharmaceutically acceptable salt or solvate thereof:

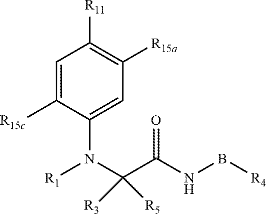

wherein,
B is a bond, C₁-C₄ alkylene, or C₃-C₆ cyclene linker;
R₁ is halo acetyl, glyoxyl, heterocyclo acyl or acrylo acyl;
R₃ is a heteroaryl optionally substituted with one, two, or three R₁₈;
R₄ is an aryl, heteroaryl, cycloalkyl or heterocycloalkyl, each of which is optionally substituted with one, two, three, or four R¹⁹;
R₅ is H, C₁-C₆ alkyl, or C₁-C₃ haloalkyl;
R₁₁ is amino, halogen, —CN, —OH, —OCF₃, C₁-C₆ alkyl, C₁-C₆ haloalkyl, C₁-C₆ alkoxy, cycloalkyl, heterocycloalkyl, aryl, or heteroaryl, wherein each of the alkyl, aryl, cycloalkyl, heterocycloalkyl, or heteroaryl is optionally substituted with one, two, or three R₁₇;
R₁₅ₐ and R_{15c} are each independently H, amino, halogen, —CN, —OH, —OCF₃, C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl is optionally substituted with one, two, or three R²⁰;
each R₁₇, R₁₈ , R₁₉, and R₂₀ is independently selected from oxo, halogen, —CN, —NH₂, —NH(C₁—₆ alkyl), —N(C_{1—6} alkyl)₂, —OH, —CO₂H, —CO₂—C₁—₆ alkyl, —C(═O)NH₂, —C(═O)NH(C₁—₆ alkyl), —C(═O)N(C₁—₆ alkyl)₂, —S(═O)₂NH₂, —S(═O)₂NH(C₁—₆ alkyl), —S(═O)₂N(C₁—₆ alkyl)₂, C₁-C₆ alkyl, C₁-C₆ haloalkyl, C₃₋₈ cycloalkyl, C₁-C₆ heteroalkyl, C₁-C₆ alkoxy, C₁₋₆ fluoroalkoxy, C₂-₇ heterocycloalkyl, aryl, heteroaryl, aryloxy, alkylthio, arylthio, alkylsulfoxide, arylsulfoxide, cycloalkylsulfone, alkylsulfone, and arylsulfone.

In some embodiments, the compound has the structure of Formula (XI), or a pharmaceutically acceptable salt or solvate thereof:

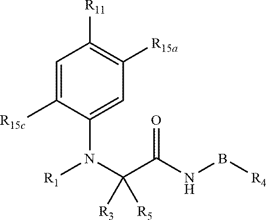

wherein,
B is a bond, C₁-C₄ alkylene, or C₃-C₆ cyclene linker;
R₁ is halo acetyl, glyoxyl, heterocyclo acyl or acrylo acyl;
R₃ is a heteroaryl optionally substituted with one, two, or three R₁₈;
R₄ is a substituted cycloalkyl or an optionally substituted heterocycloalkyl, wherein when substituted the each of which is substituted with one, two, three, or four R¹⁹;
R₅ is H, C₁-C₆ alkyl, or C₁-C₃ haloalkyl;
R₁₁ is amino, halogen, —CN, —OH, —OCF₃, C₁-C₆ alkyl, C₁-C₆ haloalkyl, C₁-C₆ alkoxy, cycloalkyl, heterocycloalkyl, aryl, or heteroaryl, wherein each of the alkyl, aryl, cycloalkyl, heterocycloalkyl, or heteroaryl is optionally substituted with one, two, or three R₁₇;
2R₁₅ₐ and R_{15c} are each independently H, amino, halogen, —CN, —OH, —OCF₃, C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl is optionally substituted with one, two, or three R²⁰;
each R₁₇, R₁₈, R₁₉, and R₂₀ is independently selected from oxo, halogen, —CN, —NH₂, —NH(C₁—₆ alkyl), —N(C_{1—6} alkyl)₂, —OH, —CO₂H, —CO₂—C₁—₆ alkyl, —C(═O)NH₂, —C(═O)NH(C₁—₆ alkyl), —C(═O)N(C₁—₆ alkyl)₂, —S(═O)₂NH₂, —S(═O)₂NH(C₁—₆ alkyl), —S(═O)₂N(C₁—₆ alkyl)₂, C₁-C₆ alkyl, C₁-C₆ haloalkyl, C₃₋₈ cycloalkyl, C₁-C₆ heteroalkyl, C₁-C₆ alkoxy, C₁-₆ fluoroalkoxy, C₂-₇ heterocycloalkyl, aryl, heteroaryl, aryloxy, alkylthio, arylthio, alkylsulfoxide, arylsulfoxide, alkylsulfone, and arylsulfone.

In some embodiments, the compound has the structure of Formula (XI), or a pharmaceutically acceptable salt or solvate thereof:

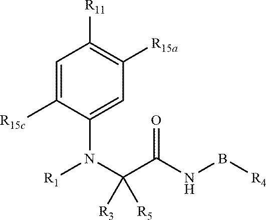

wherein,
B is a bond, C₁-C₄ alkylene, or C₃-C₆ cyclene linker;
R₁ is halo acetyl, glyoxyl, heterocyclo acyl or acrylo acyl;
R₃ is a heteroaryl optionally substituted with one, two, or three R₁₈;
R₄ is an aryl, heteroaryl, cycloalkyl or heterocycloalkyl, each of which is optionally substituted with one, two, three, or four R¹⁹;
R₅ is H, C₁-C₆ alkyl, or C₁-C₃ haloalkyl;
R₁₁ is a cycloalkyl, heterocycloalkyl, aryl, or heteroaryl, wherein each of the cycloalkyl, heterocycloalkyl, aryl, or heteroaryl is optionally substituted with one, two, or three R₁₇;
R₁₅ₐ and R_{15c} are each independently H, amino, halogen, —CN, —OH, —OCF₃, C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl is optionally substituted with one, two, or three R²⁰;
each R₁₇, R₁₈, R₁₉, and R₂₀ is independently selected from oxo, halogen, —CN, —NH₂, —NH(C₁—₆ alkyl), —N(C_{1—6} alkyl)₂, —OH, —CO₂H, —CO₂—C₁—₆ alkyl, —C(═O)NH₂, —C(═O)NH(C₁—₆ alkyl), —C(═O)N(C₁—₆ alkyl)₂, —S(═O)₂NH₂, —S(═O)₂NH(C₁—₆ alkyl), —S(═O)₂N(C₁—₆ alkyl)₂, C₁-C₆ alkyl, C₁-C₆ haloalkyl, C₃₋₈ cycloalkyl, C₁-C₆ heteroalkyl, C₁-C₆ alkoxy, C₁-₆ fluoroalkoxy, C₂-₇ heterocycloalkyl, aryl, heteroaryl, aryloxy, alkylthio, arylthio, alkylsulfoxide, arylsulfoxide, alkylsulfone, and arylsulfone.

In some embodiments, the compound has the structure of Formula (XIA), or a pharmaceutically acceptable salt or solvate thereof:

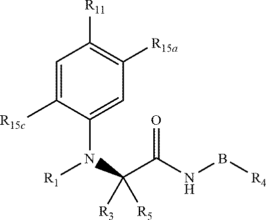

In some embodiments, the compound has the structure of Formula (XIB), or a pharmaceutically acceptable salt or solvate thereof:

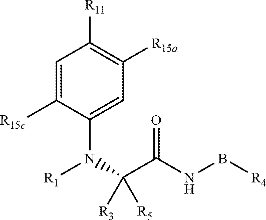

In some embodiments, B or B₁ is independently a liker. In some embodiments, B or B₁ is independently C₁-C₄ alkylene, C₁-C₄ heteroalkylene, or C₃-C₆ cyclene linker.

In some embodiments, B or B₁ is independently selected from selected from: bond,

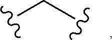

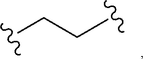

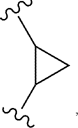

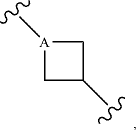

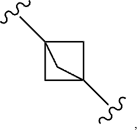

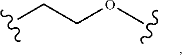

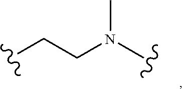

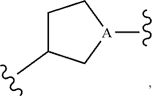

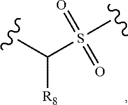

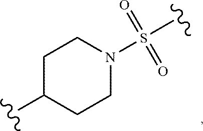

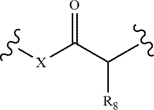

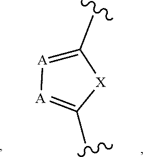

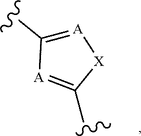

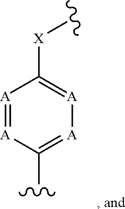

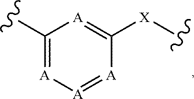

wherein each A is individually CH or N; and X is NH, O, or S.

In some embodiments, B is a optionally substituted C₁-C₄ alkylene linker. In some embodiments, B is a C2 or C3 alkylene linker. In some embodiments, B is —CH₂—, —CH₂—CH₂— or —CH₂—CH₂—CH₂—. In some embodiments, B₁ is a C₁-C₄ alkylene linker. In some embodiments, B₁ is a C2 or C3 alkylene linker. In some embodiments, B₁ is —CH₂—, —CH₂—CH₂— or —CH₂—CH₂—CH₂—.

In some embodiments, B is a C₃-C₆ cyclene linker. In some embodiments, B is a C3, C4, C5, or C6 cyclene linker. In some embodiments, B is

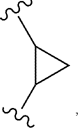

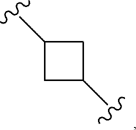

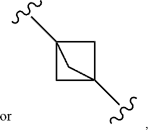

In some embodiments, B₁ is a C₃-C₆ cyclene linker. In some embodiments, B₁ is a C3, C4, C5, or C6 cyclene linker. In some embodiments, B₁ is

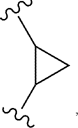

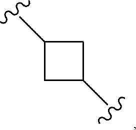

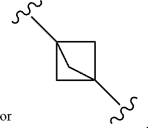

In some embodiments, B is a bond. In some embodiments, B₁ is a bond.

In some embodiments, R₃ is an optionally substituted heteroaryl. In some embodiments, R₃ is a heteroaryl optionally substituted with one, two, or three R₁₈. In some embodiments, R3 is an unsubstituted heteroaryl.

In some embodiments, R₃ is a monocyclic or bicyclic heteroaryl.

In some embodiments, R₃ is a 6-membered heteroaryl containing 1 to 3 N atoms. In some embodiments, R₃ is pyridine, pyrimidine, pyrazine, or pyridazine. In some embodiments, R³ is pyridine. R³ is pyrimidine. In some embodiments, R³ is pyrazine. In some embodiments, R³ is pyrazine. In some embodiments, R³ is pyridazine.

In some embodiments, the compound has the structure of Formula (XII), or a pharmaceutically acceptable salt or solvate thereof:

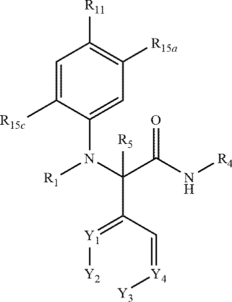

wherein,

Y₁, Y₂, Y₃ and Y₄ are each independently CH or N, provided that at least one of Y₁, Y₂, Y₃, or Y₄ is CH.

In some embodiments, Y₂ is N; and Y₁, Y₃ and Y₄ are each CH. In some embodiments, Y₂ and Y₄ are each N; and Y₁ and Y₃ are CH. In some embodiments, Y₁ and Y₄ are N; and Y₂ and Y₃ are CH. In some embodiments, Y₂ and Y₃ are N; and Y₁ and Y₄ are CH.

In some embodiments, R₅ is H, C₁-C₆ alkyl, or C₁-C₃ haloalkyl. In some embodiments, R₅ is C₁-C₆ alkyl. In some embodiments, R₅ is H, methyl, ethyl, n-propyl, or isopropyl. In some embodiments, R₅ is methyl or ethyl. In some embodiments, R₅ is ethyl. In some embodiments, R₅ is methyl. In some embodiments, R₅ is H. In some embodiments, R₅ is C₁-C₃ haloalkyl. In some embodiments, R₅ is —CF₃.

In some embodiments, the compound has the structure of Formula (XIIA), or a pharmaceutically acceptable salt or solvate thereof:

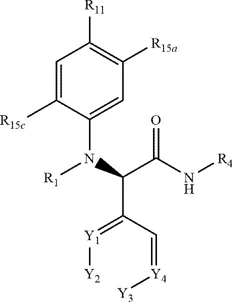

In some embodiments, the compound has the structure of Formula (XIIB), or a pharmaceutically acceptable salt or solvate thereof:

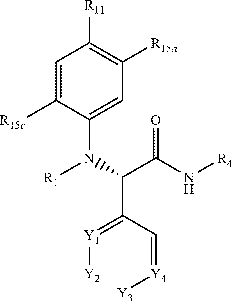

In some embodiments, the compound has an ee of at least 80%, 85%, 90%, 95%. In some embodiments, the compound has an ee of at least 80%. In some embodiments, the compound has an ee of at least 85%. In some embodiments, the compound has an ee of at least 90%. In some embodiments, the compound has an ee of at least 95%.

In some embodiments, the compound has an ee of about 80% to about 99%. In some embodiments, the compound has an ee of about 80%, about 85%, about 90%, or about 95%, In some embodiments, the compound has an ee of about 90%, about 91%, about 92%, about 93%, about 94%, about 96%, about 97%, about 98%, or about 99%.

In some embodiments, R₁₅ₐ, R_{15b}, R_{15c}, and R_{15d} are each independently H, amino, halogen, —CN, —OH, —OCF₃, C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl is optionally substituted with one, two, or three R²⁰

In some embodiments, R₁₅ₐ, R_{15b}, R_{15c}, and R_{15d} are each independently C₁-C₆ alkyl, C₂-C₆ alkenyl, C₂-C₆ alkynyl, C₁-C₆ haloalkyl, or C₁-C₆ alkoxy. In some embodiments, R₁₅ₐ, R_{15b}, R_{15c}, and R_{15d} are each independently C₁-C₆ alkyl. In some embodiments, R₁₅ₐ, R_{15b}, R_{15c}, and R_{15d} are each independently methyl or t-butyl. In some embodiments, R₁₅ₐ, R_{15b}, R_{15c}, and R_{15d} are each independently t-butyl. In some embodiments, R₁₅ₐ, R_{15b}, R_{15c}, and R_{15d} are each independently C₁-C₆ haloalkyl, or C₁-C₆ alkoxy. In some embodiments, R₁₅ₐ, R_{15b}, R_{15c}, and R_{15d} are each independently —OCF₃. In some embodiments, R₁₅ₐ, R_{15b}, R_{15c}, and R_{15d} are each independently —OCH₃.

In some embodiments, R₁₅ₐ, R_{15b}, R_{15c}, and R_{15d} are each independently H, —NH₂, Br, F, Cl, I, —CN, —OH, —OCF₃, —CF3, —CH₂CF₃, —OCH₃, methyl, ethyl, or t-butyl. In some embodiments, R₁₅ₐ, R_{15b}, R_{15c}, and R_{15d} are each independently H, F, Br, Cl, or I. In some embodiments, R₁₅ₐ, R_{15b}, R_{15c}, and R_{15d} are each independently Br. In some embodiments, R₁₅ₐ, R_{15b}, R_{15c}, and R_{15d} are each independently Cl. In some embodiments, R₁₅ₐ, R_{15b}, R_{15c}, and R_{15d} are each independently F.

In some embodiments, R₁₅ₐ is H. In some embodiments, R_{15b} is H. In some embodiments, R_{15c} is H. In some embodiments, R_{15d} is H.

In some embodiments, R₁₅ₐ and R₁₁, taken in combination with the carbon atom to which they attach, form a 5-6 membered substituted or unsubstituted ring.

In some embodiments,, R₁₅ₐ and R_{15b}, taken in combination with the carbon atom to which they attach, form a 5-6 membered substituted or unsubstituted ring.

In some embodiments, R₁₅ₐ is H; and R_{15b} is amino, halogen, —CN, —OH, —OCF₃, C₁-C₆ alkyl, C₂-C₆ alkenyl, C₂-C₆ alkynyl, C₁-C₆ haloalkyl, or C₁-C₆ alkoxy. In some embodiments, R₁₅ₐ is H; and R_{15b} is —NH₂, F, Br, Cl, I, —CN, —OH, —OCF₃, —OCH₃, —CF₃, —CH₂CF₃, methyl, ethyl, or t-butyl.

In some embodiments, R₁₅ₐ is amino, halogen, —CN, —OH, —OCF₃, C₁-C₆ alkyl, C₂-C₆ alkenyl, C₂-C₆ alkynyl, C₁-C₆ haloalkyl, or C₁-C₆ alkoxy; and R_{15b} is H. In some embodiments, R₁₅ₐ is —NH₂, F, Br, Cl, I, —CN, —OH, —OCF₃, —OCH₃, —CF₃, —CH₂CF₃, methyl, ethyl, or t-butyl; and R_{15b} is H.

In some embodiments, R₁₁ is amino, halogen, —CN, —OH, C₁-C₆ alkyl, C₁-C₆ haloalkyl, C₁-C₆ alkoxy, cycloalkyl, heterocycloalkyl, aryl, or heteroaryl, wherein each of the alkyl, alkoxy, aryl, cycloalkyl, heterocycloalkyl, or heteroaryl is optionally substituted. In some embodiments, R₁₁ is amino, halogen, —CN, —OH, —OCF₃, C₁-C₆ alkyl, C₁-C₆ haloalkyl, or C₁-C₆ alkoxy, optionally substituted with one, two, or three R₁₇.

In some embodiments, R₁₁ is C₁-C₆ alkyl, C₁-C₆ haloalkyl, or C₁-C₆ alkoxy.

In some embodiments, R₁₁ is —OCF₃, —OCH₃, methyl, ethyl, or t-butyl. In some embodiments, R₁₁ is methyl. In some embodiments, R₁₁ is t-butyl. In some embodiments, R₁₁ is —OCF₃. In some embodiments, R₁₁ is —OCH₃. In some embodiments, R₁₁ is a phenyl. In some embodiments, R₁₁ is a heterocycloalkyl. In some embodiments, R₁₁ is a 3 to 6 membered heterocycloalkyl containing 1-2 N, 1 O and/or 1S. In some embodiments, R₁₁ is halogen.

In some embodiments, R₁₁ is —NH₂, Br, Cl, I, F, —CN, —OH, —OCF₃, —OCH₃, —CF₃, —CH₂CF₃, methyl, ethyl, or t-butyl.

In some embodiments, R₁₁ is not methyl. In some embodiments, R₁₁ is not t-butyl.

In some embodiments, when R₁₅ₐ and R_{15c} are both H; then R₁₁ is not t-butyl.

In some embodiments, R₁₁ is an optionally substituted heteroaryl. In some embodiments, R₁₁ is a heteroaryl, optionally substituted with one, two, or three R₁₇. In some embodiments, R₁₁ is an unsubstituted heteroaryl.

In some embodiments, the heteroaryl is a 5-membered heteroaryl. In some embodiments, R₁₁ is furan, thiophene, oxazole, thiazole, isoxazole, triazole, oxadiazole, or thiadiazole. In some embodiments, R₁₁ is furan. In some embodiments, R₁₁ is thiophene. In some embodiments, R₁₁ is oxazole. In some embodiments, R₁₁ is thiazole. In some embodiments, R₁₁ is isoxazole. In some embodiments, R₁₁ is triazole. In some embodiments, R₁₁ is oxadiazole or thiadiazole.

In some embodiments, R₄ is C₁-C₆ alkyl, aryl, heteroaryl, cycloalkyl or heterocycloalkyl, each of which is optionally substituted. In some embodiments, R⁴ is an aryl, heteroaryl, cycloalkyl or heterocycloalkyl, each of which is optionally substituted with one, two, three, or four R₁₉. In some embodiments, R₄ is a substituted cycloalkyl or an optionally substituted heterocycloalkyl, wherein when substituted the each of which is substituted with one, two, three, or four R₁₉.

In some embodiments, R₄ is a cycloalkyl, optionally substituted with one two or three R₁₉. In some embodiments, the cycloalkyl is a spirocycloalkyl or fused cycloalkyl.

In some embodiments, R₄ is a spirocycloalkyl. In some embodiments, R₄ is a C₅-C₉ spirocycloalkyl. In some embodiments, R₄ is optionally substituted spiro[2.2]pentane. In some embodiments, R₄ is optionally substituted spiro[2.5]octane. In some embodiments, R₄ is optionally substituted spiro[3.5]nonane. In some embodiments, R₄ is a bridged cycloalkyl. In some embodiments, R₄ is a C₇-C₉ bridged cycloalkyl. In some embodiments, R₄ is a fused cycloalkyl. In some embodiments, R₄ is a C₇-C₉ fused cycloalkyl. In some embodiments, R₄ is a fused cycloalkyl. In some embodiments, R₄ is a 3-5 fused cycloalkyl. In some embodiments, R₄ is substituted. In some embodiments, the cycloalkyl is optionally substituted with one or two halogens selected from Cl, Br, or F. In some embodiments, the cycloalkyl is substituted with two F. In some embodiments, the cycloalkyl is a cyclobutyl, cyclopentyl, cyclohexyl or spiro[3,3]heptanyl. In some embodiments, R₄ is optionally substituted cyclobutyl. In some embodiments, R₄ is cyclopentyl. In some embodiments, R₄ is cyclohexyl. In some embodiments, R₄ is spiro[3,3]heptanyl.

In some embodiments, R₄ is not unsubstituted cycloalkyl. In some embodiments, R₄ is not cyclohexyl.

In some embodiments, when R₁₁ is t-butyl, the R₄ is not cyclohexyl.

In some embodiments, R₄ is heterocycloalkyl optionally substituted with one, two, or three R₁₉. In some embodiments, R₄ is a 3 to 7- membered heterocycloalkyl comprising 1,2 N, 1 O or 1 S atom, or a combination thereof.

In some embodiments, R₄ is optionally substituted heteroaryl (e.g., C₅-C₉ heteroaryl). In some embodiments, R₄ is monocyclic heteroaryl. In some embodiments, R₄ is fused heteroaryl. In some embodiments, R₄ is an optionally substituted aryl (e.g., C₆-C₁₀ aryl). In some embodiments, R₄ is an optionally substituted phenyl. In some embodiments, R₄ is an optionally substituted naphthyl.

In some embodiments, each R₁₉ is independently halogen, oxo, —CN, —NH₂, —NH(C₁—₆ alkyl), —N(C₁—₆ alkyl)₂, —OH, —CO₂H, —CO₂—C₁—₆ alkyl, —C(═O)NH₂, —C(═O)NH(C₁—₆ alkyl), —C(═O)N(C_{1—6} alkyl)₂, —S(═O)₂NH₂, —S(═O)₂NH(C₁—₆ alkyl), —S(═O)₂N(C₁—₆ alkyl)₂, C₁-C₆ alkyl, C₁-C₆ haloalkyl, C₃₋₈ cycloalkyl, C₁-C₆ heteroalkyl, C₁-C₆ alkoxy, C₁-₆ fluoroalkoxy, C₂-₇ heterocycloalkyl, aryl, heteroaryl, aryloxy, alkylthio, arylthio, alkylsulfoxide, arylsulfoxide, alkylsulfone, and arylsulfone.

In some embodiments, each R₁₉ is independently —NH(C₁—₆ alkyl), —N(C₁—₆ alkyl)₂, —CO₂—C₁—₆ alkyl, —C(═O)NH₂, —C(═O)NH(C₁—₆ alkyl), —C(═O)N(C₁—₆ alkyl)₂, —S(═O)₂NH₂, —S(═O)₂NH(C₁—₆ alkyl), —S(═O)₂N(C₁—₆ alkyl)₂, C₃₋₈ cycloalkyl, C₁-C₆ heteroalkyl, C₁-C₆ alkoxy, C₁-₆ fluoroalkoxy, C₂-₇ heterocycloalkyl, aryl, heteroaryl, aryloxy, alkylthio, arylthio, alkylsulfoxide, arylsulfoxide, alkylsulfone, and arylsulfone. In some embodiments, each R₁₉ is independently —NH(C₁—₆ alkyl), —N(C₁—₆ alkyl)₂, -CO₂-C₁₋₆ alkyl, —C(═O)NH₂, —C(═O)NH(C₁—₆ alkyl), —C(═O)N(C₁—₆ alkyl)₂, —S(═O)₂NH₂, —S(═O)₂NH(C₁—₆ alkyl), —S(═O)₂N(C₁—₆ alkyl)₂, C₃₋₈ cycloalkyl, C₁-C₆ heteroalkyl, C₁-C₆ alkoxy, C₁-₆ fluoroalkoxy, C₂-₇ heterocycloalkyl, phenyl, 5 or 6 membered heteroaryl. In some embodiments, each R₁₉ is independently C₁-C₆ alkyl, C₁-C₆ haloalkyl, C₁-C₆ alkoxy, or C₁-₆ fluoroalkoxy. In some embodiments, each R₁₉ is independently —OCF₃, —OCH₃, methyl, or ethyl. In some embodiments, each R₁₉ is independently halogen. In some embodiments, each R₁₉ is independently Cl, Br, F, or I. In some embodiments, each R₁₉ is F.

In some embodiments, R₄ is selected from:

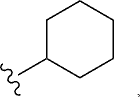

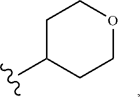

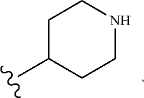

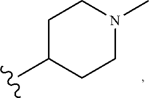

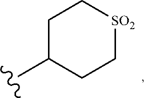

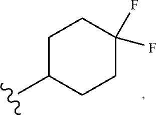

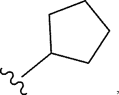

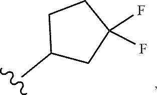

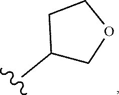

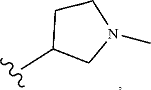

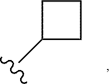

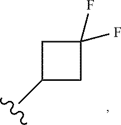

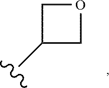

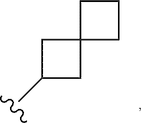

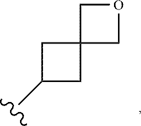

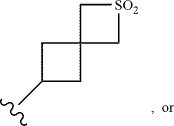

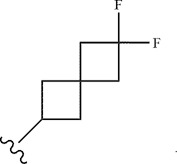

In some embodiments, R₄ is selected from:

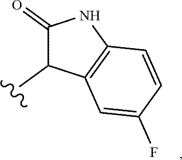

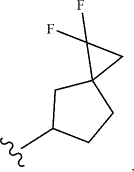

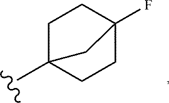

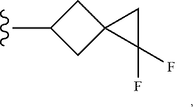

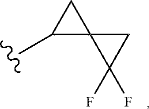

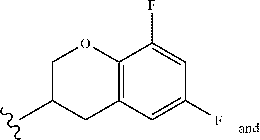

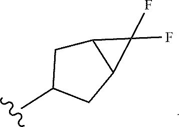

In some embodiments, R₄ is

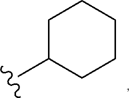

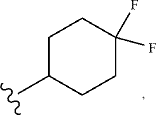

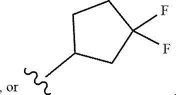

. In some embodiments, R₄ is

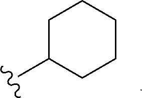

In some embodiments, R₄ is

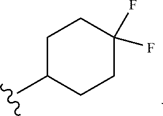

In some embodiments, R₄ is

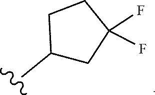

In some embodiments, R₁ can also include the foregoing substituents with the provision that the chemical moiety of R₁ is an electrophilic moiety, which is capable of forming a covalent bond with the cysteine residue. In some embodiments, the bond is reversible. In some embodiments, the bond in irreversible. In some embodiments, R₁ is a Michael acceptor. Specific examples of R₁ include acrylamide, vinyl sulfone, alpha-chloroketone, alpha-ketoamide, or other covalent modifiers described herein.

In some embodiments, R₁ is halo acetyl, glyoxyl, heterocyclo acyl, cyanide acetyl, vinylsulfonyl, vinylsulfinyl, or acrylo acyl. In some embodiments, R₁ is halo acetyl. In some embodiments, the halo acetyl is mono or di-substituted. In some embodiments, the halo acetyl is mono substituted. In some embodiments, the halo acetyl is di-substituted. In some embodiments, R₁ is acetyl chloride. In some embodiments, R₁ is acetyl fluoride. In some embodiments, R₁ is glyoxyl. In some embodiments, R₁ is heterocyclo acyl. In some embodiments, R₁ is cyanide acetyl. In some embodiments, R₁ is vinylsulfonyl or vinylsulfinyl. In some embodiments, R₁ is acrylo acyl.

In some embodiments, the R₁ can include one of the following:

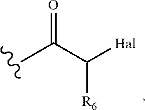

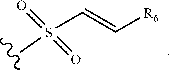

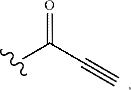

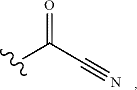

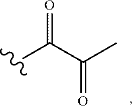

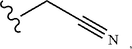

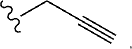

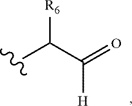

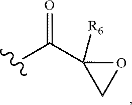

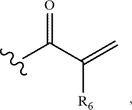

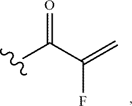

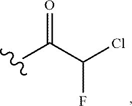

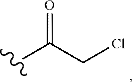

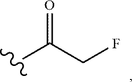

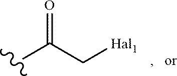

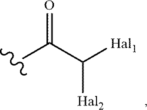

wherein Hal₁ and Hal₂ are different halogens.

In some embodiments, R₁ is:

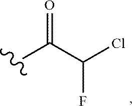

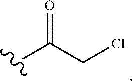

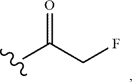

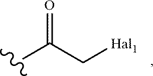

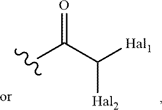

wherein Hal₁ and Ha₂ are different halogens.

In some embodiments, Hal is a halogen, such as F, Cl, Br, or I. In some embodiments, halogen is F or Cl. In some embodiments, halogen is F. In some embodiments, halogen is Cl. In some embodiments, halogen is Br. In some embodiments, halogen is I.

In some embodiments, R₁ is selected from

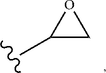

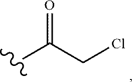

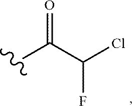

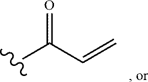

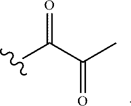

In some embodiments, R₁ is

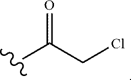

In some embodiments, R₁ is

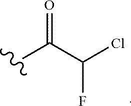

In some embodiments, R₁ is

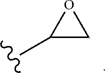

In some embodiments, R₁ is

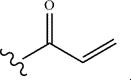

In some embodiments, R₁₆ is H, C₁-C₆ alkyl, or C₁-C₃ haloalkyl. In some embodiments, R₁₆ is C₁-C₆ alkyl. In some embodiments, R₁₆ is methyl or ethyl. In some embodiments, R₁₆ is C₁-C₃ haloalkyl. In some embodiments, R₁₆ is CF₃ or CH₂CF₃. Ins some embodiments, R₁₆ is H.

In some embodiments, each R₁₇, R₁₈ , R₁₉, and R₂₀ is independently selected from oxo, halogen, —CN, —NH₂, —NH(C₁—₆ alkyl), —N(C₁—₆ alkyl)₂, —OH, —CO₂H, —CO₂—C₁—₆ alkyl, —C(═O)NH₂, —C(═O)NH(C₁—₆ alkyl), —C(═O)N(C₁—₆ alkyl)₂, —S(═O)₂NH₂, —S(═O)₂NH(C₁—₆ alkyl), —S(═O)₂N(C₁—₆ alkyl)₂, C₁-C₆ alkyl, C₁-C₆ haloalkyl, C₃₋₈ cycloalkyl, C₁-C₆ heteroalkyl, C₁-C₆ alkoxy, C₁-₆ fluoroalkoxy, C₂-₇ heterocycloalkyl, aryl, heteroaryl, aryloxy, alkylthio, arylthio, alkylsulfoxide, arylsulfoxide, alkylsulfone, and arylsulfone.

In some embodiments, each R₁₇, R_{18 ,}R₁₉, and R₂₀ is independently —NH(C₁—₆ alkyl), —N(C₁—₆ alkyl)₂, —CO₂—C₁—₆ alkyl, —C(═O)NH₂, —C(═O)NH(C₁—₆ alkyl), —C(═O)N(C₁—₆ alkyl)₂, —S(═O)₂NH₂, —S(═O)₂NH(C₁—₆ alkyl), —S(═O)₂N(C₁—₆ alkyl)₂, C₃-₈ cycloalkyl, C₁-C₆ heteroalkyl, C₁-C₆ alkoxy, C₁-₆ fluoroalkoxy, C₂-₇ heterocycloalkyl, aryl, heteroaryl, aryloxy, alkylthio, arylthio, alkylsulfoxide, arylsulfoxide, alkylsulfone, and arylsulfone. In some embodiments, each R₁₇, R₁₈ , R₁₉, and R₂₀ is independently C₁-C₆ alkyl, C₁-C₆ haloalkyl, C₁-C₆ alkoxy, or C₁-₆ fluoroalkoxy. In some embodiments, each R₁₇, R₁₈, R₁₉, and R₂₀ is independently R₁₁ is —OCF₃, —OCH₃, methyl, or ethyl. In some embodiments, each R₁₇, R₁₈, R₁₉, and R₂₀ is independently halogen. In some embodiments, each R₁₇, R₁₈, R₁₉, and R₂₀ is independently Cl, Br, F, or I. In some embodiments, each R₁₇, R₁₈, R₁₉, and R₂₀ is independently Cl, Br, or F.

In some embodiments, the R₁, R₂, R₃, R₄, R₅, R₆, and/or R₇ subsituents shown on the structures can each indivudually be subsituted with the following subsituents, which are independently hydrogen, alkyl, alkenyl, alkynyl, aryl, alkaryl, aralkyl, halo, hydroxyl, sulfhydryl, alkoxy, alkenyloxy, alkynyloxy, aryloxy, acyl, alkylcarbonyl, arylcarbonyl, acyloxy, alkoxycarbonyl, aryloxycarbonyl, halocarbonyl, alkylcarbonato, arylcarbonato, carboxy, carboxylato, carbamoyl, mono-(alkyl)-substituted carbamoyl, di-(alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(alkyl)-substituted amino, mono- and di-(aryl)-substituted amino, alkylamido arylamido, imino, alkylimino, arylimino, nitro, nitroso, sulfo, sulfonato, alkylsulfanyl, arylsulfanyl, alkylsulfinyl, arylsulfinyl, alkylsulfonyl, arylsulfonyl, phosphono, phosphonato, phosphinato, phospho, phosphino, any with or without hetero atoms, derivatives thereof, and combinations thereof.

In some embodiments, the R₁, R₂, R₃, R₄, R₅, R₆, and/or R₇ subsituents shown on the structures can each indivudually be subsituted with the following subsituents, which are independently a hydrogen, halogens, hydroxyls, alkoxys, straight aliphatics, branched aliphatics, cyclic aliphatics, substituted aliphatics, unsubstituted aliphatics, saturated aliphatics, unsaturated aliphatics, aromatics, polyaromatics, substituted aromatics, hetero-aromatics, amines, primary amines, secondary amines, tertiary amines, aliphatic amines, carbonyls, carboxyls, amides, esters, amino acids, peptides, polypeptides, derivatives thereof, substituted or unsubstituted, or combinations thereof as well as other well-known chemical substituent.

In some embodiments, the R₁, R₂, R₃, R₄, R₅, R₆, and/or R₇ subsituents shown on the structures can each indivudually be subsituted with the following subsituents, which are independently hydrogen, alkyl, alkenyl, alkynyl, aryl, alkaryl, aralkyl, halo, hydroxyl, sulfhydryl, alkoxy, alkenyloxy, alkynyloxy, aryloxy, acyl, alkylcarbonyl, arylcarbonyl, acyloxy, alkoxycarbonyl, aryloxycarbonyl, halocarbonyl, alkylcarbonato, arylcarbonato, carboxy, carboxylato, carbamoyl, mono-(alkyl)-substituted carbamoyl, di-(alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(alkyl)-substituted amino, mono- and di-(aryl)-substituted amino, alkylamido, arylamido, imino, alkylimino, arylimino, nitro, nitroso, sulfo, sulfonato, alkylsulfanyl, arylsulfanyl, alkylsulfinyl, arylsulfinyl, alkylsulfonyl, arylsulfonyl, phosphono, phosphonato, phosphinato, phospho, phosphino, any with or without hetero atoms, any including straight chains, any including branches, and any including rings, derivatives thereof, and combinations thereof.

In some embodiments, the R₁, R₂, R₃, R₄, R₅, R₆, and/or R₇ subsituents shown on the structures can each indivudually be subsituted with the following subsituents, which are independently any one or more of the substituents selected from the group of hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂ -C₂₄ alkylcarbonyl (—CO-alkyl) and C₆-C₂₀ arylcarbonyl (—CO—aryl)), acyloxy (—O—acyl), C₂-C₂₄ alkoxycarbonyl (—(CO)— O—alkyl), C₆-C₂₀ aryloxycarbonyl (—(CO)—O—aryl), halocarbonyl (—CO)—X where X is halo), C₂-C₂₄ alkylcarbonato (—O—(CO)—O—alkyl), C₆-C₂₀ arylcarbonato (—O—(CO)—O—aryl), carboxy (— COOH), carboxylato (—COO ^{—} ), carbamoyl (—(CO)—NH₂), mono-(C₁-C₂₄ alkyl)-substituted carbamoyl (—(CO)—NH(C₁-C₂₄ alkyl)), di-(C₁-C₂₄ alkyl)-substituted carbamoyl (—(CO)—N(C₁ -C₂₄ alkyl)₂ ), mono-substituted arylcarbamoyl (—(CO)—NH—aryl), di-substituted arylcarbamoyl (— (CO)—NH—aryl)₂, thiocarbamoyl (—(CS)—NH₂), mono-(C₁-C₂₄ alkyl)-substituted thiocarbamoyl (-(CS)-NH(C₁-C₂₄ alkyl)), di-(C₁-C₂₄ alkyl)-substituted thiocarbamoyl (—(CS)—N(C₁-C₂₄ alkyl)₂ ), mono-substituted arylthiocarbamoyl (—(CS)—NH—aryl), di-substituted arylthiocarbamoyl (— (CS)—NH—aryl)₂, carbamido (—NH—(CO)—NH₂), ), mono-(C₁-C₂₄ alkyl)-substituted carbamido (—NH —(CO)—NH(C₁-C₂₄ alkyl)), di-(C₁-C₂₄ alkyl)-substituted carbamido (—NH —(CO)—N(C₁ -C₂₄ alkyl)₂ ), mono-substituted aryl carbamido (—NH —(CO)—NH—aryl), di-substituted aryl carbamido (—NH —(CO)—N—(aryl)₂) cyano(—C≡N), isocyano (—N ⁺≡C ^{—}), cyanato (—O—C≡N), isocyanato (—O—N⁺≡C^{—}), thiocyanato (—S—C≡N), isothiocyanato (—S—N⁺≡C^{—}), azido (—N═N⁺ ═N^{—}), formyl (—(CO)—H), thioformyl (—(CS)—H), amino (—NH ₂), mono- and di-(C₁-C₂₄ alkyl)-substituted amino, mono- and di-(C₆-C₂₀ aryl)-substituted amino, C₂ -C₂₄ alkylamido (—NH—(CO)—alkyl), C₅ -C₂₀ arylamido (—NH—(CO)—aryl), imino (—CR═NH where R is hydrogen, C₁-C₂₄ alkyl, C₅ -C₂₀ aryl, C₆ -C₂₄ alkaryl, C₆ -C₂₄ aralkyl, etc.), alkylimino (—CR═N(alkyl), where R=hydrogen, C₁ -C₂₄ alkyl, aryl, alkaryl, aralkyl, etc.), arylimino (—CR═N(aryl), where R=hydrogen, alkyl, aryl, alkaryl, etc.), nitro (—NO ₂), nitroso (—NO), sulfonic acid (—SO₂ —OH), sulfonato (—SO₂ —O^{—})’ C₁-C₂₄ alkylsulfanyl (—S—alkyl; also termed “alkylthio”), C₅ -C₂₀ arylsulfanyl (-S-aryl; also termed “arylthio”), C₁ -C₂₄ alkylsulfinyl (—(SO)—alkyl), C₅ -C₂₀ arylsulfinyl (—(SO)—aryl), C₁ -C₂₄ alkylsulfonyl (—SO₂ —alkyl), C₅ -C₂₀ arylsulfonyl (—SO₂—aryl), phosphono (—P(O)(OH)₂ ), phosphonato (—P(O)(O^{—})₂ ), phosphinato (—P(O)(O—)), phospho (—PO₂ ), phosphino (—PH₂ ), any with or without hetero atoms (e.g., N, O, P, S, or other) where the hetero atoms can be substituted (e.g., hetero atom substituted for carbon in chain or ring) for the carbons or in addition thereto (e.g., hetero atom added to carbon chain or ring) swapped, any including straight chains, any including branches, and any inducing rings, derivatives thereof, and combinations thereof.

In some embodiments, R₂, R₃, R₄, R₇, and/or R₈ are each independently selected from H, CH₃, CF₃, CHF₂, CH₂F, C₂H₅, Hal, —CN, or an optionally substituted moiety selected from C₃-C₁₂ alkyl, C₃-C₁₂ alkenyl, cycloalkyl, cycloalkenyl, heterocycloalkyl, heterocycloalkenyl, aryl, heteroaryl, fused heterocycle (heterocyclic), fused aryl (e.g., polyaryl), fused heterocycle-aryl, spirocycle (spirocycloalkyl, spiroheterocycle), or combinations thereof.

Any combination of the groups described above for the various variables is contemplated herein. Throughout the specification, groups and substituents thereof are chosen by one skilled in the field to provide stable moieties and compounds.

In some embodiments, the compounds made in the examples below are made from racemic starting materials (and/or intermediates) and separated into the individual enantiomers by chiral chromatography as final products or intermediates. Unless otherwise stated, it is understood that the absolute configuration of the separated intermediates and final compounds as drawn is arbitrarily assigned and was not determined.

Non-limiting examples of compounds described herein, or pharmaceutically acceptable salts or solvated are presented in Table 1.

TABLE 1

| | |
|---|---|
| Compound Structure | Compound ID |
| 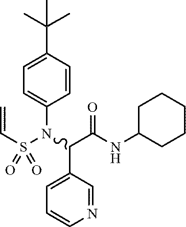 [SMILES: C=CS(=O)(=O)N(c1ccc(C(C)(C)C)cc1)C(C(=O)NC1CCCCC1)c1cccnc1] | INSCoV-110 |
| 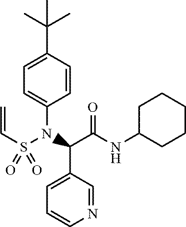 [SMILES: C=CS(=O)(=O)N(c1ccc(C(C)(C)C)cc1)[C@@H](C(=O)NC1CCCCC1)c1cccnc1] | INSCoV-110-1 |
| 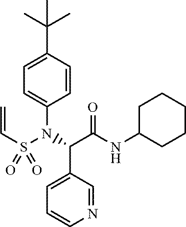 [SMILES: C=CS(=O)(=O)N(c1ccc(C(C)(C)C)cc1)[C@H](C(=O)NC1CCCCC1)c1cccnc1] | INSCoV-110-2 |
| 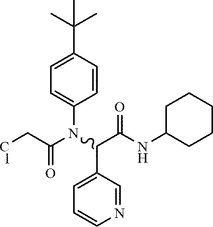 [SMILES: CC(C)(C)c1ccc(N(C(=O)CCl)C(C(=O)NC2CCCCC2)c2cccnc2)cc1] | INSCoV-11A |
| 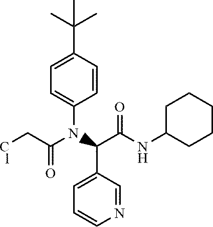 [SMILES: CC(C)(C)c1ccc(N(C(=O)CCl)[C@@H](C(=O)NC2CCCCC2)c2cccnc2)cc1] | INSCoV-11A(1) |
| 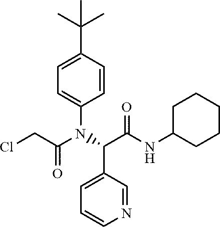 [SMILES: CC(C)(C)c1ccc(N(C(=O)CCl)[C@H](C(=O)NC2CCCCC2)c2cccnc2)cc1] | INSCoV-110A(2) |
| 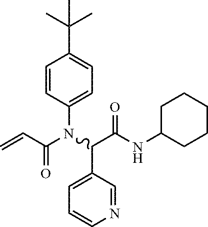 [SMILES: C=CC(=O)N(c1ccc(C(C)(C)C)cc1)C(C(=O)NC1CCCCC1)c1cccnc1] | INSCoV-110B |
| 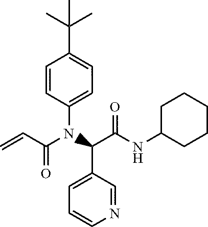 [SMILES: C=CC(=O)N(c1ccc(C(C)(C)C)cc1)[C@@H](C(=O)NC1CCCCC1)c1cccnc1] | INSCoV-110B(1) |
| 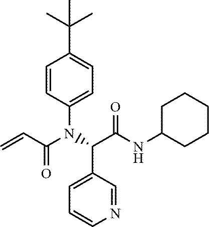 [SMILES: C=CC(=O)N(c1ccc(C(C)(C)C)cc1)[C@H](C(=O)NC1CCCCC1)c1cccnc1] | INSCoV-110B(2) |
| 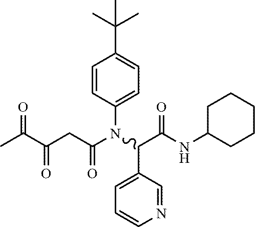 [SMILES: CC(=O)C(=O)CC(=O)N(c1ccc(C(C)(C)C)cc1)C(C(=O)NC1CCCCC1)c1cccnc1] | INSCoV-110C |
| 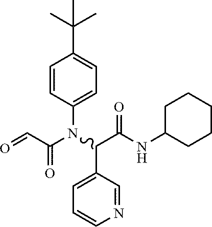 [SMILES: CC(C)(C)c1ccc(N(C(=O)C=O)C(C(=O)NC2CCCCC2)c2cccnc2)cc1] | INSCoV-110D |
| 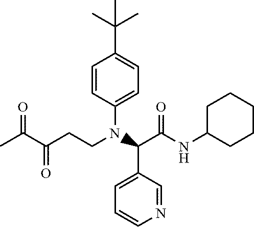 [SMILES: CC(=O)C(=O)CCN(c1ccc(C(C)(C)C)cc1)[C@@H](C(=O)NC1CCCCC1)c1cccnc1] | INSCoV-110E |
| 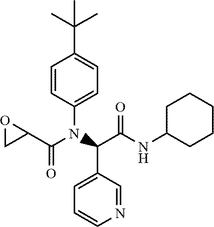 [SMILES: CC(C)(C)c1ccc(N(C(=O)C2CO2)[C@@H](C(=O)NC2CCCCC2)c2cccnc2)cc1] | INSCoV-110F |
| 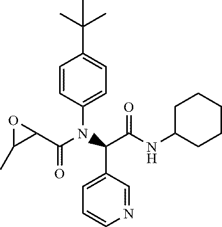 [SMILES: CC1OC1C(=O)N(c1ccc(C(C)(C)C)cc1)[C@@H](C(=O)NC1CCCCC1)c1cccnc1] | INSCoV-110G |
| 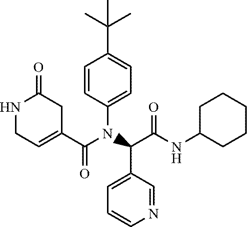 [SMILES: CC(C)(C)c1ccc(N(C(=O)C2=CCNC(=O)C2)[C@@H](C(=O)NC2CCCCC2)c2cccnc2)cc1] | INSCoV-110H |
| 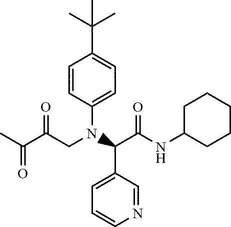 [SMILES: CC(=O)C(=O)CN(c1ccc(C(C)(C)C)cc1)[C@@H](C(=O)NC1CCCCC1)c1cccnc1] | INSCoV-110I |
| 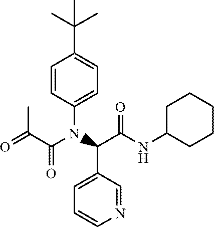 [SMILES: CC(=O)C(=O)N(c1ccc(C(C)(C)C)cc1)[C@@H](C(=O)NC1CCCCC1)c1cccnc1] | INSCoV-110J |
| 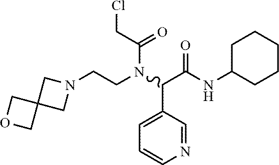 [SMILES: O=C(NC1CCCCC1)C(c1cccnc1)N(CCN1CC2(COC2)C1)C(=O)CCl] | INSCoV-500 |
| 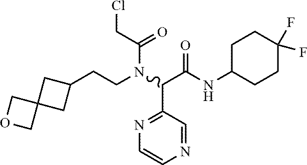 [SMILES: O=C(NC1CCC(F)(F)CC1)C(c1cnccn1)N(CCC1CC2(COC2)C1)C(=O)CCl] | INSCoV-500A |
| 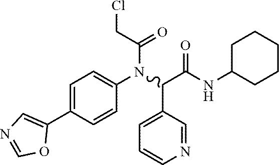 [SMILES: O=C(NC1CCCCC1)C(c1cccnc1)N(C(=O)CCl)c1ccc(-c2cnco2)cc1] | INSCoV-501 |
| 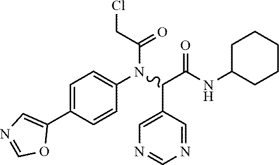 [SMILES: O=C(NC1CCCCC1)C(c1cncnc1)N(C(=O)CCl)c1ccc(-c2cnco2)cc1] | INSCoV-501A |
| 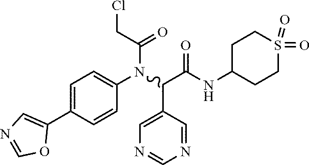 [SMILES: O=C(NC1CCS(=O)(=O)CC1)C(c1cncnc1)N(C(=O)CCl)c1ccc(-c2cnco2)cc1] | INSCoV-501B |
| 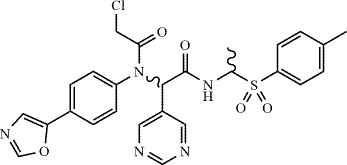 [SMILES: Cc1ccc(S(=O)(=O)C(C)NC(=O)C(c2cncnc2)N(C(=O)CCl)c2ccc(-c3cnco3)cc2)cc1] | INSCoV-501C(2) |
| 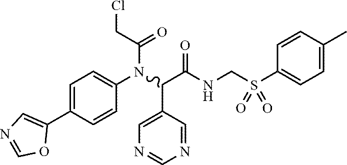 [SMILES: Cc1ccc(S(=O)(=O)CNC(=O)C(c2cncnc2)N(C(=O)CCl)c2ccc(-c3cnco3)cc2)cc1] | INSCoV-501D |
| 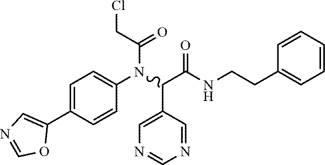 [SMILES: O=C(NCCc1ccccc1)C(c1cncnc1)N(C(=O)CCl)c1ccc(-c2cnco2)cc1] | INSCoV-501E |
| 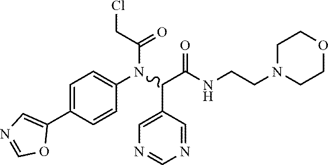 [SMILES: O=C(NCCN1CCOCC1)C(c1cncnc1)N(C(=O)CCl)c1ccc(-c2cnco2)cc1] | INSCoV-501F |
| 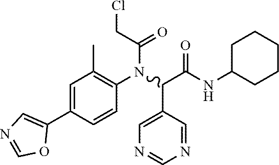 [SMILES: Cc1cc(-c2cnco2)ccc1N(C(=O)CCl)C(C(=O)NC1CCCCC1)c1cncnc1] | INSCoV-501G |
| 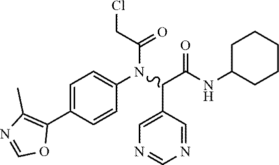 [SMILES: Cc1ncoc1-c1ccc(N(C(=O)CCl)C(C(=O)NC2CCCCC2)c2cncnc2)cc1] | INSCoV-501G(1) |
| 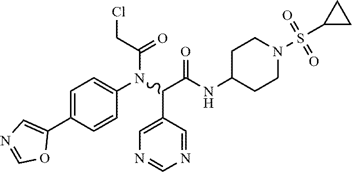 [SMILES: O=C(NC1CCN(S(=O)(=O)C2CC2)CC1)C(c1cncnc1)N(C(=O)CCl)c1ccc(-c2cnco2)cc1] | INSCoV-501H |
| 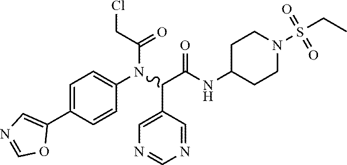 [SMILES: CCS(=O)(=O)N1CCC(NC(=O)C(c2cncnc2)N(C(=O)CCl)c2ccc(-c3cnco3)cc2)CC1] | INSCoV-501H(1) |
| 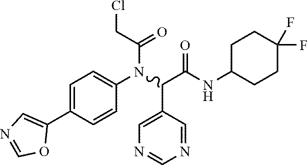 [SMILES: O=C(NC1CCC(F)(F)CC1)C(c1cncnc1)N(C(=O)CCl)c1ccc(-c2cnco2)cc1] | INSCoV-501I |
| 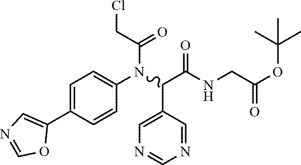 [SMILES: CC(C)(C)OC(=O)CNC(=O)C(c1cncnc1)N(C(=O)CCl)c1ccc(-c2cnco2)cc1] | INSCoV-501J |
| 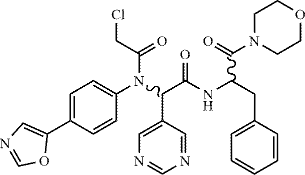 [SMILES: O=C(NC(Cc1ccccc1)C(=O)N1CCOCC1)C(c1cncnc1)N(C(=O)CCl)c1ccc(-c2cnco2)cc1] | INSCov-501K |
| 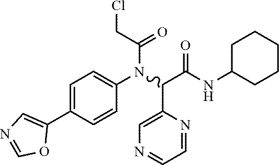 [SMILES: O=C(NC1CCCCC1)C(c1cnccn1)N(C(=O)CCl)c1ccc(-c2cnco2)cc1] | INSCoV-501L |
| 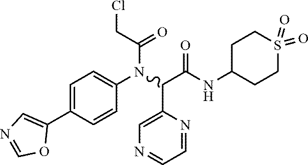 [SMILES: O=C(NC1CCS(=O)(=O)CC1)C(c1cnccn1)N(C(=O)CCl)c1ccc(-c2cnco2)cc1] | INSCoV-501M |
| 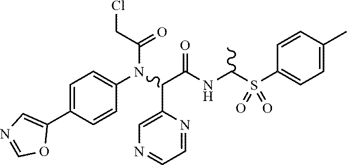 [SMILES: Cc1ccc(S(=O)(=O)C(C)NC(=O)C(c2cnccn2)N(C(=O)CCl)c2ccc(-c3cnco3)cc2)cc1] | INSCoV-501N |
| 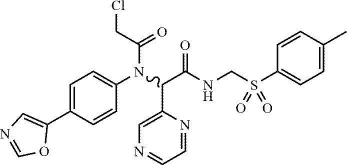 [SMILES: Cc1ccc(S(=O)(=O)CNC(=O)C(c2cnccn2)N(C(=O)CCl)c2ccc(-c3cnco3)cc2)cc1] | INSCoV-501O |
| 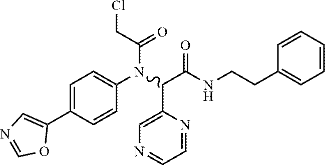 [SMILES: O=C(NCCc1ccccc1)C(c1cnccn1)N(C(=O)CCl)c1ccc(-c2cnco2)cc1] | INSCoV-501P |
| 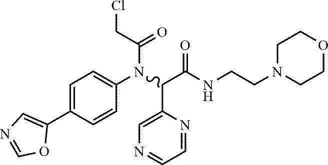 [SMILES: O=C(NCCN1CCOCC1)C(c1cnccn1)N(C(=O)CCl)c1ccc(-c2cnco2)cc1] | INSCoV-501Q |
| 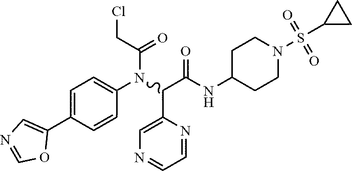 [SMILES: O=C(NC1CCN(S(=O)(=O)C2CC2)CC1)C(c1cnccn1)N(C(=O)CCl)c1ccc(-c2cnco2)cc1] | INSCoV-501R |
| 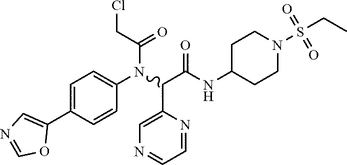 [SMILES: CCS(=O)(=O)N1CCC(NC(=O)C(c2cnccn2)N(C(=O)CCl)c2ccc(-c3cnco3)cc2)CC1] | INSCoV-501R(1) |
| 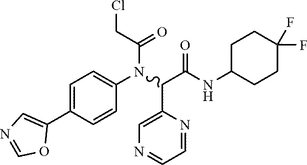 [SMILES: O=C(NC1CCC(F)(F)CC1)C(c1cnccn1)N(C(=O)CCl)c1ccc(-c2cnco2)cc1] | INSCoV-501S |
| 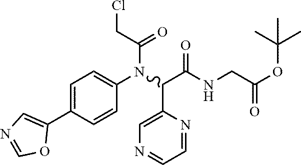 [SMILES: CC(C)(C)OC(=O)CNC(=O)C(c1cnccn1)N(C(=O)CCl)c1ccc(-c2cnco2)cc1] | INSCoV-501T |
| 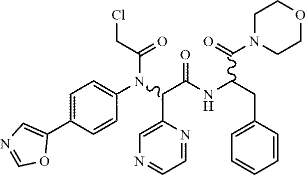 [SMILES: O=C(NC(Cc1ccccc1)C(=O)N1CCOCC1)C(c1cnccn1)N(C(=O)CCl)c1ccc(-c2cnco2)cc1] | INSCoV-501U |
| 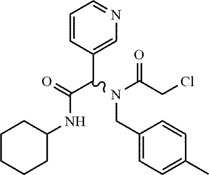 [SMILES: Cc1ccc(CN(C(=O)CCl)C(C(=O)NC2CCCCC2)c2cccnc2)cc1] | INSCoV-502 |
| 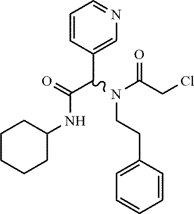 [SMILES: O=C(NC1CCCCC1)C(c1cccnc1)N(CCc1ccccc1)C(=O)CCl] | INSCoV-503 |
| 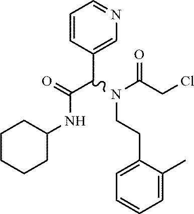 [SMILES: Cc1ccccc1CCN(C(=O)CCl)C(C(=O)NC1CCCCC1)c1cccnc1] | INSCoV-503A |
| 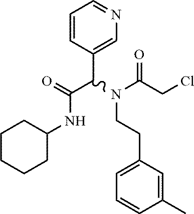 [SMILES: Cc1cccc(CCN(C(=O)CCl)C(C(=O)NC2CCCCC2)c2cccnc2)c1] | INSCoV-503B |
| 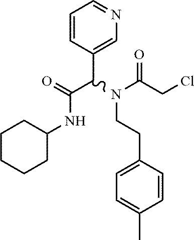 [SMILES: Cc1ccc(CCN(C(=O)CCl)C(C(=O)NC2CCCCC2)c2cccnc2)cc1] | INSCoV-503C |
| 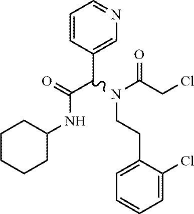 [SMILES: O=C(NC1CCCCC1)C(c1cccnc1)N(CCc1ccccc1Cl)C(=O)CCl] | INSCoV-503D |
| 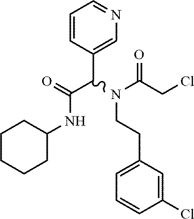 [SMILES: O=C(NC1CCCCC1)C(c1cccnc1)N(CCc1cccc(Cl)c1)C(=O)CCl] | INSCoV-503E |
| 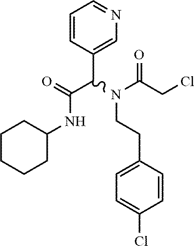 [SMILES: O=C(NC1CCCCC1)C(c1cccnc1)N(CCc1ccc(Cl)cc1)C(=O)CCl] | INSCoV-503F |
| 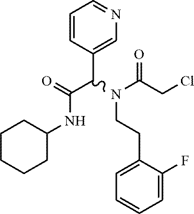 [SMILES: O=C(NC1CCCCC1)C(c1cccnc1)N(CCc1ccccc1F)C(=O)CCl] | INSCoV-503G |
| 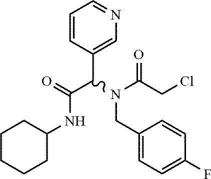 [SMILES: O=C(NC1CCCCC1)C(c1cccnc1)N(Cc1ccc(F)cc1)C(=O)CCl] | INSCoV-504 |
| 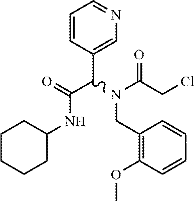 [SMILES: COc1ccccc1CN(C(=O)CCl)C(C(=O)NC1CCCCC1)c1cccnc1] | INSCoV-505 |
| 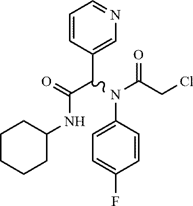 [SMILES: O=C(NC1CCCCC1)C(c1cccnc1)N(C(=O)CCl)c1ccc(F)cc1] | INSCoV-506 |
| 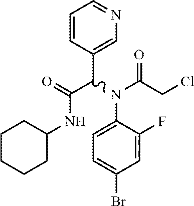 [SMILES: O=C(NC1CCCCC1)C(c1cccnc1)N(C(=O)CCl)c1ccc(Br)cc1F] | INSCoV-507 |
| 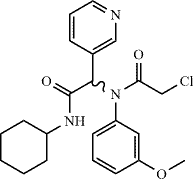 [SMILES: COc1cccc(N(C(=O)CCl)C(C(=O)NC2CCCCC2)c2cccnc2)c1] | INSCoV-508 |
| 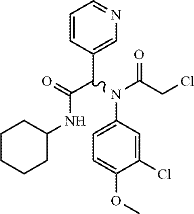 [SMILES: COc1ccc(N(C(=O)CCl)C(C(=O)NC2CCCCC2)c2cccnc2)cc1Cl] | INSCoV-509 |
| 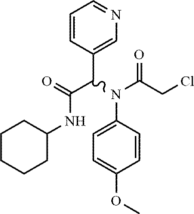 [SMILES: COc1ccc(N(C(=O)CCl)C(C(=O)NC2CCCCC2)c2cccnc2)cc1] | INSCoV-510 |
| 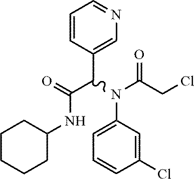 [SMILES: O=C(NC1CCCCC1)C(c1cccnc1)N(C(=O)CCl)c1cccc(Cl)c1] | INSCoV-511 |
| 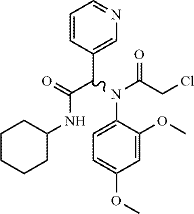 [SMILES: COc1ccc(N(C(=O)CCl)C(C(=O)NC2CCCCC2)c2cccnc2)c(OC)c1] | INSCoV-512 |
| 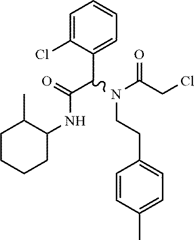 [SMILES: Cc1ccc(CCN(C(=O)CCl)C(C(=O)NC2CCCCC2C)c2ccccc2Cl)cc1] | INSCoV-513 |
| 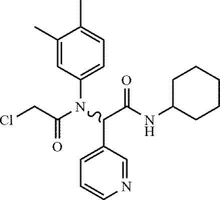 [SMILES: Cc1ccc(N(C(=O)CCl)C(C(=O)NC2CCCCC2)c2cccnc2)cc1C] | INSCoV-514 |
| 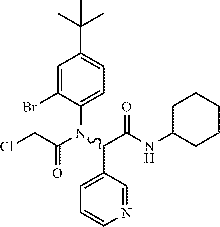 [SMILES: CC(C)(C)c1ccc(N(C(=O)CCl)C(C(=O)NC2CCCCC2)c2cccnc2)c(Br)c1] | INSCoV-515 |
| 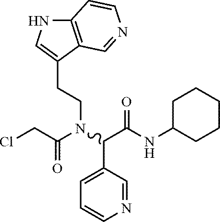 [SMILES: O=C(NC1CCCCC1)C(c1cccnc1)N(CCc1c[nH]c2ccncc12)C(=O)CCl] | INSCoV-516 |
| 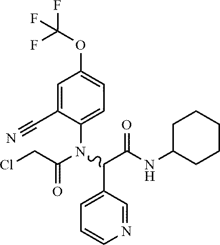 [SMILES: N#Cc1cc(OC(F)(F)F)ccc1N(C(=O)CCl)C(C(=O)NC1CCCCC1)c1cccnc1] | INSCoV-517 |
| 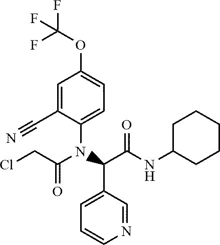 [SMILES: N#Cc1cc(OC(F)(F)F)ccc1N(C(=O)CCl)[C@@H](C(=O)NC1CCCCC1)c1cccnc1] | INSCoV-517(1) |
| 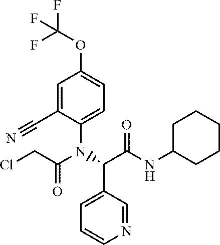 [SMILES: N#Cc1cc(OC(F)(F)F)ccc1N(C(=O)CCl)[C@H](C(=O)NC1CCCCC1)c1cccnc1] | INSCoV-517(2) |
| 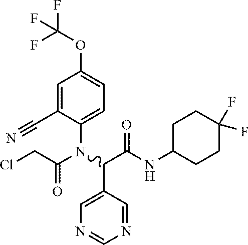 [SMILES: N#Cc1cc(OC(F)(F)F)ccc1N(C(=O)CCl)C(C(=O)NC1CCC(F)(F)CC1)c1cncnc1] | INSCoV-517A |
| 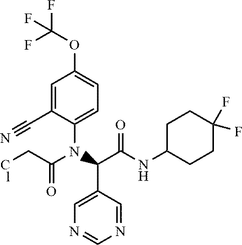 [SMILES: N#Cc1cc(OC(F)(F)F)ccc1N(C(=O)CCl)[C@@H](C(=O)NC1CCC(F)(F)CC1)c1cncnc1] | INSCoV-517-A(1A) |
| 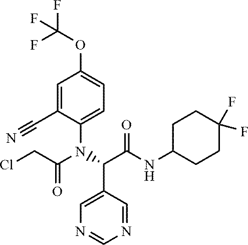 [SMILES: N#Cc1cc(OC(F)(F)F)ccc1N(C(=O)CCl)[C@H](C(=O)NC1CCC(F)(F)CC1)c1cncnc1] | INSCoV-517-A(1B) |
| 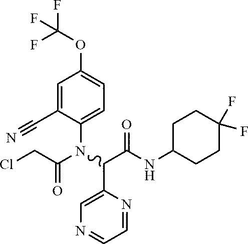 [SMILES: N#Cc1cc(OC(F)(F)F)ccc1N(C(=O)CCl)C(C(=O)NC1CCC(F)(F)CC1)c1cnccn1] | INSCoV-517B |
| 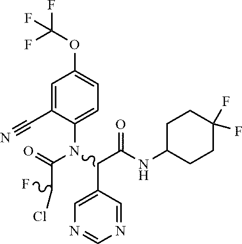 [SMILES: N#Cc1cc(OC(F)(F)F)ccc1N(C(=O)C(F)Cl)C(C(=O)NC1CCC(F)(F)CC1)c1cncnc1] | INSCoV-517C |
| 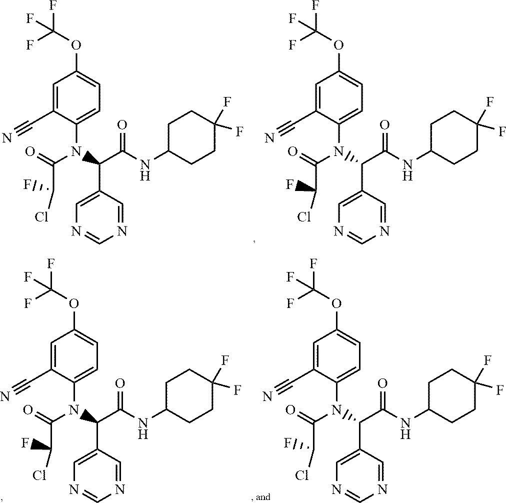 | INSCoV-517-C(1), INSCoV-517-C(2), INSCoV-517-C(3), and INSCoV-517-C(4) |
| 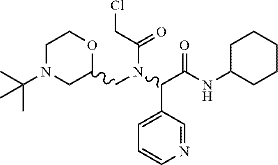 [SMILES: CC(C)(C)N1CCOC(CN(C(=O)CCl)C(C(=O)NC2CCCCC2)c2cccnc2)C1] | INSCoV-518 |
| 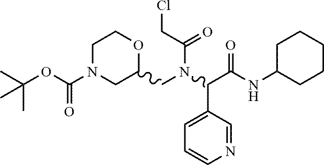 [SMILES: CC(C)(C)OC(=O)N1CCOC(CN(C(=O)CCl)C(C(=O)NC2CCCCC2)c2cccnc2)C1] | INSCoV-518A |
| 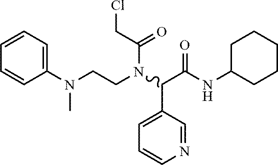 [SMILES: CN(CCN(C(=O)CCl)C(C(=O)NC1CCCCC1)c1cccnc1)c1ccccc1] | INSCoV-519 |
| 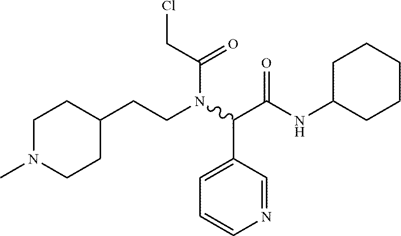 [SMILES: CN1CCC(CCN(C(=O)CCl)C(C(=O)NC2CCCCC2)c2cccnc2)CC1] | INSCoV-520 |
| 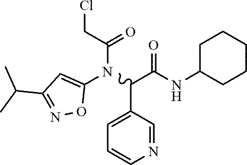 [SMILES: CC(C)c1cc(N(C(=O)CCl)C(C(=O)NC2CCCCC2)c2cccnc2)on1] | INSCoV-521 |
| 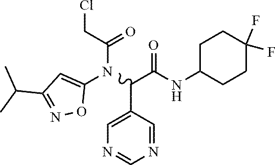 [SMILES: CC(C)c1cc(N(C(=O)CCl)C(C(=O)NC2CCC(F)(F)CC2)c2cncnc2)on1] | INSCoV-521A |
| 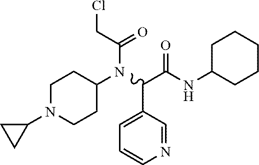 [SMILES: O=C(NC1CCCCC1)C(c1cccnc1)N(C(=O)CCl)C1CCN(C2CC2)CC1] | INSCoV-522 |
| 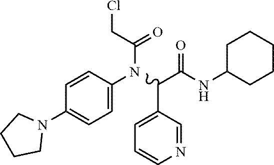 [SMILES: O=C(NC1CCCCC1)C(c1cccnc1)N(C(=O)CCl)c1ccc(N2CCCC2)cc1] | INSCoV-523 |
| 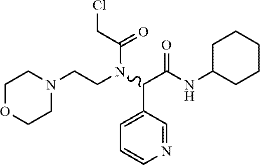 [SMILES: O=C(NC1CCCCC1)C(c1cccnc1)N(CCN1CCOCC1)C(=O)CCl] | INSCoV-524 |
| 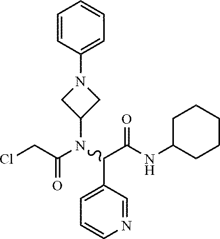 [SMILES: O=C(NC1CCCCC1)C(c1cccnc1)N(C(=O)CCl)C1CN(c2ccccc2)C1] | INSCoV-525 |
| 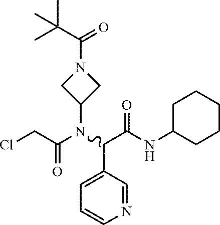 [SMILES: CC(C)(C)C(=O)N1CC(N(C(=O)CCl)C(C(=O)NC2CCCCC2)c2cccnc2)C1] | INSCoV-526 |
| 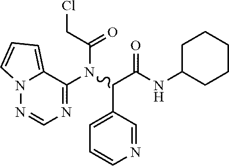 [SMILES: O=C(NC1CCCCC1)C(c1cccnc1)N(C(=O)CCl)c1ncnn2cccc12] | INSCoV-527 |
| 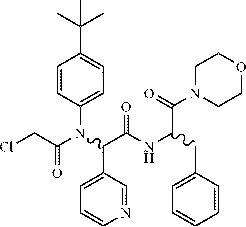 [SMILES: CC(C)(C)c1ccc(N(C(=O)CCl)C(C(=O)NC(Cc2ccccc2)C(=O)N2CCOCC2)c2cccnc2)cc1] | INSCoV-528 |
| 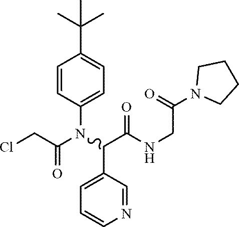 [SMILES: CC(C)(C)c1ccc(N(C(=O)CCl)C(C(=O)NCC(=O)N2CCCC2)c2cccnc2)cc1] | INSCoV-529 |
| 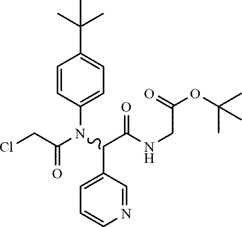 [SMILES: CC(C)(C)OC(=O)CNC(=O)C(c1cccnc1)N(C(=O)CCl)c1ccc(C(C)(C)C)cc1] | INSCoV-530 |
| 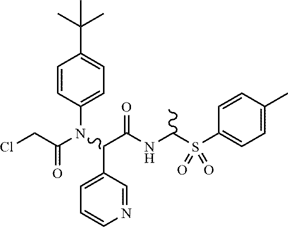 [SMILES: Cc1ccc(S(=O)(=O)C(C)NC(=O)C(c2cccnc2)N(C(=O)CCl)c2ccc(C(C)(C)C)cc2)cc1] | INSCoV-531 |
| 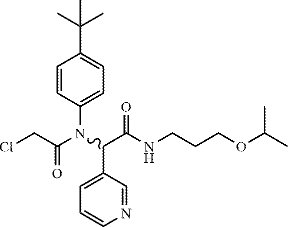 [SMILES: CC(C)OCCCNC(=O)C(c1cccnc1)N(C(=O)CCl)c1ccc(C(C)(C)C)cc1] | INSCoV-532 |
| 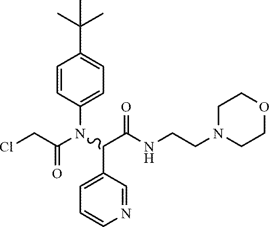 [SMILES: CC(C)(C)c1ccc(N(C(=O)CCl)C(C(=O)NCCN2CCOCC2)c2cccnc2)cc1] | INSCoV-533 |
| 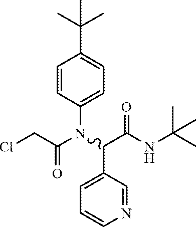 [SMILES: CC(C)(C)NC(=O)C(c1cccnc1)N(C(=O)CCl)c1ccc(C(C)(C)C)cc1] | INSCoV-534 |
| 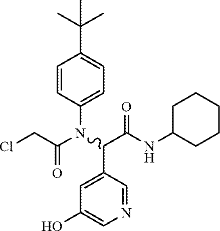 [SMILES: CC(C)(C)c1ccc(N(C(=O)CCl)C(C(=O)NC2CCCCC2)c2cncc(O)c2)cc1] | INSCoV-535 |
| 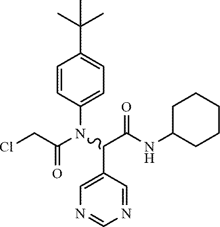 [SMILES: CC(C)(C)c1ccc(N(C(=O)CCl)C(C(=O)NC2CCCCC2)c2cncnc2)cc1] | INSCoV-536 |
| 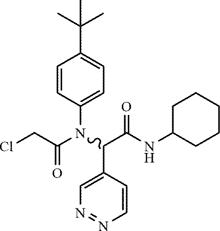 [SMILES: CC(C)(C)c1ccc(N(C(=O)CCl)C(C(=O)NC2CCCCC2)c2ccnnc2)cc1] | INSCoV-537 |
| 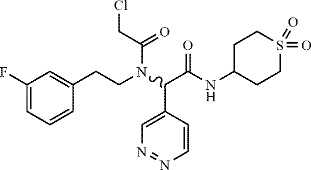 [SMILES: O=C(NC1CCS(=O)(=O)CC1)C(c1ccnnc1)N(CCc1cccc(F)c1)C(=O)CCl] | INSCoV-537A |
| 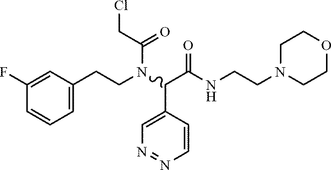 [SMILES: O=C(NCCN1CCOCC1)C(c1ccnnc1)N(CCc1cccc(F)c1)C(=O)CCl] | INSCoV-537B |
| 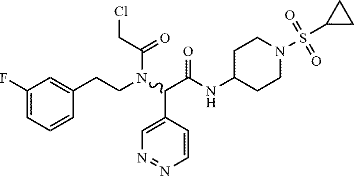 [SMILES: O=C(NC1CCN(S(=O)(=O)C2CC2)CC1)C(c1ccnnc1)N(CCc1cccc(F)c1)C(=O)CCl] | INSCoV-537C |
| 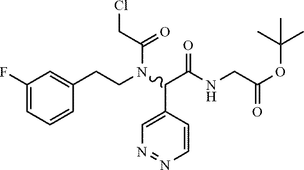 [SMILES: CC(C)(C)OC(=O)CNC(=O)C(c1ccnnc1)N(CCc1cccc(F)c1)C(=O)CCl] | INSCoV-537D |
| 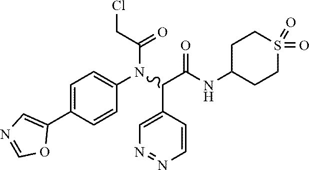 [SMILES: O=C(NC1CCS(=O)(=O)CC1)C(c1ccnnc1)N(C(=O)CCl)c1ccc(-c2cnco2)cc1] | INSCoV-537E |
| 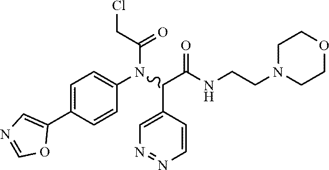 [SMILES: O=C(NCCN1CCOCC1)C(c1ccnnc1)N(C(=O)CCl)c1ccc(-c2cnco2)cc1] | INSCoV-537F |
| 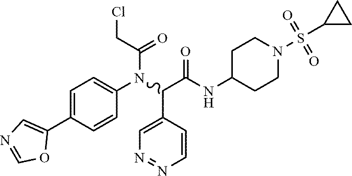 [SMILES: O=C(NC1CCN(S(=O)(=O)C2CC2)CC1)C(c1ccnnc1)N(C(=O)CCl)c1ccc(-c2cnco2)cc1] | INSCoV-537G |
| 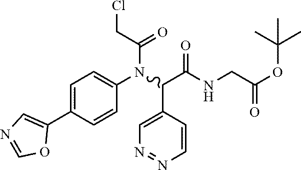 [SMILES: CC(C)(C)OC(=O)CNC(=O)C(c1ccnnc1)N(C(=O)CCl)c1ccc(-c2cnco2)cc1] | INSCoV-537H |
| 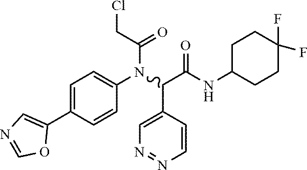 [SMILES: O=C(NC1CCC(F)(F)CC1)C(c1ccnnc1)N(C(=O)CCl)c1ccc(-c2cnco2)cc1] | INSCoV-537I |
| 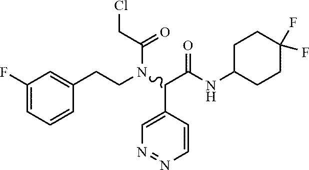 [SMILES: O=C(NC1CCC(F)(F)CC1)C(c1ccnnc1)N(CCc1cccc(F)c1)C(=O)CCl] | INSCoV-537J |
| 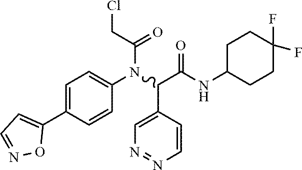 [SMILES: O=C(NC1CCC(F)(F)CC1)C(c1ccnnc1)N(C(=O)CCl)c1ccc(-c2ccno2)cc1] | INSCoV-537K |
| 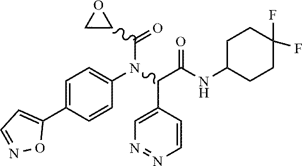 [SMILES: O=C(NC1CCC(F)(F)CC1)C(c1ccnnc1)N(C(=O)C1CO1)c1ccc(-c2ccno2)cc1] | INSCoV-537L |
| 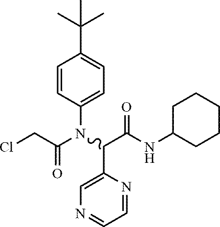 [SMILES: CC(C)(C)c1ccc(N(C(=O)CCl)C(C(=O)NC2CCCCC2)c2cnccn2)cc1] | INSCoV-538 |
| 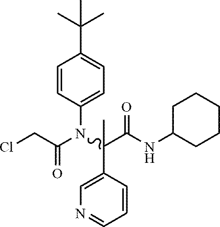 [SMILES: CC(C)(C)c1ccc(N(C(=O)CCl)C(C)(C(=O)NC2CCCCC2)c2cccnc2)cc1] | INSCoV-538A |
| 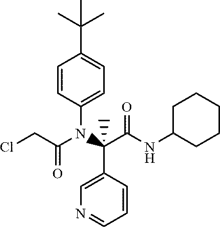 [SMILES: CC(C)(C)c1ccc(N(C(=O)CCl)[C@@](C)(C(=O)NC2CCCCC2)c2cccnc2)cc1] | INSCoV-538-A(1) |
| 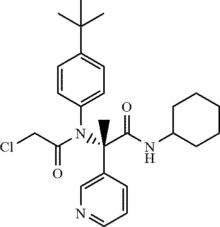 [SMILES: CC(C)(C)c1ccc(N(C(=O)CCl)[C@](C)(C(=O)NC2CCCCC2)c2cccnc2)cc1] | INSCoV-538-A(2) |
| 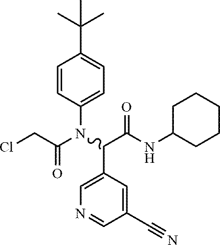 [SMILES: CC(C)(C)c1ccc(N(C(=O)CCl)C(C(=O)NC2CCCCC2)c2cncc(C#N)c2)cc1] | INSCoV-539 |
| 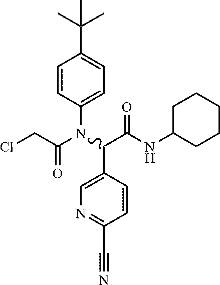 [SMILES: CC(C)(C)c1ccc(N(C(=O)CCl)C(C(=O)NC2CCCCC2)c2ccc(C#N)nc2)cc1] | INSCoV-539A |
| 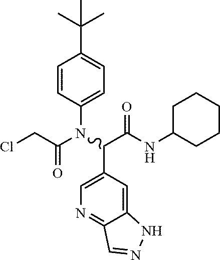 [SMILES: CC(C)(C)c1ccc(N(C(=O)CCl)C(C(=O)NC2CCCCC2)c2cnc3cn[nH]c3c2)cc1] | INSCoV-540 |
| 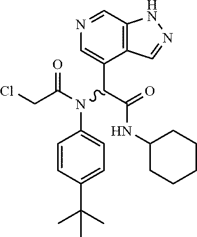 [SMILES: CC(C)(C)c1ccc(N(C(=O)CCl)C(C(=O)NC2CCCCC2)c2cncc3[nH]ncc23)cc1] | INSCoV-541 |
| 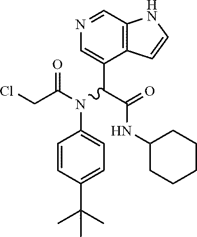 [SMILES: CC(C)(C)c1ccc(N(C(=O)CCl)C(C(=O)NC2CCCCC2)c2cncc3[nH]ccc23)cc1] | INSCoV-542 |
| 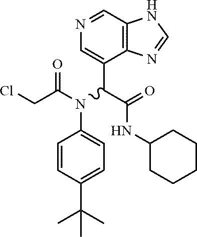 [SMILES: CC(C)(C)c1ccc(N(C(=O)CCl)C(C(=O)NC2CCCCC2)c2cncc3[nH]cnc23)cc1] | INSCoV-543 |
| 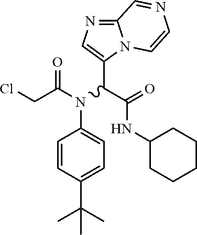 [SMILES: CC(C)(C)c1ccc(N(C(=O)CCl)C(C(=O)NC2CCCCC2)c2cnc3cnccn23)cc1] | INSCoV-544 |
| 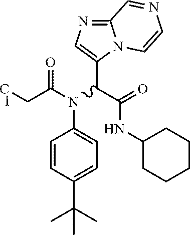 [SMILES: CC(C)(C)c1ccc(N(C(=O)CCl)C(C(=O)NC2CCCCC2)c2cnc3cnccn23)cc1] | INSCoV-545 |
| 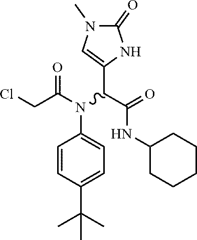 [SMILES: Cn1cc(C(C(=O)NC2CCCCC2)N(C(=O)CCl)c2ccc(C(C)(C)C)cc2)[nH]c1=O] | INSCoV-546 |
| 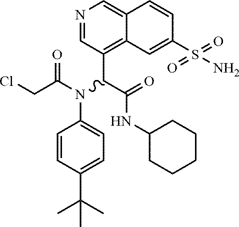 [SMILES: CC(C)(C)c1ccc(N(C(=O)CCl)C(C(=O)NC2CCCCC2)c2cncc3ccc(S(N)(=O)=O)cc23)cc1] | INSCoV-547 |
| 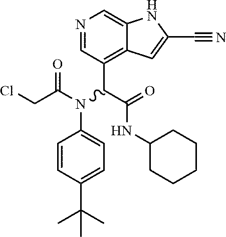 [SMILES: CC(C)(C)c1ccc(N(C(=O)CCl)C(C(=O)NC2CCCCC2)c2cncc3[nH]c(C#N)cc23)cc1] | INSCoV-548 |
| 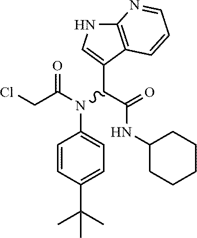 [SMILES: CC(C)(C)c1ccc(N(C(=O)CCl)C(C(=O)NC2CCCCC2)c2c[nH]c3ncccc23)cc1] | INSCoV-549 |
| 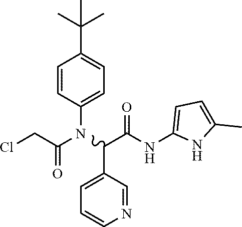 [SMILES: Cc1ccc(NC(=O)C(c2cccnc2)N(C(=O)CCl)c2ccc(C(C)(C)C)cc2)[nH]1] | INSCoV-550 |
| 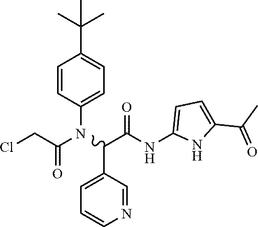 [SMILES: CC(=O)c1ccc(NC(=O)C(c2cccnc2)N(C(=O)CCl)c2ccc(C(C)(C)C)cc2)[nH]1] | INSCoV-551 |
| 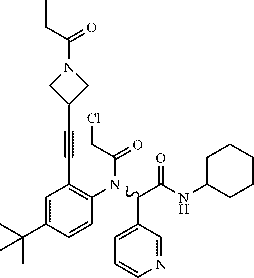 [SMILES: CCC(=O)N1CC(C#Cc2cc(C(C)(C)C)ccc2N(C(=O)CCl)C(C(=O)NC2CCCCC2)c2cccnc2)C1] | INSCoV-552 |
| 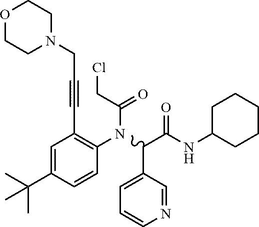 [SMILES: CC(C)(C)c1ccc(N(C(=O)CCl)C(C(=O)NC2CCCCC2)c2cccnc2)c(C#CCN2CCOCC2)c1] | INSCoV-553 |
| 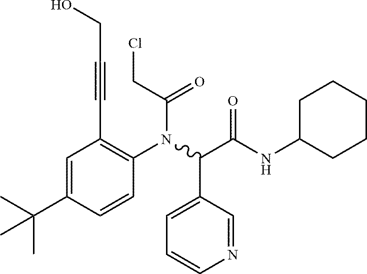 [SMILES: CC(C)(C)c1ccc(N(C(=O)CCl)C(C(=O)NC2CCCCC2)c2cccnc2)c(C#CCO)c1] | INSCoV-554 |
| 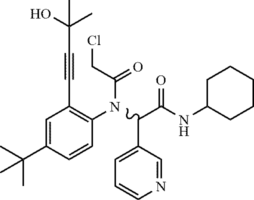 [SMILES: CC(C)(O)C#Cc1cc(C(C)(C)C)ccc1N(C(=O)CCl)C(C(=O)NC1CCCCC1)c1cccnc1] | INSCoV-555 |
| 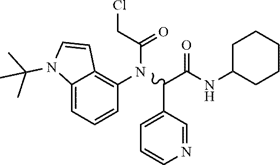 [SMILES: CC(C)(C)n1ccc2c(N(C(=O)CCl)C(C(=O)NC3CCCCC3)c3cccnc3)cccc21] | INSCoV-556 |
| 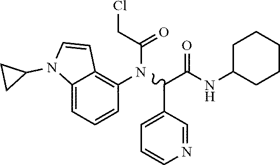 [SMILES: O=C(NC1CCCCC1)C(c1cccnc1)N(C(=O)CCl)c1cccc2c1ccn2C1CC1] | INSCoV-557 |
| 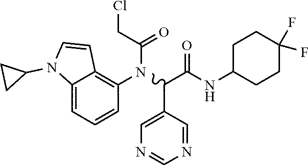 [SMILES: O=C(NC1CCC(F)(F)CC1)C(c1cncnc1)N(C(=O)CCl)c1cccc2c1ccn2C1CC1] | INSCoV-557A |
| 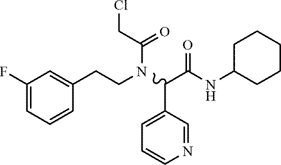 [SMILES: O=C(NC1CCCCC1)C(c1cccnc1)N(CCc1cccc(F)c1)C(=O)CCl] | INSCoV-558 |
| 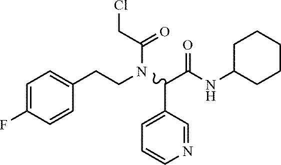 [SMILES: O=C(NC1CCCCC1)C(c1cccnc1)N(CCc1ccc(F)cc1)C(=O)CCl] | INSCoV-559 |
| 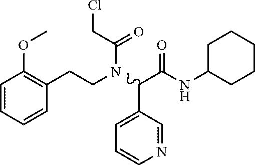 [SMILES: COc1ccccc1CCN(C(=O)CCl)C(C(=O)NC1CCCCC1)c1cccnc1] | INSCoV-560A |
| 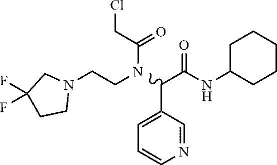 [SMILES: O=C(NC1CCCCC1)C(c1cccnc1)N(CCN1CCC(F)(F)C1)C(=O)CCl] | INSCoV-561 |
| 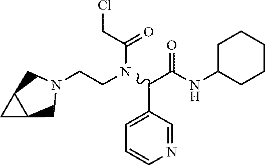 [SMILES: O=C(NC1CCCCC1)C(c1cccnc1)N(CCN1C[C@H]2C[C@H]2C1)C(=O)CCl] | INSCoV-562 |
| 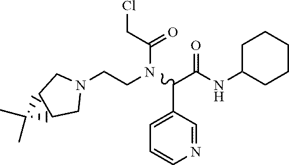 | INSCoV-563 |
| 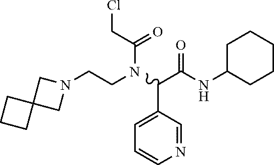 [SMILES: O=C(NC1CCCCC1)C(c1cccnc1)N(CCN1CC2(CCC2)C1)C(=O)CCl] | INSCoV-564 |
| 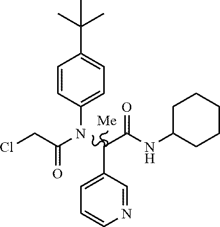 | INSCoV-565 |
| 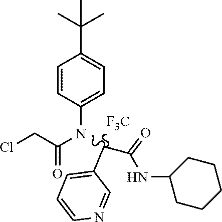 | INSCoV-565A |
| 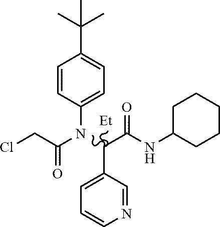 | INSCoV-566 |
| 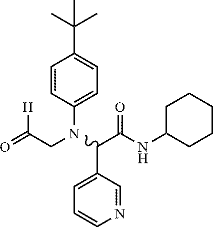 [SMILES: CC(C)(C)c1ccc(N(CC=O)C(C(=O)NC2CCCCC2)c2cccnc2)cc1] | INSCoV-567 |
| 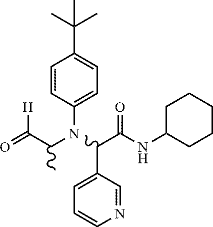 [SMILES: CC(C=O)N(c1ccc(C(C)(C)C)cc1)C(C(=O)NC1CCCCC1)c1cccnc1] | INSCoV-568 |
| 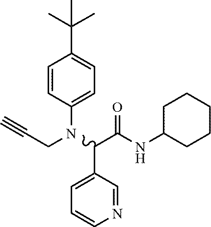 [SMILES: C#CCN(c1ccc(C(C)(C)C)cc1)C(C(=O)NC1CCCCC1)c1cccnc1] | INSCoV-569 |
| 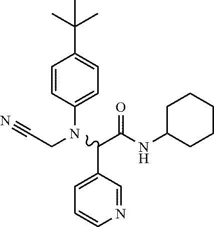 [SMILES: CC(C)(C)c1ccc(N(CC#N)C(C(=O)NC2CCCCC2)c2cccnc2)cc1] | INSCoV-569A |
| 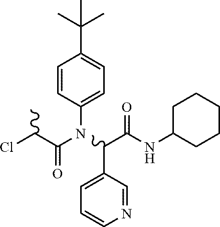 [SMILES: CC(Cl)C(=O)N(c1ccc(C(C)(C)C)cc1)C(C(=O)NC1CCCCC1)c1cccnc1] | INSCoV-570 |
| 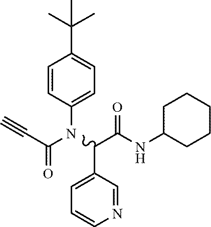 [SMILES: C#CC(=O)N(c1ccc(C(C)(C)C)cc1)C(C(=O)NC1CCCCC1)c1cccnc1] | INSCoV-571 |
| 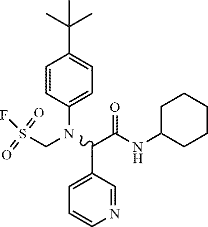 [SMILES: CC(C)(C)c1ccc(N(CS(=O)(=O)F)C(C(=O)NC2CCCCC2)c2cccnc2)cc1] | INSCoV-572 |
| 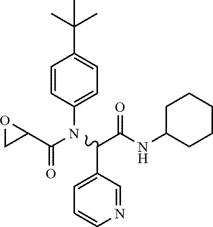 [SMILES: CC(C)(C)c1ccc(N(C(=O)C2CO2)C(C(=O)NC2CCCCC2)c2cccnc2)cc1] | INSCoV-573 |
| 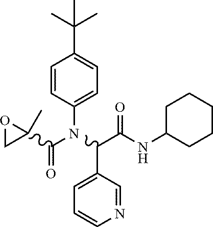 [SMILES: CC1(C(=O)N(c2ccc(C(C)(C)C)cc2)C(C(=O)NC2CCCCC2)c2cccnc2)CO1] | INSCoV-573A |
| 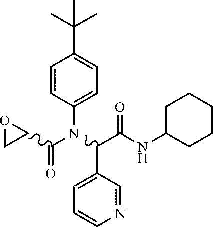 [SMILES: CC(C)(C)c1ccc(N(C(=O)C2CO2)C(C(=O)NC2CCCCC2)c2cccnc2)cc1] | INSCoV-573(2) |
| 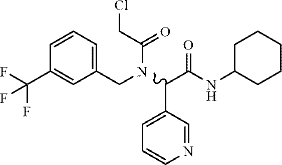 [SMILES: O=C(NC1CCCCC1)C(c1cccnc1)N(Cc1cccc(C(F)(F)F)c1)C(=O)CCl] | INSCoV-574 |
| 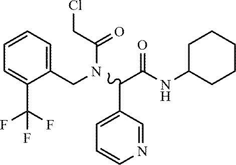 [SMILES: O=C(NC1CCCCC1)C(c1cccnc1)N(Cc1ccccc1C(F)(F)F)C(=O)CCl] | INSCoV-574A |
| 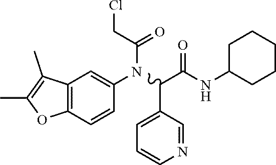 [SMILES: Cc1oc2ccc(N(C(=O)CCl)C(C(=O)NC3CCCCC3)c3cccnc3)cc2c1C] | INSCoV-575 |
| 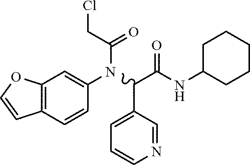 [SMILES: O=C(NC1CCCCC1)C(c1cccnc1)N(C(=O)CCl)c1ccc2ccoc2c1] | INSCoV-576 |
| 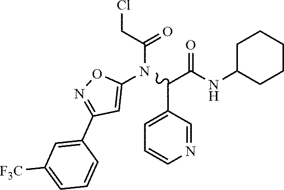 [SMILES: O=C(NC1CCCCC1)C(c1cccnc1)N(C(=O)CCl)c1cc(-c2cccc(C(F)(F)F)c2)no1] | INSCoV-579 |
| 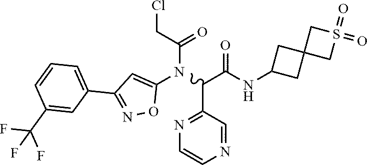 [SMILES: O=C(NC1CC2(C1)CS(=O)(=O)C2)C(c1cnccn1)N(C(=O)CCl)c1cc(-c2cccc(C(F)(F)F)c2)no1] | INSCoV-579A |
| 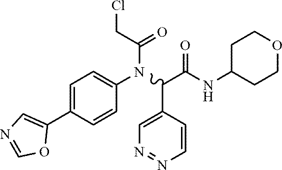 [SMILES: O=C(NC1CCOCC1)C(c1ccnnc1)N(C(=O)CCl)c1ccc(-c2cnco2)cc1] | INSCoV-600A |
| 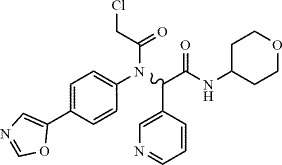 [SMILES: O=C(NC1CCOCC1)C(c1cccnc1)N(C(=O)CCl)c1ccc(-c2cnco2)cc1] | INSCoV-600A(1) |
| 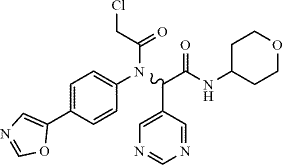 [SMILES: O=C(NC1CCOCC1)C(c1cncnc1)N(C(=O)CCl)c1ccc(-c2cnco2)cc1] | INSCoV-600A(2) |
| 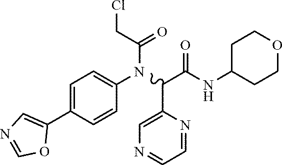 [SMILES: O=C(NC1CCOCC1)C(c1cnccn1)N(C(=O)CCl)c1ccc(-c2cnco2)cc1] | INSCoV-600A(3) |
| 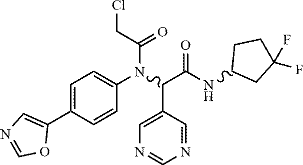 [SMILES: O=C(NC1CCC(F)(F)C1)C(c1cncnc1)N(C(=O)CCl)c1ccc(-c2cnco2)cc1] | INSCoV-600B |
| 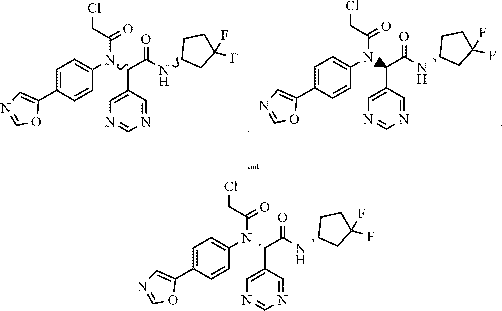 | INSCoV-600B(1), INSCoV-600B(1A), and INSCoV-600B(1B) |
| 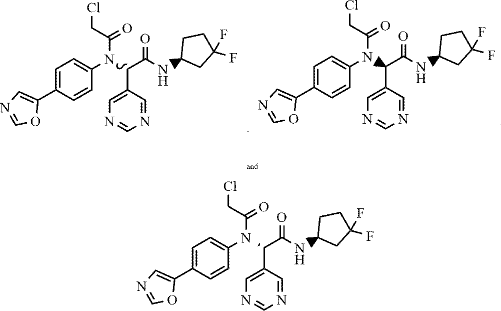 | INSCoV-600B(2), INSCoV-600B(2A), and NSCoV-600B(2B) |
| 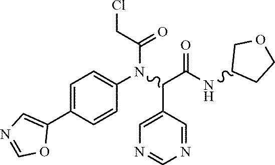 [SMILES: O=C(NC1CCOC1)C(c1cncnc1)N(C(=O)CCl)c1ccc(-c2cnco2)cc1] | INSCoV-600C |
| 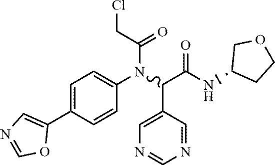 [SMILES: O=C(N[C@H]1CCOC1)C(c1cncnc1)N(C(=O)CCl)c1ccc(-c2cnco2)cc1] | INSCoV-600C(1) |
| 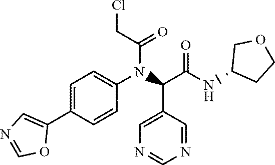 [SMILES: O=C(N[C@H]1CCOC1)[C@@H](c1cncnc1)N(C(=O)CCl)c1ccc(-c2cnco2)cc1] | INSCoV-600C(1A) |
| 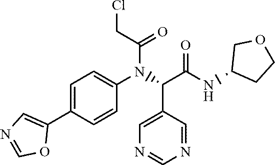 [SMILES: O=C(N[C@H]1CCOC1)[C@H](c1cncnc1)N(C(=O)CCl)c1ccc(-c2cnco2)cc1] | INSCoV-600C(1B) |
| 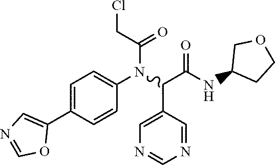 [SMILES: O=C(N[C@@H]1CCOC1)C(c1cncnc1)N(C(=O)CCl)c1ccc(-c2cnco2)cc1] | INSCoV-600C(2) |
| 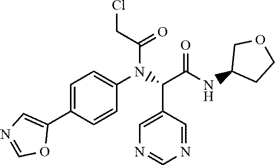 [SMILES: O=C(N[C@@H]1CCOC1)[C@H](c1cncnc1)N(C(=O)CCl)c1ccc(-c2cnco2)cc1] | INSCoV-600C(2A) |
| 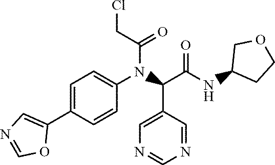 [SMILES: O=C(N[C@@H]1CCOC1)[C@@H](c1cncnc1)N(C(=O)CCl)c1ccc(-c2cnco2)cc1] | INSCoV-600C(2B) |
| 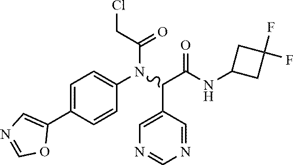 [SMILES: O=C(NC1CC(F)(F)C1)C(c1cncnc1)N(C(=O)CCl)c1ccc(-c2cnco2)cc1] | INSCoV-600D |
| 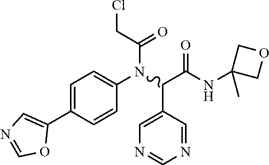 [SMILES: CC1(NC(=O)C(c2cncnc2)N(C(=O)CCl)c2ccc(-c3cnco3)cc2)COC1] | INSCoV-600E |
| 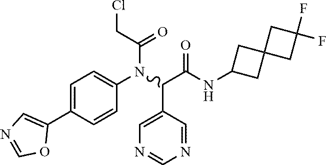 [SMILES: O=C(NC1CC2(C1)CC(F)(F)C2)C(c1cncnc1)N(C(=O)CCl)c1ccc(-c2cnco2)cc1] | INSCoV-600F |
| 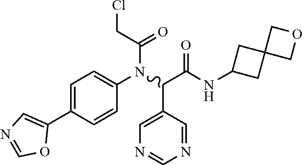 [SMILES: O=C(NC1CC2(COC2)C1)C(c1cncnc1)N(C(=O)CCl)c1ccc(-c2cnco2)cc1] | INSCoV-600G |
| 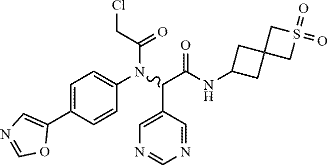 [SMILES: O=C(NC1CC2(C1)CS(=O)(=O)C2)C(c1cncnc1)N(C(=O)CCl)c1ccc(-c2cnco2)cc1] | INSCoV-600H |
| 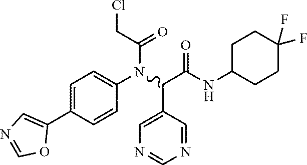 [SMILES: O=C(NC1CCC(F)(F)CC1)C(c1cncnc1)N(C(=O)CCl)c1ccc(-c2cnco2)cc1] | INSCoV-600I |
| 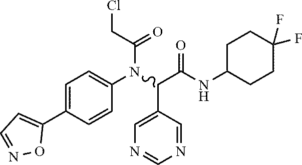 [SMILES: O=C(NC1CCC(F)(F)CC1)C(c1cncnc1)N(C(=O)CCl)c1ccc(-c2ccno2)cc1] | INSCoV-600J |
| 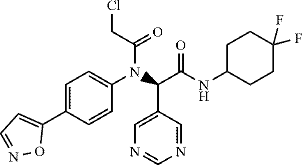 [SMILES: O=C(NC1CCC(F)(F)CC1)[C@@H](c1cncnc1)N(C(=O)CCl)c1ccc(-c2ccno2)cc1] | INSCoV-600-J(1) |
| 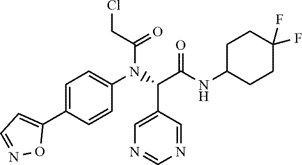 [SMILES: O=C(NC1CCC(F)(F)CC1)[C@H](c1cncnc1)N(C(=O)CCl)c1ccc(-c2ccno2)cc1] | INSCoV-600-J(2) |
| 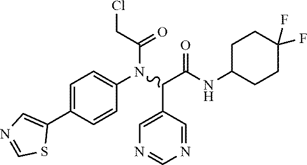 [SMILES: O=C(NC1CCC(F)(F)CC1)C(c1cncnc1)N(C(=O)CCl)c1ccc(-c2cncs2)cc1] | INSCoV-600K |
| 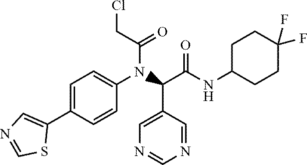 [SMILES: O=C(NC1CCC(F)(F)CC1)[C@@H](c1cncnc1)N(C(=O)CCl)c1ccc(-c2cncs2)cc1] | INSCoV-600-K(1) |
| 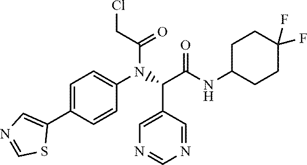 [SMILES: O=C(NC1CCC(F)(F)CC1)[C@H](c1cncnc1)N(C(=O)CCl)c1ccc(-c2cncs2)cc1] | INSCoV-600-K(2) |
| 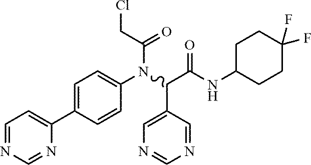 [SMILES: O=C(NC1CCC(F)(F)CC1)C(c1cncnc1)N(C(=O)CCl)c1ccc(-c2ccncn2)cc1] | INSCoV-600L |
| 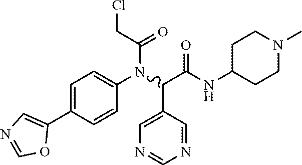 [SMILES: CN1CCC(NC(=O)C(c2cncnc2)N(C(=O)CCl)c2ccc(-c3cnco3)cc2)CC1] | INSCoV-600M |
| 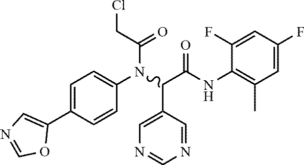 [SMILES: Cc1cc(F)cc(F)c1NC(=O)C(c1cncnc1)N(C(=O)CCl)c1ccc(-c2cnco2)cc1] | INSCoV-600N |
| 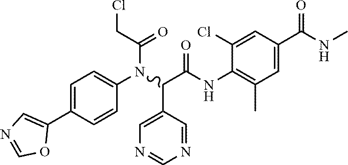 [SMILES: CNC(=O)c1cc(C)c(NC(=O)C(c2cncnc2)N(C(=O)CCl)c2ccc(-c3cnco3)cc2)c(Cl)c1] | INSCoV-600O |
| 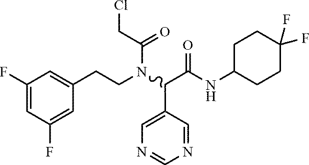 [SMILES: O=C(NC1CCC(F)(F)CC1)C(c1cncnc1)N(CCc1cc(F)cc(F)c1)C(=O)CCl] | INSCoV-600P |
| 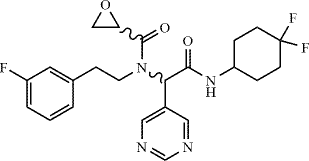 [SMILES: O=C(NC1CCC(F)(F)CC1)C(c1cncnc1)N(CCc1cccc(F)c1)C(=O)C1CO1] | INSCoV-600Q |
| 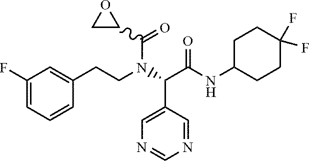 [SMILES: O=C(NC1CCC(F)(F)CC1)[C@H](c1cncnc1)N(CCc1cccc(F)c1)C(=O)C1CO1] | INSCoV-600Q(1) |
| 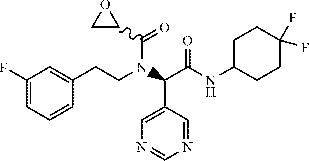 [SMILES: O=C(NC1CCC(F)(F)CC1)[C@@H](c1cncnc1)N(CCc1cccc(F)c1)C(=O)C1CO1] | INSCoV-600Q(2) |
| 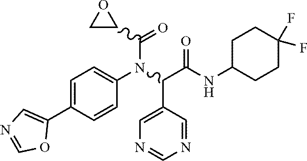 [SMILES: O=C(NC1CCC(F)(F)CC1)C(c1cncnc1)N(C(=O)C1CO1)c1ccc(-c2cnco2)cc1] | INSCoV-600R |
| 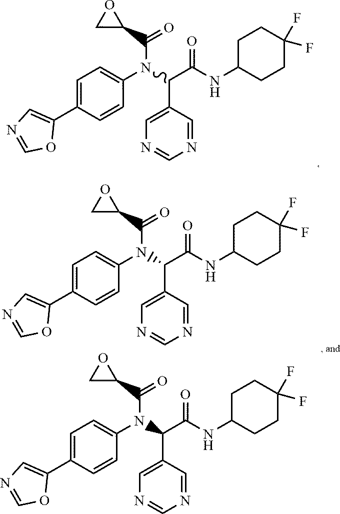 | INSCoV-600R(1), INSCoV-600R(1A) and INSCoV-600R(1B) |
| 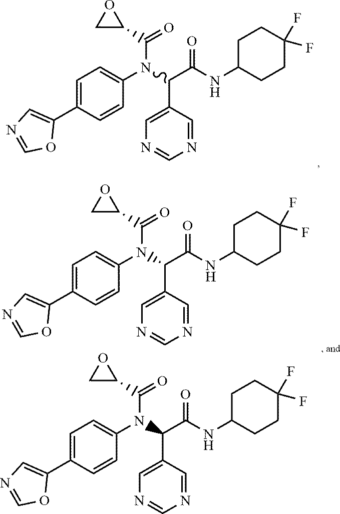 | INSCoV-600R(2), INSCoV-600R(2A), and INSCoV-600R(2B) |
| 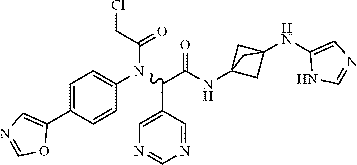 [SMILES: O=C(NC12CC(Nc3cnc[nH]3)(C1)C2)C(c1cncnc1)N(C(=O)CCl)c1ccc(-c2cnco2)cc1] | INSCoV-600S |
| 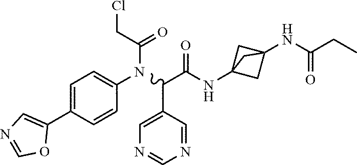 [SMILES: CCC(=O)NC12CC(NC(=O)C(c3cncnc3)N(C(=O)CCl)c3ccc(-c4cnco4)cc3)(C1)C2] | INSCoV-600T |
| 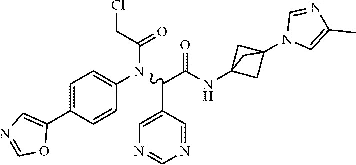 [SMILES: Cc1cn(C23CC(NC(=O)C(c4cncnc4)N(C(=O)CCl)c4ccc(-c5cnco5)cc4)(C2)C3)cn1] | INSCoV-600U |
| 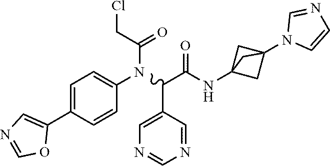 [SMILES: O=C(NC12CC(n3ccnc3)(C1)C2)C(c1cncnc1)N(C(=O)CCl)c1ccc(-c2cnco2)cc1] | INSCoV-600V |
| 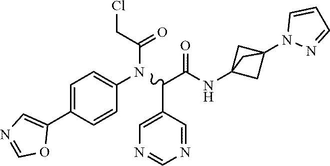 [SMILES: O=C(NC12CC(n3cccn3)(C1)C2)C(c1cncnc1)N(C(=O)CCl)c1ccc(-c2cnco2)cc1] | INSCoV-600W |
| 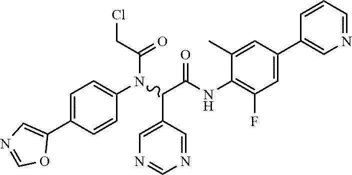 [SMILES: Cc1cc(-c2cccnc2)cc(F)c1NC(=O)C(c1cncnc1)N(C(=O)CCl)c1ccc(-c2cnco2)cc1] | INSCoV-600X |
| 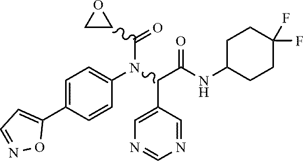 [SMILES: O=C(NC1CCC(F)(F)CC1)C(c1cncnc1)N(C(=O)C1CO1)c1ccc(-c2ccno2)cc1] | INSCoV-600Y |
| 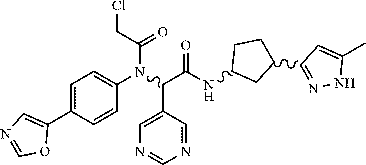 [SMILES: Cc1cc(C2CCC(NC(=O)C(c3cncnc3)N(C(=O)CCl)c3ccc(-c4cnco4)cc3)C2)n[nH]1] | INSCoV-601A |
| 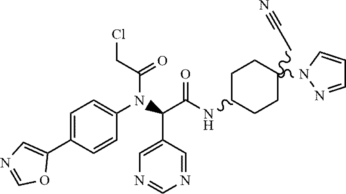 | INSCoV-601B |
| 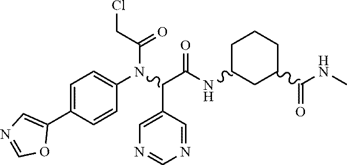 [SMILES: CNC(=O)C1CCCC(NC(=O)C(c2cncnc2)N(C(=O)CCl)c2ccc(-c3cnco3)cc2)C1] | INSCoV-601C |
| 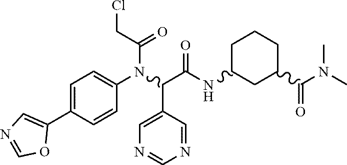 [SMILES: CN(C)C(=O)C1CCCC(NC(=O)C(c2cncnc2)N(C(=O)CCl)c2ccc(-c3cnco3)cc2)C1] | INSCoV-601C-A |
| 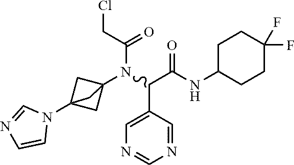 [SMILES: O=C(NC1CCC(F)(F)CC1)C(c1cncnc1)N(C(=O)CCl)C12CC(n3ccnc3)(C1)C2] | INSCoV-601D |
| 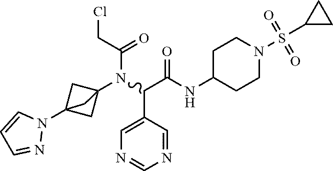 [SMILES: O=C(NC1CCN(S(=O)(=O)C2CC2)CC1)C(c1cncnc1)N(C(=O)CCl)C12CC(n3cccn3)(C1)C2] | INSCoV-601E |
| 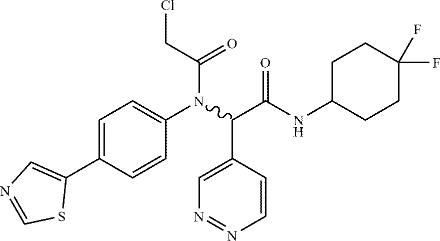 [SMILES: O=C(NC1CCC(F)(F)CC1)C(c1ccnnc1)N(C(=O)CCl)c1ccc(-c2cncs2)cc1] | INSCoV-601F |
| 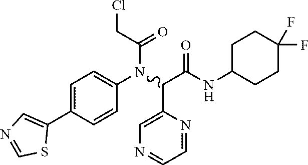 [SMILES: O=C(NC1CCC(F)(F)CC1)C(c1cnccn1)N(C(=O)CCl)c1ccc(-c2cncs2)cc1] | INSCoV-601G |
| 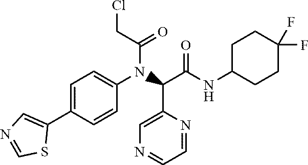 [SMILES: O=C(NC1CCC(F)(F)CC1)[C@@H](c1cnccn1)N(C(=O)CCl)c1ccc(-c2cncs2)cc1] | INSCoV-601G(1) |
| 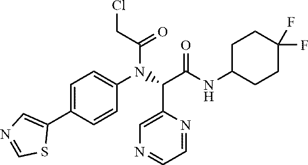 [SMILES: O=C(NC1CCC(F)(F)CC1)[C@H](c1cnccn1)N(C(=O)CCl)c1ccc(-c2cncs2)cc1] | INSCoV-601G(2) |
| 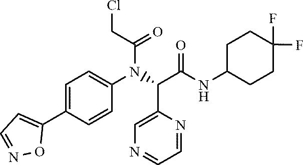 [SMILES: O=C(NC1CCC(F)(F)CC1)[C@H](c1cnccn1)N(C(=O)CCl)c1ccc(-c2ccno2)cc1] | INSCoV-601H |
| 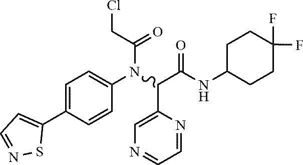 [SMILES: O=C(NC1CCC(F)(F)CC1)C(c1cnccn1)N(C(=O)CCl)c1ccc(-c2ccns2)cc1] | INSCoV-601I |
| 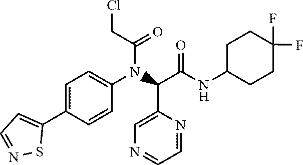 [SMILES: O=C(NC1CCC(F)(F)CC1)[C@@H](c1cnccn1)N(C(=O)CCl)c1ccc(-c2ccns2)cc1] | INSCoV-601I(1) |
| 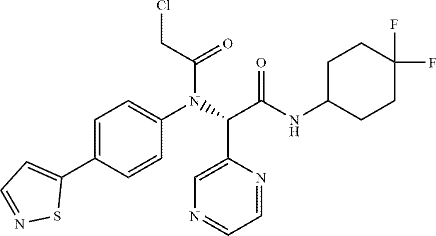 [SMILES: O=C(NC1CCC(F)(F)CC1)[C@H](c1cnccn1)N(C(=O)CCl)c1ccc(-c2ccns2)cc1] | INSCoV-601I(2) |
| 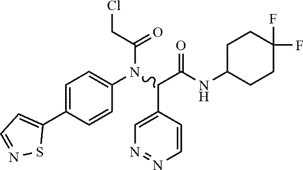 [SMILES: O=C(NC1CCC(F)(F)CC1)C(c1ccnnc1)N(C(=O)CCl)c1ccc(-c2ccns2)cc1] | INSCoV-601J |
| 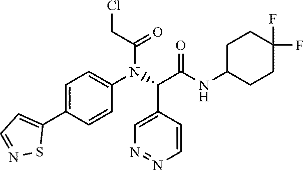 [SMILES: O=C(NC1CCC(F)(F)CC1)[C@H](c1ccnnc1)N(C(=O)CCl)c1ccc(-c2ccns2)cc1] | INSCoV-601-J(2) |
| 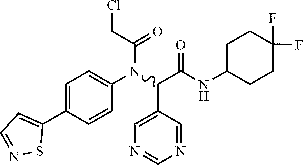 [SMILES: O=C(NC1CCC(F)(F)CC1)C(c1cncnc1)N(C(=O)CCl)c1ccc(-c2ccns2)cc1] | INSCoV-601K |
| 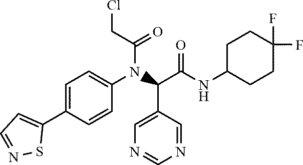 [SMILES: O=C(NC1CCC(F)(F)CC1)[C@@H](c1cncnc1)N(C(=O)CCl)c1ccc(-c2ccns2)cc1] | INSCoV-601K(1), |
| 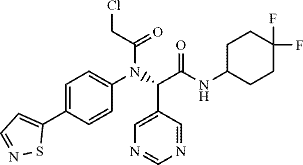 [SMILES: O=C(NC1CCC(F)(F)CC1)[C@H](c1cncnc1)N(C(=O)CCl)c1ccc(-c2ccns2)cc1] | INSCoV-601K(2) |
| 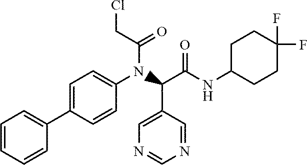 [SMILES: O=C(NC1CCC(F)(F)CC1)[C@@H](c1cncnc1)N(C(=O)CCl)c1ccc(-c2ccccc2)cc1] | INSCoV-601L |
| 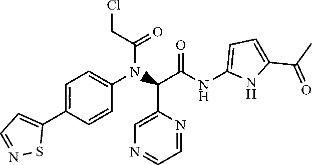 [SMILES: CC(=O)c1ccc(NC(=O)[C@@H](c2cnccn2)N(C(=O)CCl)c2ccc(-c3ccns3)cc2)[nH]1] | INSCoV-601M |
| 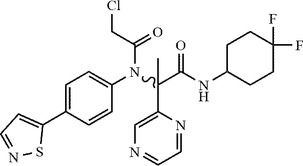 [SMILES: CC(C(=O)NC1CCC(F)(F)CC1)(c1cnccn1)N(C(=O)CCl)c1ccc(-c2ccns2)cc1] | INSCoV-601N |
| 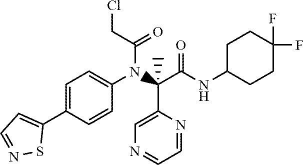 [SMILES: C[C@](C(=O)NC1CCC(F)(F)CC1)(c1cnccn1)N(C(=O)CCl)c1ccc(-c2ccns2)cc1] | INSCoV-601N(1) |
| 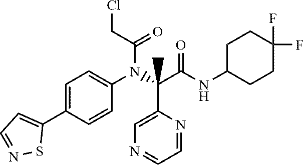 [SMILES: C[C@@](C(=O)NC1CCC(F)(F)CC1)(c1cnccn1)N(C(=O)CCl)c1ccc(-c2ccns2)cc1] | INSCoV-601N(2) |
| 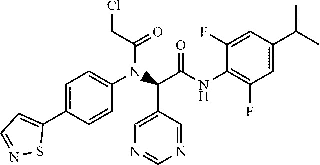 [SMILES: CC(C)c1cc(F)c(NC(=O)[C@@H](c2cncnc2)N(C(=O)CCl)c2ccc(-c3ccns3)cc2)c(F)c1] | INSCoV-601O |
| 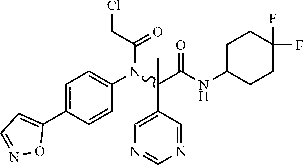 [SMILES: CC(C(=O)NC1CCC(F)(F)CC1)(c1cncnc1)N(C(=O)CCl)c1ccc(-c2ccno2)cc1] | INSCoV-601P |
| 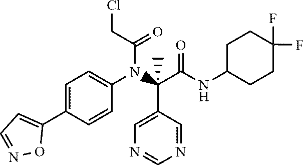 [SMILES: C[C@](C(=O)NC1CCC(F)(F)CC1)(c1cncnc1)N(C(=O)CCl)c1ccc(-c2ccno2)cc1] | INSCoV-601P(lA) |
| 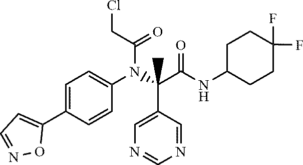 [SMILES: C[C@@](C(=O)NC1CCC(F)(F)CC1)(c1cncnc1)N(C(=O)CCl)c1ccc(-c2ccno2)cc1] | INSCoV-601P(1B) |
| 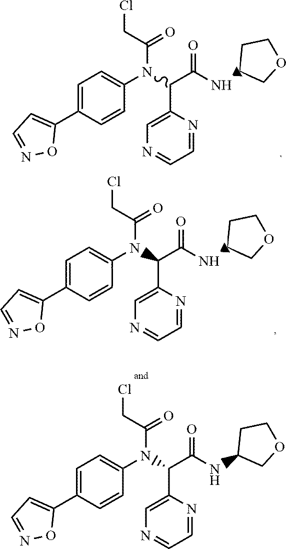 | INSCoV-601Q, INSCoV-601Q(1A) and INSCoV-601Q(1B) |
| 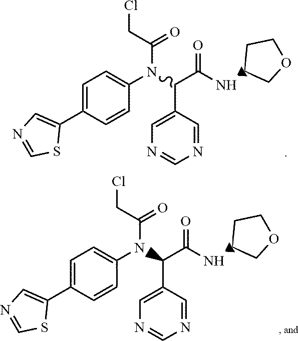 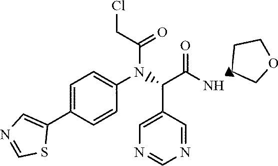 | INSCoV-601R, INSCoV-601R(1A), INSCoV-601R(1B) |
| 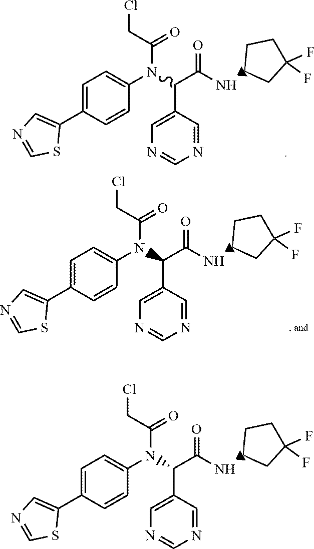 | INSCoV-601S, INS-601S(1A), and INSCoV-601S(1B) |
| 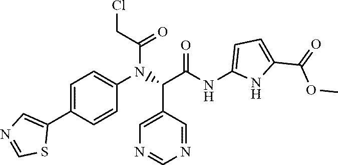 [SMILES: COC(=O)c1ccc(NC(=O)[C@H](c2cncnc2)N(C(=O)CCl)c2ccc(-c3cncs3)cc2)[nH]1] | INSCoV-601T |
| 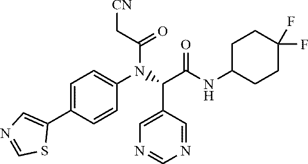 [SMILES: N#CCC(=O)N(c1ccc(-c2cncs2)cc1)[C@H](C(=O)NC1CCC(F)(F)CC1)c1cncnc1] | INSCoV-612 |
| 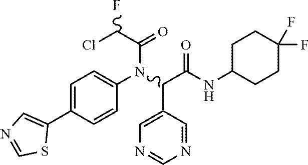 [SMILES: O=C(NC1CCC(F)(F)CC1)C(c1cncnc1)N(C(=O)C(F)Cl)c1ccc(-c2cncs2)cc1] | INSCoV-614 |
| 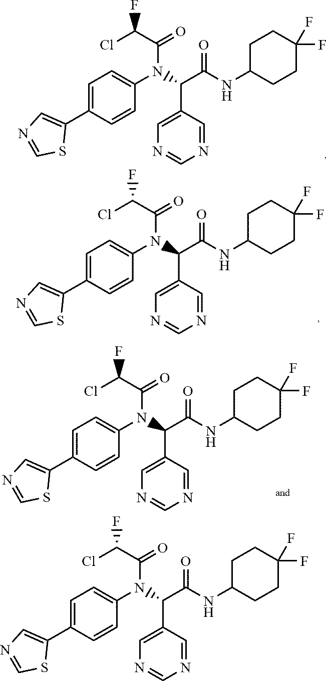 | INSCoV-614(1A), INSCoV-614(1B), INSCoV-614(2A), and INSCoV-614(2B) |
| 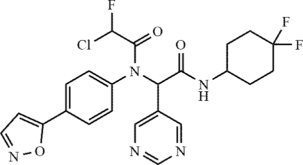 [SMILES: O=C(NC1CCC(F)(F)CC1)C(c1cncnc1)N(C(=O)C(F)Cl)c1ccc(-c2ccno2)cc1] | INSCoV-614A |
| 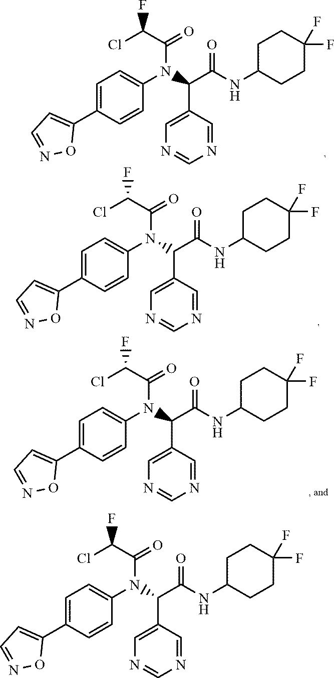 | INSCoV-614A(1A), INSCoV-614A(1B), INSCoV-614A(2A) and INSCoV-614A(2B) |
| 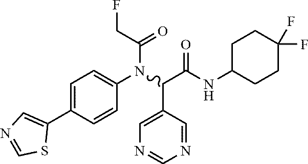 [SMILES: O=C(NC1CCC(F)(F)CC1)C(c1cncnc1)N(C(=O)CF)c1ccc(-c2cncs2)cc1] | INSCoV-615 |
| 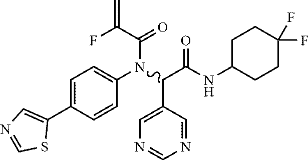 [SMILES: C=C(F)C(=O)N(c1ccc(-c2cncs2)cc1)C(C(=O)NC1CCC(F)(F)CC1)c1cncnc1] | INSCoV-616 |
| 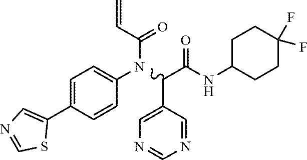 [SMILES: C=CC(=O)N(c1ccc(-c2cncs2)cc1)C(C(=O)NC1CCC(F)(F)CC1)c1cncnc1] | INSCoV-618 |
| 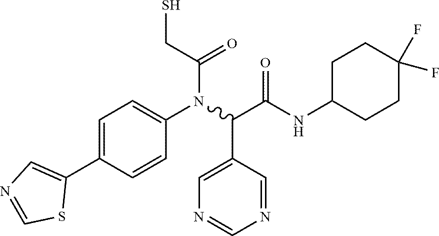 [SMILES: O=C(NC1CCC(F)(F)CC1)C(c1cncnc1)N(C(=O)CS)c1ccc(-c2cncs2)cc1] | INSCoV-620 |
| 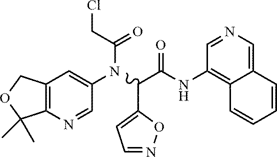 [SMILES: CC1(C)OCc2cc(N(C(=O)CCl)C(C(=O)Nc3cncc4ccccc34)c3ccno3)cnc21] | INSCoV-704 |
| 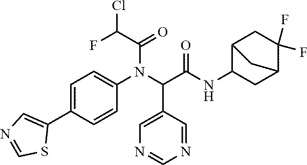 [SMILES: O=C(NC1CC2CC1CC2(F)F)C(c1cncnc1)N(C(=O)C(F)Cl)c1ccc(-c2cncs2)cc1] | INSCoV-901 |
| 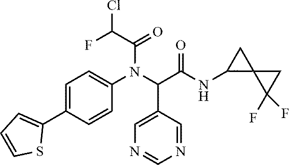 [SMILES: O=C(NC1CC12CC2(F)F)C(c1cncnc1)N(C(=O)C(F)Cl)c1ccc(-c2cccs2)cc1] | INSCoV-902 |
| 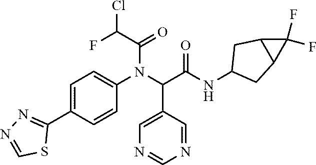 [SMILES: O=C(NC1CC2C(C1)C2(F)F)C(c1cncnc1)N(C(=O)C(F)Cl)c1ccc(-c2nncs2)cc1] | INSCoV-903 |
| 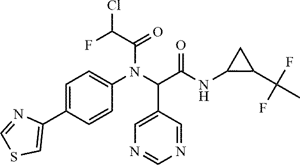 [SMILES: CC(F)(F)C1CC1NC(=O)C(c1cncnc1)N(C(=O)C(F)Cl)c1ccc(-c2cscn2)cc1] | INSCoV-904 |
| 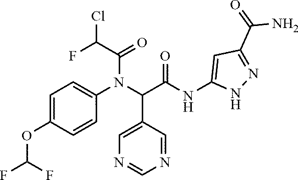 [SMILES: NC(=O)c1cc(NC(=O)C(c2cncnc2)N(C(=O)C(F)Cl)c2ccc(OC(F)F)cc2)[nH]n1] | INSCoV-905 |
| 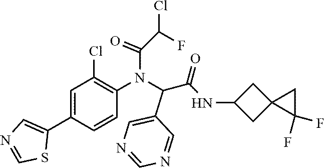 [SMILES: O=C(NC1CC2(C1)CC2(F)F)C(c1cncnc1)N(C(=O)C(F)Cl)c1ccc(-c2cncs2)cc1Cl] | INSCoV-906 |
| 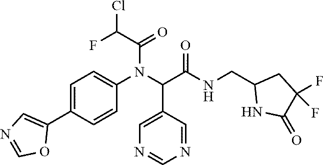 [SMILES: O=C(NCC1CC(F)(F)C(=O)N1)C(c1cncnc1)N(C(=O)C(F)Cl)c1ccc(-c2cnco2)cc1] | INSCoV-907 |
| 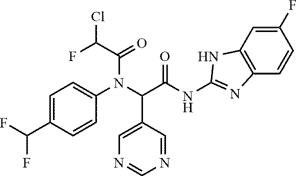 [SMILES: O=C(Nc1nc2ccc(F)cc2[nH]1)C(c1cncnc1)N(C(=O)C(F)Cl)c1ccc(C(F)F)cc1] | INSCoV-908 |
| 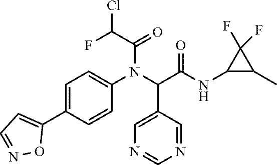 [SMILES: CC1C(NC(=O)C(c2cncnc2)N(C(=O)C(F)Cl)c2ccc(-c3ccno3)cc2)C1(F)F] | INSCoV-909 |
| 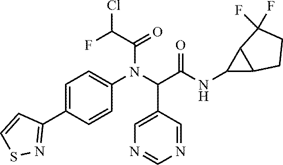 [SMILES: O=C(NC1C2CCC(F)(F)C21)C(c1cncnc1)N(C(=O)C(F)Cl)c1ccc(-c2ccsn2)cc1] | INSCoV-910 |
| 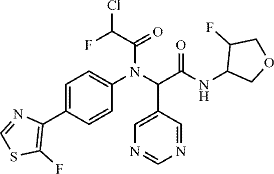 [SMILES: O=C(NC1COCC1F)C(c1cncnc1)N(C(=O)C(F)Cl)c1ccc(-c2ncsc2F)cc1] | INSCoV-911 |
| 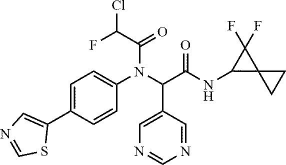 [SMILES: O=C(NC1C(F)(F)C12CC2)C(c1cncnc1)N(C(=O)C(F)Cl)c1ccc(-c2cncs2)cc1] | INSCoV-912 |
| 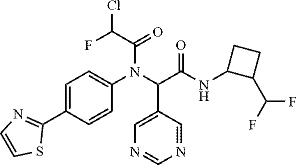 [SMILES: O=C(NC1CCC1C(F)F)C(c1cncnc1)N(C(=O)C(F)Cl)c1ccc(-c2nccs2)cc1] | INSCoV-913 |
| 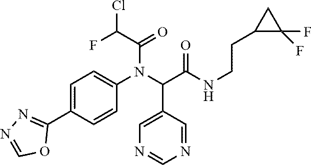 [SMILES: O=C(NCCC1CC1(F)F)C(c1cncnc1)N(C(=O)C(F)Cl)c1ccc(-c2nnco2)cc1] | INSCoV-914 |
| 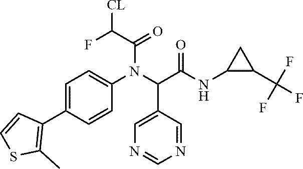 | INSCoV-915 |
| 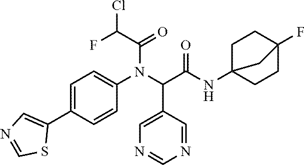 [SMILES: O=C(NC12CCC(F)(CC1)C2)C(c1cncnc1)N(C(=O)C(F)Cl)c1ccc(-c2cncs2)cc1] | INSCoV-916 |
| 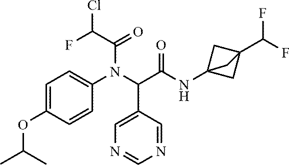 [SMILES: CC(C)Oc1ccc(N(C(=O)C(F)Cl)C(C(=O)NC23CC(C(F)F)(C2)C3)c2cncnc2)cc1] | INSCoV-917 |
| 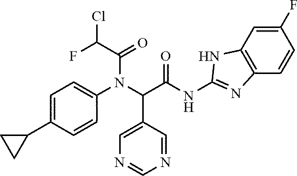 [SMILES: O=C(Nc1nc2ccc(F)cc2[nH]1)C(c1cncnc1)N(C(=O)C(F)Cl)c1ccc(C2CC2)cc1] | INSCoV-918 |
| 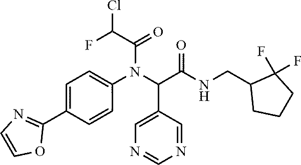 [SMILES: O=C(NCC1CCCC1(F)F)C(c1cncnc1)N(C(=O)C(F)Cl)c1ccc(-c2ncco2)cc1] | INSCoV-919 |
| 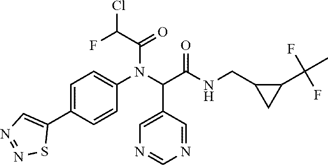 [SMILES: CC(F)(F)C1CC1CNC(=O)C(c1cncnc1)N(C(=O)C(F)Cl)c1ccc(-c2cnns2)cc1] | INSCoV-920 |
| 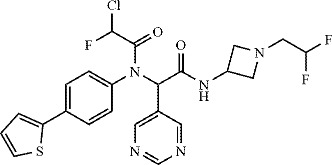 [SMILES: O=C(NC1CN(CC(F)F)C1)C(c1cncnc1)N(C(=O)C(F)Cl)c1ccc(-c2cccs2)cc1] | INSCoV-921 |
| 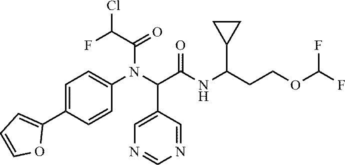 [SMILES: O=C(NC(CCOC(F)F)C1CC1)C(c1cncnc1)N(C(=O)C(F)Cl)c1ccc(-c2ccco2)cc1] | INSCoV-922 |
| 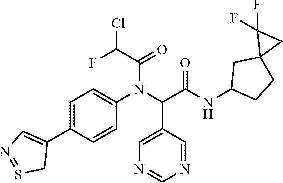 | INSCoV-923 |
| 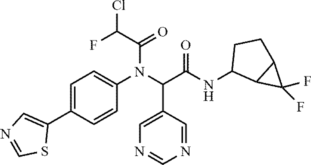 [SMILES: O=C(NC1CCC2C1C2(F)F)C(c1cncnc1)N(C(=O)C(F)Cl)c1ccc(-c2cncs2)cc1] | INSCoV-924 |
| 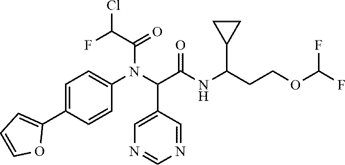 [SMILES: O=C(NC(CCOC(F)F)C1CC1)C(c1cncnc1)N(C(=O)C(F)Cl)c1ccc(-c2ccco2)cc1] | INSCoV-925 |
| 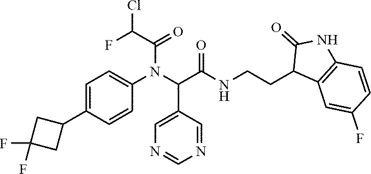 [SMILES: O=C1Nc2ccc(F)cc2C1CCNC(=O)C(c1cncnc1)N(C(=O)C(F)Cl)c1ccc(C2CC(F)(F)C2)cc1] | INSCoV-926 |
| 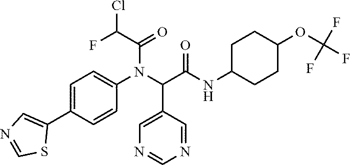 [SMILES: O=C(NC1CCC(OC(F)(F)F)CC1)C(c1cncnc1)N(C(=O)C(F)Cl)c1ccc(-c2cncs2)cc1] | INSCoV-927 |
| 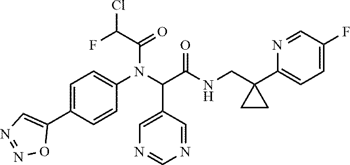 [SMILES: O=C(NCC1(c2ccc(F)cn2)CC1)C(c1cncnc1)N(C(=O)C(F)Cl)c1ccc(-c2cnno2)cc1] | INSCoV-928 |
| 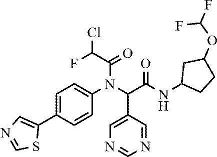 [SMILES: O=C(NC1CCC(OC(F)F)C1)C(c1cncnc1)N(C(=O)C(F)Cl)c1ccc(-c2cncs2)cc1] | INSCoV-929 |
| 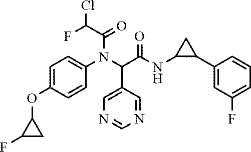 [SMILES: O=C(NC1CC1c1cccc(F)c1)C(c1cncnc1)N(C(=O)C(F)Cl)c1ccc(OC2CC2F)cc1] | INSCoV-930 |
| 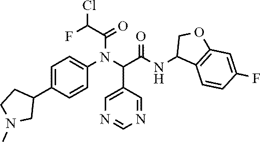 [SMILES: CN1CCC(c2ccc(N(C(=O)C(F)Cl)C(C(=O)NC3COc4cc(F)ccc43)c3cncnc3)cc2)C1] | INSCoV-931 |
| 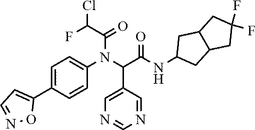 [SMILES: O=C(NC1CC2CC(F)(F)CC2C1)C(c1cncnc1)N(C(=O)C(F)Cl)c1ccc(-c2ccno2)cc1] | INSCoV-932 |
| 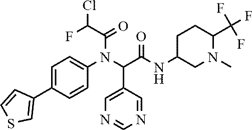 [SMILES: CN1CC(NC(=O)C(c2cncnc2)N(C(=O)C(F)Cl)c2ccc(-c3ccsc3)cc2)CCC1C(F)(F)F] | INSCoV-933 |
| 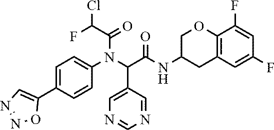 [SMILES: O=C(NC1COc2c(F)cc(F)cc2C1)C(c1cncnc1)N(C(=O)C(F)Cl)c1ccc(-c2cnno2)cc1] | INSCoV-934 |
| 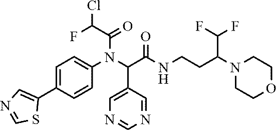 [SMILES: O=C(NCCC(C(F)F)N1CCOCC1)C(c1cncnc1)N(C(=O)C(F)Cl)c1ccc(-c2cncs2)cc1] | INSCoV-935 |
| 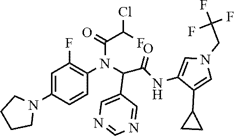 [SMILES: O=C(Nc1cn(CC(F)(F)F)cc1C1CC1)C(c1cncnc1)N(C(=O)C(F)Cl)c1ccc(N2CCCC2)cc1F] | INSCoV-936 |
| 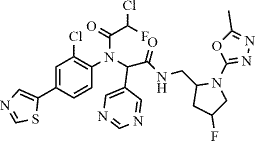 [SMILES: Cc1nnc(N2CC(F)CC2CNC(=O)C(c2cncnc2)N(C(=O)C(F)Cl)c2ccc(-c3cncs3)cc2Cl)o1] | INSCoV-937 |
| 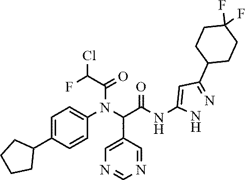 [SMILES: O=C(Nc1cc(C2CCC(F)(F)CC2)n[nH]1)C(c1cncnc1)N(C(=O)C(F)Cl)c1ccc(C2CCCC2)cc1] | INSCoV-938 |
| 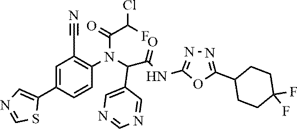 [SMILES: N#Cc1cc(-c2cncs2)ccc1N(C(=O)C(F)Cl)C(C(=O)Nc1nnc(C2CCC(F)(F)CC2)o1)c1cncnc1] | INSCoV-939 |
| 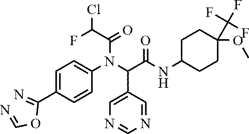 [SMILES: COC1(C(F)(F)F)CCC(NC(=O)C(c2cncnc2)N(C(=O)C(F)Cl)c2ccc(-c3nnco3)cc2)CC1] | INSCoV-940 |

### Further Forms of Compounds

In another aspect, the compounds described herein, possesses one or more stereocenters and each stereocenter exists independently in either the R or S configuration. The compounds presented herein include all diastereomeric, enantiomeric, and epimeric forms as well as the appropriate mixtures thereof. The compounds and methods provided herein include all cis, trans, syn, anti, entgegen (E), and zusammen (Z) isomers as well as the appropriate mixtures thereof. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of steroisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In one aspect, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. In some embodiments, the design of a prodrug increases the effective water solubility. An example, without limitation, of a prodrug is a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

In one aspect, prodrugs are designed to alter the metabolic stability or the transport characteristics of a drug, to mask side effects or toxicity, to improve the flavor of a drug or to alter other characteristics or properties of a drug. By virtue of knowledge of pharmacokinetic, pharmacodynamic processes and drug metabolism in vivo, once a pharmaceutically active compound is known, the design prodrugs of the compound is possible. (see, for example, Nogrady (1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401, Rooseboom et al., Pharmacological Reviews, 56:53-102, 2004; Aesop Cho, “Recent Advances in Oral Prodrug Discovery”, Annual Reports in Medicinal Chemistry, Vol. 41, 395-407, 2006; T. Higuchi and V. Stella, Pro-drugs as Novel Delivery Systems, Vol. 14 of the A.C.S. Symposium Series).

In some cases, some of the herein-described compounds may be a prodrug for another derivative or active compound.

In some embodiments, sites on the aromatic ring portion of compounds described herein are susceptible to various metabolic reactions Therefore incorporation of appropriate substituents on the aromatic ring structures will reduce, minimize or eliminate this metabolic pathway. In specific embodiments, the appropriate substituent to decrease or eliminate the susceptibility of the aromatic ring to metabolic reactions is, by way of example only, a halogen, or an alkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine and chlorine, such as, for example, ²H, ³H, ¹³C, ¹⁴C, ¹⁵N, ¹⁸O, ¹⁷O, ³⁵S, ¹⁸F, and ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In certain embodiments, the abundance of ²H atoms in the compounds disclosed herein is enriched for some or all of the ¹H atoms. In some embodiments, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In some embodiments, the level of deuterium incorporation is determined by synthetic methods in which a deuterated synthetic building block is used as a starting material.

In additional or further embodiments, the compounds described herein are metabolized upon administration to an organism in need to produce a metabolite that is then used to produce a desired effect, including a desired therapeutic effect.

“Pharmaceutically acceptable” as used herein, refers a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutically acceptable salt” refers to a formulation of a compound that does not cause significant irritation to an organism to which it is administered and does not abrogate the biological activity and properties of the compound. In some embodiments, pharmaceutically acceptable salts are obtained by reacting the compounds described herein with an acid. Pharmaceutically acceptable salts are also obtained by reacting the compound described herein with a base to form a salt.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g., lithium, sodium, or potassium), an alkaline earth ion (e.g., magnesium or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

Examples of pharmaceutically acceptable salts include those salts prepared by reaction of the compounds described herein with a mineral acid, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid, metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts, and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

It should be understood that a reference to a pharmaceutically acceptable salt includes the solvent addition forms, particularly solvates. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

In some embodiments, the compounds described herein exist as solvates. This disclosure provides for methods of treating diseases by administering such solvates. This disclosure further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein. Accordingly, one aspect of the present disclosure pertains to hydrates and solvates of compounds of the present disclosure and/or their pharmaceutical acceptable salts, as described herein, that can be isolated and characterized by methods known in the art, such as, thermogravimetric analysis (TGA), TGA-mass spectroscopy, TGA-Infrared spectroscopy, powder X-ray diffraction (PXRD), Karl Fisher titration, high resolution X-ray diffraction, and the like.

### Methods of Treatment

In another aspect, provided herein is a method of treating or preventing a SARS-CoV-2 infection in a patient in need thereof, comprising administering to the patient a compound or a pharmaceutical composition comprising a compound described herein, for example, a compound of Formulas A*, A, X, IX, XI, XII, I, II, III, or IV.

In some embodiments, the compound disclosed herein is administered to the subject prophylactically. In some embodiments, the subject is suspected of having a SARS-CoV-2 infection before the SARS-CoV-2 infection is diagnosed.

In some embodiments, the compounds of the present disclosure are administered to the subject until the infection is treated, inhibited, or reduced. In some embodiments, the compounds is administered to the subject until one or more symptoms of the SARS-CoV-2 infection is reduced.

In another aspect, provided herein is a method of inhibiting a viral infection, comprising providing a compound disclosed herein to the infection so as to inhibit the viral infection. In some embodiments, the viral infection is caused by SARS-CoV-2.

In another aspect, provided herein is a method of inhibiting SARS-CoV-2 by binding with a protein thereof, comprising providing a compound disclosed herein to a SARS-CoV-2 so as to inhibit the SARS-CoV-2. In some embodiments, the SARS-CoV-2 binds to a protease on the SARS-CoV-2. In some embodiments, the compounds disclosed herein bind with a cysteine residue of the main protease, thereby inhibiting the SARS-CoV-2. In some embodiments, the cysteine residue is at position 145 of a main protease. In some embodiments, the protease is 3CL.

### Administration and Pharmaceutical Composition

The compounds described herein can be used in pharmaceutical compositions for inhibiting SARS-CoV-2 in order to inhibit SARS-CoV-2 infections. The compounds described herein can be formulated for administration by any suitable route as described herein to a subject having or suspected of having a SARS-CoV-2 infection. The compounds described herein can be used to treat a subject by inhibiting the SARS-CoV-2.

In some embodiments, a pharmaceutical composition is provided, the pharmaceutical composition including an effective amount of the compound of any embodiments of the INSCoV compounds (or pharmaceutically acceptable salt thereof) for treating a condition; where the condition is SARS-CoV-2 infection.

“Effective amount” refers to the amount of a compound or composition required to produce a desired effect. One example of an effective amount includes amounts or dosages that yield acceptable toxicity and bioavailability levels for therapeutic (pharmaceutical) use including, but not limited to, the treatment of SARS-CoV-2 (2019-nCoV) infection referred to as COVID-19..

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. One example of an effective amount includes amounts or dosages that yield acceptable toxicity and bioavailability levels for therapeutic (pharmaceutical) use including, but not limited to, the treatment of SARS-CoV-2 (2019-nCoV) infection referred to as COVID-19. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, a “subject” or “patient” is a mammal, such as but not limited to a cat, dog, rodent or primate. Typically, the subject is a human, and, preferably, a human suffering from or suspected of suffering from a SARS-CoV-2 infection. The term “subject” and “patient” can be used interchangeably.

Thus, the instant present technology provides pharmaceutical compositions and medicaments comprising any of the INSCoV compounds, or derivative thereof, prodrug thereof, salt thereof, or stereoisomer thereof, or having any chirality at any chiral center, or tautomer, polymorph, solvate, or combination thereof as disclosed herein and optionally a pharmaceutically acceptable carrier or one or more pharmaceutically acceptable excipients or fillers. The compositions may be used in the methods and treatments described herein. Such compositions and medicaments include a therapeutically effective amount of compounds as described herein. In some embodiments, the pharmaceutical composition may be packaged in unit dosage form. The unit dosage form is effective in treating a SARS-CoV-2 infection when administered to a subject in need thereof.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant present technology.

Those skilled in the art are readily able to determine an effective amount, such as by simply administering a compound of the present technology to a patient in increasing amounts until the progression of the condition/disease state is decreased or stopped. The compounds of the present technology can be administered to a patient at dosage levels in the range of about 0.1 to about 1,000 mg per day. For a normal human adult having a body weight of about 70 kg, a dosage in the range of about 0.01 to about 100 mg per kg of body weight per day is sufficient. The specific dosage used, however, can vary or may be adjusted as considered appropriate by those of ordinary skill in the art. For example, the dosage can depend on a number of factors including the requirements of the patient, the severity of the condition being treated, and the pharmacological activity of the compound being used. The determination of optimum dosages for a particular patient is well known to those skilled in the art.

Various assays and model systems can be readily employed to determine the therapeutic effectiveness of the treatment according to the present technology.

The administration may include oral administration, parenteral administration, or nasal administration. In any of these embodiments, the administration may include subcutaneous injections, intravenous injections, intraperitoneal injections, or intramuscular injections. In any of these embodiments, the administration may include oral administration. The methods of the present technology can also comprise administering, either sequentially or in combination with one or more compounds of the present technology, a conventional therapeutic agent in an amount that can potentially or synergistically be effective for the treatment of SARS-CoV-2 infection.

In one aspect, compounds disclosed herein are administered to a patient in an amount or dosage suitable for therapeutic use. Generally, a unit dosage comprising a compound of the present technology will vary depending on patient considerations. Such considerations include, for example, age, protocol, condition, sex, extent of disease, contraindications, concomitant therapies and the like. An exemplary unit dosage based on these considerations can also be adjusted or modified by a physician skilled in the art. For example, a unit dosage for a patient comprising a compound of the present technology can vary from 1 × 10⁻⁴ g/kg to 1 g/kg, preferably, 1 × 10⁻³ g/kg to 1.0 g/kg. Dosage of a compound of the present technology can also vary from 0.01 mg/kg to 100 mg/kg or, preferably, from 0.1 mg/kg to 10 mg/kg

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington’s Pharmaceutical Sciences, Mack Publishing Co., Easton, Pennsylvania 1975; Liberman, H.A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), herein incorporated by reference for such disclosure.

A pharmaceutical composition, as used herein, refers to a mixture of a compound disclosed herein with other chemical components (i.e., pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism.

Pharmaceutical formulations described herein are administerable to a subject in a variety of ways by multiple administration routes, including but not limited to, oral, parenteral (e.g., intravenous, subcutaneous, intramuscular, intramedullary injections, intrathecal, direct intraventricular, intraperitoneal, intralymphatic, intranasal injections), intranasal, buccal, topical or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, the compounds disclosed herein are administered orally (PO). In some embodiments, the compounds disclosed herein are administered orally as a table, capsule or pill.

In some embodiments,, the compounds disclosed herein are administered by inhalation. In some embodiments, the compounds disclosed herein are formulated for intranasal administration. Such formulations include nasal sprays, nasal mists, and the like.

In some embodiments, compounds disclosed herein are prepared as transdermal dosage forms.

In some embodiments, the compounds disclosed herein are formulated into a pharmaceutical composition suitable for intramuscular, subcutaneous, or intravenous injection. In some embodiments, the compound is administered intramuscularly. In some embodiments, the compound is administered subcutaneously (SQ). In some embodiments, the compound is administered intravenously (IV).

In any of the aforementioned aspects are further embodiments comprising single administrations of the effective amount of the compound, including further embodiments in which (i) the compound is administered once; (ii) the compound is administered to the subject multiple times over the span of one day; (iii) continually; or (iv) continuously. In some embodiments, the compound is administered once a day, twice a day (BID) or three times a day (TID).

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended, or the dose of the compound being administered is temporarily reduced; at the end of the drug holiday, dosing of the compound is resumed. In one embodiment, the length of the drug holiday varies from 2 days to 1 year.

In some embodiments, the compound is administered until the SARS-CoV-2 is treated. In some embodiments, the compound is administered until one or more symptoms of SARS-CoV-2 is reduced or resolved.

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

The terms below, as used herein, have the following meanings, unless indicated otherwise:

By “substituted” as in “substituted alkyl,” “substituted aryl,” and the like, as alluded to in some of the definitions provided herein, is meant that in the alkyl, aryl, or other moiety, at least one hydrogen atom bound to a carbon (or other) atom is replaced with one or more non-hydrogen substituents.

In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated.

When the term “substituted” appears prior to a list of possible substituted groups, it is intended that the term apply to every member of that group. For example, the phrase “substituted alkyl, alkenyl, and aryl” is to be interpreted as “substituted alkyl, substituted alkenyl, and substituted aryl.” Analogously, when the term “heteroatom-containing” appears prior to a list of possible heteroatom-containing groups, it is intended that the term apply to every member of that group. For example, the phrase “heteroatom-containing alkyl, alkenyl, and aryl” is to be interpreted as “heteroatom-containing alkyl, heteroatom-containing alkenyl, and heteroatom-containing aryl.”

As used herein, “optionally substituted” indicates that a chemical structure may be optionally substituted with a substituent group, such as defined herein. That is, when a chemical structure includes an atom that is optionally substituted, the atom may or may not include the optional substituent group, and thereby the chemical structure may be considered to be substituted when having a substituent on the atom or unsubstituted when omitting a substituent from the atom. A substituted group, referred to as a “substituent” or “substituent group”, can be coupled (e.g., covalently) to a previously unsubstituted parent structure, wherein one or more hydrogens atoms (or other substituent groups) on the parent structure have been independently replaced by one or more of the substituents. The substituent is a chemical moiety that is added to a base chemical structure, such as a chemical scaffold. As such, a substituted chemical structure may have one or more substituent groups on the parent structure, such as by each substituent group being coupled to an atom of the parent structure. The substituent groups that can be coupled to the parent structure can be any possible substituent group. In examples of the present technology, the substituent groups (e.g., R groups) can be independently selected from an alkyl, —O—alkyl (e.g. —OCH₃, —OC₂H₅, —OC₃H₇, —OC₄H₉, etc.), —S—alkyl (e.g., —SCH₃, —SC₂H₅, —SC₃H₇, —SC₄H₉, etc.), —NR′R″, —OH, —SH, —CN, —NO₂, or a halogen, wherein R′ and R″ are independently H or an optionally substituted alkyl. Wherever a substituent is described as “optionally substituted,” that substituent can also be optionally substituted with the above substituents.

In examples of the present disclosure, the substituent groups can be independently selected from: halo, alkyl, alkenyl, alkynyl, aryl, heterocyclyl, thiol, alkylthio, oxo, thioxy, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and an aliphatic group. It is understood that the substituent may be further substituted. In some cases, the term “optionally substituted” or “substituted” means that the referenced group is optionally substituted with one or more additional group(s) individually and independently selected from D, oxo, halogen, —CN, —NH₂, —NH(alkyl), —N(alkyl)₂, —OH, —CO₂H, -CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, oxo, —CN, —NH₂, —NH(CH₃), —N(CH₃)₂, —OH, —CO₂H, —CO₂(C₁—C₄ alkyl), —C(═O)NH₂, —C(═O)NH(C₁-C₄ alkyl), —C(═O)N(C₁C₄ alkyl)₂, —S(═O)₂NH₂, —S(═O)₂NH(C₁-C₄ alkyl), —S(═O)₂N(C₁-C₄ alkyl)₂, C₁-C₄ alkyl, C₃-C₆ cycloalkyl, C₁-C₄ fluoroalkyl, C₁-C₄ heteroalkyl, C₁-C₄ alkoxy, C₁-C₄ fluoroalkoxy, -SC₁-C₄ alkyl, —S(═O)C₁-C₄ alkyl, and —S(═O)z(C₁-C₄ alkyl). In some embodiments, optional substituents are independently selected from D, halogen, —CN, —NH₂, —OH, —NH(CH₃), —N(CH₃)₂, - NH(cyclopropyl), —CH₃, —CH₂CH₃, —CF₃, —OCH₃, and —OCF₃.

The term amino refers to the overall charged or net uncharged chemical group, where the R group can be a substituent, such as the substituents described herein.

The term “alkyl” or “aliphatic” as used herein refers to a branched or unbranched saturated hydrocarbon group typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl, and the like. Generally, although again not necessarily, alkyl groups herein contain 1 to about 18 carbon atoms, or 1 to about 12 carbon atoms. The term “lower alkyl” intends an alkyl group of 1 to 6 carbon atoms. Substituents identified as “C ₁ -C ₆ alkyl” or “lower alkyl” contains 1 to 3 carbon atoms, and such substituents contain 1 or 2 carbon atoms (i.e., methyl and ethyl). “Substituted alkyl” refers to alkyl substituted with one or more substituent groups, and the terms “heteroatom-containing alkyl” and “heteroalkyl” refer to alkyl in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The terms “alkenyl” as used herein refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein contain 2 to about 18 carbon atoms, or 2 to 12 carbon atoms. The term “lower alkenyl” intends an alkenyl group of 2 to 6 carbon atoms, and the specific term “cycloalkenyl” intends a cyclic alkenyl group or having 5 to 8 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” as used herein refers to a linear or branched hydrocarbon group of 2 to 24 carbon atoms containing at least one triple bond, such as ethynyl, n-propynyl, and the like. Generally, although again not necessarily, alkynyl groups herein contain 2 to about 18 carbon atoms, or 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkoxy” as used herein intends an alkyl group bound through a single, terminal ether linkage; that is, an “alkoxy” group may be represented as —O—alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, etc. Substituents identified as “C₁ -C₆ alkoxy” or “lower alkoxy” herein contain 1 to 3 carbon atoms, and such substituents contain 1 or 2 carbon atoms (i.e., methoxy and ethoxy). Unless stated otherwise, an “alkoxy” group can be optionally substituted, for example, by a substituent group stated above. IN some embodiments, an alkoxy is substituted by halogen(s).

As used herein, the term “cycloalkyl” refers to a chain of carbon atoms, a portion of which forms a ring. Cycloalkyl can refer to a stable, partially or fully saturated, monocyclic or polycyclic carbocyclic ring, which may include fused (when fused with an aryl or a heteroaryl ring, the cycloalkyl is bonded through a non-aromatic ring atom), bridged, or spiro ring systems. It is understood that in embodiments that include cycloalkyl, illustrative variations of those embodiments include lower cylcoalkyl, such as C 3 -C 8 cycloalkyl, cyclopropyl, cyclohexyl, 3-ethylcyclopentyl, and the like. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbomyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example, cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

As used herein, the term “cycloalkenyl” refers to an unsaturated chain of carbon atoms, a portion of which forms a ring. It is understood that in embodiments that include cycloalkenyl, illustrative variations of those embodiments include lower cycloalkenyl, such as C₃-C₈, C₃-C₆ cycloalkenyl.

As used herein, the term “alkylene” refers to a saturated chain of carbon atoms, which may be optionally branched. It is understood that in embodiments that include alkylene, illustrative variations of those embodiments include lower alkylene, such as C₂-C₄, alkylene, methylene, ethylene, propylene, 3-methylpentylene, and the like.

“Heteroalkyl” refers to an alkyl group in which one or more skeletal atoms of the alkyl are selected from an atom other than carbon, e.g., oxygen, nitrogen (e.g., —NH—, -N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, -N(alkyl)-), sulfur, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, or —CH(CH₃)OCH₃. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

As used herein, the term “heterocyclic” or “heterocycle” refers to a chain of carbon and heteroatoms, wherein the heteroatoms are selected from nitrogen, oxygen, and sulfur, and a portion of which, at least one heteroatom, forms a ring. The term “heterocycle” may include both “aromatic heterocycles” and “non-aromatic heterocycles.” Heterocycles include 4-7 membered monocyclic and 8-12 membered fused rings, such as imidazolyl, thiazolyl, oxazolyl, oxazinyl, thiazinyl, dithianyl, dioxanyl, isoxazolyl, isothiazolyl, triazolyl, furanyl, tetra-hydrofuranyl, dihydrofuranyl, pyranyl, tetrazolyl, pyrazolyl, pyrazinyl, pyridazinyl, imidazolyl, pyridinyl, pyrrolyl, dihydropyrrolyl, pyrrolidinyl, piperidinyl, piperazinyl, pyrimidinyl, morpholinyl, tetrahydrothiophenyl, thiophenyl, azetidinyl, oxetanyl, thiiranyl, oxiranyl, aziridinyl, indolyl, and the like. “Heterocycles” may be optionally substituted at any one or more positions capable of bearing a hydrogen atom.

Unless stated otherwise specifically in the specification, the heterocycle or heterocyclyl radical can be a monocyclic, bicyclic, tricyclic or tetracyclic ring system, which optionally includes fused, bridged, or spirocyclic ring systems. The heteroatoms in the heterocycle or heterocyclyl radical are optionally oxidized. One or more nitrogen atoms, if present, are optionally quaternized. The heterocycle or heterocyclyl radical cam be partially or fully saturated. The heterocycle or heterocyclyl can be attached to the rest of the molecule through any atom of the ring(s). Examples of such heterocycle or heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term heterocycle or heterocyclyl radicals include those optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—CN, —R^{b}—O—R^{e}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)tOR^{a} (where t is 1 or 2) and —R^{b}—S(O)tN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{e} is a straight or branched alkylene or alkenylene chain, and where each of the above substituents is unsubstituted unless otherwise indicated.

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Examples of aryl groups contain 5 to 20 carbon atoms, and aryl groups contain 5 to 14 carbon atoms. Exemplary aryl groups contain one aromatic ring or two fused or linked aromatic rings, e.g., phenyl, naphthyl, biphenyl, diphenyl ether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. If not otherwise indicated, the term “aryl” includes unsubstituted, substituted, and/or heteroatom-containing aromatic substituents. The term “aryl” includes monocyclic and polycyclic aromatic carbocyclic groups, each of which may be optionally substituted. The term “optionally substituted aryl” refers to an aromatic mono or polycyclic ring of carbon atoms, such as phenyl, naphthyl, and the like, which may be optionally substituted with one or more independently selected substituents, such as halo, hydroxyl, amino, alkyl, or alkoxy, alkylsulfonyl, cyano, nitro, and the like.

The term “heteroaryl” or “aromatic heterocycle” can include substituted or unsubstituted aromatic single ring structures, in some embodiments 5- to 7-membered rings, and in some embodiments 5- to 6-membered rings, whose ring structures include at least one heteroatom, preferably one to four heteroatoms, more preferably one or two heteroatoms. The term “heteroaryl” can also include ring systems having one or two rings wherein at least one of the rings is heteroaromatic, e.g., the other cyclic rings can be cycloalkyl, cycloalkenyl, cycloalkynyl, aromatic carbocycle, heteroaryl, and/or heterocycle. Heteroaryl groups include, for example, pyrrole, furan, thiophene, imidazole, oxazole, thiazole, isoxazole, pyrazole, pyridine, pyrazine, pyridazine, indole, benzofuran, benzoxazole, benzothiazole, benzimidazole and pyrimidine.

Exemplary heteroaryl can comprise carbon atom(s) and one or more ring heteroatoms that selected from the group consisting of nitrogen, oxygen, phosphorous, and sulfur, and at least one aromatic ring. In some embodiments, a heteroaryl is a 5- to 14-membered ring system radical comprising one to thirteen carbon atoms, one to six heteroatoms selected from the group consisting of nitrogen, oxygen, phosphorous, and sulfur. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

It is understood that each of alkyl, cycloalkyl, alkenyl, cycloalkenyl, alkylene, and heterocycle may be optionally substituted with independently selected groups such as alkyl, haloalkyl, hydroxyalkyl, aminoalkyl, carboxylic acid and derivatives thereof, including esters, amides, and nitrites, hydroxy, alkoxy, acyloxy, amino, alky and dialkylamino, acylamino, thio, and the like, and combinations thereof.

The term “spiro” or “spirocyclic” refers to a compound or moiety having one atom as the only common member of two rings.

The term “aryloxy” as used herein refers to an aryl group bound through a single, terminal ether linkage, wherein “aryl” is as defined above. An “aryloxy” group may be represented as —O—aryl where aryl is as defined above. Examples of aryloxy groups contain 5 to 20 carbon atoms, and aryloxy groups contain 5 to 14 carbon atoms. Examples of aryloxy groups include, without limitation, phenoxy, o-halo-phenoxy, m-halo-phenoxy, p-halo-phenoxy, o-methoxy-phenoxy, m-methoxy-phenoxy, p-methoxy-phenoxy, 2,4-dimethoxy-phenoxy, 3,4,5-trimethoxy-phenoxy, and the like.

The term “alkaryl” refers to an aryl group with an alkyl substituent, and the term “aralkyl” refers to an alkyl group with an aryl substituent, wherein “aryl” and “alkyl” are as defined above. Examples of aralkyl groups contain 6 to 24 carbon atoms, and aralkyl groups contain 6 to 16 carbon atoms. Examples of aralkyl groups include, without limitation, benzyl, 2-phenyl-ethyl, 3-phenylpropyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. Alkaryl groups include, for example, p-methylphenyl, 2,4-dimethylphenyl, p-cyclohexylphenyl, 2,7-dimethyinaphthyl, 7-cyclooctylnaphthyl, 3-ethyl-cyclopenta-1,4-diene, and the like.

The term “cyclic” refers to alicyclic or aromatic substituents that may or may not be substituted and/or heteroatom containing, and that may be monocyclic, bicyclic, or polycyclic.

The terms “halo” and “halogen” are used in the conventional sense to refer to a chloro, bromo, and fluoro or iodo substituent.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocyclic” refers to a cyclic substituent that is heteroatom-containing, the terms “heteroaryl” and heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc.

The term “hydrocarbyl” refers to univalent hydrocarbyl radicals containing 1 to about 30 carbon atoms, or 1 to about 24 carbon atoms, or 1 to about 18 carbon atoms, or about 1 to 12 carbon atoms, including linear, branched, cyclic, saturated, and unsaturated species, such as alkyl groups, alkenyl groups, aryl groups, and the like. “Substituted hydrocarbyl” refers to hydrocarbyl substituted with one or more substituent groups, and the term “heteroatom-containing hydrocarbyl” refers to hydrocarbyl in which at least one carbon atom is replaced with a heteroatom. Unless otherwise indicated, the term “hydrocarbyl” is to be interpreted as including substituted and/or heteroatom-containing hydrocarbyl moieties.

The term “optionally substituted,” or “optionally branched”, or “optional substituents,” as used herein, means that the groups in question are either unsubstituted or substituted with one or more of the substituents specified. When the groups in question are substituted with more than one substituent, the substituents may be the same or different. Furthermore, when using the terms “independently,” “independently are,” and “independently selected from” mean that the groups in question may be the same or different. Certain of the herein defined terms may occur more than once in the structure, and upon such occurrence each term shall be defined independently of the other. In some embodiments, the term “optionally substituted” or “substituted” means that the referenced group may be substituted with one or more additional group(s) individually and independently selected from alkyl, haloalkyl, cycloalkyl, aryl, heteroaryl, heterocycloalkyl, —OH, alkoxy, aryloxy, alkylthio, arylthio, alkylsulfoxide, arylsulfoxide, alkylsulfone, arylsulfone, —CN, alkyne, C₁-C₆alkylalkyne, halogen, acyl, acyloxy, —CO₂H, —CO₂alkyl, nitro, and amino, including mono- and di-substituted amino groups (e.g., —NH₂, —NHR, —NR₂), and the protected derivatives thereof. In some embodiments, optional substituents are independently selected from alkyl, alkoxy, haloalkyl, cycloalkyl, halogen, —CN, —NH₂, —NH(CH₃), —N(CH₃)₂, —OH, —CO₂H, and —CO₂alkyl. In some embodiments, optional substituents are independently selected from fluoro, chloro, bromo, iodo, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (=O).

As used herein, C₁—Cₓ (or C₁₋ₓ) includes C₁-C₂, C₁-C₃... C₁—Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl. Also, by way of example, C₀-C₂ alkylene includes a direct bond, —CH₂—, and —CH₂CH₂— linkages.

A “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH. Some examples of tautomeric interconversions include:

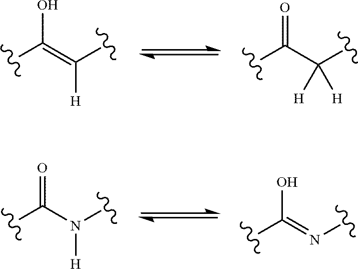

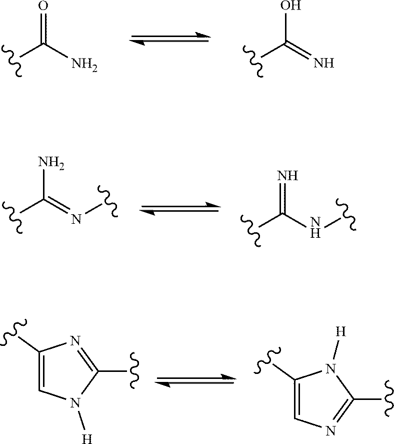

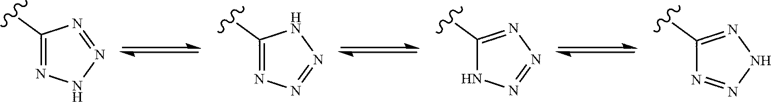

All other chemistry terms are defined as known in the art.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “ a system having at least one of A, B, and C″ would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C″ would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The terms “co-administration” or the like, as used herein, are meant to encompass administration of the selected therapeutic agents to a single patient, and are intended to include treatment regimens in which the agents are administered by the same or different route of administration or at the same or different time.

The term “pharmaceutical combination” as used herein, means a product that results from the mixing or combining of more than one active ingredient and includes both fixed and non-fixed combinations of the active ingredients. The term “fixed combination” means that the active ingredients, e.g., a compound of Formula (I) and a co-agent, are both administered to a patient simultaneously in the form of a single entity or dosage. The term “non-fixed combination” means that the active ingredients, e.g., a compound of Formula (I) and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g., the administration of three or more active ingredients.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The term “regimen” refers to a protocol for dosing and timing the administration of one or more therapies (e.g., combinations described herein or another active agent such as for example an anti-cancer agent described herein) for treating a disease, disorder, or condition described herein. A regimen can include periods of active administration and periods of rest as known in the art

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the claims.

All references recited herein are incorporated herein by specific reference in their entirety: US 2011/0269834, US 2017/0313685; and WO 2010/022455.

### EXAMPLES

It will be appreciated that the following examples are intended to illustrate but not to limit the present disclosure. Various other examples and modifications of the foregoing description and examples will be apparent to a person skilled in the art after reading the disclosure without departing from the spirit and scope of the disclosure, and it is intended that all such examples or modifications be included within the scope of the appended claims. All publications and patents referenced herein are hereby incorporated by reference in their entirety

### Example 1. General Methods of Synthesis

In other embodiments, the starting materials and reagents used for the synthesis of the compounds described herein are synthesized or are obtained from commercial sources, such as, but not limited to, Sigma-Aldrich, Fisher Scientific (Fisher Chemicals), and Acros Organics.

The compounds described herein, and other related compounds having different substituents are synthesized using techniques and materials described herein as well as those that are recognized in the field, such as described, for example, in Fieser and Fieser’s Reagents for Organic Synthesis, Volumes 1-17 (John Wiley and Sons, 1991); Rodd’s Chemistry of Carbon Compounds, Volumes 1-5 and Supplementals (Elsevier Science Publishers, 1989); Organic Reactions, Volumes 1-40 (John Wiley and Sons, 1991), Larock’s Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compounds as disclosed herein may be derived from reactions and the reactions may be modified by the use of appropriate reagents and conditions, for the introduction of the various moieties found in the formulae as provided herein. As a guide the following synthetic methods may be utilized.

Yields reported herein refer to purified products (unless specified). Analytical TLC was performed on Merck silica gel 60 F₂₅₄ aluminium-backed plates. Compounds were visualised by UV light and/or stained with iodine, ninhydrin or potassium permanganate solution followed by heating. Flash column chromatography was performed on silica gel. ¹H-NMR spectra were recorded on a Bruker 400 MHz, Avance II spectrometer with a 5 mm DUL (Dual) ¹³C probe and Bruker 400 MHz, Avance III HD spectrometer with BBFO (Broad Band Fluorine Observe) probe. Chemical shifts (δ) are expressed in parts per million (ppm) with reference to the deuterated solvent peak in which the sample is prepared. Splitting patterns are designated as s (singlet), d (doublet), t (triplet), q (quartet), m (multiplet) and br s (broad singlet).

The following solvents, reagents or scientific terminology may be referred to by their abbreviations:

| | |
|---|---|
| DCM | Dichloromethane |
| DEA | Diethylamine |
| ETOAc or EA | Ethyl Acetate |
| EtOH | Ethanol |
| HPLC | High Performance Liquid Chromatography |
| LCMS | Liquid Chromatoraphy-Mass Spectrometry |
| MeCN or ACN | Acetonitrile |
| MeOH | Methanol |
| MTBE | Methyl tert-butyl ether |
| PE | Petroleum ether |
| SFC | Supercritical Fluid Chromatography |
| THF | Tetrahydrofuran |
| TLC | Thin Layer Chromatography |
| mL | Milliliter |
| mmol | Millimoles |
| h | Hours |
| min | minutes |
| g | Grams |
| mg | Milligrams |
| eq | Equivalents |
| rt or RT | Room temperature (25 ) |
| CF₃CH₂OH | 2,2,2-Trifluoromethanol |
| TiCl₄ | Titanium (V) chloride |
| TEA or Et₃N | Triethyl amine |
| IPA or i-PrOH | Isopropyl alcohol |

In one aspect, the compounds described herein are synthesized as exemplified Ugi-type reaction for preparation of INSCoV series in Scheme 1.

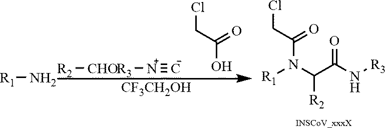

General procedure for preparation of INSCoV series. To a solution of 2-chloroacetic acid (1.00 eq) and isonitrile (1.00 eq) in 2,2,2-trifluoroethanol (7 mL / mmol) was added amine (1.00 eq) and aldehyde (1.00 eq) at 25° C. The mixture was stirred at 25° C. for 1 hr. LC-MS showed amine was consumed completely and one main peak with desired mass was detected. The solvent was evaporated under reduced pressure to give a residue. General purification methods are listed below.

Purification A: The residue was purified by prep-HPLC.

Purification B: The residue was resolved in 10 mL EtOAc and washed with 10 mL water, then separated and the organics was dried over by anhydrous Na₂SO₄, filtered. The organic phase was concentrated in vacuum to give the crude product. The crude product was triturated from a solvent.

In some embodiments, the compounds made in the examples below are made from racemic starting materials (and/or intermediates) and separated into the individual enantiomers by chiral chromatography as final products or intermediates. Unless otherwise stated, it is understood that the absolute configuration of the separated intermediates and final compounds as drawn is arbitrarily assigned and was not determined. In some embodiments, the absolute stereochemistry of the enantiomers as drawn is arbitrarily assigned. In some embodiments, both enantiomers are synthesized.

In some embodiments, the stereochemistry was assigned based on the modelling and activity data. R-isomers from the modelling perspective tend to bind the protease more readily, while S-isomers may be inactive due to the poor pose. For certain compounds, the chiral configurations were confirmed by X-ray study or by chiral synthesis. For example, INSCoV-601I(1) was confirmed to be R-configuration by X-ray of the binding mode. For example, the second chiral center for the following compounds was assigned based on chiral starting material: INSCoV-600B(1), 600B(2), 600C(1), 600C(2), 601Q, 601R, and 601S. As another example, the second chiral center from the isonitrile component for INSCoV-601Q was assigned based on chiral starting material.

### Example 2. Synthesis of INSCoV-517A, INSCoV-517(1A) and INSCoV-517A(1B)

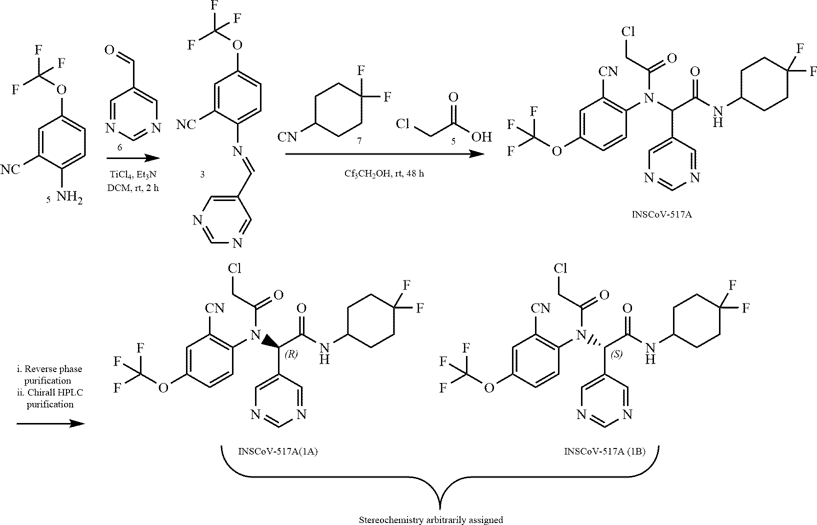

Step 1: To a stirred solution of 2-amino-5-(trifluoromethoxy)benzonitrile 1 (20 g, 98.90 mmol) and pyrimidine-5-carbaldehyde 2 (11.80 g, 109 mmol) in dichloromethane (2 L) was added triethyl amine (30 g, 297 mmol) and TiCl₄ (9.38 g, 49.50 mmol) at 0° C. under inert atmosphere. The reaction mixture was stirred at room temperature for 2 h. After completion of reaction (TLC monitoring), the reaction mixture was diluted with ice cold water (2 L) and extracted with dichloromethane (2 x 2 L). The combined organic layer was washed with brine solution (3 L), dried over Na₂SO₄, filtered and evaporated under reduced pressure to get crude (E)-2-((pyrimidin-5-ylmethylene)amino)-5-(trifluoromethoxy)benzonitrile 7 which was purified by flash chromatography (silica gel, 120 g SNAP) using eluent 5% ethyl acetate in heptane to get the desired product as pale yellow solid (18 g, 56%).

¹H NMR (400 MHz, DMSO d₆): δ 9.38 (s, 1H), 9.30 (s, 2H), 8.87 (s, 1H), 8.10 (s, 1H), 7.88-7.86 (d, J= 8.8 Hz, 1H) and 7.66-7.64 (d, J= 9.2 Hz, 1H. LCMS= [M+H] ⁺: (293.05), Purity =93%.

Step 2: To a stirred solution of 2-chloroacetic acid 5 (4.85 g, 51.3 mmol) in CF₃CH₂OH (50 mL) were added (E)-2-((pyrimidin-5-ylmethylene)amino)-5-(trifluoromethoxy)benzonitrile 7 (5.0 g, 17.1 mmol) and 1,1-difluoro-4-isocyanocyclohexane 8 (4.97 g, 34.2 mmol) at room temperature under inert atmosphere. The reaction mixture was stirred at room temperature for 48 h. After completion of reaction (TLC monitoring), the reaction mixture was diluted with water (50 mL) and extracted with ethyl acetate (2 x 250 mL). The combined organic layer was washed with brine solution (250 mL), dried over Na₂SO₄, filtered and evaporated under reduced pressure to get crude product, which was purified by column chromatography (silica gel, 100-200 mesh) using eluent 35% ethyl acetate in heptane to obtain 2-chloro-N-(2-cyano-4-(trifluoromethoxy)phenyl)-N-(2-((4,4-difluorocyclohexyl)amino)-2-oxo-1-(pyrimidin-5-yl)ethyl)acetamide (INSCoV-517A) as brown solid (350 mg, 74% purity), Obtained compound was further purified by reverse-phase purification to get desired product INSCoV-517A as white solid (150 mg, 2%).

¹H NMR (400 MHz, DMSO d₆): δ 9.15-8.78 (m, 1H), 8.47 (s, 2H), 8.37-8.35 (d, J= 7.2 Hz, 1H), 8.27-8.25 (d, J= 8.0 Hz, 1H), 7.92-7.81 (m, 2H), 6.19-6.04 (m, 1H), 4.23-4.05 (m, 2H), 3.82-3.62 (m, 1H), 2.02-1.95 (m, 1H), 1.92-1.79 (m, 4H), 1.71-1.64 (m, 1H), 1.55-1.46 (m, 1H) and 1.34-1.22 (m, 1H). LCMS= [M+H] ⁺: (532.16), Purity =99.60%.

Step 3: Chiral HPLC purification of INSCoV-517A: INSCoV-517A(1A) and INSCo V-517A (1B). 2-Chloro-N-(2-cyano-4-(trifluoromethoxy)phenyl)-N-(2-((4,4-difluorocyclohexyl)amino)-2-oxo-1-(pyrimidin-5-yl)ethyl)acetamide (INSCoV-517A, 180 mg) was purified by chiral-HPLC using (column: CHIRALPAK IG (250*21) mm, 5 µm; mobile phase: A- i-PrOH (25%) and B- hexane (75%); flow mode: isocratic, loading: 5 mg/injection, run time: 20 mins, wavelength:234 nm, sample preparation: Acetonitrile and i-PrOH to give INSCoV-517A(1A) (65 mg, 72%) and INSCoV-517A(1B) (80 mg, 80%) as white solid.

INSCoV-517A(1A): ¹H NMR (400 MHz, DMSO d₆): δ 9.15-8.78(m, 1H), 8.48 (s, 2H), 8.36-8.34 (d, J= 7.2 Hz, 1H), 8.27-8.25 (d, J= 8.0 Hz, 1H), 7.91-7.82 (m, 2H), 6.29-6.07 (m, 1H), 4.23-4.05 (m, 2H), 3.82-3.62 (m, 1H), 2.02-1.95 (m, 1H), 1.92-1.79 (m, 4H), 1.71-1.64 (m, 1H), 1.55-1.46 (m, 1H), 1.34-1.22 (m, 1H). LCMS= [M+H] ⁺: (532.16), Purity =97.20%. Chiral Purity: 99.2% ee.

INSCoV-517A(1B): ¹H NMR (400 MHz, DMSO d₆): δ 9.15-8.78(m, 1H), 8.46 (s, 2H), 8.36-8.34 (d, J= 7.2 Hz, 1H), 8.27-8.25 (d, J= 8.0 Hz, 1H), 7.92-7.81 (m, 2H), 6.29-6.07 (m, 1H), 4.23-4.05 (m, 2H), 3.82-3.62 (m, 1H), 2.02-1.95 (m, 1H), 1.92-1.79 (m, 4H), 1.71-1.64 (m, 1H), 1.55-1.46 (m, 1H), 1.34-1.22 (m, 1H). LCMS= [M+H] ⁺: (532.16), Purity =97.8%. Chiral Purity: 99.70% ee.

### Example 3. Synthesis of INSCoV-517C, INSCoV-517C (1), INSCoV-517C (2), INSCoV-517C (3) and INSCoV-517C (4)

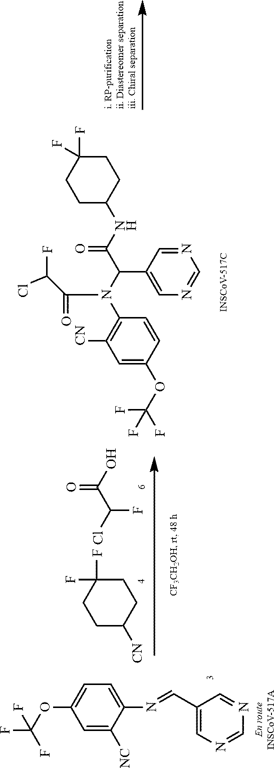

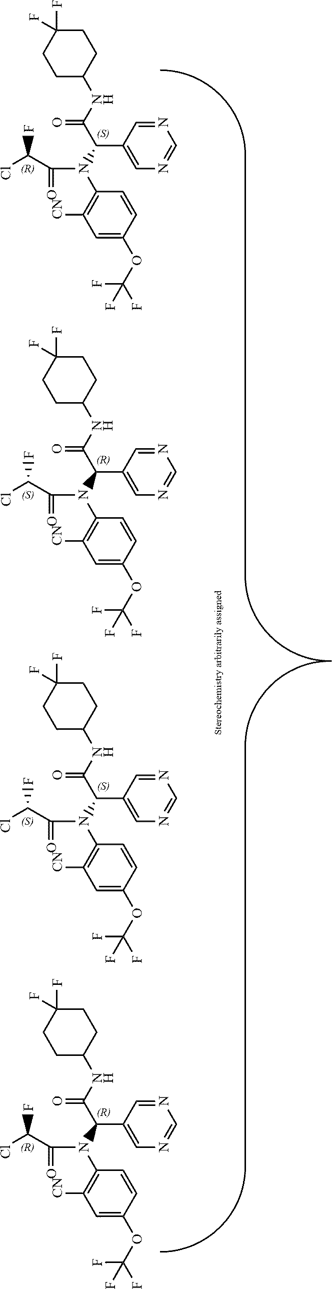

Step 1: To a stirred solution of 2-chloro-2-fluoroacetic acid 6 (5.39 g, 47.9 mmol) in CF₃CH₂OH (50 mL) were added (E)-2-((pyrimidin-5-ylmethylene)amino)-5-(trifluoromethoxy)benzonitrile 3 (7.0 g, 47.9 mmol) and 1,1-difluoro-4-isocyanocyclohexane 4 (6.95 g, 47.9 mmol) at room temperature under inert atmosphere. The reaction mixture was stirred at room temperature for 48 h. After completion of reaction (TLC monitoring), the reaction mixture was diluted with water (250 mL) and extracted with ethyl acetate (2 x 250 mL). The combined organic layer was washed with brine solution (300 mL), dried over Na₂SO₄, filtered and evaporated under reduced pressure to get crude product, which was purified by column chromatography (silica gel, 100-200 mesh) using eluent 25% ethyl acetate in heptane to get the desired product as brown solid (1.40 g, 72% purity), which was further purified by reverse phase purification to get desired product INSCoV-517C (mixture of diastereomers) as white solid (822 mg, 6.27%)

¹H NMR (400 MHz, DMSO d₆): δ9.17-8.79 (m, 1H), 8.45-8.40 (m, 2H), 8.38-8.36 (m, 1H), 8.30-8.17 (m, 1H), 7.94-7.88 (m, 2H), 6.89-6.75 (m, 1H), 6.24-6.00 (m, 1H), 3.81 (br s, 1H), 1.99-1.66 (m, 6H), 1.48-1.36 (m, 1H), 1.26-1.23 (m, 1H). LCMS = [M+H]⁺: (550.18), Purity =99.09%

Step 2: Diastereomeric separation of INSCoV-517C to get INSCoV-517C (D1) and INSCoV-517C (D2). 2-Chloro-N-(2-cyano-4-(trifluoromethoxy)phenyl)-N-(2-((4,4-difluorocyclohexyl)amino)-2-oxo-1-(pyrimidin-5-yl)ethyl)-2-fluoroacetamide (INSCoV-517C, 822 mg) was purified through reverse phase purification and both diastereomers INSCoV-517C (D1) [305 mg, 74%] and INSCoV-517C (D2) [317 mg, 77%] were isolated.

INSCoV-517C (D1): ¹H NMR (400 MHz, DMSO d₆): δ 9.24-8.79 (m, 1H), 8.45 (s, 2H), 8.38-8.36 (d, J= 7.2 Hz, 1H), 8.30-8.28 (d, J= 7.2 Hz, 1H), 7.95-7.92 (m, 2H), 6.89-6.72 (m, 1H), 6.11 (s, 1H), 3.77 (br s, 1H), 1.98-1.68 (m, 6H), 1.52-1.45 (m, 1H), 1.41-1.23 (m, 1H). LCMS = [M+H]⁺: (550.18), Purity =99.72%.

INSCoV-517C (D2): ¹H NMR (400 MHz, DMSO d₆): δ 9.24-8.84 (m, 1H), 8.44 (s, 2H), 8.41-8.39 (d, J= 7.2 Hz, 1H), 8.28-8.26 (d, J= 7.2 Hz, 1H), 7.95-7.88 (m, 2H), 6.87-6.56 (m, 1H), 6.24 (s, 1H), 3.83 (br s, 1H), 1.98-1.68 (m, 6H), 1.52-1.45 (m, 1H), 1.41-1.23 (m, 1H). LCMS = [M+H]⁺: (550.18), Purity =98.8%.

Step 3: Chiral separation of INSCoV-517C (D1): INSCoV-517C (1) and INSCoV-517C (2). Chiral HPLC purification of INSCoV-517C (D1) [305 mg] was done using (column: CHIRALPAK IG (250*30) mm, 5 µm; mobile phase: A- EtOH (15%) and B- 0.1% formic acid in hexane (85%); flow mode: isocratic, loading: 20 mg/injection, run time: 35 mins, wavelength: 230 nm, sample preparation: Acetonitrile and i-PrOH to give INSCoV-517C(1) [88 mg, 58%] and INSCoV-517C (2) [55 mg, 35%] as white solids.

INSCoV-517C (1): ¹H NMR (400 MHz, DMSO d₆): δ9.22-8.88 (m, 1H), 8.45 (s, 2H), 8.38-8.36 (d, J= 7.2 Hz, 1H), 8.30-8.28 (d, J= 7.2 Hz, 1H), 7.95-7.63 (m, 2H), 6.89-6.72 (m, 1H), 6.11 (s, 1H), 3.77 (br s, 1H), 1.98-1.87 (m, 6H), 1.70-1.66 (m, 1H), 1.51-1.45 (m, 1H). LCMS = [M+H]⁺: (550.18), Purity =99.8%. Chiral purity: 99.4% ee.

INSCoV-517C (2): ¹H NMR (400 MHz, DMSO d₆): δ9.17-8.79 (m, 1H), 8.45 (s, 2H), 8.38-8.36 (d, J= 7.2 Hz, 1H), 8.30-8.28 (d, J= 7.2 Hz, 1H), 7.95-7.88 (m, 2H), 6.89-6.72 (m, 1H), 6.11 (s, 1H), 3.77 (br s, 1H), 1.98-1.68 (m, 6H), 1.52-1.45 (m, 1H), 1.41-1.23 (m, 1H). LCMS = [M+H]⁺: (550.18), Purity =98.8%. Chiral purity: 99.7% ee.

Step 4: Chiral separation of INSCoV-517C (D2): INSCoV-517C (3) and INSCoV-517C (4). Chiral-HPLC purification of INSCoV-517C (D2) [317 mg] was done using (column: CHIRALPAK IG (250*30) mm, 5 µm; mobile phase: A- EtOH (20%) and B- 0.1% formic acid in hexane (80%); flow mode: isocratic, loading: 20 mg/injection, run time: 20 mins, wavelength:230 nm, sample preparation: Acetonitrile and i-PrOH to give INSCoV-517C(3) [110 mg, 70%] andINSCoV-517C (4) [105 mg, 66%] as white solids.

INSCoV-517C (3): ¹H NMR (400 MHz, DMSO d₆): δ9.17-8.84 (m, 1H), 8.44 (s, 2H), 8.40-8.38 (d, J= 7.2 Hz, 1H), 8.28-8.26 (d, J= 7.2 Hz, 1H), 7.94-7.88 (m, 2H), 6.87-6.68 (m, 1H), 6.27 (s, 1H), 3.82 (br s, 1H), 1.98-1.68 (m, 6H), 1.52-1.45 (m, 1H), 1.41-1.23 (m, 1H). LCMS = [M+H]⁺: (550.18), Purity =97.41%. Chiral purity: 98.5% ee.

INSCoV-517C (4): ¹H NMR (400 MHz, DMSO d₆): δ9.24-8.87 (m, 1H), 8.44 (s, 2H), 8.41-8.39 (d, J= 7.2 Hz, 1H), 8.28-8.26 (d, J= 7.2 Hz, 1H), 7.95-7.88 (m, 2H), 6.87-6.68 (m, 1H), 6.24 (s, 1H), 3.81 (br s, 1H), 1.98-1.68 (m, 6H), 1.52-1.45 (m, 1H), 1.41-1.23 (m, 1H). LCMS = [M+H]⁺: (550.18), Purity =98.90%. Chiral purity: 99.82% ee.

### Example 4. Synthesis of 2-Chloro-N-(2-(Cyclohexylamino)-2-oxo-1-(Pyrimidin-5-yl)Ethyl)-N-(4-(Oxazol-5-yl)Phenyl)Acetamide (INSCoV-501A)

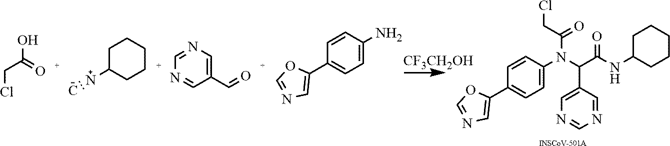

To a solution of 2-chloroacetic acid (87 mg, 0.925 mmol) and isocyanocyclohexane (101 mg, 0.925 mmol) in 2,2,2-trifluoroethanol (7 mL) was added 4-(oxazol-5-yl)aniline (148 mg, 0.925 mmol) and pyrimidine-5-carbaldehyde (100 mg, 0.925 mmol) at 25° C. The mixture was stirred at 25° C. for 1 hr. LC-MS showed 4-(oxazol-5-yl)aniline was consumed completely and one main peak with desired mass was detected. The solvent was evaporated under reduced pressure to give a residue. The crude product was triturated with MeOH (15 mL) and washed with MeOH (3 mL × 3). The filter cake was concentrated under vacuum. The residue was diluted with water (10 mL) and under lyophilization to give the product. INSCoV-501A (264.74 mg, 574.76 µmol, 62.13% yield) was obtained as white solid.

¹H NMR (400 MHz, DMSO-d₆) δ = 8.95 (s, 1H), 8.51 - 8.40 (m, 3H), 8.20 (d, J =7.6 Hz, 1H), 7.72 (s, 1H), 7.67 - 7.61 (m, 2H), 7.49 - 7.43 (m, 1H), 6.10 (s, 1H), 4.15 - 3.94 (m, 2H), 3.68 -3.52 (m, 1H), 1.81 - 1.46 (m, 5H), 1.35 - 0.94 (m, 5H). LCMS: m/z 454.3 [M+H]⁺, Purity =98.5%

### Example 5. Synthesis of 2-Chloro-N-(2-((4,4-Difluorocyclohexyl)Amino)-2-Oxo-1-(Pyrimidin-5-yl)Ethyl)-N-(4-(Oxazol-5-yl)Phenyl)Acetamide (INSCoV-5011)

2-Chloro-N-(2-((4,4-difluorocyclohexyl)amino)-2-oxo-1-(pyrimidin-5-yl)ethyl)-N-(4-(oxazol-5-yl)phenyl)acetamide was synthesized according to the procedure for the preparation of INSCoV-501A (Example 4). The crude product was triturated with MTBE (20 mL × 2) and filtered. Then it was triturated with MeOH (6 mL) and filtered. INSCoV-501I (214.09 mg, 426.40 µmol, 46.09% yield) was obtained as off-white solid.

¹H NMR: (400 MHz, DMSO-d₆) δ = 8.96 (s, 1H), 8.49 (s, 2H), 8.45 (s, 1H), 8.31 (d, J= 7.6 Hz, 1H), 7.71 (s, 1H), 7.65-7.63 (m,2H), 7.43 (br s, 1H), 6.07 (s, 1H), 4.11 - 3.97 (m, 2H), 3.90 - 3.74 (m, 1H), 2.05 - 1.71 (m, 6H), 1.59 - 1.30 (m, 2H). LCMS: m/z 490.3 [M+H]⁺, Purity =98.9%.

### Example 6. Synthesis of INSCoV-600J, INSCoV-600J(1) and INSCoV-600J(2)

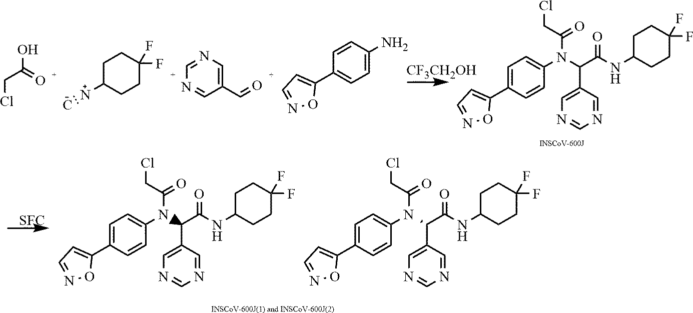

Step 1: 2-Chloro-N-(2-((4,4-difluorocyclohexyl)amino)-2-oxo-1-(pyrimidin-5-yl)ethyl)-N-(4-(isoxazol-5-yl)phenyl)acetamide was synthesized according to the procedure for the preparation of INSCoV-501A (Example 4). The crude product was triturated with MTBE (20 mL × 2) and filtered. INSCoV-600J (165.79 mg, 338.42 µmol, 36.58% yield) was obtained as yellow solid.

¹H NMR: (400 MHz, DMSO-d₆) δ = 8.97 (s, 1H), 8.66 (d, J= 2.0 Hz, 1H), 8.50 (s, 2H), 8.34 (d, J= 7.6 Hz, 1H), 7.82-7.80 (m, 2H), 7.50-7.49 (m, 2H), 7.06 (d, J= 1.9 Hz, 1H), 6.09 (s, 1H), 4.16 - 4.00 (m, 2H), 3.92 - 3.75 (m, 1H), 2.06 - 1.72 (m, 6H), 1.61 - 1.45 (m, 1H), 1.43 - 1.29 (m, 1H). LCMS: m/z 490.2 [M+H]⁺, Purity =100%.

Step 2: Chiral SFCpurification of INSCo V-600J: INSCo V-600J(1) and INSCoV-600J(2). INSCoV-600J. (100 mg, 204.12 µmol, 1 eq) was separated by chiral SFC (column: Daicel ChiralPak IG (250*30 mm, 10 µm); mobile phase: [Neu-MeOH]; B%: 40%-40%, 6.2; 60 min) and concentrated under vacuum. First peak INSCoV-600J(1) (28.97 mg, 59.13 µmol, 28.97% yield) was obtained as yellow solid. Second peak INSCoV-600J(2) (22.10 mg, 45.11 µmol, 22.10% yield) was obtained as yellow solid.

INSCoV-600J(1): ¹H NMR: (400 MHz, DMSO-d₆) δ = 8.97 (s, 1H), 8.66 (d, J =2.0 Hz, 1H), 8.50 (s, 2H), 8.33 (d, J =7.4 Hz, 1H), 7.82-7.79 (m, 2H), 7.50 (br s, 2H), 7.05 (d, J =1.8 Hz, 1H), 6.09 (s, 1H), 4.15 - 3.99 (m, 2H), 3.89 - 3.78 (m, 1H), 2.04 - 1.73 (m, 6H), 1.59 -1.47 (m, 1H), 1.42 - 1.30 (m, 1H). LCMS: m/z 490.3 [M+H]⁺, Purity =96.3%. Chiral Purity: 98.5% ee.

INSCoV-600J(2): ¹H NMR: (400 MHz, DMSO-d₆) δ = 8.97 (s, 1H), 8.66 (d, J =2.0 Hz, 1H), 8.50 (s, 2H), 8.33-8.31 (m, 1H), 7.82-7.79 (m, 2H), 7.61 - 7.35 (m, 2H), 7.05 (d, J= 1.8 Hz, 1H), 6.09 (s, 1H), 4.13 - 3.97 (m, 2H), 3.91 - 3.77 (m, 1H), 2.02 - 1.72 (m, 6H), 1.59 -1.47 (m, 1H), 1.43 -1.32 (m, 1H). LCMS: m/z 490.3 [M+H]⁺, Purity =99.6%. Chiral Purity: 99.0% ee.

### Example 7. Synthesis of INSCoV-600K, INSCoV-600K(1) and INSCoV-600K(2)

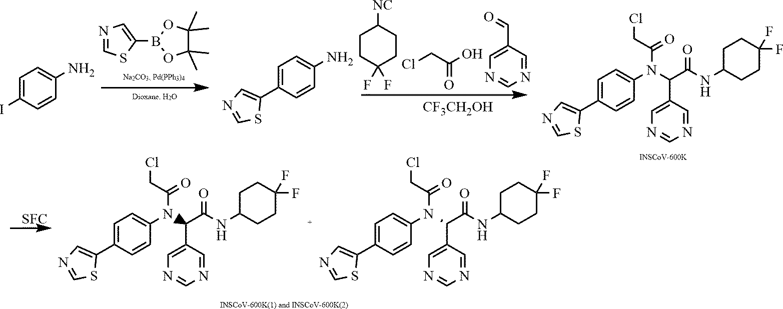

Step 1: To the solution of 4-iodoaniline (216.16 mg, 986.95 µmol, 1 eq) and 5-(4,4,5,5-tetramethyl-1,3,2-dioxaborolan-2-yl)thiazole (250 mg, 1.18 mmol, 1.2 eq) in Dioxane (7.5 mL) and H₂O (2.5 mL) was added Na₂CO₃ (261.51 mg, 2.47 mmol, 2.5 eq) and Pd(PPh₃)₄ (57.02 mg, 49.35 µmol, 0.05 eq) . The mixture was stirred at 80° C. for 12 hrs under N₂. LCMS showed one peak with desired mass was detected. TLC (PE/EA=3/1) showed 4-iodoaniline was consumed and three new spots formed. The reaction mixture was diluted with water (20 mL) and extracted with EtOAc (20 mL * 3). The combined organic layers were dried over anhydrous Na₂SO₄, filtered and concentrated under reduced pressure to give a residue. The residue was purified by column chromatography (SiO₂, PE: EA=20:1 to 2:1). 4-(Thiazol-5-yl)aniline (0.15 g, 92.1% purity, 79.5% yield) was obtained as yellow solid.

LCMS: m/z 177.2 [M+H]⁺, Purity =92.1%.

Step 2: To a solution of 2-chloroacetic acid (188.82 mg, 2.00 mmol, 224.79 µL, 1.2 eq) and pyrimidine-5-carbaldehyde (0.18 g, 1.67 mmol, 1 eq) in CF₃CH₂OH (10 mL) was added 1,1-difluoro-4-isocyanocyclohexane (241.70 mg, 1.67 mmol, 1 eq) and 4-(thiazol-5-yl)aniline (293.46 mg, 1.67 mmol, 1 eq). The reaction mixture was stirred at 25° C. for 1 hr. LCMS showed 4-(thiazol-5-yl)aniline was consumed completely and one new peak with desired mass was detected. The reaction mixture was concentrated under vacuum. The reaction mixture was triturated with MTBE (20 mL) and washed with MTBE (10 mL*3). The filter cake was concentrated under vacuum. The residue was diluted with MTBE (20 mL) and washed with MTBE (10 mL*3). The filter cake was concentrated under vacuum. 2-Chloro-N-(2-((4,4-difluorocyclohexyl)amino)-2-oxo-1-(pyrimidin-5-yl)ethyl)-N-(4-(thiazol-5-yl)phenyl)acetamide INSCoV-600K (177.26 mg, 332.75 µmol, 19.98% yield) was obtained as a yellow solid.

¹H NMR (400 MHz, DMSO-d₆) δ = 9.09 (s, 1H), 8.97 (s, 1H), 8.50 (s, 2H), 8.37 - 8.28 (m, 2H), 7.64 (d, J= 8.8 Hz, 2H), 7.51 - 7.09 (m, 2H), 6.07 (s, 1H), 4.12 - 3.97 (m, 2H), 3.90 - 3.78 (m, 1H), 2.04 - 1.70 (m, 6H), 1.58 - 1.25 (m, 2H). LCMS: m/z 506.3 [M+H]⁺, Purity =93.0%.

Step 3: Chiral SFC purification of INSCoV-600K: INSCoV-600K(1) and INSCoV-600 K(2). INSCoV-600K (49 mg) was purified by SFC separation (column: DAICEL CHIRALPAK AD(250 mm*30 mm,10 µm);mobile phase: [Neu-MeOH];B%: 50%-50%,4 min;20 min) and concentrated under vacuum (<35° C.). First peak INSCoV-600K(1) (13 mg, 24.49 µmol, 25% yield, 95.32% purity) was obtained as yellow solid. Second peak INSCoV-600K(2) (10 mg,19.11 µmol, 19.7% yield, 96.71% purity) was obtained as yellow solid.

INSCoV-600K(1): ¹H NMR (400 MHz, DMSO-d₆) δ = 9.19 - 8.84 (m, 2H), 8.51 (s, 2H), 8.33 (s, 2H), 7.64 (d, J= 6.4 Hz, 2H), 7.40 (s, 2H), 6.08 (s, 1H), 4.09 - 4.01 (m, 2H), 3.83 (d, J= 1.6 Hz, 1H), 2.05 - 1.71 (m, 6H), 1.61 - 1.29 (m, 2H). LCMS: m/z 506.3 [M+H]⁺, Purity =99.1%. Chiral Purity: 100% ee.

INSCoV-600K(2): ¹H NMR (400 MHz, DMSO-d₆) δ = 9.18 - 8.87 (m, 2H), 8.51 (s, 2H), 8.40 - 8.24 (m, 2H), 7.63 (d, J = 8.4 Hz, 2H), 7.40 (s, 2H), 6.08 (s, 1H), 4.20 - 3.96 (m, 2H), 3.92 -3.72 (m, 1H), 2.05 - 1.68 (m, 6H), 1.62 - 1.47 (m, 1H), 1.43 - 1.30 (m, 1H). LCMS: m/z 506.2 [M+H]⁺, Purity =100%. Chiral Purity: 100% ee.

### Example 8. Synthesis of INSCoV-601G, INSCoV-601G(1) and INSCoV-601G(2)

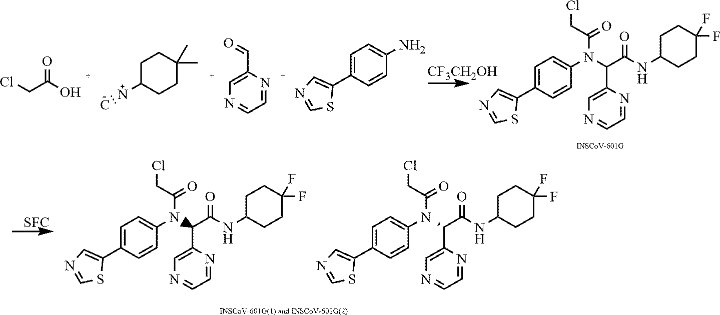

Step 1: To a solution of 4-(thiazol-5-yl)aniline (150 mg, 851.12 µmol, 1 eq) and 4,4-difluorocyclohexane-1-carbonitrile (123.54 mg, 851.12 µmol, 1 eq) in CF₃CH₂OH (4 mL) was added 2-chloroacetic acid (80.43 mg, 851.12 µmol, 95.75 µL, 1 eq) and pyrazine-2-carbaldehyde (92.00 mg, 851.12 µmol, 1 eq). The reaction mixture was stirred at 25° C. for 1 hr. LCMS showed reactant was consumed and one peak of desired mass was detected. The reaction was concentrated under vacuum. The crude product was triturated with MTBE (20 mL) and filtered. 2-Chloro-N-(2-((4,4-difluorocyclohexyl)amino)-2-oxo-1-(pyrazin-2-yl)ethyl)-N-(4-(thiazol-5-yl)phenyl)acetamide INSCoV-601G (303.51 mg, 584.83 µmol, 68.71% yield) was obtained as yellow solid.

¹H NMR (400 MHz, DMSO-d₆): δ = 9.09 (d, J= 0.6 Hz, 1H), 8.52 (d, J= 1.2 Hz, 1H), 8.49-8.48 (m, 1H), 8.44 (d, J= 2.4 Hz, 1H), 8.34 - 8.26 (m, 2H), 7.60-7.58 (m, 2H), 7.55 - 7.30 (m, 2H), 6.23 (s, 1H), 4.21 - 3.98 (m, 2H), 3.88 - 3.68 (m, 1H), 2.01 -1.82 (m, 4H), 1.80 - 1.70 (m, 2H), 1.53 -1.37 (m, 2H). LCMS: m/z 506.0 [M+H]⁺, Purity =100%.

Step 2: Chiral SFC purification of INSCoV-601G: INSCoV-601G(1) and INSCoV-601G(2). INSCoV-601G (100 mg, 197.64 µmol, 1 eq) was separated by chiral SFC (column: DAICEL CHIRALPAK AD (250 mm*30 mm, 10 µm); mobile phase: [Neu-MeOH]; B%: 55%-55%, 4.4 min; 45 min) and concentrated under vacuum. First peak INSCoV-601G(1) (34.34 mg, 62.33 µmol, 31.54% yield, 91.837% purity) as brown solid was obtained. Second peak INSCoV-601G(2) (28.63 mg, 52.47 µmol, 26.55% yield, 92.721% purity) was obtained as brown solid.

INSCoV-601G(1): ¹H NMR: (400 MHz, DMSO-d₆) δ = 9.08 (d, J= 0.6 Hz, 1H), 8.55 - 8.47 (m, 2H), 8.43 (d, J =2.6 Hz, 1H), 8.34 - 8.24 (m, 2H), 7.60-7.58 (m, 2H), 7.53 - 7.28 (m, 2H), 6.23 (s, 1H), 4.19 - 3.98 (m, 2H), 3.85 - 3.74 (m, 1H), 2.02 - 1.83 (m, 4H), 1.79 - 1.70 (m, 2H), 1.52 - 1.35 (m, 2H). LCMS: m/z 506.3 [M+H]⁺, Purity =100%. Chiral Purity: 100% ee.

INSCoV-601G(2): ¹H NMR: (400 MHz, DMSO-d₆) δ = 9.08 (s, 1H), 8.52 (d, J =1.2 Hz, 1H), 8.49-8.48 (m, 1H), 8.43 (d, J =2.4 Hz, 1H), 8.33 -8.27 (m, 2H), 7.60-7.58 (m, 2H), 7.52 - 7.33 (m, 2H), 6.23 (s, 1H), 4.19 - 4.00 (m, 2H), 3.88 - 3.73 (m, 1H), 1.99 - 1.81 (m, 4H), 1.80 - 1.70 (m, 2H), 1.52 - 1.37 (m, 2H). LCMS: m/z 506.2 [M+H]⁺, Purity =100%. Chiral Purity: 86.5% ee.

### Example 9. Synthesis of INSCoV-601H

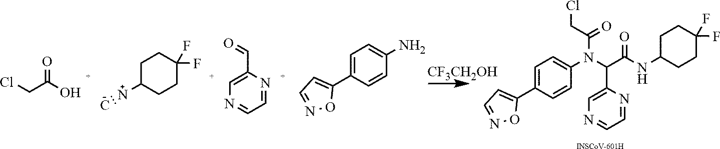

To a solution of 4-(isoxazol-5-yl)aniline (148.17 mg, 925.09 µmol, 1 eq) and 4,4-difluorocyclohexane-1-carbonitrile (134.28 mg, 925.09 µmol, 1 eq) in CF₃CH₂OH (4 mL) was added 2-chloroacetic acid (87.42 mg, 925.09 µmol, 104.07 µL, 1 eq) and pyrazine-2-carbaldehyde (100 mg, 925.09 µmol, 1 eq). The reaction mixture was stirred at 25° C. for 1 hr. LCMS showed reactant was consumed and one peak of desired mass was detected. The reaction was concentrated under vacuum. The crude product was triturated with MTBE (20 mL) and filtered. 2-Chloro-N-(2-((4,4-difluorocyclohexyl)amino)-2-oxo-1-(pyrazin-2-yl)ethyl)-N-(4-(isoxazol-5-yl)phenyl)acetamide INSCoV-601H (398.64 mg, 797.28 µmol, 86.18% yield) was obtained as off-white solid.

¹H NMR (400 MHz, DMSO-d₆): δ = 8.65 (d, J= 2.0 Hz, 1H), 8.54 (d, J= 0.8 Hz, 1H), 8.50 - 8.46 (m, 1H), 8.43 (d, J =2.4 Hz, 1H), 8.31 (d, J = 7.6 Hz, 1H), 7.78-7.76 (m, 2H), 7.54 (br s, 2H), 7.04 (d, J= 2.0 Hz, 1H), 6.25 (s, 1H), 4.23 - 4.01 (m, 2H), 3.85 - 3.72 (m, 1H), 2.02 - 1.81 (m, 4H), 1.80 - 1.70 (m, 2H), 1.52 - 1.36 (m, 2H). LCMS: m/z 490.3 [M+H]⁺, Purity =98.23%.

### Example 10. Synthesis of INSCoV-601I, INSCoV-601I(1) and INSCoV-601I(2)

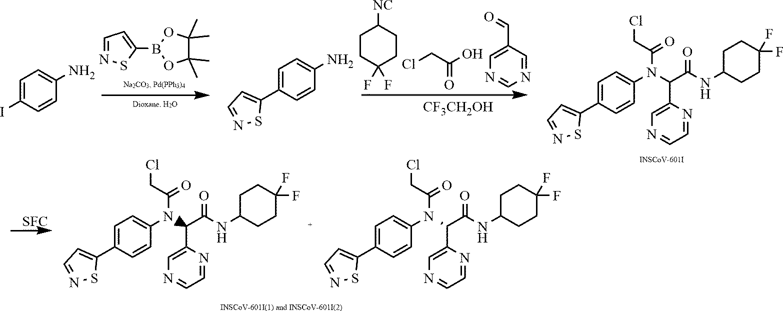

Step 1: To a solution of 4-iodoaniline (432.33 mg, 1.97 mmol, 1 eq) and 5-(4,4,5,5-tetramethyl-1,3,2-dioxaborolan-2-yl)isothiazole (0.5 g, 2.37 mmol, 1.2 eq) in dioxane (24 mL) and H₂O (8 mL) was added Pd(PPh₃)₄ (228.09 mg, 197.39 µmol, 0.1 eq) and Na₂CO₃ (523.03 mg, 4.93 mmol, 2.5 eq). The mixture was stirred at 80° C. for 12 hrs under N₂. LCMS showed the 4-iodoaniline (Rt= 0.866 min) remained and one peak (Rt=0.809 min) with desired mass was detected. TLC (PE: EA = 5: 1) showed the 4-iodoaniline (R_{f}= 0.6) remained and two spots (R_{f}=0.9, R_{f}=0.4) formed. The reaction mixture was diluted with water (10 mL) and extracted with EA (20 ml × 3). The combined organic phase was concentrated under vacuum. The residue was purified by flash silica gel chromatography (ISCO®; 20 g SepaFlash® Silica Flash Column, Eluent: 0 \~50% Ethylacetate in Petroleum ether @ 60 mL/min). The combined organic phase was concentrated under vacuum. 4-(isothiazol-5-yl)aniline (0.1 g, 567.41 µmol, 28.75% yield) was obtained as white solid.

¹H NMR (400 MHz, DMSO-d₆): δ = 8.44 (d, J= 1.6 Hz, 1H), 7.49 (d, J= 2.0 Hz, 1H), 7.43 - 7.36 (m, 2H), 6.67 - 6.55 (m, 2H), 5.63 (s, 2H).

Step 2: The compound was synthesized according to the procedure for the preparation of INSCoV-601H (Example 9). MTBE (20 mL) was added to the reaction mixture, filtered and washed with MTBE (10 mL × 3) to get the crude product. The residue was triturated with MTBE (20 mL), filtered and washed with MTBE (10 mL × 3). The filter cake was concentrated under vacuum. 2-Chloro-N-(2-((4,4-difluorocyclohexyl)amino)-2-oxo-1-(pyrazin-2-yl)ethyl)-N-(4-(isothiazol-5-yl)phenyl)acetamide INSCoV-601I (166.44 mg, 322.55 µmol, 58.11% yield) was obtained as white solid.

¹H NMR (400 MHz, DMSO-d₆): δ = 8.59 (d, J= 1.8 Hz, 1H), 8.54 (d, J= 1.2 Hz, 1H), 8.52 - 8.46 (m, 1H), 8.44 (d, J =2.4 Hz, 1H), 8.29 (d, J =7.6 Hz, 1H), 7.78 (d, J =1.6 Hz, 1H), 7.66 (d, J =8.8 Hz, 2H), 7.50 (s, 2H), 6.24 (s, 1H), 4.22 - 4.01 (m, 2H), 3.87 - 3.75 (m, 1H), 2.06 - 1.68 (m, 6H), 1.54 - 1.34 (m, 2H). LCMS: m/z 506.2 [M+H]⁺, Purity =100%.

Step 3: Chiral SFC purification of INSCoV-601I: INSCoV-601I(1) and INSCoV-601I(2). 2-chloro-N-(2-((4,4-difluorocyclohexyl)amino)-2-oxo-1-(pyrazin-2-yl)ethyl)-N-(4-(isothiazol-5-yl)phenyl)acetamide INSCoV-601I (0.1 g, 197.64 µmol) was separated by chiral SFC (column: DAICEL CHIRALP AKAD (250 mm × 30 mm, 10 µm);mobile phase: [Neu-MeOH]; B%: 45%-45%,4 min; 20 min) to give first peak INSCoV-601I(1) (23.1 mg, 44.66 µmol, 22.59% yield, 97.81% purity) as a yellow solid. Second peak INSCoV-601I(2) (9.94 mg,19.52 µmol, 9.87% yield, 99.346% purity) was obtained as a yellow solid.

INSCoV-601I(1): ¹H NMR (400 MHz, DMSO-d₆): δ = 8.61 - 8.57 (m, 1H), 8.54 (d, J = 1.2 Hz, 1H), 8.52 - 8.46 (m, 1H), 8.44 (d, J= 2.4 Hz, 1H), 8.29 (d, J =7.6 Hz, 1H), 7.78 (d, J= 1.6 Hz, 1H), 7.66 (d, J =8.8 Hz, 2H), 7.61 - 7.39 (m, 2H), 6.24 (s, 1H), 4.23 - 4.01 (m, 2H), 3.85 - 3.73 (m, 1H), 1.98 - 1.71 (m, 6H), 1.54 - 1.35 (m, 2H). LCMS: m/z 506.3 [M+H]⁺, Purity =100%. Chiral Purity: 97.2% ee.

INSCoV-601I(2): ¹H NMR (400 MHz, DMSO-d₆) δ = 8.59 (d, J= 1.8 Hz, 1H), 8.54 (d, J= 1.2 Hz, 1H), 8.52 - 8.46 (m, 1H), 8.44 (d, J =2.4 Hz, 1H), 8.29 (d, J =7.6 Hz, 1H), 7.78 (d, J= 1.6 Hz, 1H), 7.66 (d, J =8.8 Hz, 2H), 7.58 - 7.42 (m, 2H), 6.24 (s, 1H), 4.22 - 4.02 (m, 2H), 3.88 - 3.75 (m, 1H), 2.05 - 1.71 (m, 6H), 1.56 - 1.34 (m, 2H). LCMS: m/z 506.2 [M+H]⁺, Purity =100%. Chiral Purity: 91.76% ee.

### Example 11. Synthesis of INSCoV-601K, INSCoV-601K(1) and INSCoV-601K(2)

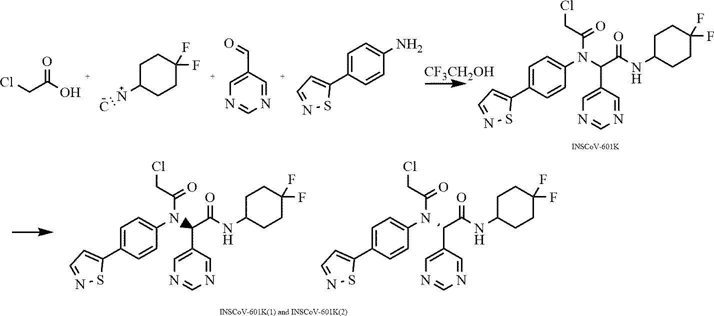

Step 1: The compound was synthesized according to the procedure for the preparation of INSCoV-601H (Example 9). The mixture was concentrated under vacuum. The crude was dissolved in MTBE (10 mL), stirred for a moment and filter cake was concentrated under vacuum. Then it was dissolved in EtOAc (10 mL), stirred for a moment and filter cake was concentrated under vacuum. 2-Chloro-N-(2-((4,4-difluorocyclohexyl)amino)-2-oxo-1-(pyrimidin-5-yl)ethyl)-N-(4-(thiazol-5-yl)phenyl)acetamide INSCoV-601K (100 mg,181.83 µmol, 19.66% yield) was obtained as a yellow solid.

¹H NMR (400 MHz, DMSO-d₆): δ = 8.98 (s, 1H), 8.60 (d, J= 1.8 Hz, 1H), 8.51 (s, 2H), 8.33 (d, J =7.6 Hz, 1H), 7.81 (d, J =1.7 Hz, 1H), 7.71 (br d, J =8.8 Hz, 2H), 7.55 - 7.30 (m, 2H), 6.09 (s, 1H), 4.14 - 4.09 (m, 2H), 3.93 - 3.76 (m, 1H), 2.07 -1.72 (m, 6H), 1.63 - 1.21 (m, 2H). LCMS: m/z 506.1 [M+H]⁺, Purity =92.0%.

Step 2: Chiral SFC purification of INSCoV-601K: INSCoV-601K(1) and INSCoV-601 K(2). INSCoV-601K (102 mg) was separated by chiral SFC(column: Daicel ChiralPak IG (250*30 mm, 10 µm);mobile phase: [Neu-MeOH];B%: 60%-60%, 4.7;35 min). First peak INSCoV-601 K(1) (9.97 mg, 18.94 µmol, 9.4% yield, 96.138% purity) was obtained as yellow solid. Second peak INSCoV-601K(2) (54.54 mg, 104.52 µmol, 51.8% yield, 96.965% purity) was obtained as a yellow solid.

INSCoV-601K(1): ¹H NMR (400 MHz, DMSO-d₆) δ = 8.98 (s, 1H), 8.59 (d, J =1.8 Hz, 1H), 8.51 (s, 2H), 8.32 (br d, J= 7.5 Hz, 1H), 7.80 (d, J=1.8 Hz, 1H), 7.70 (br d, J= 8.7 Hz, 2H), 7.46 (br d, J= 1.8 Hz, 2H), 6.08 (s, 1H), 4.12 - 4.03 (m, 2H), 3.91 - 3.75 (m, 1H), 3.17(d, J= 5.3 Hz, 1H), 2.08 - 1.73 (m, 7H), 1.61 - 1.48 (m, 1H), 1.45 - 1.30 (m, 1H). LCMS: m/z 506.1 [M+H]⁺, Purity =97.97%. Chiral Purity: 87.96% ee.

INSCoV-601K(2): ¹H NMR (400 MHz, DMSO-d₆) δ = 8.97 (s, 1H), 8.58 (d, J= 1.8 Hz, 1H), 8.50 (s, 2H), 8.31 (br d, J= 7.5 Hz, 1H), 7.79 (d, J=1.8 Hz, 1H), 7.70 (br d, J= 8.7 Hz, 2H), 7.45 (br s, 2H), 6.08 (s, 1H), 4.14 - 4.03 (m, 2H), 3.91 - 3.76 (m, 1H), 3.16 (d, J= 5.0 Hz, 1H), 2.09 -1.71 (m, 7H), 1.61 - 1.34 (m, 2H). LCMS: m/z 506.1 [M+H]⁺, Purity =96.12%. Chiral Purity: 97.71% ee.

### Example 12. Synthesis of INSCoV-601N, INSCoV-601N(1) and INSCoV-601N(2)

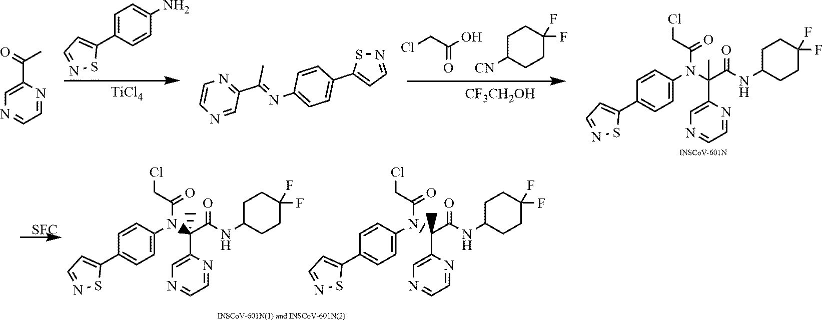

Step 1: To a solution of 1-(pyrazin-2-yl)ethan-1-one (500 mg, 4.09 mmol, 1 eq) and 4-isothiazol-5-ylaniline (649.40 mg, 3.68 mmol, 2.33 mL, 0.9 eq) in DCM (8 mL) was added TEA (1.24 g, 12.28 mmol, 1.71 mL, 3 eq) and TiCl₄ (1 M, 2.05 mL, 0.5 eq) at 0° C. under N₂, the mixture was stirred at 0° C. for 1 hr, then warmed up to 30° C. and stirred at 30° C. for 11 hrs. LCMS showed the 4-isothiazol-5-ylaniline was consumed and 62% of desired mass was detected. The mixture was diluted with NH₄Cl (10 mL), extracted with EtOAc (10 mL x 2) and washed with brine (30 mL). The organic layer was dried with anhydrous Na₂SO₄, filtered and concentrated under vacuum. The crude product was purified by Al₂O₃ chromatography eluted with DCM/Ethyl acetate=1:0 -0:1. N-(4-(isothiazol-5-yl)phenyl)-1-(pyrazin-2-yl)ethan-1-imine (0.9 g, 1.64 mmol, 39.99% yield, 51% purity) was obtained as a yellow solid.

¹H NMR (400 MHz, CHLOROFORM-d): δ = 9.50 (br s, 1H), 8.82 - 8.58 (m, 3H), 7.66 (d, J=8.4 Hz, 2H), 6.93 (d, J=8.4 Hz, 2H), 6.72 (br d, J=8.4 Hz, 1H), 2.39 (s, 3H).

Step 2: To a solution of N-(4-(isothiazol-5-yl)phenyl)-1-(pyrazin-2-yl)ethan-1-imine (0.9 g, 3.21 mmol, 1 eq) in CF₃CH₂OH (10 mL) was added 1,1-difluoro-4-isocyano-cyclohexane (465.97 mg, 3.21 mmol, 1 eq) and 2-chloroacetic acid (0.42 g, 4.44 mmol, 500.00 µL, 1.38 eq) at 0° C., the mixture was stirred at 0° C. for 1 hr. LCMS showed the N-(4-(isothiazol-5-yl)phenyl)-1-(pyrazin-2-yl)ethan-1-imine was consumed and 25% of desired product was detected. The mixture was diluted with water (10 mL), extracted with EtOAc (10 mL x 2) and washed with brine (20 mL). The organic layer was dried with anhydrous Na₂SO₄, filtered and concentrated under vacuum. The residue was purified by silica gel chromatography (column height: 250 mm, diameter: 100 mm, 100-200 mesh silica gel, Petroleum ether/Ethyl acetate=10/1, 1/1). 2-(2-Chloro-N-(4-(isothiazol-5-yl)phenyl)acetamido)-N-(4,4-difluorocyclohexyl)-2-(pyrazin-2-yl)propanamide INSCoV-601N (283.73 mg, 529.92 µmol, 16.51% yield, 97.119% purity) was obtained as a yellow solid.

¹H NMR (400 MHz, DMSO-d₆): δ= 9.06 (d, J=1.2 Hz, 1H), 8.69 - 8.53 (m, 3H), 8.25 (d, J=8.0 Hz, 1H), 7.97 - 7.79 (m, 4H), 7.58 (br d, J=8.0 Hz, 1H), 4.13 - 3.94 (m, 2H), 3.87 (br d, J=7.2 Hz, 1H), 2.11 - 1.50 (m, 8H), 1.42 (s, 3H). LCMS: m/z 520.1 [M+H]⁺, Purity =98.61%.

Step 3: Chiral SFC purification of INSCoV-601N: INSCoV-601N(1) and INSCoV-601N(2). INSCoV-601N (200 mg) was purified by SFC (column: DAICEL CHIRALCEL OD(250 mm x 30 mm, 10 µm); mobile phase: [Neu-IPA]; B%: 40%-40%, 4.0 min; 25 min) to give two peaks. First peak INSCoV-601N(1) (52.24 mg, 91.42 µmol, 23.77% yield, 91% purity) was obtained as an off-white solid. Second peak INSCoV-601N(2) (51.09 mg, 92.36 µmol, 24.01% yield, 94% purity) was obtained as an off-white solid.

INSCoV-601N(1): ¹H NMR (400 MHz, DMSO-d₆): δ = 9.06 (d, J=1.2 Hz, 1H), 8.68 - 8.59 (m, 2H), 8.57 (d, J=2.4 Hz, 1H), 8.25 (d, J=8.0 Hz, 1H), 7.96 - 7.80 (m, 4H), 7.58 (d, J=8.0 Hz, 1H), 4.12 - 3.94 (m, 2H), 3.87 (br d, J=7.6 Hz, 1H), 2.14 - 1.51 (m, 8H), 1.42 (s, 3H). LCMS: m/z 520.1 [M+H]⁺, Purity =94.81%. Chiral Purity: 100% ee.

INSCoV-601N(2): ¹H NMR (400 MHz, DMSO-d₆): δ = 9.06 (d, J=1.2 Hz, 1H), 8.68 - 8.60 (m, 2H), 8.57 (d, J=2.5 Hz, 1H), 8.25 (d, J=8.0 Hz, 1H), 7.94 - 7.80 (m, 4H), 7.58 (br d, J=8.0 Hz, 1H), 4.09 - 3.95 (m, 2H), 3.88 (br d, J=7.2 Hz, 1H), 2.11 - 1.52 (m, 8H), 1.42 (s, 3H). LCMS: m/z 520.1 [M+H]⁺, Purity =100%. Chiral Purity: 100% ee.

### Example 13. Synthesis of INSCoV-601P, INSCoV-601P(1A) and INSCoV-601P(1B)

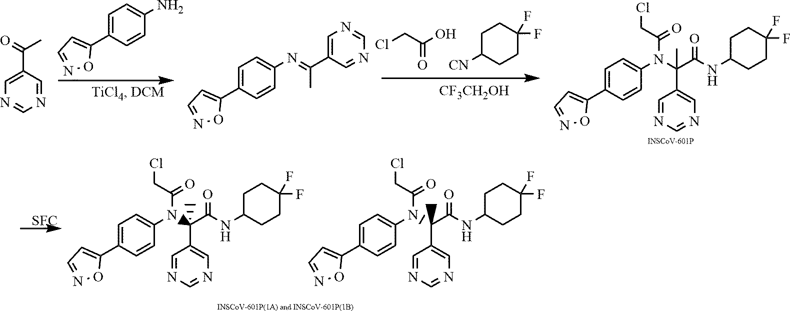

Step 1: To a solution of 4-(isoxazol-5-yl)aniline (2.6 g, 16.23 mmol, 1 eq) and 1-(pyrimidin-5-yl)ethan-1-one (2.38 g, 19.48 mmol, 1.2 eq) in DCM (26 mL) was added TEA (4.93 g, 48.70 mmol, 6.78 mL, 3 eq) and TiCl₄ (1 M, 8.12 mL, 0.5 eq) at 0° C. The reaction mixture was stirred at 25° C. for 12 hrs under N₂. LCMS showed reactant was consumed completely and one peak (Rₜ= 0.884 min) with desired mass was detected. The reaction mixture was filtered through a pad of Celite and washed with DCM (40 mL*2). The filtrate was concentrated under vacuum. The residue was used in next step without further purification. N-(4-(isothiazol-5-yl)phenyl)-1-(pyrazin-2-yl)ethan-1-imine (4.5 g, crude) was obtained as a yellow solid.

LCMS: m/z 265.2 [M+H]⁺, Purity =63.77%.

Step 2: To a solution of 2-chloroacetic acid (1.93 g, 20.43 mmol, 2.30 mL, 1.2 eq) and N-(4-(isothiazol-5-yl)phenyl)-1-(pyrazin-2-yl)ethan-1-imine (4.5 g, 17.03 mmol, 1 eq) in CF₃CH₂OH (15 mL) was added 1,1-difluoro-4-isocyanocyclohexane (2.47 g, 17.03 mmol, 1 eq). The reaction mxiture was stirred at 25° C. for 12 hrs. LCMS showed one peak (Rt =0.929 min) with desired mass was detected. The reaction was concentrated under vacuum. The residue was purified by prep-HPLC (column: Phenomenex luna C18 (250*70 mm,15 µm);mobile phase: [water(0.05%HCl)- ACN]; B%: 35ACN%-65ACN%,22 min), diluted with water (800 mL) and dried by lyophilization to get crude product. The residue was purified by normol phase-HPLC (column: Welch Ultimate XB-SiOH 250*50*10 µm;mobile phase: [Hexane- EtOH];B%: 1%-40%,20 min) and concentrated under vacuum. 2-(2-Chloro-N-(4-(isoxazol-5-yl)phenyl)acetamido)-N-(4,4-difluorocyclohexyl)-2-(pyrimidin-5-yl)propanamide (INSCoV-601P) (0.2 g, 360.54 µmol, 2.12% yield) was obtained as yellow solid.

¹H NMR (400 MHz, DMSO-d₆): δ = 9.04 (s, 1H), 8.89 - 8.83 (m, 2H), 8.69 (d, J =2.0 Hz, 1H), 8.03 - 7.79 (m, 3H), 7.66 - 7.59 (m, 1H), 7.37 (d, J =7.6 Hz, 1H), 7.13 (d, J =2.0 Hz, 1H), 4.07 - 3.91 (m, 2H), 3.90 - 3.81 (m, 1H), 2.07 - 1.56 (m, 11H). LCMS: m/z 504.2 [M+H]⁺, Purity =97.55%.

Step 3: Chiral SFC purification of INSCoV-601P: INSCoV-601P(lA) and INSCoV-601P(1B). The INSCoV-601P (0.2 g, 396.88 µmol, 1 eq) was separated by chiral SFC (column: DAICEL CHIRALPAK AD(250 mm*30 mm,10 µm);mobile phase: [Ne-EtOH];B%: 45%-45%,5.2;40 min) and concentrated under vacuum. Two peaks were then separated by chiral SFC (column: DAICEL CHIRALPAK IG(250 mm*50mm,10 µm);mobile phase: [Neu-MeOH];B%: 40%-40%,4.3 min;25 min) and chiral SFC (column: Daicel Chiral Pak IG (250*30 mm, 10 µm);mobile phase: [MeOH-ACN];B%: 50%-50%,4.1;40 min), respectively, and concentrated under vacuum. First peak INSCoV-601P(1A) (45.6 mg, 85.98 µmol, 22% yield) was obtained as a yellow solid. Second peak INSCoV-601P(1B) (53.71 mg, 97.77 µmol, 25% yield) was obtained as an orange solid.

INSCoV-601P(1A): ¹H NMR (400 MHz, CHLOROFORM-d) µ = 9.17 (s, 1H), 8.86 (s, 2H), 8.35 (d, J =2.0 Hz, 1H), 7.92 (s, 2H), 7.57 - 7.34 (m, 2H), 6.63 (d, J= 1.6 Hz, 1H), 6.51 (d, J= 7.2 Hz, 1H), 4.10 - 3.94 (m, 1H), 3.88 - 3.73 (m, 2H), 2.18 - 1.86 (m, 9H), 1.66 - 1.57(m, 2H). LCMS: m/z 504.2 [M+H]⁺, Purity =96.07%. Chiral Purity: 100% ee.

INSCoV-601P(1B): ¹H NMR (400 MHz, CHLOROFORM-d) δ = 9.13 (s, 1H), 8.82 (s, 2H), 8.34 (d, J =2.0 Hz, 1H), 7.90 (s, 2H), 7.56 - 7.30 (m, 2H), 6.62 (d, J =2.0 Hz, 1H), 6.50 (d, J =7.6 Hz, 1H), 4.01 - 3.95 (m, 1H), 3.85 - 3.73 (m, 2H), 2.23 - 1.83 (m, 9H), 1.60 (d, J= 10.0 Hz, 2H). LCMS: m/z 504.1 [M+H]⁺, Purity =97.39%. Chiral Purity: 100% ee.

### Example 14. Synthesis of INSCoV-601Q, INSCoV-601Q(1A) and INSCoV-601Q(1B)

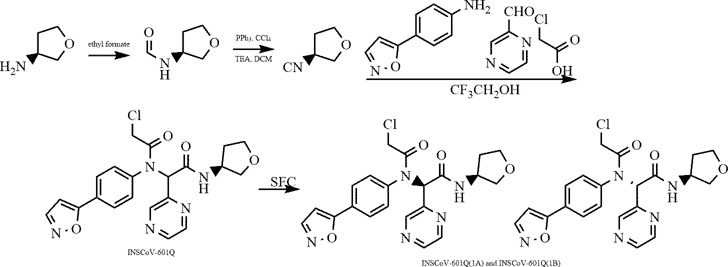

Step 1: To a solution of (S)-tetrahydrofuran-3-amine (4 g, 32.37 mmol, 1 eq, HCl) in ethyl formate (36.84 g, 497.31 mmol, 40.00 mL, 15.36 eq) was added TEA (9.83 g, 97.10 mmol, 13.52 mL, 3 eq) . The mixture was stirred at 80° C. for 17 hrs. TLC (DCM:MeOH = 10:1) showed (S)-tetrahydrofuran-3-amine (R_{f}=0.4) consumed and a new spot(R_{f} = 0.6) formed. The mixture was concentrated under vacuum. The residue was dissolved in DCM (80 mL), washed with H₂O (30 mL*3). The organic layer was dried over anhydrous Na₂SO₄, filtered and concentrated under vacuum. The crude was used in next step directly without further purification. (S)-N-(tetrahydrofuran-3-yl)formamide (3.8 g, crude) was obtained as a yellow oil.

¹H NMR (400 MHz, CHLOROFORM-d): δ = 8.06 (s, 1H), 6.85 (br s, 1H), 4.56 - 4.46 (m, 1H), 3.93 - 3.83 (m, 1H),3.81 - 3.70 (m, 2H), 3.67 - 3.56 (m, 1H), 2.30 - 2.15 (m, 1H), 1.87 - 1.72 (m, 1H).

Step 2: To a solution of (S)-N-(tetrahydrofuran-3-yl)formamide (4 g, 34.74 mmol, 1 eq) in DCM (40 mL) was added PPh₃ (9.11 g, 34.74 mmol, 1 eq) ,CCl₄ (5.34 g, 34.74 mmol, 3.34 mL, 1 eq) and TEA (3.87 g, 38.22 mmol, 5.32 mL, 1.1 eq) . The mixture was stirred at 45° C. for 17 hrs under N₂. TLC (PE: EA = 5:1) showed (S)-N-(tetrahydrofuran-3-yl)formamide consumed and a new spot (R_{f} = 0.5) formed. The mixture was concentrated under vacuum. The residue was dissolved in Et₂O (300 mL), stirred for 30 min, filtered and the filter cake was washed with Et₂O (100 mL*3).The combined organic layer was concentrated under vacuum. (S)-3-isocyanotetrahydrofuran (4 g, crude) was obtained as a yellow oil.

¹H NMR (400 MHz, CHLOROFORM-d): δ= 4.16 (br d, J= 4.0 Hz, 1H), 4.00 (q, J= 7.9 Hz, 1H), 3.95 - 3.84 (m, 2H), 2.24 - 2.17 (m, 1H), 1.92 (br s, 2H).

Step 3: To a solution of (S)-3-isocyanotetrahydrofuran (179.68 mg, 1.85 mmol, 1 eq), 4-(isoxazol-5-yl)aniline (296.35 mg, 1.85 mmol, 1 eq) in CF₃CH₂OH (6 mL) was added pyrazine-2-carbaldehyde (0.2 g, 1.85 mmol, 1 eq) , 2-chloroacetic acid (174.84 mg,1.85 mmol, 208.14 µL, 1 eq) . The mixture was stirred at 30° C. for 1 h. LCMS showed reactant consumed and desired mass was detected. To the mixture was added 30 mL MTBE, stirred for 30 min, filtered and the filter cake was concentrated under vacuum. To the mixture was added 30 mL MTBE, stirred for 30 min, filtered and the filter cake was concentrated under vacuum. 2-Chloro-N-(4-(isoxazol-5-yl)phenyl)-N-(2-oxo-1-(pyrazin-2-yl)-2-(((S)-tetrahydrofuran-3-yl)amino)ethyl)acetamid (INSCoV-601Q) (461.14 mg, 990.78 µmol, 53.55% yield) was obtained as a yellow solid.

¹H NMR (400 MHz, DMSO-d₆): δ= 8.65 (d, J= 2.0 Hz, 1H), 8.59 - 8.52 (m, 2H), 8.50 - 8.46 (m, 1H), 8.42 (d, J= 2.4 Hz, 1H),7.77 (br d, J= 7.7 Hz, 2H), 7.57 - 7.50 (m, 1H), 7.03 (d, J= 1.8 Hz, 1H), 6.26 (d, J = 2.1 Hz, 1H), 4.33 - 4.23 (m, 1H), 4.18 -4.02 (m, 2H), 3.87 (dq, J = 6.6, 9.7 Hz, 1H), 3.77 - 3.61 (m, 3H), 2.06 (td, J = 7.5, 12.7 Hz, 1H), 1.75 - 1.59 (m, 1H). LCMS: m/z 442.2 [M+H]⁺, Purity =85.55%.

Step 4: Chiral SFC purification of INSCoV-601Q: INSCoV-601Q(1A) and INSCoV-601Q(1B). Compound INSCoV-601Q (103 mg) was purified by SFC (Column: Cellucoat 50x4.6 mm I.D., 3 µm ^{,} Mobile phase: Phase A for CO₂, and Phase B for MeOH(0.05% DEA); Gradient elution: MeOH (0.05% DEA) in CO₂ from 5% to 40%; Flow rate: 3 mL /min; Detector: PDA; Column Temp: 35° C.; Back Pressure: 100Bar). The solution was concentrated under vacuum. First peak INSCoV-601Q (1A) (10.30 mg, 22.43 µmol, 9.91% yield, 96.243% purity) was obtained as a yellow solid. Second peak INSCoV-601Q (1B) (28.04 mg, 60.92 µmol, 26.92%yield, 96.007% purity) was obtained as a yellow solid.

INSCoV-601Q(1A): ¹H NMR (400 MHz, DMSO-d₆): δ = 8.65 (d, J = 2.0 Hz, 1H), 8.58 -8.53 (m, 2H), 8.50 - 8.46 (m, 1H), 8.42 (d, J= 2.4 Hz, 1H),7.77 (br d, J= 8.7 Hz, 2H), 7.65 - 7.38 (m, 2H), 7.03 (d, J = 1.8 Hz, 1H), 6.26 (s, 1H), 4.29 - 4.22 (m, 1H), 4.18 - 4.03 (m, 2H), 3.76 - 3.63 (m, 3H), 3.42 - 3.38 (m, 1H), 2.08 - 1.99 (m, 1H), 1.73 - 1.63 (m, 1H). LCMS: m/z 442.2 [M+H]⁺, Purity =96.27%. Chiral Purity: 69.84% ee.

INSCoV-601Q(1B): ¹H NMR (400 MHz, DMSO-d₆): δ = 8.65 (d, J =2.0 Hz, 1H), 8.59 -8.54 (m, 2H), 8.48 (dd, J= 1.5, 2.4 Hz, 1H), 8.43 (d, J= 2.6 Hz, 1H), 7.78 (br d, J= 8.8 Hz, 2H), 7.67 - 7.41 (m, 2H), 7.04 (d, J= 1.8 Hz, 1H), 6.27 (s, 1H), 4.33 - 4.25 (m, 1H), 4.18 - 4.03(m, 2H), 3.76 -3.62 (m, 2H), 3.41 (dd, J = 3.5, 9.0 Hz, 1H), 2.08 - 2.02 (m, 1H), 1.71 - 1.62 (m, 1H). LCMS: m/z 442.2 [M+H]⁺, Purity =98.23%. Chiral Purity: 74.77% ee.

### Example 15. Synthesis of INSCoV-614, INSCoV-614(1A), INSCoV-614(1B), INSCoV-614(2A) and INSCoV-614(2B)

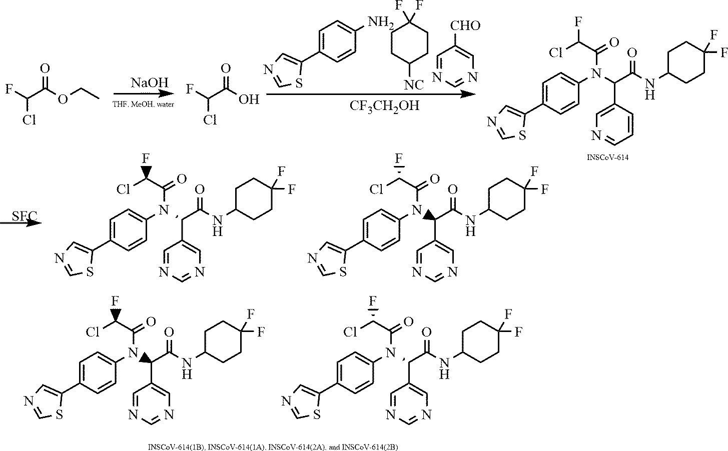

Step 1: To a solution of ethyl 2-chloro-2-fluoroacetate (1 g, 7.12 mmol, 826.45 µL, 1 eq) in THF (4 mL), MeOH (4 mL) and H₂O (2 mL) was added NaOH (426.92 mg, 10.67 mmol, 1.5 eq). The reaction mixture was stirred at 25° C. for 12 hrs. TLC (PE: EA = 5:1) showed one new spot formed. The reaction mixture was concentrated under vacuum, adjusted pH=2 by 1 N HCl solution and extracted with EtOAc (50 mL*3). The combined organic phases were dried over anhydrous Na₂SO₄ and concentrated under vacuum. The reaction mixture was used in next step directly without further purification. 2-chloro-2-fluoroacetic acid (0.6 g, crude) was obtained as a colorless oil.

¹H NMR (400 MHz, DMSO-d₆): δ = 7.03 - 6.65 (m, 1H).

Step 2: The compound was synthesized according to the procedure for the preparation of INSCoV-601H (Example 9). MTBE (20 mL) was added to the reaction mixture and cooled to 0° C. for 12 hrs. The mixture was filtered and washed with MTBE (10 mL*3). The filter cake was concentrated under vacuum. 2-Chloro-N-(2-((4,4-difluorocyclohexyl)amino)-2-oxo-1-(pyrimidin-5-yl)ethyl)-2-fluoro-N-(4-(thiazol-5-yl)phenyl)acetamide INSCoV-614 (0.5 g, 910.33 µmol, 32.75% yield) was obtained an orange solid.

¹H NMR (400 MHz, DMSO-d₆): δ= 9.11 (d, J= 0.8 Hz, 1H), 9.10 (d, J= 0.8 Hz, 1H), 8.99 (s, 1H), 8.96 (s, 1H), 8.53 (s, 2H), 8.51 (s, 1H), 8.39 - 8.35 (m, 1H), 8.34 (d, J= 4.4 Hz, 2H), 7.64 (s, 4H), 6.66 - 6.52 (m, 1H), 6.51 - 6.40 (m, 1H), 6.09 (s, 1H), 6.03 (s, 1H), 3.89 - 3.83 (m, 2H), 2.13 -1.64 (m, 12H), 1.60 - 1.46 (m, 2H), 1.42 - 1.26 (m, 2H). LCMS: m/z 524.1 [M+H]⁺, Purity =100%.

Step 3: Chiral SFC purification of INSCoV-614: INSCoV-614(1A), INSCoV-614(1B), INSCoV-614(2A) and INSCoV-614(2B). The compound INSCoV-614 (0.4 g, 763.42 µmol, 1 eq) was separated by chiral SFC (column: Daicel ChiralPak IG (250*30 mm, 10 µm); mobile phase: [Neu-MeOH]; B%: 40%-40%,5.6;90 min) and concentrated under vacuum. First peak INSCoV-614(1A) (50.34 mg, 93.52 µmol, 12.25% yield) was obtained as yellow solid. Second peak INSCoV-614(1B) (66.09 mg, 119.03 µmol, 15.59% yield) was obtained as a yellow solid. Third peak INSCoV-614(2A) (33.89 mg, 60.34 µmol, 7.90% yield) was obtained as a yellow solid. Fourth peak INSCoV-614(2B) (72.98 mg, 133.83 µmol, 17.53% yield) was obtained as a yellow solid.

INSCoV-614(1A): ¹H NMR (400 MHz, DMSO-d₆): δ = 9.11 (s, 1H), 8.99 (s, 1H), 8.53 (s, 2H), 8.38 - 8.31 (m, 2H), 7.68 - 7.62 (m, 3H), 7.49 - 7.24 (m, 1H), 6.72 - 6.38 (m, 1H), 6.03 (s, 1H), 3.89 - 3.76 (m, 1H), 2.01 - 1.71 (m, 6H), 1.59 - 1.45 (m, 1H), 1.43 - 1.28 (m, 1H). LCMS: m/z 524.1 [M+H]⁺, Purity =97.28%. Chiral Purity: 100% ee.

INSCoV-614(1B): ¹H NMR (400 MHz, DMSO-d₆): δ = 9.09 (s, 1H), 8.96 (s, 1H), 8.51 (s, 2H), 8.38 (d, J =7.6 Hz, 1H), 8.33 (s, 1H), 7.74 - 7.55 (m, 3H), 7.48 - 7.11 (m, 1H), 6.59 - 6.38 (m, 1H), 6.09 (s, 1H), 3.85 (d, J= 6.4 Hz, 1H), 2.04 - 1.73 (m, 6H), 1.62 - 1.44 (m, 1H), 1.42 - 1.25 (m, 1H). LCMS: m/z 524.1 [M+H]⁺, Purity =98.36%. Chiral Purity: 96.75% ee.

INSCoV-614(2A): ¹H NMR (400 MHz, DMSO-d₆): δ = 9.10 (s, 1H), 8.96 (s, 1H), 8.51 (s, 2H), 8.36 (d, J =7.6 Hz, 1H), 8.34 (s, 1H), 7.66 - 7.60 (m, 3H), 7.49 - 7.22 (m, 1H), 6.58 - 6.41 (m, 1H), 6.09 (s, 1H), 3.89 - 3.83 (m, 1H), 2.03 - 1.71 (m, 6H), 1.60 - 1.45 (m, 1H), 1.41 - 1.25 (m, 1H). LCMS: m/z 524.1 [M+H]⁺, Purity =98.01%. Chiral Purity: 100% ee.

INSCoV-614(2B): ¹H NMR (400 MHz, DMSO-d₆): δ = 9.12 (s, 1H), 9.00 (s, 1H), 8.55 (s, 2H), 8.45 - 8.32 (m, 2H), 7.69 - 7.63 (m, 3H), 7.27 - 7.06 (m, 1H), 6.73 - 6.41 (m, 1H), 6.04 (s, 1H), 3.93 - 3.73 (m, 1H), 2.06 - 1.70 (m, 6H), 1.61 - 1.45 (m, 1H), 1.43 - 1.26 (m, 1H). LCMS: m/z 524.1 [M+H]⁺, Purity =98.39%. Chiral Purity: 99.26% ee.

According to modelling and activity data, INSCoV-614(1B) is expected to have a structure of

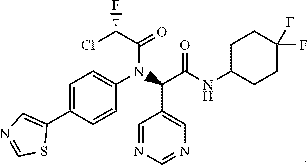

### Example 16. Synthesis of INSCoV-614A, INSCoV-614A(1A). INSCoV-614A(1B), INSCoV-614A(2A) and INSCoV-614A(2B)

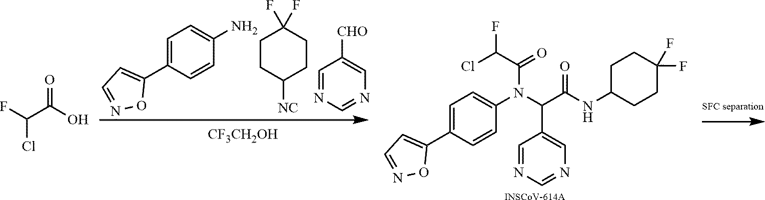

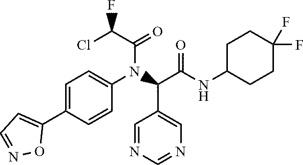 [SMILES: O=C(NC1CCC(F)(F)CC1)[C@@H](c1cncnc1)N(C(=O)[C@H](F)Cl)c1ccc(-c2ccno2)cc1]

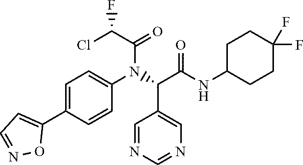 [SMILES: O=C(NC1CCC(F)(F)CC1)[C@H](c1cncnc1)N(C(=O)[C@@H](F)Cl)c1ccc(-c2ccno2)cc1]

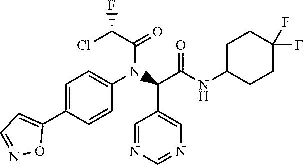 [SMILES: O=C(NC1CCC(F)(F)CC1)[C@@H](c1cncnc1)N(C(=O)[C@@H](F)Cl)c1ccc(-c2ccno2)cc1]

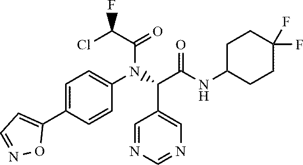 [SMILES: O=C(NC1CCC(F)(F)CC1)[C@H](c1cncnc1)N(C(=O)[C@H](F)Cl)c1ccc(-c2ccno2)cc1]

INSCoV-614A(1A), INSCoV-614A(1B), INSCoV-614A(2A) and INSCoV-614A(2B)

Step 1: To a solution of 2-chloro-2-fluoroacetic acid (421 mg, 3.74 mmol, 1.2 eq) and pyrimidine-5-carbaldehyde (338 mg, 3.13 mmol, 1.00 eq) in CF₃CH₂OH (10 mL) was added 4-(isoxazol-5-yl)aniline (500 mg, 3.12 mmol, 1 eq) and 1,1-difluoro-4-isocyanocyclohexane (453 mg, 3.12 mmol, 1 eq). The mixture was stirred at 25° C. for 1 hr. LCMS showed the starting material was consumed completely and about 72% of desired product were detected. The reaction mixture was concentrated under reduced pressure to give a crude product. The residue was purified by flash silica gel chromatography (ISCO®; 25 g SepaFlash® Silica Flash Column, Eluent: 0-85% Ethyl acetate in Petroleum ether @ 40 mL/min). 2-Chloro-N-(2-((4,4-difluorocyclohexyl)amino)-2-oxo-1-(pyrimidin-5-yl)ethyl)-2-fluoro-N-(4-(isoxazol-5-yl)phenyl)acetamide (INSCoV-614A) (1.05 g, 1.96 mmol, 63% yield) was obtained as a yellow solid.

¹H NMR (400 MHz, DMSO-d₆): δ =9.02 - 8.92 (m, 1H), 8.72 - 8.63 (m, 1H), 8.56 - 8.48 (m, 2H), 8.43 - 8.32 (m, 1H), 7.81 (brs, 2H), 7.69 - 7.19 (m, 2H), 7.12 - 7.02 (m, 1H), 6.68 - 6.43 (m, 1H), 6.14 - 6.00 (m, 1H), 3.86 (br s, 1H), 2.12 - 2.00 (m, 1H), 1.96 - 1.68 (m, 5H), 1.62 - 1.45 (m, 1H), 1.40 - 1.25 (m, 1H). LCMS: m/z 508.2 [M+H]⁺, Purity =100%.

Step 2: Chiral SFC purification of INSCoV-614A: INSCo-V614A(1A), INSCoV-614A(1B), INSCoV-614A(2A) andINSCoV-614A(2B). The compound INSCoV-614A (1.05 g, 1.96 mmol, 95% purity, 1 eq) was separated by chiral SFC (Phenomenex-Cellulose-2 (250 mm*50mm,10 µm); Mobile phase: A: Supercritical CO₂, B: Neu-EtOH; Isocratic: A:B = 70:30; Flow rate: 200 mL/min) and concentrated under vacuum to afford four fractions. First peak INSCoV-614A(1B): (112 mg, 216.54 µmol, 98.194% purity, 11% yield) was obtained as a yellow solid. Second peak INSCoV-614A(1A): (144 mg, 280.18 µmol, 98.82% purity, 14.3% yield) was obtained as a white solid. Third peak INSCoV-614A(2B) (120 mg, 231.17 µmol, 97.841% purity, 11.8% yield) was obtained as a yellow solid. Fourth peak INSCoV-614A(2A) (202 mg, 389.26 µmol, 97.872% purity, 19.9% yield) was obtained as a yellow solid.

INSCoV-614A(1A): ¹H NMR (400 MHz, DMSO-d₆): 8.98 (s, 1H), 8.67 (d, J= 1.6 Hz, 1H), 8.53 (s, 2H), 8.36 (d, J =7.6 Hz, 1H), 7.83 (d, J =6.8 Hz, 2H), 7.74 - 7.18 (m, 2H), 7.08 (d, J =1.6 Hz, 1H), 6.69 - 6.47 (m, 1H), 6.04 (s, 1H), 3.91 - 3.75 (m, 1H), 2.05 - 1.71 (m, 6H), 1.60 - 1.45 (m, 1H), 1.40 - 1.27 (m, 1H). LCMS: m/z 508.1 [M+H]⁺, Purity =100%. Chiral Purity: 95.48% ee.

INSCoV-614A(1B): ¹H NMR (400 MHz, DMSO-d₆) : 8.97 (s, 1H), 8.67 (d, J= 2.0 Hz, 1H), 8.53 (s, 2H), 8.36 (d, J =7.6 Hz, 1H), 7.90 - 7.75 (m, 2H), 7.74 - 7.17 (m, 2H), 7.07 (d, J =1.6 Hz, 1H), 6.69 - 6.49 (m, 1H), 6.04 (s, 1H), 3.91 - 3.76 (m, 1H), 2.06 - 1.68 (m, 6H), 1.59 - 1.43 (m, 1H), 1.39 - 1.26 (m, 1H). LCMS: m/z 508.0 [M+H]⁺, Purity =100%. Chiral Purity: 98.72% ee.

INSCoV-614A(2A): ¹H NMR (400 MHz, DMSO-d₆): 8.95 (s, 1H), 8.67 (d, J= 2.0 Hz, 1H), 8.51 (s, 2H), 8.39 (d, J= 7.6 Hz, 1H), 7.95 - 7.75 (m, 2H), 7.73 - 7.14 (m, 2H), 7.06 (d, J= 1.8 Hz, 1H), 6.67 - 6.40 (m, 1H), 6.11 (s, 1H), 3.87 (br s, 1H), 2.07 - 1.71 (m, 6H), 1.60 - 1.45 (m, 1H), 1.42 - 1.25 (m, 1H). LCMS: m/z 508.1 [M+H]⁺, Purity =96.46%. Chiral Purity: 95.82% ee.

INSCoV-614A(2B): ¹H NMR (400 MHz, DMSO-d₆) : 8.95 (s, 1H), 8.66 (d, J= 2.0 Hz, 1H), 8.51 (s, 2H), 8.39 (d, J= 7.6 Hz, 1H), 7.97 - 7.75 (m, 2H), 7.74 - 7.24 (m, 2H), 7.06 (d, J= 1.6 Hz, 1H), 6.63 - 6.42 (m, 1H), 6.11 (s, 1H), 3.94 - 3.79 (m, 1H), 2.04 - 1.72 (m, 6H), 1.60 - 1.45 (m, 1H), 1.41 - 1.25 (m, 1H). LCMS: m/z 508.1 [M+H]⁺, Purity =97.34%. Chiral Purity: 92.96% ee.

According to modeling, INSCoV-614A(2A) is expected to have a structure of

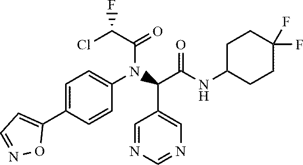 [SMILES: O=C(NC1CCC(F)(F)CC1)[C@@H](c1cncnc1)N(C(=O)[C@@H](F)Cl)c1ccc(-c2ccno2)cc1]

### Example 17. Synthesis of INSCoV-110A, INSCoV-110A(1) and INSCoV-110A(2)

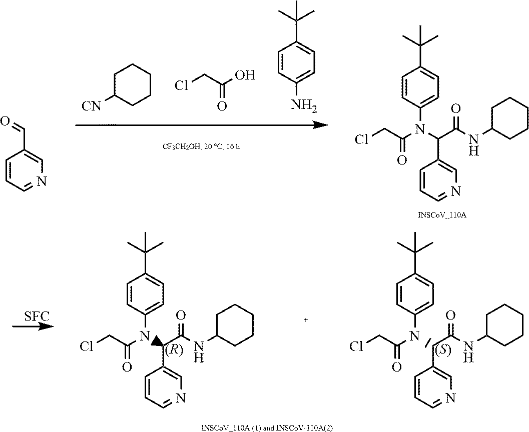

To a solution of 2-chloroacetic acid (88.22 mg, 933.62 µmol, 105.03 µL, 1 eq) and isocyanocyclohexane (101.92 mg, 933.62 µmol, 116.08 µL, 1 eq) in 75-89-8 (5 mL) was added 4-tert-butylaniline (139.33 mg, 933.62 µmol, 147.44 µL, 1 eq) and pyridine-3-carbaldehyde (0.1 g, 933.62 µmol, 87.72 µL, 1 eq). The reaction mixture was stirred at 15° C. for 1 hr. LCMS was showed SM consumed and DP formed. The solvent was evaporated under reduced pressure. The crude product was reflux with PE/EA(10/1, 50 mL) and filtered to give 2-(4-tert-butyl-N-(2-chloroacetyl)anilino)-N-cyclohexyl-2-(3-pyridyl)acetamide (0.4 g, 904.99 µmol, 96.93% yield) as a white solid.

INSCoV-110A (1): (511.15 mg, 1.16 mmol, 34.08% yield) was obtained as yellow solid via SFC resolution of INSCoV-110A, which was confirmed by HNMR, LCMS, SFC and HPLC.

LCMS: Retention time: 1.537 min, [M+H⁺] = 442.2. HPLC: Retention time: 2.778 min. SFC: Retention time: 1.163 min. ¹H NMR (400 MHz, DMSO-d₆): δ = 8.30 (dd, J= 1.6, 4.8 Hz, 1H), 8.26 (d, J= 1.9 Hz, 1H), 8.05 (d, J =7.6 Hz, 1H), 7.29 (td, J= 1.9, 7.9 Hz, 1H), 7.21 (br d, J= 6.6 Hz, 2H), 7.09 (dd, J= 4.8, 7.8 Hz, 1H), 6.01 (s, 1H), 4.00 - 3.86 (m, 2H), 3.61 - 3.50 (m, 1H), 1.78 - 1.47 (m, 5H), 1.34 - 0.91 (m, 16H).

INSCoV-110A (2): (76.78 mg, 173.71 µmol, 19.19% yield, 100% purity) was obtained as white solid via SFC resolution of INSCoV-110A, which was confirmed by HNMR, LCMS, SFC and HPLC.

LCMS: Retention time: 1.509 min, [M+H⁺] = 442.2, 1. HPLC: Retention time: 2.080 min. SFC: Retention time: 2.087 min. ¹H NMR (400 MHz, DMSO-d₆): δ = 8.47 - 8.36 (m, 2H), 8.16 - 8.05 (m, 1H), 7.61 - 7.49 (m, 1H), 7.35 - 7.28 (m, 1H), 7.28 - 7.22 (m, 2H), 7.22 - 7.08 (m, 1H), 6.07 - 6.04 (m, 1H), 4.02 - 3.90 (m, 2H), 1.75 - 1.48 (m, 5H), 1.28 - 1.00 (m, 17H).

### Example 18. Synthesis of INSCoV-110B, INSCoV-110B(1) and INSCoV-110B(2)

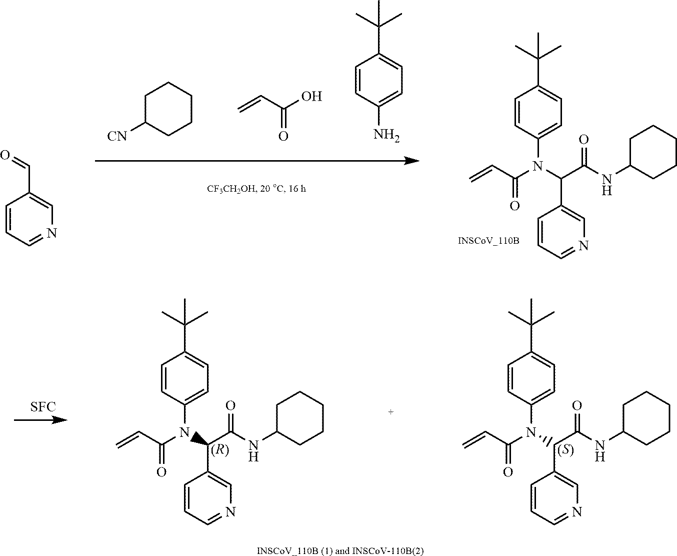

To a solution of acrylic acid (67.28 mg, 933.62 µmol, 64.08 µL, 1 eq) and isocyanocyclohexane (101.92 mg, 933.62 µmol, 116.08 µL, 1 eq) in 75-89-8 (5 mL) was added 4-tert-butylaniline (139.33 mg, 933.62 µmol, 147.44 µL, 1 eq) and pyridine-3-carbaldehyde (0.1 g, 933.62 µmol, 87.72 µL, 1 eq). The reaction mixture was stirred at 15° C. for 1 hr. LCMS was showed SM consumed and DP formed. The solvent was evaporated under reduced pressure. The crude product was purified by pre-HPLC (TFA) to give INSCoV-110B (0.23 g, 548.20 µmol, 58.72% yield) as a white solid.

LCMS: Retention time: 0.902 min, [M+H⁺] = 420.0. HPLC: Retention time: 2.779 min, 1. ¹H NMR (400 MHz, DMSO-d₆): δ = 8.47 - 8.25 (m, 2H), 8.08 (d, J= 7.7 Hz, 1H), 7.39 (br d, J= 7.9 Hz, 1H), 7.23 (br d, J = 8.3 Hz, 2H), 7.17 (dd, J = 4.8, 7.9 Hz, 1H), 7.09 (br d, J = 2.2 Hz, 1H), 6.26 -6.10 (m, 2H), 5.86 (dd, J= 10.3, 16.8 Hz, 1H), 5.66 -5.48 (m, 1H), 3.62 - 3.52 (m, 1H), 1.81 - 1.45 (m, 5H), 1.32 - 0.92 (m, 15H).

INSCoV-110B (1): (56.13 mg, 133.78 µmol, 24.40% yield) was obtained as white solid via SFC resolution of INSCoV-110B, which was confirmed by HNMR, LCMS, SFC and HPLC.

LCMS: Retention time: 0.909 min, [M+H⁺] = 420.0. HPLC: Retention time: 3.011 min. SFC: Retention time: 0.946 min. ¹H NMR (400 MHz, DMSO-d₆): δ = 8.36 - 8.24 (m, 2H), 8.06 (br d, J =7.7 Hz, 1H), 7.33 (br d, J =6.5 Hz, 1H), 7.21 (br d, J= 7.3 Hz, 2H), 7.15 - 6.97 (m, 2H), 6.22 -6.09 (m, 2H), 5.92 - 5.78 (m, 1H), 5.56 (br d, J= 10.6 Hz, 1H), 3.62 - 3.51 (m, 1H), 1.77 - 1.47 (m, 5H), 1.32 - 0.95 (m, 15H).

INSCoV-110B (2): (59.71 mg, 142.32 µmol, 25.96% yield) was obtained as white solid via SFC resolution of INSCoV-110B, which was confirmed by HNMR, LCMS, SFC and HPLC.

LCMS: Retention time: 0.905 min, [M+H⁺] = 442.2. HPLC: Retention time: 3.029 min. SFC: Retention time: 1.905 min, ¹HNMR: (400 MHz, DMSO-d₆): δ = 8.40 - 8.28 (m, 2H), 8.07 (br d, J =7.7 Hz, 1H), 7.39 (br d, J =7.9 Hz, 1H), 7.22 (br d, J =8.1 Hz, 2H), 7.17 (br dd, J = 5.0, 7.6 Hz, 1H), 7.13 -7.01 (m, 1H), 6.22 - 6.10 (m, 2H), 5.92 - 5.79 (m, 1H), 5.62 - 5.51 (m, 1H), 3.56 (br s, 1H), 1.73 - 1.50 (m, 5H), 1.32 - 0.97 (m, 16H).

### Example 19. Synthesis of INSCoV-110, INSCoV-110-1, and INSCoV-110-2

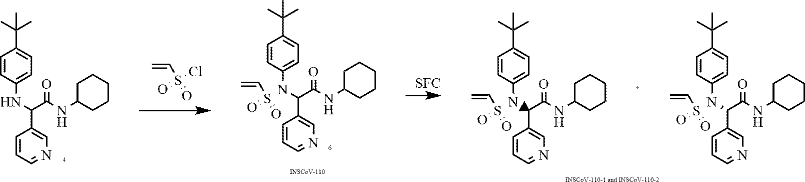

To a solution of compound 4 (200 mg, 547.18 µmol, 1 eq) and TEA (166.11 mg, 1.64 mmol, 228.48 µL, 3 eq) in DCM (8 mL) was added ethenesulfonyl chloride (138.50 mg, 1.09 mmol, 2.0 eq) dropwise at 0° C., the reaction was stirred at 20° C. for 2 hr. LCMS showed compound 4 was consumed, and desired mass was detected as main peak. The reaction mixture was concentrated. The residue was purified by flash silica gel chromatography (ISCO®; 25 g SepaFlash® Silica Flash Column, Eluent of 0 \~45% Ethyl acetate/Petroleum ether gradient @ 50 mL/min). Compound 6 (INSCoV-110) (110 mg, 229.60 µmol, 41.96% yield, 95.1% purity) was obtained as off-white solid, which was confirmed by LCMS.

LCMS: Retention time: 0.929 min, (M+H) = 456.1

Compound 6 (INSCoV-110) (110 mg, 241.43 µmol, 1 eq) was separated by Prep-SFC (column: DAICEL CHIRALCEL OD(250 mm*30 mm,10 um); mobile phase: [0.1%NH3H2O MEOH]; B%: 30%-30%,4.7 min;35 min).

INSCoV-110-1: (15 mg, 32.04 µmol, 13.27% yield, 97.304% purity) was obtained as white solid, which was confirmed by HNMR, LCMS, SFC and HPLC.

LCMS: Retention time: 1.085 min, (M+H) = 456.5. HPLC: Retention time: 2.738 min. ¹H NMR (400 MHz, CHLOROFORM-d): δ = 8.49 (dd, J=1.7, 4.8 Hz, 1H), 8.40 (d, J=2.2 Hz, 1H), 7.39 - 7.32 (m, 1H), 7.24 - 7.16 (m, 2H), 7.10 (dd, J=4.9, 7.9 Hz, 1H), 7.07 - 6.98 (m, 2H), 6.82 (dd, J=9.9, 16.6 Hz, 1H), 6.11 (d, J=16.6 Hz, 1H), 5.93 (d, J=9.9 Hz, 1H), 5.89 - 5.80 (m, 2H), 3.92 - 3.72 (m, 1H), 2.06 - 1.93 (m, 1H), 1.93 - 1.82 (m, 1H), 1.81 - 1.68 (m, 2H), 1.48 - 0.97 (m, 16H).

INSCoV-110-2: (20 mg, 42.90 µmol, 17.77% yield, 97.721% purity) was obtained as white solid, which was confirmed by HNMR, LCMS, SFC and HPLC.

LCMS: Retention time: 1.085 min, (M+H) = 456.5. HPLC: Retention time: 2.742 min. ¹H NMR (400 MHz, CHLOROFORM-d): δ = 8.50 (dd, J=1.6, 4.8 Hz, 1H), 8.40 (d, J=2.2 Hz, 1H), 7.35 (td, J=1.9, 8.0 Hz, 1H), 7.24 - 7.17 (m, 2H), 7.10 (dd, J=4.8, 7.9 Hz, 1H), 7.06 - 7.00 (m, 2H), 6.82 (dd, J=9.9, 16.5 Hz, 1H), 6.11 (d, J=16.6 Hz, 1H), 5.93 (d, J=9.9 Hz, 1H), 5.84 (s, 1H), 3.91 - 3.75 (m, 1H), 1.98 (br d, J=8.6 Hz, 1H), 1.88 (br d, J=11.0 Hz, 1H), 1.79 - 1.69 (m, 2H), 1.47 - 1.02 (m, 17H).

### Example 20. Synthesis of 2-Chloro-N-(2-((1,1-Dioxidotetrahydro-2H-Thiopyran-4-yl)Amino)-2-Oxo-1-(Pyrimidin-5-yl)Ethyl)-N-(4-(Oxazol-5-yl)Phenyl)Acetamide (INSCoV-501B)

INSCoV-501B was obtained according to the General procedure for INSCoV series and purification by Purification B: The crude product was triturated with MTBE (20 mL × 2) and filtered. Then was triturated with MeOH (6 mL) and filtered. INSCoV-501B (8.64 mg, 16.26 µmol, 2.59% yield, 94.862% purity) as orange solid was obtained, which was confirmed by HNMR, LCMS, SFC and HPLC.

LCMS: Retention time: 0.581 min, (M+H) = 504.0, HPLC: Retention time: 1.332 min, SFC: Retention time: 1.718 min, 2.024 min, ¹H NMR (400 MHz, DMSO-d₆): δ = 8.98 (s, 1H), 8.51 (s, 2H), 8.46 (s, 1H), 8.43 (d, J= 7.8 Hz, 1H), 7.72 (s, 1H), 7.66-7.64 (m, 2H), 7.42-7.41 (m, 1H), 6.04 (s, 1H), 4.10 - 3.96 (m, 3H), 3.28 - 3.19 (m, 2H), 3.13 - 3.04 (m, 1H), 3.03 - 2.94 (m, 1H), 2.12 - 1.90 (m, 3H), 1.86 - 1.73 (m, 1H).

### Example 21. Synthesis of 2-Chloro-N-(4-(Oxazol-5-yl)Phenyl)-N-(2-Oxo-1-(Pyrimidin-5-yl)-2-((1-Tosylethyl)Amino)Ethyl)Acetamide (INSCoV-501C(2))

INSCoV-501C(2) was synthesized according to the General procedure for INSCoV series and purification by method Purification A: The residue was dissolved in MeOH (2 mL) and purified by Pre-HPLC (column: 3_Phenomenex Luna C18 75 × 30 mm × 3 µm; mobile phase: [water (0.05%HCl)-ACN]; B%: 32%-52%, 6.5 min) and concentrated to remove MeCN, the liquid was under lyophilization to give the product. INSCoV-501C(2) (28.59 mg, 47.35 µmol, 5.12% yield, 91.759% purity) was obtained as yellow solid, which was confirmed by HNMR, LCMS, SFC and HPLC.

LCMS: Retention time: 0.831 min, (M+H) =554.2, HPLC: Retention time: 1.982 min, SFC: Retention time: 1.802 min, 2.474 min, ¹H NMR (400 MHz, DMSO-d₆): δ = 9.32 (d, J= 9.2 Hz, 1H), 8.94 (s, 1H), 8.47 - 8.43 (m, 1H), 8.34 (s, 2H), 7.80 (d, J= 8.2 Hz, 2H), 7.75 - 7.70 (m, 1H), 7.60 (d, J= 6.8 Hz, 2H), 7.49 - 7.26 (m, 3H), 6.21 (s, 1H), 5.25 - 5.19 (m, 1H), 3.94 (s, 2H), 2.42 (s, 3H), 1.35 (d, J = 7.0 Hz, 3H).

### Example 22. Synthesis of INSCoV-501G

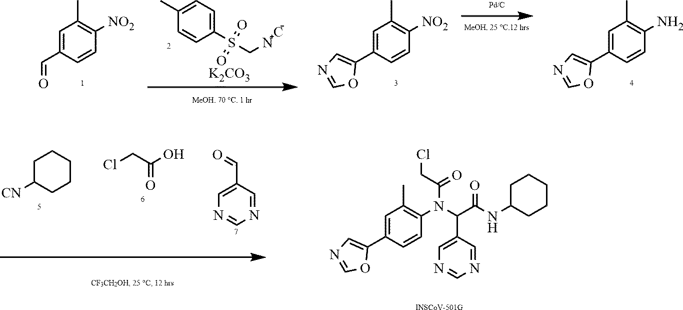

Step 1: To a solution of Compound 1 (3 g, 18.17 mmol, 1 eq) and Compound 2 (4.26 g, 21.80 mmol, 1.2 eq) in MeOH (70 mL) was added K₂CO₃ (5.02 g, 36.33 mmol, 2 eq). The reaction mixture was stirred at 70° C. for 1 hrs under N₂. TLC (PE: EA=4:1) showed Compound 1 (Rf= 0.8) was consumed completely and two new spots (Rf=0.4, Rf=0.0) formed. The reaction mixture was concentrated under vacuum. The residue was diluted with NaHCO₃ solution (20 mL) and extracted with EA (30 mL × 2). The combined organic phase was washed with water (30 mL), dried with anhydrous Na₂SO₄ and concentrated under vacuum. The residue was purified by flash silica gel chromatography (ISCO®; 80 g SepaFlash® Silica Flash Column, Eluent of 0 \~50% Ethyl acetate/Petroleum ether gradient @ 100 mL/min) and concentrated under vacuum. Compound 3 (3.5 g, 16.93 mmol, 93.22% yield, 98.79% purity) was obtained as yellow solid, which was confirmed by LCMS and HNMR.

LCMS: Retention time: 0.930 min, (M+H) =205.2. ¹H NMR (400 MHz, DMSO-d6) δ = 8.58 (d, J= 1.2 Hz, 1H), 8.11 (d, J= 8.6 Hz, 1H), 7.94 (d, J= 1.2 Hz, 1H), 7.88 (s, 1H), 7.79 (d, J= 8.8 Hz, 1H), 2.58 (s, 3H).

Step 2: To a solution of Compound 3 (1.5 g, 7.35 mmol, 1 eq) in MeOH (15 mL) was added Pd/C (1.5 g, 7.35 mmol, 10% purity, 1 eq). The reaction mixture was stirred at 25° C. for 12 hr under H₂ (15 PSI). LCMS showed Compound 3 was consumed completely and one peak (Rt= 0.827 min) with desired mass was detected. The reaction mixture was filtered through a pad of Celite and washed with MeOH (20 mL × 2). The filtrate was concentrated under vacuum. The reaction was used to next step and no purification. Compound 4 (1 g, crude) was obtained as off-white solid.

Step 3: INSCoV-501G was obtained according to the General procedure for INSCoV series and purification by Purification B: MTBE (20 mL) was added the reaction mixture and cooled to 0° C. for 1 hr. The mixture was filtered and washed with MTBE (10 mL × 3). The filter cake was concentrated under vacuum. INSCoV-501G (192.57 mg, 409.06 µmol, 29.48% yield, 99.403% purity) was obtained as yellow solid, which was confirmed by HNMR, LCMS, SFC and HPLC.

LCMS: Retention time: 0.933 min, (M+H) =468.3. HPLC: Retention time: 2.126 min. SFC: Retention time: 1.002 min, 1.136 min. ¹H NMR (400 MHz, DMSO-d₆) δ = 8.93 (s, 1H), 8.48 (s, 2H), 8.45 (s, 1H), 7.99 (d, J= 8.4 Hz, 1H), 7.74 - 7.67 (m, 1H), 7.67 - 7.61 (m, 1H), 7.42 (d, J = 1.6 Hz, 1H), 6.04 (s, 1H), 4.02 - 3.86 (m, 2H), 3.63 - 3.51 (m, 1H), 1.89 (s, 3H), 1.83 - 1.74 (m, 1H), 1.72 - 1.65 (m, 1H), 1.60 - 1.48 (m, 3H), 1.33 - 1.12 (m, 4H), 0.99 - 0.87 (m, 1H).

### Example 23. Synthesis of INSCoV-501H

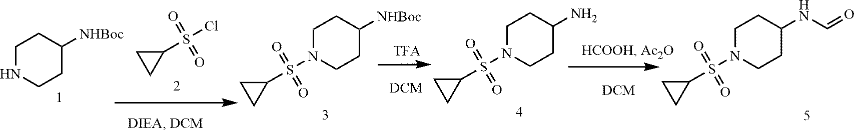

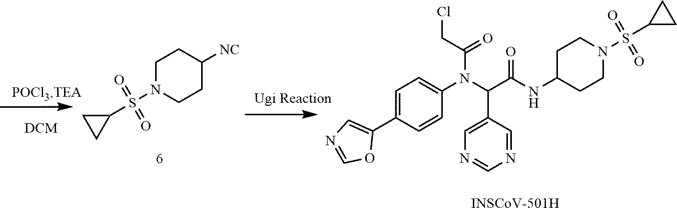

Step 1: To a solution of compound 1 (40 g, 199.72 mmol, 1 eq) and DIPEA (77.44 g, 599.17 mmol, 104.36 mL, 3 eq) in DCM (600 mL) was added compound 2 (33.69 g, 239.67 mmol, 1.2 eq) at 0° C. The mixture was stirred at 20° C. for 16 hrs. TLC (PE:EA=1:1) showed compound 1 (Rf=0.05) consumed and a new spot (Rf=0.5) was observed. The mixture was poured into sat.NaHCO₃ (400 mL), and then extracted with DCM (200 mL × 2). The combined organic layers were washed with water (200 mL), brine (100 mL), dried over Na₂SO₄, filtered and concentrated in vacuum to give a residue. The residue was triturated with MTBE (400 mL). The filter cake was washed with MTBE (100 mL×2), and then dried in vacuum. Compound 3 (56 g, 183.97 mmol, 92.11% yield) was obtained as white solid, which was determined by HNMR.

¹H NMR (400 MHz, CHLOROFORM-d): δ = 4.59 - 4.37 (m, 1H), 3.76 (br d, J= 12.2 Hz, 2H), 3.57 (br s, 1H), 2.92 (br t, J =11.1 Hz, 2H), 2.26 (tt, J= 4.8, 8.0 Hz, 1H), 2.10 - 1.96 (m, 2H), 1.56 - 1.38 (m, 11H), 1.21 - 1.11 (m, 2H), 1.04 - 0.93 (m, 2H).

Step 2: TFA (15 mL) was added to a solution of compound 3 (10 g, 32.85 mmol, 1 eq) in DCM (100 mL) at 0° C. The reaction was stirred at 25° C. for 5 hr. TLC (PE:EA=3:1,12) indicated compound 3 (Rf=0.3) was consumed completely and three new spots (Rf=0.05,Rf=0.6, Rf=0.8) were formed. The reaction mixture was added to H₂O 100 mL at 25° C. and extracted with DCM (100 mL × 2). The pH of aqueous phase was adjusted to 9 by sat. NaHCO₃, and it was extracted with DCM (100 mL × 6). The combined organic layers dried over Na₂SO₄, filtered and concentrated under reduced pressure to give a crude. The crude product was used into the next step without further purification. Compound 4 (1 g, 4.90 mmol, 14.90% yield) was obtained as a white solid, which was checked by HNMR.

¹H NMR (400 MHz, CHLOROFORM-d): δ = 3.69 (td, J= 3.2, 12.4 Hz, 2H), 2.90 - 2.70 (m, 3H), 2.19 (tt, J= 4.8, 8.0 Hz, 1H),1.55 - 1.30 (m, 6H), 1.12 - 1.03 (m, 2H), 0.94 - 0.84 (m, 2H).

Step 3: A mixture of HCOOH (261.02 mg, 5.43 mmol, 3.0 eq) and Ac2O (221.88 mg, 2.17 mmol, 203.56 µL, 1.2 eq) was stirred at 25° C. for 10 min. A solution of compound 4 (370 mg, 1.81 mmol, 1 eq) in DCM (5 mL) was added above mixture slowly. The mixture was stirred at 25° C. for 15 h. TLC (MeOH:DCM=1:10, 12) showed that the compound 4 (Rf=0) was consumed and a new spots (Rf=0.3) was observed. The mixture was poured into sat.NaHCO₃ (100 mL) slowly at 10° C., and then extracted with DCM (80 mL × 2). The combined organic layers were washed with brine (40 mL), dried over Na₂SO₄, filtered and concentrated in vacuum to give a crude. The crude product was used in next step without further purification. Compound 5 (370 mg, 1.59 mmol, 87.94% yield) was obtained as light yellow solid, which was checked by HNMR.

¹H NMR (400 MHz, CHLOROFORM-d): δ = 8.23 - 8.05 (m, 1H), 5.73 (br s, 1H), 4.14 -3.97 (m, 1H), 3.89 - 3.74 (m, 2H), 3.09- 2.84 (m, 2H), 2.37 - 2.20 (m, 1H), 2.12 - 2.00 (m, 2H),

Step 4: To a solution of compound 5 (350 mg, 1.51 mmol, 1 eq) and Et3N (457.38 mg, 4.52 mmol, 629.14 µL, 3.0 eq) in DCM (30 mL) was added POCl₃ (693.06 mg, 4.52 mmol, 420.03 µL, 3.0 eq) at 0° C. drop-wise slowly. The mixture was stirred at 0° C. for 1 h. TLC (DCM:MeOH=10:1) showed that compound 5 (Rf=0) was remained and a new spots (Rf=0.3) was observed. The mixture was poured into sat.NaHCO₃ (60 mL) slowly at 10° C., and then extracted with DCM (45 mL × 2). The combined organic layers were washed with brine (20 mL × 2), dried over Na₂SO₄, filtered and concentrated in vacuo to give a crude. The crude product was purified by silica gel chromatography eluted with Petroleum ether/Ethyl acetate=1:1. Compound 6 (220 mg, 1.03 mmol, 68.14% yield) was obtained as yellow solid, which was checked by HNMR.

¹H NMR (400 MHz, CHLOROFORM-d): δ = 3.93 (br s, 1H), 3.56 - 3.45 (m, 2H), 3.44 -3.29 (m, 2H), 2.28 (tt, J = 4.9, 8.0 Hz,1H), 2.03 - 1.91 (m, 4H), 1.23 - 1.14 (m, 2H), 1.07 - 0.97 (m, 2H).

Step 5: INSCoV-501H was obtained according to the General procedure for INSCoV series and purification by Purification B: The reaction was concentrated under vacuum. The crude was dissolved in ethyl acetate (6 mL), stirred for a moment and filtered and the filter cake was concentrated under vacuum. INSCoV-501H (102.99 mg, 174.30 µmol, 37.35% yield, 94.609% purity) was obtained as yellow solid, which was confirmed by HNMR, LCMS and HPLC.

LCMS: Retention time: 0.793 min, (M+H) = 559.1/561.1; Retention time: 0.787 min, (M+H) = 559.1/561.1. HPLC: Retention time: 1.784 min. ¹H NMR (400 MHz, DMSO-d6): δ = 8.98 (s, 1H), 8.51 (s, 2H), 8.46 (s, 1H), 8.38 (d, J= 7.5 Hz, 1H), 7.72 (s, 1H), 7.66 (br d, J=8.4 Hz, 2H), 7.53 - 7.26 (m, 2H), 6.10 (s, 1H), 4.10 - 4.00 (m, 2H), 3.86 - 3.75 (m, 1H), 3.60 - 3.46 (m, 2H), 3.01 - 2.87 (m, 2H), 2.62 - 2.52 (m, 2H), 1.93 - 1.73 (m, 2H), 1.57 - 1.40 (m, 1H), 1.32 (dt, J= 8.3, 11.2 Hz, 1H), 1.00 - 0.89 (m, 4H).

### Example 24. Synthesis of INSCoV-501H(1)

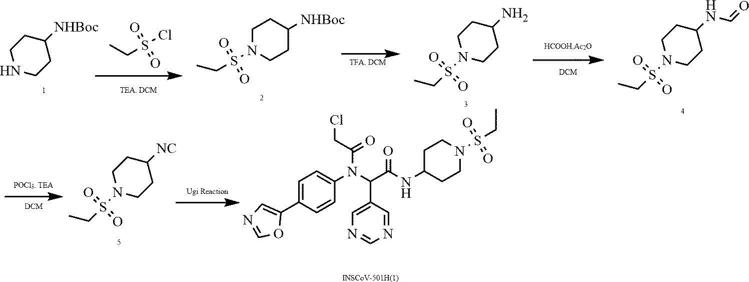

Step 1: To a solution of Compound 1 (10 g, 49.93 mmol, 1 eq) and Et₃N (7.58 g, 74.90 mmol, 10.42 mL, 1.5 eq) in DCM (60 mL) was added ethanesulfonyl chloride (7.06 g, 54.92 mmol, 5.19 mL, 1.1 eq) at 0° C. The mixture was stirred at 25° C. for 1 h. TLC(PE:EA=1:1,Ninhydrin) indicated Compound 1 (Rf--0.2) was consumed completely and two new spots (Rf=0.7,Rf =0.4)formed. The reaction mixture was quenched by addition H₂O 100 mL at 0° C., and extracted with DCM (100 mL × 3). The combined organic layers were washed with brine (40 mL × 3), dried over Na₂SO₄, filtered and concentrated under reduced pressure to give a residue. The crude product was triturated with EA(100 mL) at 25° C. for 20 min, then filtered to give a white solid. Compound 2 (12 g, 41.04 mmol, 82.20% yield) was obtained as a white solid, which was checked by HNMR.

¹H NMR (400 MHz, CHLOROFORM-d): δ = 4.68 - 4.29 (m, 1H), 3.78 (br d, J =12.3 Hz, 2H), 3.68 - 3.50 (m, 1H), 3.01 - 2.87(m, 4H), 2.03 (br dd, J= 2.7, 12.8 Hz, 2H), 1.55 - 1.49 (m, 1H), 1.55 - 1.48 (m, 1H), 1.46 (s, 9H), 1.37 (t, J= 7.5 Hz, 3H).

Step 2: To a solution of compound 2 (8 g, 27.36 mmol, 1 eq) in DCM (50 mL) was added TFA(7.5 mL) at 0° C. The reaction was stirred at 25° C. for 5 hr. TLC (PE:EA=1:1,Ninhydrin) indicated compound 2 was consumed completely and one new spot formed. The reaction mixture was quenched by addition H₂O 80 mL at 0° C. The reaction mixture was extracted with DCM (100 mL × 10). The combined organic layers were dried over Na₂SO₄, filtered and concentrated under reduced pressure to give a residue. Compound 3 (3.5 g, 18.20 mmol, 66.53% yield) was obtained as a light yellow oil, which was checked by HNMR.

¹H NMR (400 MHz, CHLOROFORM-d): δ = 3.65 (br dd, J= 2.8, 12.2 Hz, 2H), 2.96 - 2.69 (m, 5H), 1.88 - 1.74 (m, 2H), 1.57 -1.42 (m, 2H), 1.39 - 1.21 (m, 4H), 1.22 - 1.12 (m, 1H).

Step 3: A mixture of HCOOH (1.50 g, 31.20 mmol, 3.0 eq) and Ac₂O (1.27 g, 12.48 mmol, 1.17 mL, 1.2 eq) was stirred at 25° C. for 10 min. A solution of compound 3 (2.0 g, 10.40 mmol, 1 eq) in DCM (20 mL) was added above mixture slowly. The mixture was stirred at 25° C. for 3 h. TLC (MeOH:DCM=1:10, 12) showed that compound 3 (Rf=0) was consumed and a new spots (Rf=0.3) was observed. The mixture was poured into sat.NaHCO₃ (100 mL) slowly at 10° C., and then extracted with DCM (80 mL×2). The combined organic layers were washed with brine (40 mL), dried over Na₂SO₄, filtered and concentrated in vacuum to give a crude. The crude product was used in next step without further purification. Compound 4 (2.25 g, 10.21 mmol, 98.20% yield) was obtained as light yellow solid, which was checked by HNMR.

¹H NMR (400 MHz, CHLOROFORM-d): δ = 8.15 (s, 1H), 5.82 - 5.56 (m, 1H), 4.17 - 3.94 (m, 1H), 3.83 (br d, J = 12.8 Hz, 2H), 3.05 - 2.82 (m, 4H), 2.12 - 1.99 (m, 2H), 1.62 - 1.49 (m, 2H), 1.37 (t, J= 7.4 Hz, 3H).

Step 4: To a solution of compound 4 (0.3 g, 1.36 mmol, 1 eq) and Et₃N (413.41 mg, 4.09 mmol, 568.66 µL, 3.0 eq) in DCM (30 mL) was added POCl₃ (626.45 mg, 4.09 mmol, 379.66 µL, 3.0 eq) at 0° C. drop-wise slowly. The mixture was stirred at 0° C. for 1 h. TLC (PE:EA=1:1, 12) showed that compound 4 (Rf=0) was consumed, and a new spot (Rf=0.45) was observed. The mixture was poured into sat.NaHCO₃ (100 mL) slowly at 10° C., and then extracted with DCM (60 mL × 2). The combined organic layers were washed with brine (30 mL × 2), dried over Na₂SO₄, filtered and concentrated in vacuum to give a crude. The crude product was purified by silica gel chromatography eluted with Petroleum ether/Ethyl acetate=1:1 to give desired product. Compound 5 (165 mg, 815.73 µmol, 59.90% yield) was obtained as colorless oil, which was checked by HNMR.

¹H NMR (400 MHz, CHLOROFORM-d): δ = 3.94 (br s, 1H), 3.63 - 3.46 (m, 2H), 3.46 -3.30 (m, 2H), 2.98 (q, J = 7.4 Hz, 2H), 2.04 - 1.89 (m, 4H), 1.38 (t, J =7.4 Hz, 3H).

Step 5: INSCoV-501H(1) was obtained according to the General procedure for INSCoV series and Purification B: The reaction was concentrated under vacuum. The crude was dissolved in ethyl acetate (2 mL), stirred for a moment and filtered and the filter cake was concentrated under vacuum. INSCoV-501H(1) (83.93 mg, 144.18 µmol, 46.19% yield, 93.968% purity) was obtained as orange solid, which was confirmed by LCMS, HPLC and HNMR.

LCMS: Retention time: 0.652 min, (M+H) = 547.1; Retention time: 0.790 min, (M+H) = 547.1 HPLC: Retention time: 1.752 min. ¹H NMR (400 MHz, DMSO-d6): δ = 8.97 (s, 1H), 8.49 (s, 2H), 8.46 (s, 1H), 8.36 (d, J= 7.5 Hz, 1H), 7.73 - 7.70 (m, 1H), 7.65(br d, J= 8.6 Hz, 2H), 7.49 - 7.33 (m, 2H), 6.08 (s, 1H), 4.05 (br d, J= 6.2 Hz, 2H), 3.84 - 3.74 (m, 1H), 3.60 - 3.39 (m, 3H), 3.07 - 3.01 (m, 2H), 2.95 - 2.86 (m, 2H), 1.88 - 1.74 (m, 2H), 1.50 - 1.40 (m, 1H), 1.33 - 1.25 (m, 1H), 1.21 - 1.17 (m, 3H).

### Example 25. Synthesis of 2-Chloro-N-(2-((1,1-Dioxidotetrahydro-2H-Thiopyran-4-yl)Amino)-2-Oxo-1-(Pyrazin-2-yl)Ethyl)-N-(4-(Oxazol-5-yl)Phenyl)Acetamide (INSCoV-501M)

INSCoV-501M was synthesized according to the General procedure for INSCoVseries and Purification A: The residue was purified by Pre-HPLC (column: Phenomenex Luna C18 150 × 25 mm × 10 µm; mobile phase: [water (0.225%FA)-ACN]; B%: 18%-48%, 11 min) and dried by lyophilization. INSCoV-501M (9.55 mg, 18.40 µmol, 6.63% yield, 97.122% purity) as yellow solid was obtained, which was confirmed by HNMR, LCMS, SFC and HPLC.

LCMS: Retention time: 0.807 min, (M+H) = 504.3; Retention time: 0.808 min, (M+H) = 504.2. HPLC: Retention time: 1.351 min. SFC: Retention time: 1.815 min, 2.285 min. ¹H NMR (400 MHz, DMSO-d₆): δ = 8.74 - 8.33 (m, 5H), 8.13 (s, 1H), 7.69 (s, 1H), 7.65 - 7.36 (m, 4H), 6.23 (s, 1H), 4.28 - 3.62 (m, 3H), 3.12 - 2.92 (m, 2H), 2.10 - 1.81 (m, 4H).

### Example 26. Synthesis of 2-Chloro-N-(4-(Oxazol-5-yl)Phenyl)-N-(2-Oxo-1-(Pyrazin-2-yl)-2-((Tosylmethyl)Amino)Ethyl)Acetamide (INSCoV-501O)

INSCoV-501O was obtained according to the General procedure for INSCoV series and Purification A: The residue was dissolved in MeOH (2 mL) and purified by Pre-HPLC (column: 3_Phenomenex Luna C18 75 × 30 mm × 3 µm; mobile phase: [water (0.05%HCl)-ACN]; B%: 30%-50%, 6.5 min) and concentrated to remove MeCN, the liquid was under lyophilization to give the product. INSCoV-501O (274.41 mg, 486.27 µmol, 52.56% yield, 95.689% purity) was obtained as yellow solid, which was confirmed by HNMR, LCMS, SFC and HPLC.

LCMS: Retention time: 0.873 min, (M+H) =540.1; Retention time: 0.864 min, (M+H) =540.1, HPLC: Retention time: 1.830 min. SFC: Retention time: 0.846 min, 1.337 min. ¹H NMR (400 MHz, DMSO-d6): δ = 9.50 - 9.32 (m, 1H), 8.51 - 8.45 (m, 1H), 8.46 - 8.40 (m, 2H), 8.25 (s, 1H), 7.73 - 7.63 (m, 3H), 7.60 - 7.54 (m, 2H), 7.40 - 7.26 (m, 3H), 6.26 (s, 1H), 4.93 - 4.63 (m, 2H), 4.14 - 3.93 (m, 2H), 2.39 (s, 3H).

### Example 27. Synthesis of 2-Chloro-N-(4-(Oxazol-5-vl)Phenyl)-N-(2-Oxo-2-(Phenethylamino)-1-(Pyrazin-2-yl)Ethyl)Acetamide (INSCoV-501P)

INSCoV-501P was obtained according to the General procedure of INSCoV series. The residue was diluted with MeOH (4 mL), purified by prep-HPLC (column: 3_Phenomenex Luna C18 75 × 30 mm × 3 µm; mobile phase: [water (0.05% HCl)-ACN]; B%: 34%-54%, 6.5 min) and concentrated to remove MeCN, the liquid was under lyophilization to give the product. INSCoV-501P (89.34 mg, 174.23 µmol, 18.83% yield, 92.815% purity) was obtained as yellow solid, which was confirmed by HNMR, LCMS, SFC and HPLC.

LCMS: Retention time: 0.879 min, (M+H) = 476.2; Retention time: 0.883 min, (M+H) =476.1. HPLC: Retention time: 1.913 min. SFC: Retention time: 0.786 min, 1.306 min. ¹H NMR (400 MHz, DMSO-d₆): δ = 8.50 - 8.37 (m, 4H), 7.70 (s, 1H), 7.60 (d, J= 8.4 Hz, 2H), 7.49 - 7.35 (m, 1H), 7.31 - 7.10 (m, 6H), 6.19 (s, 1H), 4.18 - 3.97 (m, 2H), 3.45 - 3.26 (m, 2H), 2.77 - 2.63 (m, 2H).

### Example 28. Synthesis of 2-Chloro-N-(2-((1-(Cyclopropylsulfonyl)Piperidin-4-yl)Amino)-2-Oxo-1-(Pyrazin-2-yl)Ethyl)-N-(4-(Oxazol-5-yl)Phenyl)Acetamide (INSCoV-501R)

INSCoV-501R was obtained according to the General procedure INSCoV series. Purification B: The reaction was concentrated under vacuum. The crude was dissolved in ethyl acetate (4 mL), stirred for a moment and filtered and the filter cake was concentrated under vacuum. INSCoV-501R (139.16 mg, 239.21 µmol, 51.26% yield, 96.095% purity) was obtained as off-white solid, which was determined by LCMS, HPLC and HNMR.

LCMS: Retention time: 0.793 min, (M+H) = 547.1; Retention time: 0.780 min, (M+H) = 547.1 HPLC: Retention time: 1.805 min. ¹H NMR (400 MHz, DMSO-d₆): δ = 8.56 - 8.48 (m, 2H), 8.46 - 8.41 (m, 2H), 8.35 (d, J= 7.4 Hz, 1H), 7.70 (s, 1H), 7.61 (br d, J= 8.5 Hz, 2H), 7.57 - 7.28 (m, 2H), 6.24 (s, 1H), 6.30 - 6.19 (m, 1H), 4.18 - 4.09 (m, 1H), 4.09 - 4.00 (m, 1H), 3.81 - 3.69 (m,1H), 3.53 - 3.43 (m, 2H), 3.00 - 2.88 (m, 2H), 2.58 - 2.51 (m, 2H), 1.79 (br d, J= 11.7 Hz, 2H), 1.51 - 1.30 (m, 2H), 0.97 - 0.84(m, 4H).

### Example 29. Synthesis of 2-Chloro-N-(2-((1-(Ethylsulfonyl)Piperidin-4-yl)Amino)-2-Oxo-1-(Pyrazin-2-yl)Ethyl)-N-(4-(Oxazol-5-yl)Phenyl)Acetamide (INSCoV-501R(1))

INSCoV-501R(1) was obtained according to the General procedure for INSCoV series. Purification B: The reaction was concentrated under vacuum. The crude was dissolved in ethyl acetate (2 mL), stirred for a moment and filtered and the filter cake was concentrated under vacuum. INSCoV-501R(1) (73.31 mg,131.92 µmol, 42.26% yield, 98.439% purity) was obtained as orange solid, which was confirmed by LCMS, HPLC and HNMR.

LCMS: Retention time: 0.642 min, (M+H) = 547.1; Retention time: 0.780 min, (M+H) = 547.1. HPLC: Retention time: 1.735 min. ¹H NMR (400 MHz, DMSO-d₆): δ = 8.53 - 8.47 (m, 2H), 8.45 - 8.42 (m, 2H), 8.33 (d, J= 7.6 Hz, 1H), 7.69 (s, 1H), 7.60 (br d, J= 8.4 Hz, 2H), 7.46 (br d, J= 2.4 Hz, 2H), 6.23 (s, 1H), 4.17 - 4.10 (m, 1H), 4.07 - 3.99 (m, 1H), 3.81 - 3.71 (m, 1H), 3.48 (br t, J= 13.3 Hz, 2H), 3.08 - 2.99 (m, 2H), 2.95 - 2.88 (m, 2H), 1.82 - 1.73 (m, 2H), 1.40 - 1.26 (m, 2H), 1.21 - 1.14 (m, 4H).

### Example 30. Synthesis of 2-Chloro-N-(2-((4,4-Difluorocyclohexyl)Amino)-2-Oxo-1-(Pyrazin-2-yl)Ethyl)-N-(4-(Oxazol-5-yl)Phenyl)Acetamide (INSCoV-501S)

INSCoV-501S was obtained according to the General procedure for INSCoV series. Purification A: The reaction mixture was concentrated in vacuum to give a residue. The residue was purified by Prep-HPLC (column: Phenomenex Synergi C18 150 × 25 mm × 10 µm; mobile phase: [water (0.1%TFA)-ACN]; B%: 35%-65%, 10 min) to give INSCoV-501S (100 mg, 204 µmol, 29% yield, 99.9% purity) as a yellow solid, which was confirmed by LCMS, HPLC and HNMR.

LCMS: Retention time: 0.842 min, (M+H) = 490.4. HPLC: Retention time: 1.796 min.¹H NMR (400 MHz, CDCl₃): δ = 8.75 (s, 1H), 8.54 (m, 1H), 8.51 (m, 1H), 7.95 (s, 1H), 7.67 (s, 1H), 7.65 (s, 1H), 7.45-7.44 (m, 2H), 7.41 (s, 1H), 7.33 (d, J =7.6 Hz, 1H), 5.89 (s, 1H), 3.98-3.87 (m, 3H), 2.10-1.97 (m, 3H), 1.94-1.79 (m, 3H), 1.63-1.47 (m, 2H).

### Example 31. Synthesis of 2-Chloro-N-(3-Chloro-4-Methoxyphenyl)-N-(2-(Cvclohexylamino)-2-Oxo-1-(Pyridin-3-yl)Ethyl)Acetamide (INSCoV-509)

INSCoV-509 was obtained according to the General procedure for INSCoV series. Purification A: The residue was purified by prep-HPLC (column: Phenomenex Luna C18 150 × 25 mm × 10um; mobile phase: [water (0.225%FA)-ACN]; B%: 36%-66%,10 min) to get solution. Crude product was purified by prep-HPLC (column: Phenomenex luna C18 150 × 25 mm × 10 um; mobile phase: [water (0.225%FA)-ACN]; B%: 36%-66%,10 min) to get solution 2. Combined the solution 1 and solution 2 and dried by lyophilization. INSCoV-509 (162.68 mg, 353.59 µmol, 27.86% yield, 97.886% purity) as white solid was obtained, which was confirmed by LCMS, HPLC and HNMR.

LCMS: Retention time: 0.696 min, [M+H] = 450.0; Retention time: 0.858 min, [M+H] = 450.2. HPLC: Retention time: 1.882 min. ¹H NMR (400 MHz, DMSO-d6): δ = 8.40 - 8.24 (m, 2H), 8.14 (d, J= 7.2 Hz, 1H), 8.00 - 7.54 (m, 1H), 7.41 - 7.30 (m, 1H), 7.23 - 7.13 (m, 1H), 7.11 - 6.68 (m, 2H), 6.10 - 5.97 (m, 1H), 4.09 - 3.90 (m, 2H), 3.86 - 3.71 (m, 3H), 3.66 - 3.52 (m, 1H), 1.79 - 1.49 (m, 5H), 1.31 - 0.94 (m, 5H).

### Example 32. Synthesis of 2-Chloro-N-(2-(Cyclohexylamino)-2-Oxo-1-(Pyridin-3-yl)Ethyl)-N-(2,4-Dimethoxyphenyl)Acetamide (INSCoV-512)

INSCoV-512 was obtained according to the General procedure for INSCoV series. Purification A: The crude product was purified by reversed-phase HPLC (0.1%FA condition) and concentrated under vacuum to remove MeCN and dried by lyophilization. INSCoV-512 (255.93 mg, 553.41 µmol, 59.28% yield, 96.427 % purity) as yellow solid was obtained, which was confirmed by HNMR, LCMS, SFC and HPLC.

LCMS: Retention time: 0.737 min, [M+H] = 446.0; Retention time: 0.831 min, [M+H] = 446.1. HPLC: Retention time: 1.682 min. SFC: Retention time: 1.143 min and 1.441 min. ¹H NMR (400 MHz, DMSO-d6): δ = 8.28-8.27 (m, 1H), 8.20 (d, J= 1.8 Hz, 1H), 7.99 (d, J= 7.8 Hz, 1H), 7.61 (d, J =8.8 Hz, 1H), 7.30-7.28 (m, 1H), 7.10-7.08 (m, 1H), 6.48-6.45 (m, 1H), 6.23 (d, J= 2.8 Hz, 1H), 5.88 (s, 1H), 3.96 (d, J =14.2 Hz, 1H), 3.79 (d, J =14.2 Hz, 1H), 3.67 (s, 3H), 3.60 - 3.52 (m, 1H), 3.45 (s, 3H), 1.79 - 1.52 (m, 5H), 1.25 - 0.93 (m, 5H).

### Example 33. Synthesis of INSCoV-517B

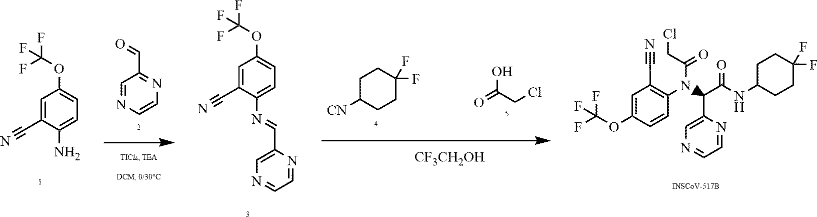

Step 1: To a solution of Compound 1 (500 mg, 2.47 mmol, 1 eq) and Compound 2(267.39 mg, 2.47 mmol, 1 eq) in DCM (0.5 mL) was added TiCl₄ (1 M, 1.24 mL, 0.5 eq), TEA (750.91 mg, 7.42 mmol, 1.03 mL, 3 eq) at 0° C. under N₂. The mixture was stirred at 0° C. for 1 h. Then the mixture was warmed to 30° C. and stirred at for 11 h. LCMS showed Compound 1 was consumed and desired mass was detected. The mixture was dissolved in DCM (50 mL) and quenched with sat. NH₄Cl (50 mL), separated and aqueous was extracted with DCM (3 × 20 mL). The organic layers were washed with brine, dried over anhydrous Na₂SO₄, filtered and concentrated under vacuum. Compound 3 (210 mg, crude) was obtained as yellow oil. It was used for next step directly.

LCMS: Retention time: 0.961/0.989 min, (M+H) = 293.1.

Step 2: INSCoV-517B was prepared by General methods for INSCoVseries. Purification B: The mixture was concentrated under vacuum. The residue was dissolved in METB (8 mL), stirred for 15 min, filtered and the filter cake was dissolved in ethyl acetate (8 mL), stirred for 15 min, filtered and the filter cake was concentrated under vacuum. INSCoV-517B (65.13 mg, 115.00 µmol, 16.69% yield, 93.909% purity) was obtained as off-white solid. Which was indicated by HNMR, FNMR, LCMS and HPLC.

LCMS: Retention time: 1.000 min, (M+H) = 532.3; Retention time: 0.903 min, (M+H) = 532.3. HPLC: Retention time: 2.219 min. ¹H NMR (400 MHz, DMSO-d₆): δ = 8.75 - 8.60 (m, 2H), 8.48 (d, J= 2.4 Hz, 1H), 8.39 - 8.33 (m, 1H), 8.23 (br d, J= 3.5 Hz,1H), 8.13 (br s, 1H), 7.88 - 7.72 (m, 2H), 6.32 (s, 1H), 4.14 (br d, J= 2.1 Hz, 2H), 3.87 - 3.57 (m, 1H), 2.04 - 1.62 (m, 8H), 1.53- 1.27 (m, 2H). ¹⁹F NMR (377 MHz, DMSO-d6): δ = -93.00 - -94.10 (m, 1F), -96.71 - -98.22 (m, 1F). ¹H NMR (400 MHz, DMSO-d6, T=50): δ= 8.60 - 8.52 (m, 1H), 8.45 - 8.39 (m, 1H), 8.31 (br s, 1H), 8.22 - 8.14 (m, 1H), 8.13 - 8.08(m, 1H), 8.06 - 7.95 (m, 1H), 7.79 - 7.66 (m, 2H), 6.29 (s, 1H), 4.20 - 3.98 (m, 2H), 3.82 - 3.70 (m, 1H), 1.99 - 1.65 (m, 8H), 1.50 - 1.30 (m, 2H). ¹⁹F NMR (376 MHz, DMSO-d6, T=50): δ = -93.35 - -94.20 (m, 1F), -96.66 - -97.90 (m, 1F).

### Example 34. Synthesis of N-(Tert-Butyl)-2-(N-(4-(Tert-Butyl)Phenyl)-2-Chloroacetamido)-2-(Pyridin-3-yl)Acetamide (INSCoV-534)

INSCoV-534 was obtained according to the General procedure for INSCoV series. Purification A: The residue was purified by prep-HPLC (column: Phenomenex luna C18 150 × 40 mm × 15 µm; mobile phase: [water (0.225% FA)- ACN]; B%: 37%-67%, 10 min). N-tert-butyl-2-(4-tert-butyl-N-(2-chloroacetyl)anilino)-2-(3-pyridyl)acetamide (50.79 mg, 121.23 µmol, 11.46% yield, 99.286% purity) was obtained as white solid, which was confirmed by HNMR, LCMS and HPLC.

HPLC: Retention time: 1.912 min. LCMS: Retention time: 0.886 min, (M+H) = 416.4; Retention time: 0.870 min, (M+H) = 416.3. ¹H NMR (400 MHz, DMSO-d₆): δ = 8.33 - 8.24 (m, 2H), 7.84 (s, 1H), 7.38 - 7.01 (m, 5H), 6.00 (s, 1H), 4.00 - 3.89 (m, 2H), 1.21 (s, 9H), 1.17 (s, 9H).

### Example 35. Synthesis of N-(4-(Tert-Butyl)Phenyl)-2-Chloro-N-(2-(Cyclohexylamino)-1-(5-Hydroxypyridin-3-yl)-2-Oxoethyl)Acetamide (INSCoV-535)

INSCoV-535 was obtained according to the General procedure for INSCoV series. Purification A: The crude was purified by Prep-HPLC (column: Phenomenex luna C18 150 × 40 mm × 15um; mobile phase: [water (0.225% FA)-ACN]; B%: 30%-60%, 10 min) and Prep-HPLC (column: Welch MLtimate XB-SiOH 250 × 50 × 10 µm; mobile phase: [Hexane-EtOH (0.1% NH₃•H₂)]; B%: 1%-30%, 15 min). 2-(4-Tert-butyl-N-(2-chloroacetyl)anilino)-N-cyclohexyl-2-(5-hydroxy-3-pyridyl)acetamide (16.39 mg, 33.43 µmol, 3.16% yield, 93.404% purity) was obtained as a white solid, which was confirmed by HNMR, LCMS and HPLC.

HPLC: Retention time: 2.238 min. LCMS: Retention time: 0.799 min, (M+H) = 458.3; Retention time: 0.883 min, (M+H) = 458.1. ¹H NMR (400 MHz, DMSO-d₆): δ = 9.71 (s, 1H), 8.04 (br d, J=7.2 Hz, 1H), 7.85 (d, J=2.4 Hz, 1H), 7.77 (d, J=1.2 Hz, 1H), 7.24 (br d, J=6.0 Hz, 4H), 6.71 (s, 1H), 5.95 (s, 1H), 4.01 - 3.85 (m, 2H), 3.63 - 3.50 (m, 1H), 1.79 - 1.48 (m, 5H), 1.28 - 1.05 (m, 14H).

### Example 36. Synthesis of N-(4-(Tert-Butyl)Phenyl)-2-Chloro-N-(2-(Cyclohexylamino)-2-Oxo-1-(Pyrimidin-5-yl)Ethyl)Acetamide (INSCoV-536)

INSCoV-536 was obtained according to the General procedure for INSCoV series. Purification A: The crude was purified by Prep-HPLC (column: 3_Phenomenex Luna C18 75 × 30 mm × 3 µm; mobile phase: [water (0.1% TFA)-ACN]; B%: 57%-87%, 7 min. INSCoV-536 (45.49 mg, 97.79 µmol, 9.24% yield, 95.224% purity) was obtained as a as a yellow solid, which was confirmed by LCMS, HPLC and HNMR.

HPLC: Retention time: 2.451 min. LCMS: Retention time: 0.998 min, (M+H) = 443. ¹H NMR (400 MHz, DMSO-d₆): δ = 8.95 (s, 1H), 8.43 (s, 2H), 8.13 (br d, J=7.2 Hz, 1H), 7.39 - 7.11 (m, 4H), 6.03 (s, 1H), 4.06 - 3.91 (m, 2H), 3.65 - 3.49 (m, 1H), 1.78 - 1.54 (m, 5H), 1.35 - 1.13 (m, 14H).

### Example 37. Synthesis of N-(4-(Tert-Butyl)Phenyl)-2-Chloro-N-(2-(Cyclohexylamino)-2-Oxo-1-(Pyridazin-4-yl)Ethyl)Acetamide (INSCoV-537)

INSCoV-537 was obtained according to the General procedure for INSCoV series. Purification A: The crude was purified by Prep-HPLC (column: Phenomenex luna C18 150 × 40 mm × 15 µm; mobile phase: [water (0.225%FA)-ACN]; B%: 45%-75%, 10 min) and Prep-HPLC (column: Welch MLtimate XB-CN 250 × 70 × 10 um; mobile phase: [Hexane-EtOH (0.1% NH₃•H₂O) ]; B%: 10%-50%,15 min), INSCoV-537 (15.87 mg, 34.62 µmol, 3.27% yield, 96.637% purity) was obtained as a white solid, which was confirmed by HNMR, LCMS and HPLC.

HPLC: Retention time: 2.595 min. LCMS: Retention time: 0.973 min, (M+H) = 443.2.¹H NMR (400 MHz, DMSO-d6): δ = 9.07 - 9.02 (m, 1H), 9.00 (s, 1H), 8.18 (br d, J=7.2 Hz, 1H), 7.40 -7.20 (m, 5H), 5.99 (s, 1H), 4.09 - 3.94 (m, 2H), 3.59 - 3.48 (m, 1H), 1.64 (br d, J=4.8 Hz, 4H), 1.53 (br d, J=12.0 Hz, 1H), 1.21 (s, 10H), 1.16 - 1.00 (m, 3H).

### Example 38. Synthesis of 2-Chloro-N-(2-((4,4-Difluorocyclohexyl)Amino)-2-Oxo-1-(Pyridazin-4-yl)Ethyl)-N-(4-(Oxazol-5-yl)Phenyl)Acetamide (INSCoV-5371)

INSCoV-537I was synthesized according to the General procedure for INSCoV series. Purification B: The crude was triturated with EtOH (2 ml), it was filtered, the cake was washed with PE (5 ml), dried in vacuum. 2-chloro-N-(2-((4,4-difluorocyclohexyl)amino)-2-oxo-1-(pyridazin-4-yl)ethyl)-N-(4-(oxazol-5-yl)phenyl)acetamide (194.18 mg, 364.45 µmol, 43.05% yield, 91.948% purity) was obtained as a yellow solid, which was confirmed by HNMR, LCMS and HPLC.

HPLC: Retention time: 2.064 min. LCMS: Retention time: 0.771 min, (M+H) = 490.2; Retention time: 0.842 min, (M+H) = 490.0. ¹H NMR (400 MHz, DMSO-d₆): δ = 9.15 - 8.98 (m, 2H), 8.46 (s, 1H), 8.38 (br d, J=6.8 Hz, 1H), 7.79 - 7.60 (m, 1H), 7.81 - 7.57 (m, 2H), 7.55 - 7.32 (m, 1H), 7.57 - 7.31 (m, 1H), 6.06 (s, 1H), 4.20 - 3.99 (m, 2H), 3.94 - 3.75 (m, 1H), 2.13 - 1.68 (m, 7H), 1.60 -1.30 (m, 2H).

### Example 39. Synthesis of 2-Chloro-N-(2-((4,4-Difluorocyclohexyl)Amino)-2-Oxo-1-(Pyridazin-4-yl)Ethyl)-N-(4-(Isoxazol-5-yl)Phenyl)Acetamide (INSCoV-537K)

INSCoV-537K was synthesized according to the General procedure for INSCoV series. Purification B: The crude was triturated with EtOH (2 ml), it was filtered, the cake was washed with PE (5 ml), dried in vacuum. 2-chloro-N-(2-((4,4-difluorocyclohexyl)amino)-2- oxo-1-(pyridazin-4-yl)ethyl)-N-(4-(oxazol-5-yl)phenyl)acetamide (194.18 mg, 364.45 µmol, 43.05% yield, 91.948% purity) was obtained as a yellow solid, which was confirmed by HNMR, LCMS and HPLC.

HPLC: Retention time: 2.064 min. LCMS: Retention time: 0.771 min, (M+H) = 490.2; Retention time: 0.842 min, (M+H) = 490.0. ¹H NMR (400 MHz, DMSO-d₆): δ = 9.15 - 8.98 (m, 2H), 8.46 (s, 1H), 8.38 (br d, J=6.8 Hz, 1H), 7.79 - 7.60 (m, 1H), 7.81 - 7.57 (m, 2H), 7.55 - 7.32 (m, 1H), 7.57 - 7.31 (m, 1H), 6.06 (s, 1H), 4.20 - 3.99 (m, 2H), 3.94 - 3.75 (m, 1H), 2.13 - 1.68 (m, 7H), 1.60 -1.30 (m, 2H).

### Example 40. Synthesis of N-(4-(Tert-Butyl)Phenyl)-2-Chloro-N-(2-(Cyclohexylamino)-2-Oxo-1-(Pyrazin-2-yl)Ethyl)Acetamide (INSCoV-538)

INSCoV-538 was synthesized according to the General procedure for INSCoV series. Purification A: The crude was triturated with MTBE (5 ml), it was filtered, the cake was triturated with EtOH (2 ml), it was filtered, the cake was dried in vacuum. N-(4-(tert-butyl)phenyl)-2-chloro-N-(2-(cyclohexylamino)-2-oxo-1-(pyrazin-2-yl)ethyl)acetamide (272.7 mg, 599.17 µmol, 56.62% yield, 97.330% purity) was obtained as a white solid, which was confirmed by HNMR, LCMS and HPLC.

HPLC: Retention time: 2.804 min. LCMS: Retention time: 1.029 min, (M+H) = 443.2, Retention time: 1.014 min, (M+H) = 443.2. ¹H NMR (400 MHz, DMSO-d₆): δ = 8.58 - 8.36 (m, 3H), 8.14 (br d, J=7.2 Hz, 1H), 7.28 (br s, 4H), 6.13 (s, 1H), 4.13 - 3.92 (m, 2H), 3.58 - 3.42 (m, 1H), 1.73 - 1.44 (m, 5H), 1.21 (s, 11H), 1.13 - 0.97 (m, 3H).

### Example 41. Synthesis of N-(4-(Tert-Butyl)Phenyl)-2-Chloro-N-(1-(5-Cyanopyridin-3-yl)-2-(Cyclohexylamino)-2-Oxoethyl)Acetamide (INSCoV-539)

INSCoV-539 was synthesized according to the General procedure for INSCoV series. Purification B: The mixture was poured into EtOH (5 ml). The mixture was filtered. The cake was washed with EtOH (1 ml) and MTBE (1 ml), it was dried in vacuum. INSCoV-539 (309.39 mg, 648.42 µmol, 61.27% yield, 97.874% purity) was obtained as a white solid, which was confirmed by HNMR, LCMS and HPLC.

HPLC: Retention time: 2.895 min. LCMS: Retention time:1.029 min, (M+H) = 467.1. ¹H NMR (400 MHz, DMSO-d6): δ = 8.84 (d, J=1.8 Hz, 1H), 8.57 (d, J=2.0 Hz, 1H), 8.13 (d, J=8.0 Hz, 1H), 7.75 (t, J=2.0 Hz, 1H), 7.43 - 7.03 (m, 4H), 6.06 (s, 1H), 4.01 (s, 2H), 3.61 - 3.48 (m, 1H), 1.76 - 1.47 (m, 5H), 1.31 - 1.05 (m, 14H).

### Example 42. Synthesis of N-(4-(Tert-Butyl)Phenyl)-2-Chloro-N-(1-(6-Cyanopyridin-3-yl)-2-(Cyclohexylamino)-2-Oxoethyl)Acetamide (INSCoV-539A)

INSCoV-539A was synthesized according to the General procedure for INSCoV series. Purification B: The crude product was triturated with ACN at 25° C. for 30 min. Compound (2R)-2-(4-tert-butyl-N-(2-chloroacetyl)anilino)-2-(6-cyano-3-pyridyl)-N-cyclohexyl-acetamide (350.65 mg, 737.50 µmol, 55.03% yield, 98.222% purity) was obtained as a white solid. ¹HNMR, LCMS and HPLC confirmed the right structure.

LCMS: Retention time: 1.102 min, (M+H) = 467.2. HPLC: Retention time: 2.939 min. ¹H NMR (400 MHz, CHLOROFORM-d): δ = 8.55 (s, 1H), 7.65 (br d, J=8.1 Hz, 1H), 7.48 (s, 1H), 7.37-7.29 (m, 2H), 7.27 (s, 1H), 7.09-6.79 (m, 1H), 6.22-6.14 (m, 1H), 6.09 (s, 1H), 3.87-3.84 (m, 2H), 3.84-3.77 (m, 1H), 2.00 (br d, J=10.8 Hz, 1H), 1.88 (br dd, J=1.7, 10.8 Hz, 1H), 1.80-1.66 (m, 2H), 1.61 (s, 5H), 1.46-1.33 (m, 2H), 1.29 (s, 9H), 1.19 (br d, J=12.2 Hz, 3H).

### Example 43. Synthesis of INSCoV-549

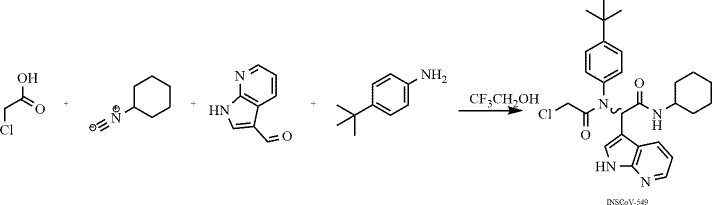

INSCoV-549 was synthesized according to the General procedure for INSCoV series. Purification B: The mixture was poured into water (20 ml) and DCM (20 ml) was added, the organic layer was washed with brine (20 ml), dried over Na₂SO₄ and concentrated in vacuum. The crude was triturated with MTBE (5 ml), it was filtered, the cake was triturated with EtOH (2 ml), it was filtered, the cake was dried in vacuum. INSCoV-549 (154.05 mg, 309.88 µmol, 29.28% yield, 96.763% purity) was obtained as a white solid, which was confirmed by HNMR, LCMS and HPLC.

HPLC: Retention time: 2.462 min. LCMS: Retention time: 0.937 min, (M+H) = 481.2. ¹H NMR (400 MHz, DMSO-d₆): δ = 11.46 (br s, 1H), 8.07 (d, J=4.0 Hz, 1H), 7.81 (br d, J=78.0 Hz, 1H), 7.55 (br d, J=8.0 Hz, 1H), 7.24 - 6.77 (m, 4H), 6.30 (s, 1H), 4.01 - 3.85 (m, 2H), 3.66 - 3.52 (m, 1H), 1.82 - 1.46 (m, 5H), 1.34 - 0.94 (m, 14H).

### Example 44. Synthesis of N-(4-(Tert-Butyl)-2-(3-Morpholinoprop-1-yn-1-yl)Phenyl)-2-Chloro-N-(2-(Cyclohexylamino)-2-Oxo-1-(Pyridin-3-yl)Ethyl)Acetamide (INSCoV-553)

INSCoV-553 was synthesized according to the General procedure for INSCoV series. Purification B: The residue was triturated with MeCN (3 mL), and then filtered. The filter cake was washed with MeCN (2 mL×2) and dried in vacuum. INSCoV-553 (72.6 mg, 123 µmol, 33.6% yield, 96.0% purity) was obtained as white solid, which was confirmed by HNMR, LCMS and HPLC.

LCMS: Retention time: 1.025 min, [M+H⁺] = 565.3. HPLC: Retention time: 2.741 min. ¹H NMR (400 MHz, DMSO-d₆): δ = 8.31 (d, J = 2.1 Hz, 1H), 8.27 (dd, J = 1.2, 4.8 Hz, 1H), 8.08 (d, J = 7.6 Hz, 1H), 7.85 (d, J =8.4 Hz, 1H), 7.45 - 7.40 (m, 1H), 7.27 (d, J =8.1 Hz, 1H), 7.14 (d, J =2.4 Hz, 1H), 7.03 (dd, J = 4.8, 8.0 Hz, 1H), 6.02 (s, 1H), 4.02 - 3.92 (m, 1H), 3.90 - 3.81 (m, 1H), 3.63 (t, J =4.8 Hz, 4H), 3.60 - 3.54 (m, 1H), 3.50 (d, J =2.8 Hz, 2H), 3.31 (s, 2H), 1.85 - 1.73 (m, 1H), 1.73 - 1.65 (m, 1H), 1.63 - 1.44 (m, 3H), 1.28 - 1.12 (m, 13H), 1.11 - 0.86 (m, 2H).

### Example 45. Synthesis of INSCoV-557A

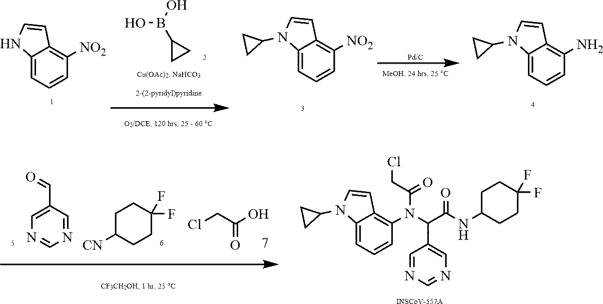

Step 1: To a solution of Compound 1 (3 g, 18.50 mmol, 1 eq) and Compound 2 (1.91 g, 22.20 mmol, 1.2 eq) in DCE (120 mL) was added NaHCO₃ (3.11 g, 37.00 mmol, 1.44 mL, 2 eq), Cu(OAc)₂ (3.36 g, 18.50 mmol, 1 eq) and 2-(2-pyridyl)pyridine (2.89 g, 18.50 mmol, 1 eq). The reaction was stirred at 25° C. for 4 days under O₂ (15PSI). Then the mixture was heated to 60° C. for 24 hr under O₂ (15 PSI). LCMS showed Compound 1 remained and one new peak (Rt= 0.917 min) with desired mass was detected. TLC (PE: EA=5:1) showed Compound 1 (Rf= 0.2) remained and two new spots (Rf= 0.02, Rf= 0.7) formed. The mixture was diluted with water (50 mL) and extracted with DCM (70 mL × 3). The organic layer was dried with anhydrous Na₂SO₄, filtered and concentrated under vacuum. The residue was purified by flash silica gel chromatography (ISCO®; 80 g SepaFlash® Silica Flash Column, Eluent of 0~30% Ethyl acetate/Petroleum ether gradient @ 100 mL/min) and concentrated under vacuum. Compound 3 (1.6 g, 7.91 mmol, 42.77% yield) was obtained as yellow solid, which was confirmed by HNMR.

LCMS: Retention time: 0.972 min, (M+H) =203.1. ¹H NMR (400 MHz, DMSO-d6) δ = 8.13 - 8.07 (m, 2H), 7.78 - 7.72 (m, 1H), 7.43 - 7.37 (m, 1H), 6.98 (d, J= 3.2 Hz, 1H), 3.62 - 3.56 (m, 1H), 1.18 - 1.07 (m, 2H), 1.07 - 0.95 (m, 2H).

Step 2: To a solution of Compound 3 (1 g, 4.95 mmol, 1 eq) in MeOH (20 mL) was added Pd/C (0.1 g, 2.97 mmol, 10% purity, 0.6 eq). The reaction mixture was stirred at 25° C. for 24 hrs under H₂ (50 PSI). LCMS showed Compound 3 was consumed completely and one new peak (Rf= 0.903 min) with desired mass was detected. TLC (PE: EA=5:1) showed Compound 3 (Rf= 0.5) was consumed completely and one new spot (Rf= 0.3) formed. The reaction mixture was combined with to work-up. The reaction mixture was adjusted pH=10 by NH₃•H₂O, filtered through a pad of Celite and washed with MeOH (40 mL × 2). The filtrate was concentrated under vacuum. The residue was purified by flash silica gel chromatography (ISCO®; 40 g SepaFlash® Silica Flash Column, Eluent of 0~80% Ethyl acetate/Petroleum ether gradient @ 80 mL/min). The combined organic phase was concentrated under vacuum. Compound 4 (0.9 g, 5.23 mmol, 105.67% yield) was obtained as red oil, which was confirmed by HNMR.

LCMS: Retention time: 0.903 min, (M+H) =173.2. ¹H NMR (400 MHz, DMSO-d6) δ = 7.03 (d, J = 3.3 Hz, 1H), 6.88 - 6.80 (m, 1H), 6.73 (d, J = 8.0 Hz, 1H), 6.45 (d, J = 2.8 Hz,1H), 6.20 - 6.14 (m, 1H), 5.17 (s, 2H), 3.31 - 3.27 (m, 1H), 1.04 - 0.95 (m, 2H), 0.90 - 0.82 (m, 2H).

Step 3: INSCoV-557A was synthesized according to the General procedure for INSCoV series. Purification A: The reaction was concentrated under vacuum. The crude product was purified by reversed-phase HPLC (0.1%FA) and concentrated under vacuum to remove MeCN. The aqueous phase was under lyophilization to give the crude product. The residue was dissolved in DMF (2 mL) and purified by Pre-HPLC (column: Waters Xbridge 150 × 25 mm × 5 µm; mobile phase: [water (10 mM NH₄HCO₃)-ACN]; B%: 35%-65%, 8 min) and diluted with water (30 mL), the liquid was under lyophilization to give the product. INSCoV-557-A (7.16 mg, 13.79 µmol, 1.49% yield, 96.703% purity) was obtained as yellow solid, which was confirmed by LCMS, HPLC, SFC, HNMR and FNMR.

LCMS: Retention time: 0.913 min, (M+H) =502.0. HPLC: Retention time: 2.196 min. SFC: Retention time: 3.036 min, 3.281 min. ¹H NMR (400 MHz, DMSO-d₆) δ = 8.88 (s, 1H), 8.72 (s, 1H), 8.52 (s, 1H), 8.41 (s, 2H), 8.25 - 8.11 (m, 1H), 7.53 (d, J =7.6 Hz, 1H), 7.48 (d, J = 8.4 Hz, 1H), 7.42 (d, J= 3.2 Hz, 1H), 7.24 (d, J= 3.2 Hz, 1H), 7.22 - 7.14 (m, 1H), 7.04 - 6.98 (m, 1H), 6.86 - 6.77 (m, 1H), 6.24 (d, J =3.2 Hz, 1H), 6.10 (s, 1H), 5.88 - 5.78 (m, 1H), 5.82 (s, 1H), 4.07 - 3.67 (m, 5H), 3.49 - 3.42 (m, 1H), 2.08 - 1.47 (m, 11H), 1.39 - 1.22 (m, 2H), 1.08 - 0.77 (m, 7H). ¹⁹F NMR (377 MHz, DMSO-d₆) δ = -92.05 - -95.13 (m, 1F), -96.91 - -99.42 (m, 1F).

### Example 46. Synthesis of 2-Chloro-N-(2-(Cyclohexylamino)-2-Oxo-1-(Pyridin-3-yl)Ethyl)-N-(3-Fluorophenethyl)Acetamide (INSCoV-558)

INSCoV-558 was synthesized according to the General procedure for INSCoV series. Purification B: The crude product was triturated by ACN (10 mL) at 4° C. to give the pure product 2-[(2-chloroacetyl)-[2-(3-fluorophenyl)ethyl]amino]-N-cyclohexyl-2-(3-pyridyl) acetamide (105.75 mg, 236.21 µmol, 16.44% yield, 96.477% purity) obtained as a white solid. Compound 2-[(2-chloroacetyl)-[2-(3-fluorophenyl)ethyl]amino]-N-cyclohexyl-2-(3-pyridyl) acetamide (105.75 mg, 236.21 µmol, 16.44% yield, 96.477% purity) was obtained as a white solid. LCMS, HPLC, ¹HNMR and FNMR checked the right structure.

LCMS: Retention time: 0.970 min, (M+H) = 432.1. HPLC: Retention time: 2.482 min.¹H NMR (400 MHz, CHLOROFORM-d): δ = 8.71-8.61 (m, 2H), 7.89 (br d, J=7.7 Hz, 1H), 7.38 (dd, J=4.8, 7.9 Hz, 1H), 7.26-7.16 (m, 1H), 6.92 (br t, J=8.1 Hz, 1H), 6.82 (br d, J=7.7 Hz, 1H), 6.73 (br d, J=9.5 Hz, 1H), 6.02 (br d, J=6.2 Hz, 1H), 5.80 (s, 1H), 3.99 (s, 2H), 3.90-3.77 (m, 1H), 3.60 (br t, J=7.6 Hz, 2H), 2.88-2.75 (m, 1H), 2.58-2.45 (m, 1H), 1.99-1.88 (m, 2H), 1.74-1.66 (m, 2H), 1.61-1.56 (m, 1H), 1.44-1.31 (m, 2H), 1.23-1.08 (m, 3H).

### Example 47. Synthesis of 2-Chloro-N-(2-((1,1-Dioxidotetrahydro-2H-Thiopyran-4-yl)Amino)-2-Oxo-1-(Pyrimidin-5-yl)Ethyl)-N-(3-Fluorophenethyl)Acetamide (INSCoV-558A)

INSCoV-558A was synthesized according to the General procedure for INSCoV series. Purification A: The crude product was purified by prep-HPLC ( column: Waters Xbridge 150 × 25 mm × 5 µm; mobile phase: [water (10 Mm NH₄HCO₃)-ACN]; B%:24%-54%, 10 min. 2-[(2-Chloroacetyl)-[2-(3-fluorophenyl)ethyl]amino]-N-(1,1-dioxothian-4-yl)-2- pyrimidin-5-yl-acetamide (5.7 mg, 10.81 µmol, 1.77% yield, 91.597% purity) was obtained as white solid, which was detected by LCMS, HPLC and HNMR.

LCMS: Retention time: 0.861 min, [M+H+] = 483.2. HPLC: Retention time: 1.896 min. ¹H NMR: (400 MHz, DMSO-d₆): δ = 9.14 (s, 1H), 8.74 (s, 2H), 8.05 (br d, J = 7.8 Hz, 1H), 7.42 - 7.20 (m, 1H), 7.15 - 6.88 (m, 3H), 5.68 (s, 1H), 4.60 - 4.50 (m, 2H), 4.14 - 3.97 (m, 1H), 3.58 (br t, J = 8.3 Hz, 2H), 3.27 - 3.20 (m, 2H), 3.11 - 2.98 (m, 3H), 2.92 - 2.75 (m, 1H), 2.64 - 2.59 (m, 1H), 2.03 - 1.88 (m, 4H).

### Example 48. Synthesis of 2-Chloro-N-(2-(Cyclohexylamino)-2-Oxo-1-(Pyridin-3-yl)Ethyl)-N-(4-Fluorophenethyl)Acetamide (INSCoV-559)

INSCoV-559 was synthesized according to the General procedure for INSCoV series. Purification A: The residue was purified by prep-HPLC (column: Phenomenex Synergi C18 150 × 25 mm × 10um; mobile phase: [water (0.1%TFA)-ACN]; B%: 25%-55%,10 min), then LCMS checked and 73% of desired mass was detected. The crude was repurified by prep-HPLC (column: Waters Xbridge 150 × 25 mm × 5 µm; mobile phase: [water (10 mM NH₄HCO₃)-ACN]; B%: 39%-69%, 9 min), then LCMS checked and 77% of desired mass was detected. The crude was repurified by prep-HPLC(column: Phenomenex Gemini-NX C18 75 × 30 mm × 3 um; mobile phase: [water (0.05% ammonia hydroxide v/v)-ACN]; B%: 31%-61%,11.5 min), then LCMS checked and 100% of desired mass was detected. Compound 2-[(2-chloroacetyl)-[2-(4-fluorophenyl)ethyl]amino]-N-cyclohexyl-2-(3-pyridyl)acetamide (15.23 mg, 35.26 µmol, 4.91e-1% yield) was obtained as a white solid. ¹HNMR checked the structure.

LCMS: Retention time: 0.978 min, (M+H) = 432.1. HPLC: Retention time: 2.498 min. ¹H NMR (400 MHz, CHLOROFORM-d): δ = 8.71-8.61 (m, 2H), 7.93-7.83 (m, 1H), 7.41-7.33 (m, 1H), 7.02-6.93 (m, 4H), 6.02-5.93 (m, 1H), 5.81-5.75 (m, 1H), 4.01-3.93 (m, 2H), 3.87-3.78 (m, 1H), 3.64-3.52 (m, 2H), 2.86-2.71 (m, 1H), 2.57-2.42 (m, 1H), 2.01-1.86 (m, 2H), 1.78-1.64 (m, 3H), 1.46-1.30 (m, 3H), 1.23-1.10 (m, 4H).

### Example 49. Synthesis of 2-Chloro-N-(2-(Cyclohexylamino)-2-Oxo-1-(Pyridin-3-yl)Ethyl)-N-(2-Methoxyphenethyl)Acetamide (INSCoV-560A)

INSCoV-560A was synthesized according to the General procedure for INSCoV series. The residue was triturated from MeOH (10 mL) to give the pure product 2-[(2-chloroacetyl)-[2-(2-methoxyphenyl)ethyl]amino]-N-cyclohexyl-2-(3-pyridyl)acetamide (800 mg, 1.80 mmol, 27.25% yield, 100% purity) as a white solid. Compound 2-[(2-chloroacetyl)-[2-(2-methoxyphenyl)ethyl]amino]-N-cyclohexyl-2-(3-pyridyl)acetamide (800 mg, 1.80 mmol, 27.25% yield, 100% purity) was obtained as a white solid. LCMS, HPLC and ¹HNMR checked the right compound.

LCMS: Retention time: 0.980 min, (M+H) = 444.2. HPLC: Retention time: 2.522 min. ¹H NMR (400 MHz, CHLOROFORM-d): δ = 8.70-8.62 (m, 2H), 7.92-7.86 (m, 1H), 7.39-7.33 (m, 1H), 7.24-7.17 (m, 1H), 6.83 (d, J=5.1 Hz, 2H), 6.12-6.04 (m, 1H), 5.92-5.89 (m, 1H), 4.28 (d, J=9.0 Hz, 2H), 3.84 (s, 4H), 3.66-3.55 (m, 1H), 3.55-3.44 (m, 1H), 2.84-2.73 (m, 1H), 2.37-2.27 (m, 1H), 1.98-1.87 (m, 2H), 1.67 (br d, J=3.5 Hz, 1H), 1.69 (br s, 1H), 1.63 - 1.55 (m, 1H), 1.44-1.29 (m, 2H), 1.23-1.08 (m, 3H).

### Example 50. Synthesis of N-(4-(Tert-Butyl)Phenyl)-2-Chloro-N-(2-(Cyclohexylamino)-2-Oxo-1-(Pyridin-3-yl)Ethyl)Propenamide (INSCoV-570)

INSCoV-570 was synthesized according to the General procedure for INSCoV series. Purification B: The crude product was triturated with EtOAc (1 mL) and petroleum ether (15 mL) at 20° C. for 60 min. INSCoV-570 (142 mg, 297 µmol, 44.36% yield, 95.204% purity) was obtained as white solid, checked by LCMS, HPLC and ¹HNMR.

LCMS: Retention time: 1.070 min, [M] = 456.2; Retention time: 3.029 min, [M] = 456.2. HPLC: Retention time: 2.939 min. ¹H NMR (400 MHz, DMSO-d₆): δ = 8.34 - 8.24 (m, 2H), 8.01 (dd, J = 7.6, 15.2 Hz, 1H), 7.69 - 6.98 (m, 5H), 5.98 (d, J = 17.6 Hz, 1H), 4.18 (qd, J = 6.6, 13.6 Hz, 1H), 3.63 - 3.48 (m, 1H), 1.78 - 1.41 (m, 8H), 1.32 - 0.87 (m, 15H).

### Example 51. Synthesis of 2-Chloro-N-(2-(Cyclohexylamino)-2-Oxo-1-(Pyridin-3-yl)Ethyl)-N-(3-(Trifluoromethyl)Benzyl)Acetamide (INSCoV-574)

INSCoV-574 was synthesized according to the General procedure for INSCoV series. Purification B: The crude product was purified by prep-HPLC (column: Waters Xbridge 150 × 25 mm × 5 µm; mobile phase: [water (10 mM NH₄HCO₃)-ACN]; B%: 36%-69%, 9 min). INSCoV-574 (153.85 mg, 327.55 µmol, 28.69% yield, 99.62% purity) was obtained as white solid, which was detected by LCMS, HPLC, HNMR and FNMR.

LCMS: Retention time: 0.974 min, [M+H⁺] = 468.2; Retention time: 2.449 min, [M+H⁺] = 468.2. HPLC: Retention time: 2.715 min. ¹H NMR (400 MHz, DMSO-d₆, T=80): δ = 8.50 - 8.34 (m, 2H), 7.93 (br d, J =6.8 Hz, 1H), 7.66 (br d, J =8.0 Hz, 1H), 7.54 - 7.10 (m, 5H), 6.08 - 5.83 (m, 1H), 4.96 (d, J =17.2 Hz, 1H), 4.67 (br d, J =17.0 Hz, 1H), 4.50 - 4.25 (m, 2H), 3.66 - 3.51 (m, 1H), 1.78 -1.50 (m, 5H), 1.31 - 1.09 (m, 5H). ¹⁹F NMR (400 MHz, DMSO-d6): -61.52 (s, 3F).

### Example 52. Synthesis of 2-Chloro-N-(2-(Cyclohexylamino)-2-Oxo-1-(Pyridin-3-yl)Ethyl)-N-(2-(Trifluoromethyl)Benzyl)Acetamide (INSCoV-574A)

INSCoV-574A was synthesized according to the General procedure for INSCoV series. Purification B: The crude product was purified by prep-HPLC (column: Waters Xbridge 150×25mm× 5 µm; mobile phase: [water (10 mM NH4HCO3)-ACN];B%: 38%-68%, 9 min). INSCoV-574A (254.01 mg, 533.37 µmol, 46.71% yield, 98.252% purity) was obtained as yellow solid, which was detected by LCMS, HPLC, HNMR and FNMR.

LCMS: Retention time: 0.974 min, [M+H⁺] = 468.2. HPLC: Retention time: 2.705 min. ¹H NMR (400 MHz, DMSO-d₆, T=80): δ = 8.64 - 8.26 (m, 2H), 8.21 - 7.89 (m, 1H), 7.79 - 7.04 (m, 6H), 6.24 - 5.91 (m, 1H), 5.16 -4.67 (m, 2H), 4.62 - 4.23 (m, 2H), 3.76 - 3.38 (m, 1H), 1.88 - 1.43 (m, 5H), 1.33 - 1.08 (m, 5H). ¹⁹F NMR: (400 MHz, DMSO-d6): -60.52 (s, 3F).

### Example 53. Synthesis of INSCoV-575

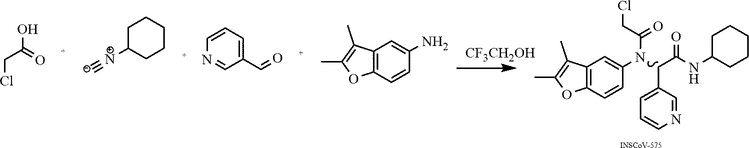

INSCoV-575 was synthesized according to the General procedure for INSCoV series. Purification B: The residue was poured into ACN (5 mL) and stirred for 5 min, collect the crystalline solid by suction filtration. The filtrate was adjusted to PH=7~8 and discarded. INSCoV-575 (365.95 mg, 785.73 µmol, 63.33% yield, 97.47% purity) was obtained as white solid, which was detected by LCMS, HPLC and HNMR.

LCMS: Retention time: 1.005 min, [M+H⁺] = 454.2. HPLC: Retention time: 2.777 min. ¹H NMR (400 MHz, DMSO-d₆): δ = 8.39 - 8.22 (m, 2H), 8.11 (br d, J =6.7 Hz, 1H), 8.02 - 7.55 (m, 1H), 7.47 - 7.29 (m, 1H),7.22 - 7.00 (m, 2H), 7.00 - 6.46 (m, 1H), 6.07 (s, 1H), 4.11 - 3.82 (m, 2H), 3.68 -3.51 (m, 1H), 2.33 (br s, 3H), 2.19 - 1.85 (m,3H), 1.83 - 1.46 (m, 5H), 1.38 - 0.86 (m, 5H).

### Example 54. Synthesis of INSCoV-576

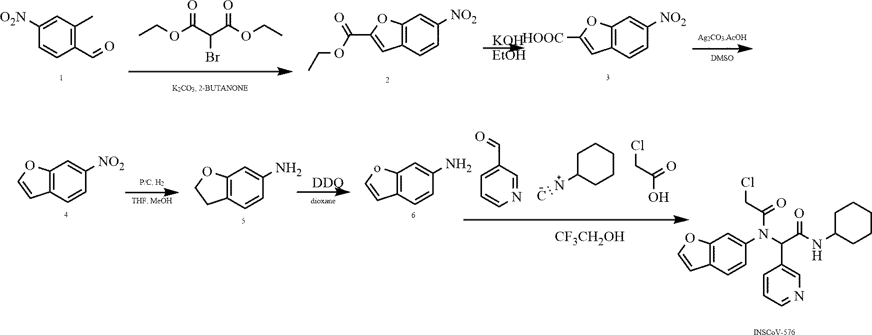

Step 1: To a mixture of 2-hydroxy-4-nitro-benzaldehyde (2 g, 11.97 mmol, 1 eq) and diethyl 2-bromopropanedioate (2.86 g, 11.97 mmol, 2.04 mL, 1 eq) in 2-butanone (50 mL) was added K₂CO₃ (4.96 g, 35.90 mmol, 3 eq) in one portion at 25° C. under N₂, then heated to 90° C. and stirred for 16 hours. LCMS showed the reaction was completed. The mixture was poured into ice-water (100 mL) and stirred for 5 min. The aqueous phase was extracted with ethyl acetate (100 mL × 3), dried with anhydrous Na₂SO₄, filtered and concentrated in vacuum. The residue was purified by flash silica gel chromatography (ISCO®; 40 g SepaFlash® Silica Flash Column, Eluent of 0~100% Ethylacetate/Petroleum ether gradient @ 60 mL/min). TLC (PE:EA=2:1, RF=0.6). Ethyl 6-nitrobenzofuran-2-carboxylate (2.7 g, 10.91 mmol, 91.13% yield, 95% purity) was obtained as light yellow solid, which was detected by LCMS and ¹HNMR.

LCMS: Retention time: 0.909 min, [M+H+] = 236.2: Retention time: 1.207 min, [M+H+] = 236.2. ¹H NMR (400 MHz, CHLOROFORM-d): δ = 8.53 - 8.47 (m, 1H), 8.25 (dd, J = 2.0, 8.8 Hz, 1H), 7.83 (d, J =8.4 Hz, 1H), 7.61(d, J =1.0 Hz, 1H), 4.49 (q, J =7.2 Hz, 2H), 1.58 (s, 3H).

Step 2: To a mixture of ethyl 6-nitrobenzofuran-2-carboxylate (2 g, 8.50 mmol, 1 eq) in EtOH (20 mL) was added KOH (715.65 mg, 12.76 mmol, 1.5 eq) in one portion at 25° C. under N₂, then heated to 70° C. and stirred for 1 hours. LCMS and HPLC showed the reaction was completed. The solvent was evaporated under reduced pressure and the residue was dissolved in water (10 mL) and acidified to pH4 with concentrated hydrochloric acid. The resulting precipitate was collected by filtration, washed with water and dried. 6-Nitrobenzofuran-2-carboxylic acid (1.6 g, crude) was obtained as light yellow solid, which was detected by ¹HNMR.

LCMS: Retention time: 0.200 min, [M+H⁺] = 208.1. HPLC: Retention time: 0.249 min. ¹H NMR (400 MHz, DMSO-d₆): δ = 8.67 (s, 1H), 8.37 - 8.15 (m, 1H), 8.03 (br d, J =8.7 Hz, 1H), 7.83 (s, 1H).

Step 3: To a mixture of 6-nitrobenzofuran-2-carboxylic acid (500 mg, 2.41 mmol, 1 eq) in DMSO (5 mL) under nitrogen at room temperature was added Ag₂CO₃ (332.80 mg, 1.21 mmol, 54.74 µL, 0.5 eq) and AcOH (14.50 mg, 241.38 µmol, 13.81 µL, 0.1 eq). The reaction mixture was stirred at 120° C. for 3 hours. LC-MS showed the reaction was finished and desired product was found. The mixture was filtered. The mixture was poured into ice-water (20 mL). The aqueous phase was extracted with ethyl acetate (50 mL × 3), dried with anhydrous Na2SO4, filtered and concentrated in vacuum. The residue was purified by flash silica gel chromatography (ISCO®; 40 g SepaFlash® Silica Flash Column, Eluent of 0 \~100% Ethylacetate/Petroleum ether gradient @ 60 mL/min). TLC (PE/EA=3:1, RF=0.5). 6-Nitrobenzofuran (390 mg, 2.22 mmol, 92.11% yield, 93% purity) was obtained as white solid, which was detected by ¹HNMR.

LCMS: Retention time: 0.915 min, [M+H⁺] = 164.1. ¹H NMR (400 MHz, CHLOROFORM-d): δ = 8.44 (s, 1H), 8.20 (dd, J = 2.0, 8.6 Hz, 1H), 7.90 (d, J =2.4 Hz, 1H), 7.71 (d, J =8.6 Hz, 1H), 6.92 (dd, J = 0.9, 2.1 Hz, 1H).

Step 4: 6-Nitrobenzofuran (350 mg, 2.15 mmol, 1 eq) was dissolved in a mixed solvent of MeOH (20 mL) and THF (20 mL) and then Pd/C (350 mg, 10% purity) was added thereto. The reaction mixture was stirred at 25° C. under H₂ (50 psi) for 3 hours. LC-MS showed the reaction was finished and desired product was found. After filtering and concentrating in the reduced pressure. 2,3-Dihydrobenzofuran-6-amine (270 mg, crude) was obtained as brown oil.

LCMS: Retention time: 0.777 min, [M+H⁺] = 136.2.

Step 5: To a mixture of 2,3-dihydrobenzofuran-6-amine (250 mg, 1.85 mmol, 1 eq) in dioxane (10 mL) was added DDQ (461.86 mg, 2.03 mmol, 1.1 eq) in one portion at 25° C. under N₂, then heated to 90° C. and stirred for 2 hours. LC-MS showed 39% of 2,3-dihydrobenzofuran-6-amine was remained and 14% of desired compound. The mixture was filtered and concentrated in vacuum. The residue was purified by flash silica gel chromatography (ISCO®; 40 g SepaFlash® Silica Flash Column, Eluent of 0 \~100% Ethyl acetate / Petroleum ether gradient @ 50 mL/min). TLC (PE/EA=3/1, RF=0.5). Benzofuran-6-amine (120 mg, 865.21 µmol, 46.78% yield, 96% purity) was obtained as brown oil, which was detected by LCMS.

LCMS: Retention time: 0.703 min, [M+H⁺] = 134.2.

Step 6: INSCoV-576 was synthesized according to the General procedure for INSCoV series. Purification A: The crude product was purified by prep-HPLC (column: Waters Xbridge 150 × 25 mm × 5 µm; mobile phase: [water (10 mM NH₄HCO₃)-ACN]; B%: 36%-66%, 8 min). INSCoV-576, 2-[Benzofuran-6-yl-(2-chloroacetyl)amino]-N-cyclohexyl-2-(3-pyridyl)acetamide (80.51 mg, 184.23 µmol, 30.66% yield, 97.46% purity) was obtained as white solid, which was detected by LCMS, HPLC and HNMR.

LCMS: Retention time: 0.920 min, [M+H⁺] = 426.2. HPLC: Retention time: 2.452 min. ¹H NMR (400 MHz, DMSO-d₆): δ = 8.44 - 8.23 (m, 2H), 7.92 (d, J =2.1 Hz, 1H), 7.83 - 7.54 (m, 1H), 7.82 - 7.51 (m, 1H), 7.51- 7.37 (m, 2H), 7.21 - 7.00 (m, 2H), 6.86 (dd, J = 0.9, 2.2 Hz, 1H), 6.14 (s, 1H), 4.07 - 3.78 (m, 2H), 3.71 - 3.49 (m, 1H), 1.94 -1.46 (m, 5H), 1.41 - 1.00 (m, 5H).

### Example 55. Synthesis of INSCoV-600A

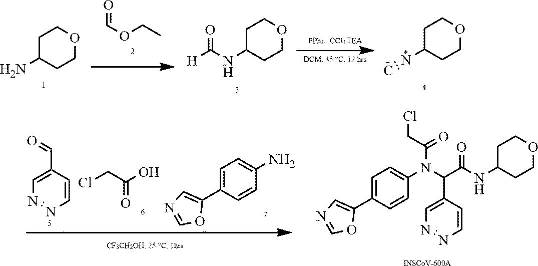

Step 1: The mixture of Compound 1 (5.0 g, 49.43 mmol, 1 eq) and Compound 2 (46.05 g, 621.64 mmol, 50 mL, 12.58 eq) was stirred at 70° C. for 12 h under N₂. TLC (PE/EA=3/1) indicated Compound 1 (Rf =0.0) was consumed and one small spot formed (Rf = 0.1). The mixture was concentrated under vacuum. The reaction was used to next and no purification. Compound 3 (7.0 g, crude) was obtained as white solid.

¹H NMR (400 MHz, CHLOROFORM-d): δ = 8.14 (s, 1H), 5.79 - 5.17 (m, 1H), 4.19 - 4.05 (m, 1H), 4.03 - 3.92 (m, 2H), 3.52 - 2.46 (m, 2H), 2.02 - 1.89 (m, 2H), 1.56 - 1.41 (m, 2H).

Step 2: To a solution of Compound 3 (1.0 g, 7.74 mmol, 1 eq) and TEA (783.46 mg, 7.74 mmol, 1.08 mL, 1.0 eq) in DCM (10 mL) was added PPh₃ (2.23 g, 8.52 mmol, 1.1 eq). The mixture was stirred at 45° C. for 12 hrs under N₂. TLC (PE/EA = 3/1) indicated Compound 3 (Rf = 0.1) was consumed and one small spot formed (Rf = 0.6). The resulting mixture was evaporated in vacuum (<20° C.). The residue was suspended in Et20 (100 ml) at 20° C. for 12 h under N₂. The solid was filtered, washed with Et₂O (100 mL × 2) and the filtrate was concentrated under vacuum (< 20° C.). The combined organic phase was concentrated under vacuum (< 20° C.). The reaction was used to next step and no purification. Compound 4 (0.8 g, 7.20 mmol, 92.97% yield) was obtained as brown oil, which structure was confirmed by ¹H NMR.

¹H NMR (400 MHz, CHLOROFORM-d): δ = 3.90 - 3.71 (m, 3H), 3.58 - 3.45 (m, 2H), 1.99 - 1.86 (m, 2H), 1.83 - 1.73 (m, 2H).

Step 3: INSCoV-600A was synthesized according to the General procedure for INSCoV series. Purification B: To the residue was added MTBE (20 mL), the suspension was filtered and washed with MTBE (10 mL × 3) to get the crude product. The residue was triturated with MTBE (20 mL), filtered and washed with MTBE (10 mL × 3). The filter cake was concentrated under vacuum. INSCoV-600A (147.51 mg, 305.05 µmol, 32.97% yield, 94.278% purity) was obtained as yellow solid, which was confirmed by HNMR, LCMS, SFC and HPLC.

LCMS: Retention time: 0.737 min, (M+H) =456.1. HPLC: Retention time: 1.460 min. SFC: Retention time: 1.469 min, 1.796 min. ¹H NMR (400 MHz, DMSO-d6): δ = 9.09 - 9.00 (m, 2H), 8.45 (s, 1H), 8.39 (d, J = 7.6 Hz, 1H), 7.71 (s, 1H), 7.63 (d, J = 8.8 Hz, 2H), 7.54 - 7.42 (m, 2H), 7.40 -7.34 (m, 1H), 6.07 (s, 1H), 4.11 - 4.05 (m, 2H), 3.87 - 3.74 (m, 3H), 3.34 (s, 1H), 3.32 - 3.26 (m, 1H), 1.75 - 1.61 (m, 2H), 1.48 - 1.21 (m, 2H).

### Example 56. Synthesis of 2-Chloro-N-(4-(Oxazol-5-yl)Phenyl)-N-(2-Oxo-1-(Pyridin-3-yl)-2-((Tetrahydro-2H-Pyran-4-yl)Amino)Ethyl)Acetamide (INSCoV-600A(l))

INSCoV-600A(1) was synthesized according to the General procedure for INSCoV series. Purification B: The residue was triturated with MTBE (20 mL), filtered and washed with MTBE (10 mL × 3). The filter cake was concentrated under vacuum. INSCoV_ 600A(1) (123.82 mg, 267.11 µmol, 28.61% yield, 98.136% purity) was obtained as white solid, which was confirmed by HNMR, LCMS, SFC and HPLC.

LCMS: Retention time: 0.693 min, (M+H) =455.1. HPLC: Retention time: 1.251 min. SFC: Retention time: 1.452 min, 1.678 min. ¹H NMR (400 MHz, DMSO-d₆): δ = 8.43 (s, 1H), 8.36 - 8.24 (m, 3H), 7.69 (s, 1H), 7.66 - 7.44 (m, 3H), 7.41 - 7.35 (m, 1H), 7.18 - 7.12 (m, 1H), 6.08 (s, 1H), 4.10 - 3.94 (m, 2H), 3.87 - 3.70 (m, 3H), 3.40 - 3.35 (m, 1H), 3.32 - 3.28 (m, 1H), 1.77 - 1.71 (m, 1H), 1.66 - 1.60 (m, 1H), 1.52 - 1.37 (m, 1H), 1.31 - 1.16 (m, 1H).

### Example 57. Synthesis of 2-Chloro-N-(4-(Oxazol-5-yl)Phenyl)-N-(2-Oxo-1-(Pyrimidin-5-yl)-2-((Tetrahydro-2H-Pyran-4-yl)Amino)Ethyl)Acetamide(INSCoV-600A(2))

INSCoV-600A(2) was synthesized according to the General procedure for INSCoV series. Purification B: MTBE (20 mL) was added the reaction mixture, filtered and washed with MTBE (10 mL × 3) to get the crude product. The residue was triturated with MTBE (20 mL), filtered and washed with MTBE (10 mL × 3). The filter cake was concentrated under vacuum. INSCoV_600A(2) (202.45 mg, 435.58 µmol, 47.09% yield, 98.087% purity) was obtained as yellow solid, which was confirmed by HNMR, LCMS, SFC and HPLC.

LCMS: Retention time: 0.743 min, (M+H) =456.1. HPLC: Retention time: 1.455 min. SFC: Retention time: 0.515 min, 0.945 min. ¹H NMR (400 MHz, DMSO-d₆): δ = 8.97 (s, 1H), 8.53 - 8.44 (m, 3H), 8.34 (d, J =7.2 Hz, 1H), 7.72 (s, 1H), 7.68 - 7.62 (m, 2H), 7.44 (d, J= 3.6 Hz, 2H), 6.10 (s, 1H), 4.16 - 3.99 (m, 2H), 3.88 - 3.68 (m, 3H), 3.36 (d, J= 2.0 Hz, 1H), 3.30 (d, J= 2.0 Hz, 1H), 1.81 - 1.59 (m, 2H), 1.52 - 1.38 (m, 1H), 1.34 - 1.20 (m, 1H).

### Example 58. Synthesis of 2-Chloro-N-(4-(Oxazol-5-yl)Phenyl)-N-(2-oxo-1-(Pyrazin-2-yl)-2-((Tetrahydro-2H-Pyran-4-yl)Amino)Ethyl)Acetamide (INSCoV-600A(3))

INSCoV-600A(3) was synthesized according to the General procedure for INSCoV series. Purification A: The residue was triturated with MTBE (20 mL), filtered and washed with MTBE (10 mL × 3). The filter cake was concentrated under vacuum. INSCoV_600A(3) (177.52 mg, 375.02 µmol, 40.54% yield, 96.309% purity) was obtained as off-white solid, which was confirmed by HNMR, LCMS, SFC and HPLC.

LCMS: Retention time: 0.746 min, (M+H) =456.1. HPLC: Retention time: 1.502 min SFC: Retention time: 1.720 min, 2.182 min. ¹H NMR (400 MHz, DMSO-d₆): δ = 8.49 (s, 2H), 8.46 -8.40 (m, 2H), 8.32 (d, J =7.6 Hz, 1H), 7.70 (s, 1H), 7.61 (d, J =8.4 Hz, 2H), 7.50 - 7.44 (m, 1H), 6.23 (s, 1H), 4.21 - 4.00 (m, 2H), 3.87 - 3.71 (m, 3H), 3.35 (d, J =2.8 Hz, 1H), 3.30 (d, J =3.2 Hz, 1H), 1.66 (d, J= 11.2 Hz, 2H), 1.45 - 1.24 (m, 2H).

### Example 59. Synthesis of INSCoV-600B

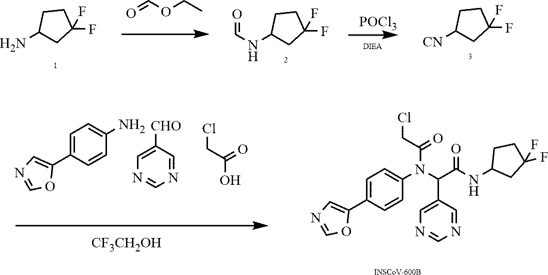

Step 1: To a mixture of 3,3-difluorocyclopentanamine hydrochloride (1 g, 6.35 mmol, 1 eq) and TEA (1.28 g, 12.69 mmol, 1.77 mL, 2 eq) in ethyl formate (2.35 g, 31.73 mmol, 2.55 mL, 5 eq) was stirred at 80° C. for 16 hr. TLC (EA:EtOH=3:1) showed the starting material (Rf=0.3) was consumed completely. And a new spot (Rf=0.7) was detected. The mixture was concentrated in reduced pressure. The crude compound was used into the next step without further purification. Compound 2 (940 mg, 6.30 mmol, 99.33% yield) was obtained as yellow oil, checked by ¹HNMR.

¹H NMR: (400 MHz, CDCl₃): δ = 8.17 - 8.06 (m, 1H), 4.59 - 4.40 (m, 1H), 2.56 - 2.47 (m, 2H), 1.57 (br t, J = 7.2 Hz, 2H), 1.24 (br t, J =7.2 Hz, 2H).

Step 2: To a mixture of compound 2 (500 mg, 3.35 mmol, 1 eq) and DIEA (2.17 g, 16.7 mmol, 2.92 mL, 5 eq) in DCM (100 mL) was added POCl₃ (616.86 mg, 4.02 mmol, 373.86 µL, 1.2 eq) in one portion at -10° C. under N₂, then heated to 20° C. and stirred for 2 hours. TLC (plate 1, PE:EA=1:1) showed compound 2 (Rf=0.1) was consumed and a new spot (Rf=0.8) was observed. The mixture was poured into sat. NaHCO3 (500 mL) slowly at 0° C., and then extracted with DCM (300 mL × 2), dried over Na₂SO₄. The mixture was concentrated in reduced pressure at 25° C. The residue was purified by flash silica gel chromatography (ISCO®; 40 g SepaFlash® Silica Flash Column, Eluent of 0 \~50% Ethyl acetate/Petroleum ether gradient @ 50 mL/min). The mixture was concentrated in reduced pressure at 25° C. TLC (plate 2, PE:EA=1:1, Rf=0.1) compound 3 (200 mg, 1.53 mmol, 45.5% yield) was obtained as colorless oil.

Step 3: INSCoV-600B was synthesized according to the General procedure for INSCoV series. Purification B & Purification A: The residue was triturated with MeCN (5 mL) at 25° C. for 30 min and filtered. The filter cake was re-purified by prep-HPLC (column: Welch MLtimate XB-CN 250 × 50 × 10 um; mobile phase: [Hexane-IPA]; B%: 25%-65%, 15 min) and concentrated in vacuum to give desired compound. The filtrate was concentrated in vacuum to give a residue. The residue was re-purified by prep-HPLC (column: Welch MLtimate XB-CN 250 × 50 × 10 um; mobile phase: [Hexane-IPA]; B%: 25%-65%, 15 min) but it failed to give desired compound. 2-(N-(2-chloroacetyl)-4-oxazol-5-yl-anilino)-N-(3,3-difluorocyclopentyl)-2-pyrimidin-5-yl-acetamide (73.31 mg, 148 µmol, 15.8% yield, 96.04% purity) was obtained as yellow solid, which was checked by HNMR, FNMR, LCMS and HPLC.

LCMS: Retention time: 0.812 min, (M+H) = 442.2. HPLC: Retention time: 1.560 min. ¹H NMR: (400 MHz, DMSO-d₆): δ = 8.98 (d, J= 2.0 Hz, 1H), 8.60 - 8.51 (m, 1H), 8.51 - 8.48 (m, 2H), 8.46 (s, 1H), 7.75 - 7.70 (m, 1H), 7.66 (br d, J =8.4 Hz, 2H), 7.53 - 7.31 (m, 2H), 6.07 (d, J =2.4 Hz, 1H), 4.31 - 4.18 (m, 1H), 4.13 - 3.99 (m, 2H), 3.82- 3.72 (m, 1H), 2.46 - 2.35 (m, 1H), 2.25 - 1.80 (m, 4H), 1.75 - 1.63 (m, 1H). ¹⁹F NMR (377 MHz, DMSO-d₆): δ = -88.33 (s, 1F), -88.47 - -88.63 (m, 1F).

### Example 60. Synthesis of INSCoV-600C

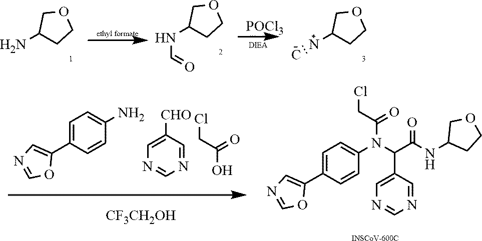

Step 1: To a mixture of tetrahydrofuran-3-amine (2 g, 22.96 mmol, 1 eq) in ethyl formate (8.50 g, 114.78 mmol, 9.23 mL, 5 eq) was stirred at 80° C. for 16 hr. TLC (EA:EtOH=3:1) showed the starting material (Rf=0.3) was consumed completely. And a new spot (Rf=0.7) was detected. The mixture was concentrated in reduced pressure. The crude compound was used into the next step without further purification. N-tetrahydrofuran-3-ylformamide (2.5 g, 21.71 mmol, 94.59% yield) was obtained as yellow oil, checked by ¹H NMR.

¹H NMR (400 MHz, CDCl₃): δ = 8.10 (s, 1H), 6.53 - 6.25 (m, 1H), 4.61 - 4.53 (m, 1H), 3.83 - 3.76 (m, 2H), 3.74 -3.62 (m, 2H), 2.42 - 2.12 (m, 2H).

Step 2: To a mixture of N-tetrahydrofuran-3-ylformamide (1.5 g, 13.03 mmol, 1 eq) and DIEA (8.42 g, 65.1 mmol, 11.4 mL, 5 eq) in DCM (100 mL) was added POCl₃ (2.40 g, 15.63 mmol, 1.45 mL, 1.2 eq) in one portion at -10° C. under N₂, then heated to 20° C. and stirred for 2 hours. TLC (PE:EA=1:1) showed N-tetrahydrofuran-3-ylformamide (Rf=0.1) was consumed and a new spot (Rf=0.8) was observed. The mixture was poured into sat. NaHCO₃ (500 mL) slowly at 0° C., and then extracted with DCM (300 mL × 2), dried over Na₂SO₄. The mixture was concentrated in reduced pressure at 25° C. The residue was purified by flash silica gel chromatography (ISCO®; 40 g SepaFlash® Silica Flash Column, Eluent of 0 \~50% Ethyl acetate/Petroleum ether gradient @ 50 mL/min). The mixture was concentrated in reduced pressure at 25° C. Compound 3 (500 mg, 5.15 mmol, 39.52% yield) was obtained as colorless oil and it was used to the next step directly.

Step 3: INSCoV-600C was synthesized according to the General procedure for INSCoV series. Purification B: The crude product was triturated with MeCN (5 mL) at 25° C. for 30 min. INSCoV-600C (198.49 mg, 438.65 µmol, 35.1% yield, 97.65% purity) was obtained as white-off solid, which was checked by HNMR, LCMS and HPLC.

LCMS: Retention time: 0.812 min, (M+H) =442.2. HPLC: Retention time: 1.560 min. ¹H NMR (400 MHz, DMSO-d₆): δ = 9.02 (s, 1H), 8.66 (dd, J = 4.0, 6.4 Hz, 1H), 8.59 - 8.44 (m, 3H), 7.77 (s, 1H), 7.66 (br s,1H), 7.70 (br d, J= 8.4 Hz, 1H), 7.61 -7.33 (m, 2H), 6.13 (s, 1H), 4.34 (tdd, J = 3.6, 6.4, 10.0 Hz, 1H), 4.18 - 3.99 (m, 2H), 3.87 - 3.63 (m, 3H), 3.56 (dd, J = 3.6, 9.0 Hz, 1H), 2.28 - 2.02 (m, 1H), 1.93 - 1.56 (m, 1H).

### Example 61. Synthesis of INSCoV-600D

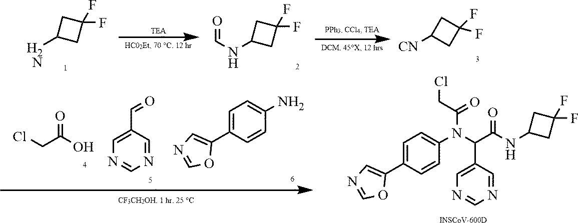

Step 1: The mixture of Compound 1 (5 g, 34.83 mmol, 1 eq, HCl) in ethyl formate (46.05 g, 621.64 mmol, 50 mL, 17.85 eq) and TEA (7.05 g, 69.66 mmol, 9.70 mL, 2 eq) were stirred at 70° C. for 12 h under N₂. TLC (EA/PE=1/1) indicated Compound 1 (Rf =0.0) was consumed and one new spot formed (Rf=0.1). The mixture was filtered, washed with PE (10 mL × 3). The filtrate was concentrated under vacuum. The reaction was used to next step and without purification. Compound 2 (5 g, crude) was obtained as white solid.

¹H NMR (400 MHz, CHLOROFORM-d): δ = 8.11 (s, 1H), 6.92 (s, 1H), 4.40 - 4.24 (m, 1H), 3.01 - 2.91 (m, 2H), 2.63 - 2.46 (m, 2H).

Step 2: To a solution of Compound 2 (2.0 g, 14.80 mmol, 1 eq) and TEA (1.50 g, 14.80 mmol, 2.06 mL, 1.0 eq) in DCM (20 mL) was added PPh₃ (4.27 g, 16.28 mmol, 1.1 eq) and CCl₄ (2.28 g, 14.80 mmol, 1.42 mL, 1.0 eq). The mixture was stirred at 45° C. for 12 h under N₂. TLC (PE/EA = 3/1) indicated Compound 2 (Rf = 0.1) was consumed and one small spot formed (Rf = 0.6). The resulting mixture was evaporated in vacuum (<20° C.). The residue was suspended in Et20 (100 ml) at 20° C. for 12 hrs under N₂. The mixture was filtered, washed with Et2O (50 mL × 2) and the filtrate was concentrated under vacuum ( < 20° C.). The reaction was used to next step and without purification. Compound 3 (1.0 g, crude) was obtained as yellow oil.

¹H NMR (400 MHz, DMSO-d6) δ = 4.39 - 4.20 (m, 1H), 3.22 - 3.06 (m, 2H), 3.00 - 2.83 (m, 2H).

Step 3: INSCoV-600D was synthesized according to the General procedure for INSCoV series. Purification B: MTBE (20 mL) was added the reaction mixture and cooled to 0° C. for 12 hrs. The mixture was filtered and washed with MTBE (10 mL × 3) to get the product. The filter cake was concentrated under vacuum. INSCoV_600D (181.18 mg, 382.10 µmol, 41.30% yield, 97.402% purity) was obtained as yellow solid, which was confirmed by HNMR, FNMR, LCMS, SFC and HPLC.

LCMS: Retention time: 0.869 min, (M+H) =462.2. HPLC: Retention time: 1.657 min. SFC: Retention time: 1.255 min, 1.487 min. ¹H NMR (400 MHz, DMSO-d₆): δ = 8.99 (s, 1H), 8.78 (d, J = 6.0 Hz, 1H), 8.51 (s, 2H), 8.46 (s, 1H), 7.72 (s, 1H), 7.66 (d, J= 8.8 Hz, 2H), 7.42 (s, 2H), 6.05 (s, 1H), 4.15 - 3.97 (m, 3H), 3.01 - 2.82 (m, 2H), 2.63 - 2.51 (m, 2H). ¹⁹F NMR (377 MHz, DMSO-d6): δ = -82.27 (d, J= 194.6 Hz, 1F), -95.79 (d, J= 197.4 Hz, 1F).

### Example 62. Synthesis of INSCoV-600E

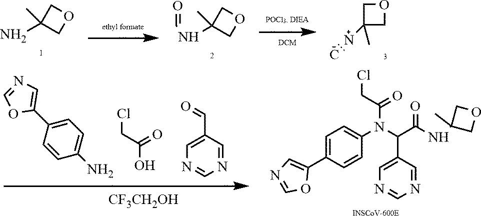

Step 1: A mixture of 3-methyloxetan-3-amine (1 g, 11.48 mmol, 1 eq) in ethyl formate (3 g, 40.50 mmol, 3.26 mL, 3.53 eq) was stirred at 90° C. for 16 hrs. TLC (PE:EA=1:1) showed the starting material (Rf=0.4) was consumed completely and a new spot (Rf=0.2) was observed. The mixture was concentrated in reduced pressure. N-(3-methyloxetan-3-yl)formamide (1.3 g, 11.29 mmol, 98.37% yield) was obtained as brown solid, which was detected by HNMR.

¹H NMR (400 MHz, CHLOROFORM-d): δ = 8.14 (d, J =0.9 Hz, 1H), 7.27 (s, 1H), 4.81 (d, J =6.4 Hz, 2H), 4.51 (d, J =6.6 Hz,2H), 1.69 (s, 3H).

Step 2: To a mixture of N-(3-methyloxetan-3-yl)formamide (1 g, 8.69 mmol, 1 eq) and DIEA (5.61 g, 43.43 mmol, 7.56 mL, 5 eq) in DCM (100 mL) was added POCl₃ (2.00 g, 13.03 mmol, 1.21 mL, 1.5 eq) in one portion at -10° C. under N₂, then heated to 25° C. and stirred for 2 hours. TLC (plate1, PE:EA=1:1) showed the starting material (Rf=0.2) was consumed completely and a new spot (Rf=0.8) was observed. The mixture was poured into ice-Saturated sodium bicarbonate solution(w/w = 1/1) (100 mL) at 0° C. and stirred for 10 min. The aqueous phase was extracted with DCM(100 mL × 2), dried with anhydrous Na₂SO₄. The mixture was concentrated in reduced pressure at 20° C. The residue was purified by flash silica gel chromatography (ISCO®; 40 g SepaFlash® Silica Flash Column, Eluent of 0 \~50% Ethyl acetate/Petroleum ether gradient @ 50 mL/min). The mixture was concentrated in reduced pressure at 20° C. TLC(plate2, PE:EA=1:1, Rf=0.8). 3-isocyano-3-methyl-oxetane (350 mg, 3.60 mmol, 41.49% yield) was obtained as colorless oil.

Step 3: INSCoV-600E was synthesized according to the General procedure for INSCoV series. Purification A: The crude product was purified by prep-HPLC (column: Welch MLtimate XB-CN 250 × 50 × 10 um; mobile phase: [Hexane-IPA]; B%: 30%-70%, 15 min). 2-(N-(2-chloroacetyl)-4-oxazol-5-yl-anilino)-N-(3-methyloxetan-3-yl)-2-pyrimidin-5-yl-acetamide (105.94 mg, 228.59 µmol, 24.41% yield, 95.342% purity) was obtained as yellow solid, which was detected by LCMS, HPLC and HNMR.

LCMS: Retention time: 0.750 min, [M+H⁺] = 442.2. HPLC: Retention time: 1.580 min. ¹H NMR (400 MHz, DMSO-d₆): δ = 8.99 (s, 1H), 8.84 (s, 1H), 8.52 (s, 2H), 8.47 (s, 1H), 7.73 (s, 1H), 7.66 (br d, J =8.6 Hz, 2H), 7.54 - 7.33 (m, 2H), 6.06 (s, 1H), 4.56 (dd, J = 6.2, 15.8 Hz, 2H), 4.33 -4.27 (m, 2H), 4.18 - 4.00 (m, 2H), 1.50 (s, 3H).

### Example 63. Synthesis of N-(4-(lH-Imidazol-5-vl)Phenyl)-2-Chloro-N-(2-((4,4-Difluorocyclohexyl)Amino)-2-Oxo-l-(Pyrimidin-5-yl)Ethyl)Acetamide (INSCoV-6001)

INSCoV-600I was synthesized according to the General procedure for INSCoV series. Purification A: The residue was dissolved in MeOH (2 mL) and purified by Pre-HPLC (column: 3_Phenomenex Luna C18 75 × 30 mm × 3 µm; mobile phase: [water (0.05% HCl)-ACN]; B%: 12%-32%, 6.5 min) and concentrated to remove MeCN, the liquid was under lyophilization to give the crude product. The residue was dissolved in DMF (2 mL) and purified by Pre-HPLC (column: 3_Phenomenex Luna C18 75 × 30 mm × 3 µm; mobile phase: [water (0.05%HCl)-ACN]; B%: 13%-33%, 6.5 min) and concentrated to remove MeCN, the liquid was under lyophilization to give the crude product. INSCoV-600I (13.3 mg, 25.58 µmol, 2.76% yield, 94.024% purity) was obtained as yellow solid, which was confirmed by LCMS, HPLC, HNMR and FNMR.

LCMS: Retention time: 0.840 min, (M+H) =489.2; Retention time: 0.724 min, (M+H) =489.0. HPLC: Retention time: 1.767 min. ¹H NMR (400 MHz, METHANOL-d4): δ = 9.02 (d, J = 1.2 Hz, 1H), 8.94 (s, 1H), 8.57 (s, 2H), 7.96 (d, J= 1.2 Hz, 1H), 7.84 - 7.21 (m, 4H), 6.15 (s, 1H), 4.08 - 3.81 (m, 3H), 2.12 - 1.80 (m, 6H), 1.72 - 1.41 (m, 2H). ¹⁹F NMR (376 MHz, METHANOL-d4): δ = -88.44 - -112.41 (m, 1F).

### Example 64. Synthesis of INSCoV-600L

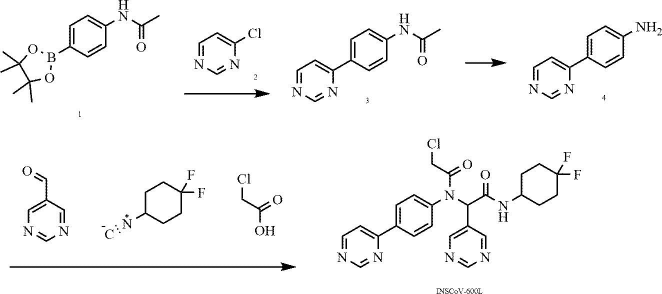

Step 1: To a solution of 4-chloropyrimidine (500 mg, 3.31 mmol, 1 eq, HCl) and N-[4-(4,4,5,5-tetramethyl-1,3,2-dioxaborolan-2-yl)phenyl]acetamide (1.04 g, 3.97 mmol, 1.2 eq) in dioxane (5 mL) and H₂O (1 mL) was added Pd(dppf)Cl₂ (242.30 mg, 331.14 µmol, 0.1 eq) and NaHCO₃ (834.57 mg, 9.93 mmol, 386.38 µL, 3.0 eq), it was charged with N₂ three times and stirred at 100° C. for 16 h. LCMS showed 4-chloropyrimidine was consumed and desired mass was detected. Water (20 ml) and EA (20 ml) was added, the organic layer was washed with brine (20 ml), dried over Na₂SO₄ and concentrated in vacuum. The crude was purified by column chromatography (SiO₂, PE:EA 10:1-1:2). N-(4-pyrimidin-4-ylphenyl)acetamide (400 mg, 1.83 mmol, 55.29% yield, 97.596% purity) was obtained as a yellow solid, which was confirmed by HNMR and LCMS.

LCMS: Retention time: 0.602 min, (M+H) = 214.1. ¹H NMR (400 MHz, CHLOROFORM-d): δ = 9.16 (d, J=1.1 Hz, 1H), 8.66 (d, J=5.4 Hz, 1H), 8.06 - 7.95 (m, 2H), 7.67 - 7.56 (m, 3H), 7.44 (br s, 1H), 2.15 (s, 3H).

Step 2: To a solution of N-(4-pyrimidin-4-ylphenyl)acetamide (350 mg, 1.64 mmol, 1 eq) in MeOH (10 mL) was added HCl (10 mL) (2 M) at 25° C., it was stirred at 70° C. for 2 h. LCMS showed N-(4-pyrimidin-4-ylphenyl)acetamide was consumed and desired mass was detected. It was poured into water (50 ml) , the pH of the mixture was adjusted to 9 by solid Na₂CO₃, EA (50 ml) was added, the organic layer was washed with brine (50 ml), dried over Na₂SO₄ and concentraed in vacuum. The crude was used in the next step directly. 4-Pyrimidin-4-ylaniline (280 mg, 1.64 mmol, 99.64% yield) was obtained as a yellow soild, which was confirmed by HNMR.

LCMS: Retention time: 0.702 min, (M+H) = 172.3. ¹HNMR (400 MHz, DMSO-d₆): δ = 9.03 (d, J=1.3 Hz, 1H), 8.62 (d, J= 5.6 Hz, 1H), 7.99 - 7.90 (m, 2H), 7.82 (dd, J=1.4, 5.5 Hz, 1H), 6.72 -6.61 (m, 2H), 5.81 (s, 2H).

Step 3: INSCoV-600L was synthesized according to the General procedure for INSCoV series. Purification B: The crude was trituated with EtOH (1 ml) and filtered, the cake was washed with PE (1 ml) and dried in vacuum. 2-(N-(2-chloroacetyl)-4-pyrimidin-4-yl-anilino)-N- (4,4-difluorocyclohexyl)-2-pyrimidin-5-yl-acetamide (144.91 mg, 279.56 µmol, 26.42% yield, 96.640% purity) was obtained as a yellow solid, which was confirmed by LCMS, HPLC, HNMR and FNMR.

LCMS: Retention time: 0.850 min, (M+H) = 501.1. HPLC: Retention time: 1.769 min. ¹H NMR (400 MHz, DMSO-d₆) δ = 9.24 (s, 1H), 8.96 (s, 1H), 8.87 (d, J= 5.3 Hz, 1H), 8.51 (s, 2H), 8.33 (br d, J=7.3 Hz, 1H), 8.24 - 8.02 (m, 3H), 7.53 (br s, 2H), 6.19 - 6.05 (m, 1H), 4.16 - 4.00 (m, 2H), 3.85 (br s, 1H), 2.07 - 1.69 (m, 6H), 1.62 - 1.46 (m, 1H), 1.46 - 1.29 (m, 1H), 1.17 (t, J= 7.2 Hz, 1H).

### Example 65. Synthesis of INSCoV-600M

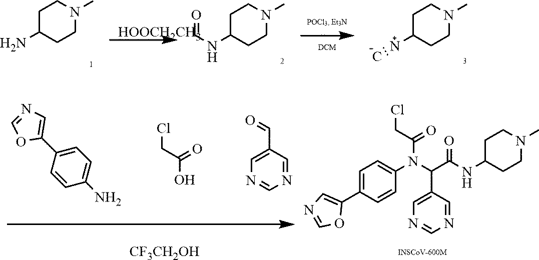

Step 1: A mixture of 1-methylpiperidin-4-amine (2 g, 17.51 mmol, 1 eq) in ethyl formate (12.97 g, 175.15 mmol, 14.09 mL, 10 eq) was stirred at 60° C. for 16 hrs. LC-MS showed the reaction was finished and desired product was found. The mixture was concentrated in reduced pressure. N-(1-methyl-4-piperidyl)formamide (2.45 g, crude) was obtained as brown oil.

LCMS: Retention time: 0.197 min, [M+H⁺] = 143.3.

Step 2: To a solution of N-(1-methyl-4-piperidyl)formamide (1 g, 7.03 mmol, 1 eq) and TEA (2.13 g, 21.10 mmol, 2.94 mL, 3 eq) in DCM (50 mL) was added POCl₃ (3.23 g, 21.10 mmol, 1.96 mL, 3 eq) at 0° C. The mixture was stirred at 20° C. for 2 hrs. LCMS showed the reaction was finished and desired product was found. The mixture was poured into Saturated sodium bicarbonate (50 mL) at 0° C. The aqueous phase was extracted with ethyl acetate (100 mL × 3), dried with anhydrous Na₂SO₄, filtered and concentrated in vacuum. 4-Isocyano-1-methyl-piperidine (120 mg, crude) was obtained as brown oil, which was detected by LCMS.

LCMS: Retention time: 0.697 min, [M+H⁺] = 125.2.

Step 3: INSCoV-600M was synthesized according to the General procedure for preparation of INSCoV series. The crude product was purified by prep-HPLC (column: Welch MLtimate XB-CN 250 × 50 × 10 um; mobile phase: [Hexane-IPA]; B%:35%-75%, 15 min). 2-(N-(2-chloroacetyl)-4-oxazol-5-yl-anilino)-N-(1-methyl-4-piperidyl)-2-pyrimidin-5-yl-acetamide (22.04 mg, 44.07 µmol, 4.71% yield, 93.756% purity) was obtained as yellow solid, which was detected by LCMS, HPLC and HNMR.

LCMS: Retention time: 0.824 min, [M+H⁺] = 469.2. HPLC: Retention time: 1.646 min. ¹H NMR (400 MHz, DMSO-d₆): δ = 9.01 - 8.92 (m, 1H), 8.57 - 8.43 (m, 3H), 8.42 - 8.33 (m, 1H), 7.71 (s, 1H), 7.68 - 7.59 (m, 2H), 7.57 - 7.33 (m, 2H), 6.07 (s, 1H), 4.04 (d, J =8.1 Hz, 2H), 3.73 - 3.63 (m, 1H), 3.09 - 2.78 (m, 2H), 2.69 - 2.64 (m, 1H), 2.70 - 2.62 (m, 1H), 2.42 - 2.34 (m, 3H), 1.89 - 1.67 (m, 2H), 1.61 - 1.45 (m, 1H), 1.42 - 1.28 (m, 1H).

### Example 66. Synthesis of INSCoV-600O

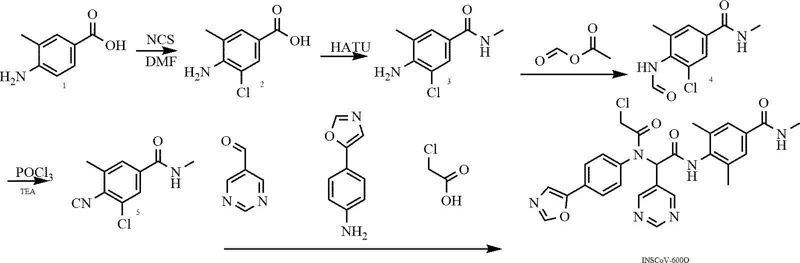

Step 1: To a mixture of 4-amino-3-methyl-benzoic acid (5.00 g, 33.1 mmol, 1.00 eq) in DMF (50.0 mL) was added NCS (4.42 g, 33.1 mmol, 1.00 eq) in one portion at 25° C. The mixture was stirred at 100° C. for 1 hr. LCMS showed 4-amino-3-methyl-benzoic acid consumed and desired Mass was detected. The mixture was poured into water (100 mL), and then filtered. The filter cake was washed with water (50 mL), and then dried in vacuum. It was not purified and used for the next step. 4-amino-3-chloro-5-methyl-benzoic acid (5.50 g, 29.6 mmol, 89.6% yield) was obtained as brown solid, which was determined by HNMR.

LCMS: Retention time: 0.702 min, [M+H⁺] = 186.1. ¹H NMR (400 MHz, DMSO-d6): δ = 12.34 (s, 1H), 7.61 (d, J =1.6 Hz, 1H), 7.52 (s, 1H), 5.81 (s, 2H), 2.16 (s, 3H).

Step 2: To a solution of 4-amino-3-chloro-5-methyl-benzoic acid (3.00 g, 16.2 mmol, 1.00 eq) and methanamine hydrochloride (2.18 g, 32.3 mmol, 2.0 eq) in DMF (30.0 mL) was added HATU (12.3 g, 32.3 mmol, 2.00 eq) and DIPEA (4.18 g, 32.3 mmol, 5.63 mL, 2.00 eq) at 20° C. The mixture was stirred at 20° C. for 6 hrs. TLC (PE:EA=1:1) showed 4-amino-3-chloro-5-methyl-benzoic acid (Rf=0.5) consumed and a new spot (Rf=0.4) was observed. The mixture was poured into water (100 mL), and then extracted with EA (50 mL × 3). The combined organic layers were dried over Na₂SO₄, filtered and concentrated in vacuum to give a residue. The residue was purified by silica gel chromatography (PE:EA=1:1-0:1). 4-Amino-3-chloro-N,5-dimethyl-benzamide (3.20 g, 16.1 mmol, 99.7% yield) was obtained as white solid, which was determined by HNMR.

¹H NMR (400 MHz, CHLOROFORM-d): δ = 7.58 (d, J =1.8 Hz, 1H), 7.43 - 7.39 (m, 1H), 2.96 (s, 3H), 2.21 (s, 3H).

Step 3: A mixture of formic acid (405 mg, 8.81 mmol, 332 µL, 3.50 eq) in acetic anhydride (308 mg, 3.02 mmol, 283 µL, 1.20 eq) in one portion at 20° C. under N₂. The mixture was stirred at 20° C. for 10 min. A solution of 4-amino-3-chloro-N,5- dimethyl-benzamide (500 mg, 2.52 mmol, 1.00 eq) in DCM (5 mL) was added to the mixture at 20° C. The resulting mixture was stirred at 30° C. for 3 hrs. LCMS showed 4-amino-3-chloro-N,5-dimethyl-benzamide consumed and desired Mass was detected. The mixture was concentrated in vacuum to give a residue. The residue was triturated with EA (10 mL), and then filtered. The filter cake was dried in vacuum. 3-chloro-4-formamido-N,5-dimethyl-benzamide (263 mg, 1.16 mmol, 46.10% yield) was obtained as yellow solid, which was determined by HNMR.

LCMS: Retention time: 0.209 min, [M+H⁺] = 227.2. ¹H NMR (400 MHz, DMSO-d₆): δ = 8.29 (d, J =1.3 Hz, 1H), 7.76 - 7.68 (m, 1H), 2.77 (d, J =4.5 Hz, 3H), 2.23 (s, 3H).

Step 4: To a solution of 3-chloro-4-formamido-N,5-dimethyl-benzamide (260 mg, 1.15 mmol, 1.00 eq) and TEA (116 mg, 1.15 mmol, 160 µL, 1.00 eq) in DCM (4.00 mL) was added POCl₃ (176 mg, 1.15 mmol, 107 µL, 1.00 eq) at 0° C. The mixture was stirred at 20° C. for 1 hr. TLC (PE:EA=1:1) showed 3-chloro-4-formamido-N,5-dimethyl-benzamide (Rf=0.1) consumed and a new spot (Rf=0.7) was observed. The mixture was diluted with DCM (50 mL), and then poured into NaHCO₃ (50 mL). The resulting mixture was separated by separating funnel. The organic layer was dried over Na₂SO₄, filtered and concentrated in vacuum to give a residue. The residue was purified by silica gel chromatography (PE:EA=10:1-5:1). 3-Chloro-4-isocyano-N,5-dimethyl-benzamide (60.0 mg, 288 µmol, 25.1% yield) was obtained as yellow solid, which was determined by HNMR.

¹H NMR (400 MHz, DMSO-d₆): δ = 8.66 (d, J =4.0 Hz, 1H), 7.92 (d, J =1.2 Hz, 1H), 7.84 (s, 1H), 2.78 (d, J =4.4 Hz, 3H), 2.46 (s, 3H).

Step 5: INSCoV-600O was synthesized according to the General procedure for INSCoV series. Purification C: The residue was purified by silica gel chromatography (PE:EA=10:1-0:1). 3-chloro-4-[[2-(N-(2-chloroacetyl)-4-oxazol-5-yl-anilino)-2-pyrimidin-5-yl-acetyl]amino]-N,5-dimethyl-benzamide (15.54 mg, 25.55 µmol, 13.33% yield, 91% purity) was obtained as yellow solid, which was determined by HNMR, LCMS and HPLC.

LCMS: Retention time: 0.823 min, [M+H⁺] = 554.9. HPLC: Retention time: 1.585 min. ¹H NMR (400 MHz, DMSO-d₆): δ = 10.20 (s, 1H), 9.00 (s, 1H), 8.62 (s, 2H), 8.57 - 8.51 (m, 1H), 8.46 (s, 1H), 7.81 - 7.63 (m, 5H), 7.55 - 7.32 (m, 2H), 6.33 (s, 1H), 4.10 (s, 2H), 2.78 (d, J =4.4 Hz, 3H), 2.33 - 2.23 (m, 3H).

### Example 67 Synthesis of INSCoV-600R(2), 138 INSCoV-600R(2A) and 139 INSCoV-600R(2B)

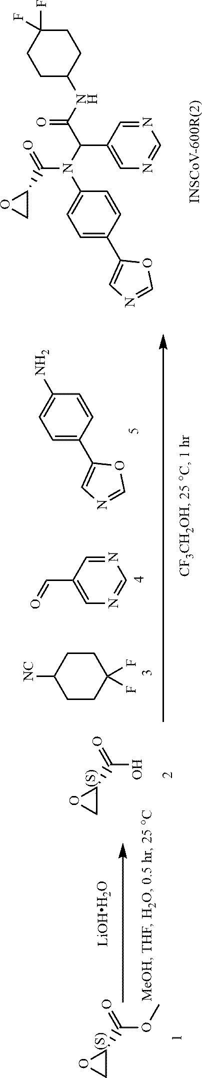

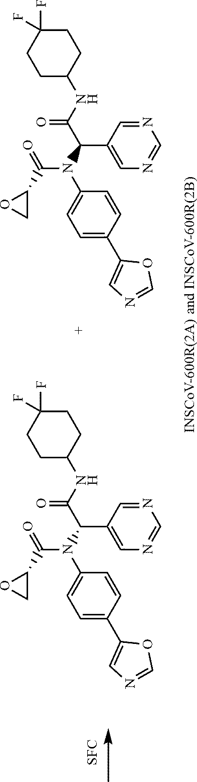

Step 1: To a solution of Compound 1 (0.5 g, 4.90 mmol, 1 eq) in THF (2 mL), MeOH (2 mL) and H₂O (1 mL) was added LiOH*H₂O (411.05 mg, 9.80 mmol, 2 eq). The reaction mixture was stirred at 25° C. for 1 hr. TLC (PE: EA=3:1) showed one new spot (Rf= 0.0) formed. The reaction mixture was concentrated under vacuum to remove MeOH and THF. Then the mixture was adjusted to pH=2 by 1N HCl solution and extracted with EA (50 mL × 3). The combined organic phase was concentrated under vacuum (<25° C.). The reaction mixture was used to next step and no purification. Compound 2 (0.3 g, 3.41 mmol, 69.56% yield) was obtained as colorless oil, which was confirmed by HNMR.

¹H NMR (400 MHz, DMSO-d6): δ = 3.41 - 3.85 (m, 1H), 2.93 - 2.87 (m, 1H), 2.82 - 2.76 (m, 1H).

Step 2: INSCoV-600R(2) was synthesized according to the General procedure for INSCoV series . The reaction was concentrated under vacuum. The crude product was triturated with MTBE (20 mL) and washed with MTBE (10 mL × 2). The residue was diluted with MTBE (20 mL), washed with MTBE (10 mL × 2) and concentrated under vacuum. INSCoV-600R(2) (212.57 mg, 400.82 µmol, 43.33% yield, 91.161% purity) was obtained as yellow solid, which was confirmed by HNMR, FNMR, LCMS, SFC and HPLC.

LCMS: Retention time: 0.878 min, (M+H) =484.3. SFC: Retention time: 1.492 min, 2.151 min. ¹H NMR (400 MHz, DMSO-d6) δ = 8.97 (d, J =8.8 Hz, 1H), 8.51 (d, J =5.6 Hz, 2H), 8.45 (d, J =0.8 Hz, 1H), 8.42 - 8.27 (m, 1H), 7.76 - 7.61 (m, 3H), 7.52 - 7.36 (m, 2H), 6.24 - 5.96 (m, 1H), 3.93 - 3.75 (m, 1H), 3.17 - 3.08 (m, 1H), 2.87 - 2.75 (m, 1H), 2.75 - 2.69 (m, 1H), 2.05 - 1.69 (m, 6H), 1.61 - 1.29 (m, 2H). ¹⁹F NMR (377 MHz, DMSO-d6) δ = -87.22 - 103.36 (m, 1F)

Step 3: General procedure for preparation of INSCoV-600R(2A) and INSCoV-600R(2B). The INSCoV-600R(2) (0.1 g, 206.84 µmol, 1 eq) was separated by chiral SFC (column: DAICEL CHIRALPAK AD(250 mm × 30 mm, 10 um); mobile phase: [0.1%NH₃•H₂O MEOH]; B%: 40%-40%, 4.3 min; 45 min) and concentrated under vacuum. INSCoV-600R(2A) (21.32 mg, 41.08 µmol, 19.86% yield, 93.149% purity) was obtained as yellow solid, which was confirmed by HNMR, FNMR, LCMS, SFC and HPLC. INSCoV-600R(2B) (10.33 mg, 20.82 µmol, 10.07% yield, 97.456% purity) was obtained as yellow solid, which was confirmed by HNMR, FNMR, LCMS, SFC and HPLC.

INSCoV-600R(2A): LCMS: Retention time: 0.876 min, (M+H) =484.3, HPLC: Retention time: 1.601 min, SFC: Retention time: 1.533 min, ¹H NMR (400 MHz, DMSO-d6) δ = 8.96 (d, J = 1.2 Hz, 1H), 8.50 (s, 2H), 8.45 (d, J= 1.2 Hz, 1H), 8.36 (d, J= 7.6 Hz, 1H), 7.71 (s, 1H), 7.67 (d, J= 8.0 Hz, 2H), 7.47 (s, 2H), 6.14 (s, 1H), 3.90 - 3.80 (m, 1H), 3.13 - 3.08 (m, 1H), 2.87 - 2.81 (m, 1H), 2.77 - 2.71 (m, 1H), 2.01 - 1.75 (m, 6H), 1.62 - 1.47 (m, 1H), 1.45 - 1.31 (m, 1H), ¹⁹F NMR (377 MHz, DMSO-d6) δ = 91.06 -95.14 (m, 1F), -96.46-100.47 (m, 1F).

INSCoV-600R(2A): LCMS: Retention time: 0.874 min, (M+H) =484.3, HPLC: Retention time: 1.593 min, SFC: Retention time: 2.139 min, ¹H NMR (400 MHz, DMSO-d6) δ = 8.98 (s, 1H), 8.51 (s, 2H), 8.45 (s, 1H), 8.31 (d, J= 7.6 Hz, 1H), 7.71 (s, 1H), 7.67 (d, J= 8.8 Hz, 2H), 7.44 (d, J= 7.2 Hz, 2H), 6.08 (s, 1H), 3.84 - 3.78 (m, 1H), 3.13 (s, 1H), 2.81 - 2.75 (m, 1H), 2.75 - 2.69 (m, 1H), 2.01 - 1.73 (m, 6H), 1.56 - 1.44 (m, 1H), 1.40 - 1.30 (m, 1H), ¹⁹F NMR (37 MHz, DMSO-d6) δ = -93.34 (d, J= 234.6 Hz, 1F), -96.19 - -99.53 (m, 1F).

According to modelling and activity data, it is expected that INSCoV-600R(2A) has a structure of

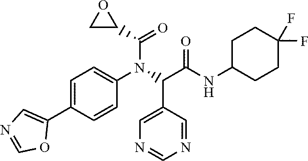

### Example 68. Synthesis of INSCoV-600X

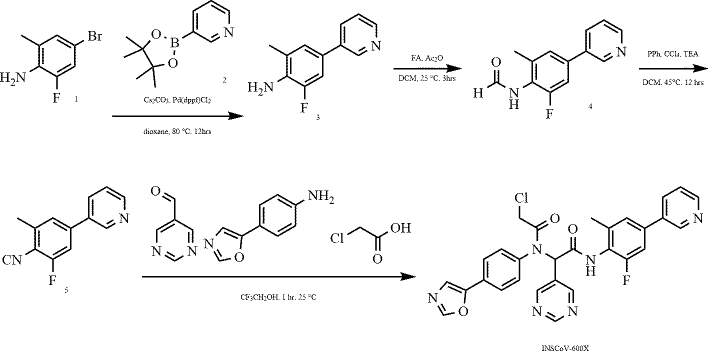

Step 1: To a solution of Compound 1 (2 g, 9.80 mmol, 1 eq) and Compound 2 (2.41 g, 11.76 mmol, 1.2 eq) in dioxane (20 mL) was added Cs₂CO₃ (9.58 g, 29.41 mmol, 3 eq) and Pd(dppf)Cl₂ (1.43 g, 1.96 mmol, 0.2 eq). The reaction mixture was stirred at 80° C. for 12 hrs under N₂. LCMS showed Compound 1 was consumed completely and one peak (Rt= 1.063 min) with desired mass was detected. TLC (PE: EA=1:1) showed Compound 1 (Rf= 0.8) was consumed completely and three new spots (Rf=0.02, Rf=0.3, Rf= 0.5) formed. The mixture was diluted with water (30 mL) and extracted with EtOAc (80 mL × 2). The organic layer was dried with anhydrous Na₂SO₄, filtered and concentrated under vacuum. The residue was purified by flash silica gel chromatography (ISCO®; 80 g SepaFlash® Silica Flash Column, Eluent of 0 \~60% Ethyl acetate/Petroleum ether gradient @ 100 mL/min) and concentrated under vacuum. Compound 3 (3.5 g, crude) was obtained as yellow solid, which was confirmed by HNMR and FNMR.

LCMS: Retention time: 1.063 min, (M+H) =203.1, ¹H NMR (400 MHz, DMSO-d₆): δ = 8.81 (d, J = 2.4 Hz, 1H), 8.47 - 8.41 (m, 1H), 7.99 - 7.83 (m, 1H), 7.41 - 7.35 (m, 1H), 7.34 - 7.28 (m, 1H), 7.24 (s, 1H), 5.10 (s, 2H), 2.19 (s, 3H), ¹⁹F NMR (377 MHz, DMSO-d6): δ = -134.656 (m, 1F).

Step 2: A mixture of HCOOH (2.41 g, 50.19 mmol, 3.5 eq) and Ac₂O (1.76 g, 17.21 mmol, 1.61 mL, 1.2 eq) was stirred at 25° C. for 10 min under N₂. A solution of Compound 3 (2.9 g, 14.34 mmol, 1 eq) in DCM (30 mL) was added to the mixture at 0° C. The resulting mixture was stirred at 25° C. for 3 hrs. LCMS showed Compound 3 was consumed completely and one peak (Rt= 0.797 min) with desired mass was detected. TLC (PE: EA = 1: 2) show the Compound 3 (Rf = 0.7) was remained and one spot (Rf=0.5) formed. The reaction mixture was diluted with water (20 mL) and extracted with DCM (30 mL × 3). The combined organic phase was dried by anhydrous Na₂SO₄ and concentrated under vacuum. The residue was purified by flash silica gel chromatography (ISCO®; 80 g SepaFlash® Silica Flash Column, Eluent of 0 \~100% Ethyl acetate/Petroleum ether gradient @ 100 mL/min). The combined organic phase was dried by anhydrous Na₂SO₄ and concentrated under vacuum. Compound 4 (1.5 g, 6.52 mmol, 45.43% yield) was obtained as white solid, which was confirmed by HNMR.

LCMS: Retention time: 0.797 min, (M+H) =231.2, ¹H NMR (400 MHz, DMSO-d₆): δ = 9.77 (s, 1H), 8.93 (d, J= 1.8 Hz, 1H), 8.62- 8.56 (m 1H), 8.31 (s, 1H), 8.14 - 8.08 (m, 1H), 7.56 - 7.46 (m, 3H), 2.28 (s, 3H).

Step 3: To a solution of Compound 4 (1.2 g, 5.21 mmol, 1 eq) in DCM (12 mL) was added TEA (527.40 mg, 5.21 mmol, 725.45 µL, 1 eq), PPh₃ (1.50 g, 5.73 mmol, 1.1 eq) and CCl₄ (801.73 mg, 5.21 mmol, 501.08 µL, 1 eq). The reaction mixture was stirred at 45° C. for 12 hrs under N₂. LCMS showed Compound 4 remained (Rt= 0.807 min) and no desired mass was detected. TLC (PE: EA=1:1) showed three new spots (Rf= 0.2, Rf= 0.3, Rf=0.7) formed. The mixture was diluted with water (10 mL) and extracted with DCM (20 mL × 3). The organic layer was dried with anhydrous Na₂SO₄, filtered and concentrated under vacuum. The residue was purified by flash silica gel chromatography (ISCO®; 80 g SepaFlash® Silica Flash Column, Eluent of 0 \~80% Ethyl acetate/Petroleum ether gradient @ 100 mL/min) and concentrated under vacuum. Compound 5 (1 g, 4.71 mmol, 90.41% yield) was obtained as white solid, which was confirmed by HNMR and FNMR.

LCMS: Retention time: 0.805 min, (M+H) =231.1, ¹H NMR (400 MHz, DMSO-d6): δ = 8.97 (d, J= 2.4 Hz, 1H), 8.66 - 8.60 (m, 1H), 8.19 - 8.13 (m, 1H), 7.80 (d, J= 10.8 Hz, 1H), 7.73 (s, 1H), 7.55 - 7.49 (m, 1H), 2.49 (s, 3H), ¹⁹F NMR (377 MHz, DMSO-d6): δ = -119.049 (m, 1F).

Step 4: INSCoV-600X was synthesized according to the General procedure for preparation of INSCoV series . Purification B: MTBE (20 mL) was added the reaction mixture, filtered and washed with MTBE (10 mL × 3) to get the crude product. The residue was triturated with MTBE (20 mL), filtered and washed with MTBE (10 mL × 3). The filter cake was concentrated under vacuum. INSCoV-600X (301.95 mg, 517.73 µmol, 55.97% yield, 95.5% purity) was obtained as yellow solid, which was confirmed by HNMR, FNMR, LCMS, SFC and HPLC.

LCMS: Retention time: 0.774 min, (M+H) =557.1, HPLC: Retention time: 1.522 min, SFC: Retention time: 1.106 min, 3.019 min, ¹H NMR (400 MHz, DMSO-d₆): δ = 10.04 (s, 1H), 9.02 (s, 1H), 8.94 (d, J = 1.8 Hz, 1H), 8.62 (s, 2H), 8.62 - 8.56 (m, 1H), 8.46 (s, 1H), 8.15 - 8.09 (m, 1H), 7.73 (s, 1H), 7.67 (d, J =8.4 Hz, 2H), 7.61 - 7.38 (m, 5H), 6.38 (s, 1H), 4.10 (s, 2H), 2.28 (s, 3H), ¹⁹F NMR (377 MHz, DMSO-d6): δ = -119.55 (s, 1F).

### Example 69. Synthesis of N-(2-((4,4-Difluorocyclohexyl)Amino)-2-Oxo-1-(Pyrimidin-5-yl)Ethyl)-N-(4-(Isoxazol-5-yl)Phenyl)Oxirane-2-Carboxamide (INSCoV-600Y)

INSCoV-600Y was synthesized according to the General procedure for INSCoV series. Purification A: The residue was dissolved in DMF (2 mL) and purified by Pre-HPLC (column: Waters Xbridge 150 × 25 mm × 5 µm; mobile phase: [water (10 mM NH₄HCO₃)-ACN]; B%: 20%-50%, 9 min) and diluted with water (20 mL), the liquid was under lyophilization to give the product. The residue was dissolved in DMF (2 mL) and purified by Pre-HPLC (column: 3_Phenomenex Luna C18 75 × 30 mm × 3 µm; mobile phase: [water (0.05%HCl)-ACN]; B%: 28%-48%, 6.5 min) and diluted with water (20 mL), the liquid was under lyophilization to give the crude product (Instability In Acid), which was confirmed by LCMS. INSCoV-600Y (31.65 mg, 59.87 µmol, 6.47% yield, 91.460% purity) was obtained as white solid, which was confirmed by LCMS, HPLC, HNMR and FNMR.

LCMS: Retention time: 0.893 min, (M+H) = 484.3, HPLC: Retention time: 1.724 min, ¹H NMR (400 MHz, DMSO-d₆): δ = 9.04 - 8.90 (m, 1H), 8.69 - 8.63 (m, 1H), 8.58 - 8.47 (m, 2H), 8.42 -8.27 (m, 1H), 7.86 - 7.80 (m, 2H), 7.65 - 7.34 (m, 2H), 7.08 - 6.99 (m, 1H), 6.19 - 6.07 (m, 1H), 3.93 - 3.69 (m, 1H), 3.15 - 3.09 (m, 1H), 2.89 - 2.70 (m, 1H), 2.03 - 1.71 (m, 6H), 1.62 - 1.27 (m, 2H), ¹⁹F NMR (377 MH_{z}, DMSO-d6): δ = -87.29 - 105.18 (m, 1F).

### Example 70. Synthesis of 2-Chloro-N-(2-((4,4-Difluorocyclohexyl)Amino)-2-Oxo-1-(Pvridazin-4-vl)Ethyl)-N-(4-(Thiazol-5-vl)Phenyl)Acetamide (INSCoV-601F)

To a solution of compound 4 (150 mg, 851.12 µmol, 1 eq) and compound 2 (123.54 mg, 851.12 µmol, 1 eq) in CF3CH2OH (6 mL) was added compound 1 (80.43 mg, 851.12 µmol, 95.75 µL, 1 eq) and compound 3 (92.00 mg, 851.12 µmol, 1 eq). The reaction mixture was stirred at 25° C. for 1 hr. LCMS showed reactant 1 was consumed and one peak of desired mass was detected. The reaction was concentrated under vacuum. The crude product was triturated with PE/EA=20/1(20 mL*3) and filtered. INSCoV-601F (224.12 mg, 417.59 µmol, 49.06% yield, 94.273% purity) as brown solid was obtained, which was confirmed by HNMR, FNMR, LCMS, HPLC and SFC.

LCMS: Retention time: 0.880 min, (M+H) = 506.3, HPLC: Retention time: 1.899 min, SFC: Retention time: 0.499 min, 1.259 min, ¹H NMR (400 MHz, DMSO-d₆): δ = 9.10 (d, J= 0.6 Hz, 1H), 9.08 - 9.04 (m, 1H), 9.15 - 8.98 (m, 1H), 8.37 (d, J= 7.8 Hz, 1H), 8.33 (d, J= 0.6 Hz, 1H), 7.63 (d, J= 8.8 Hz, 2H), 7.46 - 7.37 (m, 3H), 6.06 (s, 1H), 4.16 - 4.01 (m, 2H), 3.89 - 3.77 (m, 1H), 1.97 - 1.86 (m, 4H), 1.80 - 1.76 (m, 2H), 1.55 - 1.48 (m, 1H), 1.41-1.40 (m, 1H), ¹⁹F NMR (377 MHz, DMSO-d₆): δ = -87.22 - -104.30 (m, 1F).

### Example 71. Synthesis of INSCoV-601J and INSCoV-601J(2)

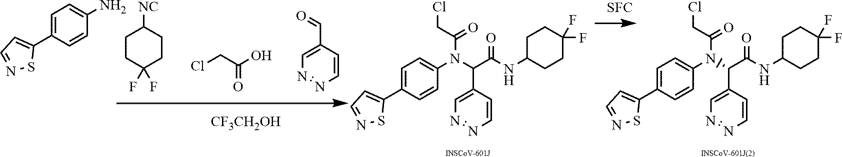

Step 1: INSCoV-601J was synthesized according to the General procedure for INSCoV series. Purification B: The mixture was concentrated under vacuum. The crude was dissolved in METB (10 mL), stirred for a moment and filter cake was concentrated under vacuum. INSCoV-601J (250 mg,427.35 µmol, 46.20% yield, 86.489% purity) was obtained as yellow solid, which was confirmed by HNMR, LCMS, SFC and HPLC.

LCMS: Retention time: 0.844 min, (M+H) = 506.1, HPLC: Retention time: 2.000 min, SFC: Retention time: peak:0.491 min, peak 2: 1.545 min, ¹H NMR (400 MHz, DMSO-d₆): δ = 9.19 -9.02 (m, 2H), 8.60 (d, J= 1.8 Hz, 1H), 8.39(d, J= 7.6 Hz, 1H), 7.80 (d, J= 1.8 Hz,1H), 7.70 (d, J = 8.8 Hz, 2H), 7.57 - 7.37 (m, 3H), 6.07 (s, 1H), 4.17 - 4.09 (m, 1H), 3.85 - 3.74 (m, 1H), 2.07 - 1.70 (m, 8H), 1.57 - 1.40 (m, 2H).

Step 2: INSCoV-601J (100 mg, 197.64 µmol,1 eq) was chiraled for SFC. The solution was concentrated under vacuum. INSCoV-601J(2) (54.54 mg, 104.52 µmol, 52.88% yield, 96.965% purity) was obtained as yellow solid. Which was confirmed by HNMR, FNMR, LCMS, HPLC; SFC showed ee%=83.00%.

LCMS: Retention time: 0.832 min, (M+H) = 506.1, SFC: Retention time: 1.564 min, ee% = 83.00%, HPLC: Retention time: 1.994 min, ¹H NMR (400 MHz, DMSO-d₆): δ = 9.14 - 9.04 (m, 2H), 8.59 (d, J= 1.8 Hz, 1H), 8.38 (br d, J= 7.5 Hz, 1H), 7.80 (d, J= 1.8 Hz, 1H), 7.70 (br d, J= 8.8 Hz, 2H), 7.49 (br d, J= 7.3 Hz, 2H), 7.39 (dd, J= 2.5, 5.3 Hz, 1H), 6.07 (s, 1H), 4.21 - 4.02 (m,2H), 3.82 (br dd, J= 2.5, 3.4 Hz, 1H), 2.09 - 1.68 (m, 8H), 1.55 - 1.37 (m, 2H)._¹⁹F NMR (376 MHz, DMSO-d6): δ = -92.54 - -94.15 (m, 1F), -96.48 - -98.66 (m, 1F).

### Example 72. Synthesis of INSCoV-612

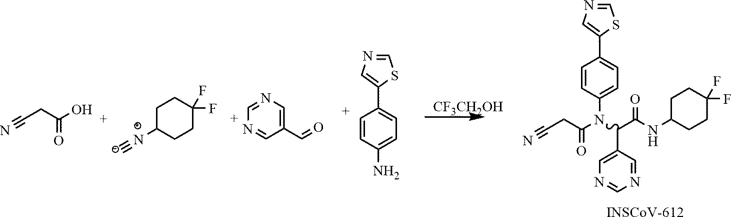

INSCoV-612 was synthesized according to the General procedure for INSCoV series. Purification B: The mixture was added H₂O (75 mL) and extracted with EA (30 mL × 3). The organic phase was washed with brine (50 mL), dried over anhydrous Na₂SO₄, concentrated in vacuum to give a yellow residue. The crude product was triturated with EtOH(10 mL) at 25° C. for 30 min to give INSCoV-612 (170 mg, 342.38 µmol, 37.01% yield, 100% purity) was obtained as a yellow solid.

LCMS: Retention time: 0.752 min, (M+H) = 497.1, HPLC: Retention time: 2.495 min, ¹H NMR (400 MHz, DMSO-d₆): δ = 9.10 (s, 1H), 8.99 (s, 1H), 8.55 - 8.50 (m, 2H), 8.37 - 8.31 (m, 2H), 7.65 (br d, J= 8.6 Hz, 2H), 7.38 (br s, 2H), 6.14 - 6.07 (m, 1H), 3.90 - 3.82 (m, 1H), 3.65 (s, 2H), 2.07 - 2.01 (m, 1H), 1.97 - 1.81 (m, 4H), 1.80 - 1.71(m, 1H), 1.60 - 1.50 (m, 1H), 1.45 - 1.33 (m, 1H), 1.18 (t, J =7.1 Hz, 1H), ¹⁹F NMR: (377 MHz, DMSO-d6): δ = -92.63 - -93.92 (m, 1F), -97.01 - -99.16 (m, 1F).

### PHARMACEUTICAL COMPOSITIONS

### Example A-1: Parenteral Pharmaceutical Composition

To prepare a parenteral pharmaceutical composition suitable for administration by injection (e.g., subcutaneous, intravenous), 1-1000 mg of a water-soluble salt of a compound described herein, or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection (i.e., a subcutaneous, SC, injection).

### Example A-2: Oral Solution

To prepare a pharmaceutical composition for oral delivery, a sufficient amount of a compound described herein, or a pharmaceutically acceptable salt thereof, is added to water (with optional solubilizer(s),optional buffer(s) and taste masking excipients) to provide a 20 mg/mL solution.

### Example A-3: Oral Tablet

A tablet is prepared by mixing 20-50% by weight of a compound described herein, or a pharmaceutically acceptable salt thereof, 20-50% by weight of microcrystalline cellulose, 1-10% by weight of low-substituted hydroxypropyl cellulose, and 1-10% by weight of magnesium stearate or other appropriate excipients. Tablets are prepared by direct compression. The total weight of the compressed tablets is maintained at 100 -500 mg.

### Example A-4: Oral Capsule

To prepare a pharmaceutical composition for oral delivery, 1-1000 mg of a compound described herein, or a pharmaceutically acceptable salt thereof, is mixed with starch or other suitable powder blend. The mixture is incorporated into an oral dosage unit such as a hard gelatin capsule, which is suitable for oral administration.

In another embodiment, 1-1000 mg of a compound described herein, or a pharmaceutically acceptable salt thereof, is placed into Size 4 capsule, or size 1 capsule (hypromellose or hard gelatin) and the capsule is closed.

### BIOLOGICAL EXAMPLES

### Example B-1: In Vitro Assay (SARS-CoV-2 M^{pro} Enzymatic Assay)

The C-His6-tagged SARS-CoV-2 M^{PRO} (NC_045512) was cloned, expressed in E. coli and purified by WuXi. The substrate of Dabcyl-KTSAVLQIISGFRKME-(Edans) was synthesized by Genscript. The assay buffer contained 20 mM of Tris-HCl (pH=7.3), 100 mM of NaCl, 1 mM of EDTA, 5 mM of TCEP and 0.1% BSA. The final concentrations of the M^{pro} protein and substrate were 25 nM and 25 µM, respectively, in the M^{PRO} enzymatic assay. Reference compound GC376 was provided by WuXi AppTec and was included in each plate to ensure assay robustness. Test compounds were tested at single dose or 10 doses titration, in duplicate. Compounds were added to an assay plate (384w format) using ECHO, in duplicate wells. The final concentration is 10 µM for the single dose experiment. As for the full dose response experiment, samples were 3-fold serially diluted starting from 25 uM for 10 doses and added to an assay plate, in duplicate wells. The final concentrations (µM) of each compound was 25, 8.33, 2.778, 0.926, 0.309, 0.103, 0.034, 0.011, 0.0038, and 0.0013. M^{PRO} protein (25 µL, 30 nM) was added to an assay plate containing test compounds using a Multidrop. The test compound and M^{PRO} protein were pre-incubated at RT for 30 min. Then, substrate (5 µL, 150 µM) was added to an assay plate. For 100% inhibition controls (HPE, high percent effect), 1 µM of GC376 was added. For no inhibition controls (ZPE, zero percent effect), the same volume of DMSO was added. The final DMSO concentration was 1%. Each activity testing point had a relevant background control without the enzyme to remove the fluorescence interference of the compound. After 60 min incubation at 30° C., the fluorescence signal (RFU) was detected using a microplate reader M2e (SpectraMax) at Eₓ/Eₘ=340 nm/490 nm.

The inhibition activity was calculated using the formula below, IC₅₀ values were calculated using the Inhibition% data.

$$I n h i b i t i o n \% = \left(\left(C P D - B {G}_{H P E}\right) - \left(Z P E - B {G}_{Z P E}\right)\right) / \left(\left(H P E - B {G}_{H P E}\right) - \left(Z P E - B {G}_{Z P E}\right)\right) \times 100$$

where, HPE is high percent effect controls (1 µM of GC376 + enzyme + substrate); ZPE is zero percent effective controls (enzyme + substrate, no compound); CPD is compound activity testing wells (compound + enzyme + substrate; and BG is background control wells (no enzyme).

IC₅₀ values of compounds were calculated with the GraphPad Prism software using the nonlinear regression model of log(inhibitor) vs. response - variable slope (four parameters).

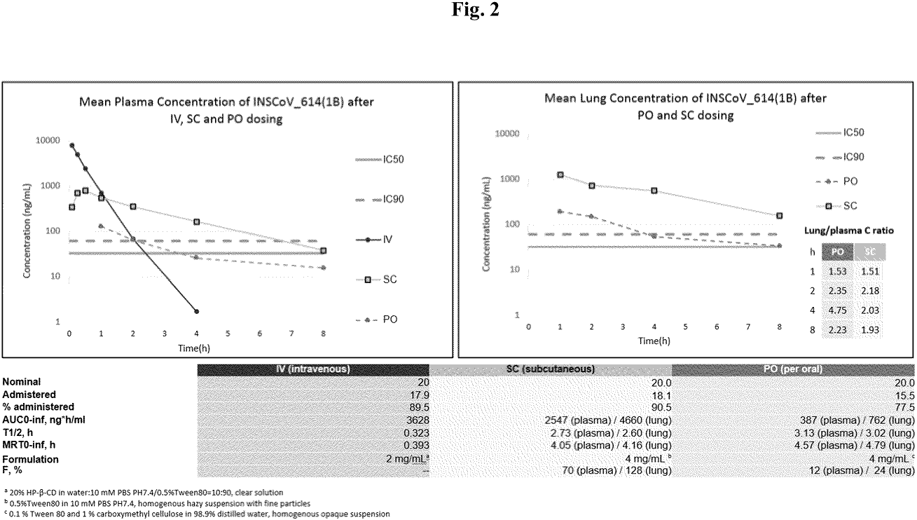

Representative biochemical data is presented in Table 2 and representative biochemical curves are shown in FIG. 2.

TABLE 2

In vitro potency data

| Compound ID | IC₅₀, µM | Compound ID | IC₅₀, µM |
|---|---|---|---|
| INSCoV-110A | 1.14 | INSCoV-574 | 1.27 |
| INSCoV-110A(1) | 1.98 | INSCoV-574A | 4.97 |
| INSCoV-110A(2) | 2.15 | INSCoV-575 | >25 |
| INSCoV-110B | >50 | INSCoV-576 | 4.67 |
| INSCoV-110-1 | 35.96 | INCoV-600A | 0.54 |
| INSCoV-110-2 | >50 | INCoV-600A(1) | 0.84 |
| INSCoV-110D | >50 | INCoV-600A(2) | 0.43 |
| INSCoV-501 | 0.68 | INCoV-600A(3) | 0.41 |
| INSCoV-501A | 0.29 | INCoV-600B | 0.29 |
| INSCoV-501B | 0.60 | INCoV-600B(1) | 0.25 |
| INSCoV-501C | 9.82 | INCoV-600B(1A) | 0.34 |
| INSCoV-501D | 2.51 | INCoV-600B(1B) | 0.31 |
| INSCoV-501E | 0.60 | INCoV-600B(2) | 0.19 |
| INSCoV-501G | 0.26 | INCoV-600B(2A) | 0.17 |
| INSCoV-501G(1) | >25 | INCoV-600B(2B) | 0.31 |
| INSCoV-501H | 0.72 | INCoV-600C | 0.22 |
| INSCoV-501H(1) | 0.63 | INCoV-600C(1) | 0.27 |
| INSCoV-501I | 0.39 | INCoV-600C(1A) | 0.34 |
| INSCoV-501K(2) | 1.07 | INCoV-600C(1B) | 0.31 |
| INSCoV-501L | 0.40 | INCoV-600C(2) | 0.20 |
| INSCoV-501M | 0.45 | INCoV-600C(2A) | 0.45 |
| INSCoV-501O | 1.50 | INCoV-600C(2B) | 0.16 |
| INSCoV-501P | 0.56 | INCoV-600D | 0.52 |
| INSCoV-501R | 0.48 | INCoV-600E | 0.57 |
| INSCoV-501R(1) | 0.57 | INCoV-600F | 0.53 |
| INSCoV-501S | 0.24 | INCoV-600G | 0.99 |
| INSCoV-502 | 4.82 | INCoV-600H | 0.66 |
| INSCoV-503 | 0.98 | INCoV-600I | 1.18 |
| INSCoV-503A | 2.63 | INCoV-600J | 0.11 |
| INSCoV-503B | 1.00 | INCoV-600J(1) | 0.063 |
| INSCoV-503C | 1.38 | INCoV-600J(2) | 0.53 |
| INSCoV-503D | 2.65 | INCoV-600K | 0.078 |
| INSCoV-503E | 0.66 | INCoV-600K(l) | 0.050 |
| INSCoV-503F | 0.94 | INCoV-600K(2) | 0.43 |
| INSCoV-503G | 2.30 | INSCoV-600L | 0.45 |
| INSCoV-504 | 16.33 | INSCoV-600M | 0.50 |
| INSCoV-505 | 8.65 | INSCoV-600N | 1.06 |
| INSCoV-507 | 2.13 | INSCoV-6000 | 1.09 |
| INSCoV-508 | >25 | INSCoV-600Q | 6.98 |
| INSCoV-514 | >25 | INSCoV-600Q(l) | 22.56 |
| INSCoV-515 | >25 | INSCoV-600Q(2) | 2.22 |
| INSCoV-516 | 1.18 | INSCoV-600R | 12.84 |
| INSCoV-517 | 0.24 | INSCoV-600R(l) | 19.91 |
| INSCoV-517(l) | 0.08 | INSCoV-600R(lA) | >25 |
| INSCoV-5 17(2) | 1.94 | INSCoV-600R(lB) | 16.66 |
| INSCoV-517A | 0.09 | INSCoV-600R(2) | 6.45 |
| INSCoV-5 17A(lA) | 0.03 | INSCoV-600R(2A) | 2.93 |
| INSCoV-5 17a(1B) | 3.25 | INSCoV-600R(2B) | >25 |
| INSCoV-517B | 0.17 | INSCoV-600X | 1.39 |
| INSCoV-517C | 0.11 | INSCoV-600Y | 1.99 |
| INSCoV-517C(l) | 11.23 | INSCoV-601CA | 1.15 |
| INSCoV-5 17C(2) | 11.36 | INSCoV-601F | 0.13 |
| INSCoV-517C(3) | 0.07 | INSCoV-601G | 0.08 |
| INSCoV-517C(4) | >25 | INSCoV-601G(l) | 0.06 |
| INSCoV-520 | >25 | INSCoV-601G(2) | 0.27 |
| INSCoV-523 | 0.98 | INSCoV-601H | 0.10 |
| INSCoV-531 | 12.72 | INSCoV-601I | 0.062 |
| INSCoV-534 | 1.28 | INSCoV-601I(l) | 0.050 |
| INSCoV-535 | 1.04 | INSCoV-601I(2) | 0.21 |
| INSCoV-536 | 0.62 | INSCoV-601J | 0.12 |
| INSCoV-537 | 0.42 | INSCoV-601J(2) | 0.53 |
| INSCoV-537I | 0.27 | INSCoV-601K | 0.10 |
| INSCoV-537K | 0.36 | INSCoV-601K(l) | 0.06 |
| INSCoV-537L | >25 | INSCoV-601K(2) | 0.19 |
| INSCoV-538 | 0.28 | INSCoV-601L | 0.30 |
| INSCoV-538A | 0.90 | INSCoV-601M | 1.15 |
| INSCoV-538A(l) | 0.23 | INSCoV-601N | 0.12 |
| INSCoV-538A(2) | 9.60 | INSCoV-601N(l) | 0.13 |
| INSCoV-539 | 0.80 | INSCoV-601N(2) | 3.49 |
| INSCoV-539A | >25 | INSCoV-6010 | 3.06 |
| INSCoV-549 | 2.49 | INSCoV-601P | 0.38 |
| INSCoV-557A | >25 | INSCoV-601P(lA) | 0.14 |
| INSCoV-558 | 0.33 | INSCoV-601P(lB) | 13.66 |
| INSCoV-558A | 0.47 | INSCoV-601Q | 0.15 |
| INSCoV-558H | 0.40 | INSCoV-601Q(IA) | 0.039 |
| INSCoV-559 | 1.08 | INSCoV-601Q(lB) | 0.071 |
| INSCoV-560A | 4.34 | INSCoV-601R | 0.12 |
| INSCoV-570 | >25 | INSCoV-601R(lA) | 0.14 |
| INSCoV-571 | 6.28 | INSCoV-601R(lB) | 0.44 |
| INSCoV-537(2) | 1.44 | INSCoV-601S | 0.09 |
| INSCoV-601S(lA) | 0.06 | INSCoV-601S(lB) | 0.98 |
| INSCoV-601T | 0.17 | INSCoV-612 | 23.20 |
| INSCoV-614 | 0.33 | INSCoV-614(lA) | >25 |
| INSCoV-614(lB) | 0.06 | INSCoV-614(2A) | 6.94 |
| INSCoV-614(2B) | 1.70 | INSCoV-614A(lA) | 6.74 |
| INSCoV-614A(lB) | 5.05 | INSCoV-614A(2A) | 0.18 |
| INSCoV-614A(2B) | 9.11 | INSCoV-615 | 11.41 |
| INSCoV-616 | 8.36 | INSCoV-618 | >25 |
| INSCoV-620 | >25 | INSCoV-704 | 19.39 |

### Example B-2: In Vitro Antiviral Cell-Based Assay (Live SARS-CoV-2 IFA)

SARS-CoV-2 was provided by Korea Centers for Disease Control and Prevention (KCDC). Vero cells were acquired from the ATCC and maintained in the Dulbecco’s Modified Eagle’s Medium (DMEM) supplemented with 10% FBS, and 1% Antibiotic-Antimycotic solution. DMEM supplemented with 2% FBS and 1% Antibiotic-Antimycotic solution are used as the assay medium. The main reagents used in this assay are Anti-SARS-CoV-2 N protein antibody, Alexa Fluor 488 goat anti-rabbit IgG (H + L) secondary antibody and Hoechst 33342. Ten-point dose-response curves (DRC) are generated for each compound. Vero cells are seeded at 1.2 × 10⁴ cells per well in black 384-well, µclear plates (Greiner Bio-One), 24 h prior to the experiment. For viral infection, SARS-CoV-2 is added at a multiplicity of infection (MOI) of ~0.0125. The cells are fixed at 24 hpi with 4% paraformaldehyde and analyzed by immunofluorescence. The acquired images are analyzed using software to quantify cell numbers and infection ratios, and antiviral activity is normalized to positive (mock) and negative (0.5% DMSO) controls in each assay plate. DRCs are fitted by sigmoidal dose-response models using XLfit 4 Software or Prism with the following equation:

Y = Bottom + ( Top - Bottom) / ( 1 + (IC₅₀/X) Hillslope)

EC₅₀ and CC₅₀ values were calculated and are represented in Table 3.

TABLE 3

Live SARS-CoV-2 in vitro cell based assay results.

| Compound ID | EC₅₀, µM, live SARS-CoV-2 in Vero cells, IFA | CC₅₀, µM, Vero cells |
|---|---|---|
| INSCoV-517 | 3.22 | >50 |
| INSCoV-517A | 11.08 | >25 |
| INSCoV-600J(l) | 14.24 | 28.87 |
| INSCoV-600K | 14.45 | 25.25 |
| INSCoV-600K(l) | 11.43 | 27.61 |
| INSCoV-601G | 10.15 | 27.77 |
| INSCoV-601I | 11.05 | 25.36 |
| INSCoV-601I(l) | 14.08 | 24.90 |
| INSCoV-614(lB) | 2.23 | >25 |
| INSCoV-614A(2A) | 4.25 | >25 |
| Remdesivir | 7.86 | >50 |
| Chloroquine | 7.35 | >50 |
| Lopinavir | 13.91 | >50 |

EC₅₀ and CC₅₀ were calculated with Remdesivir as a positive control. The structure of Remdesivir is listed below.

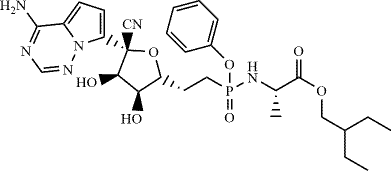

### Example B-3: ADME Characterization

### Microsomal Stability Assessment

The microsomal stability of compounds from Table 4 was assessed as follows: working solutions of tested compounds and control compounds (testosterone, diclofenac, and propafenone) were prepared. The appropriate amount of NADPH powder (β-nicotinamide adenine dinucleotide phosphate reduced form, tetrasodium salt, NADPH·4Na, catalog no. 00616; Chem-Impex International) was weighed and diluted into MgC l₂ (10 mM) solution (working solution concentration, 10 units/ml; final concentration in reaction system, 1 units/ml). The appropriate concentration of microsome working solutions (human: HLM, catalog no. 452117, Corning; CD-1 mouse: MLM, catalog no. M1000, Xenotech) was prepared with 100 mM PB. Cold ACN, including 100 ng/ml tolbutamide and 100 ng/ml labetalol as internal standard (IS), was used for the stop solution. Compound or control working solution (10 µl per well) was added to all plates (T0, T5, T10, T20, T30, T60, and NCF60), except the matrix blank. Dispensed microsome solution (80 µl per well) was added to every plate by Apricot and the mixture of microsome solution and compound was incubated at 37° C. for approximately 10 min. After prewarming, a dispensed NADPH regenerating system (10 µl per well) was added to every plate by Apricot to start a reaction. The solution was then incubated at 37° C. Stop solution (300 µl per well, 4° C.) was then added to terminate the reaction. The sampling plates were shaken for approximately 10 minutes. The samples were centrifuged at 4,000 rpm. for 20 min at 4° C. While centrifuging, new 8×96-well plates were loaded with 300 µl HPLC water, and then 100 µl supernatant was transferred and mixed for liquid chromatography-tandem mass spectrometry (LC/MS/MS).

### CYP Inhibition Assay

The following reagents/reactants were used in the assay: water was purified by ELGA Lab purification systems, buffer solution: PB (100 mM), MgCl₂ (33 mM), organic solvents were AR or HPLC grade, CYP substrates: phenacetin (10 µM, 25 µL, 1A2), diclofenac (5 µM, 25 µL, 2C9), S-mephenytoin (30 µM, 75 µL, 2C19), dextromethorphan (5 µM, 12,5, 2D6), and midazolam (2 µM, 10 µL, 3A4), stock solutions were prepared in MeOH (20, 10, 20, 20, and 10 mM, respectively). Positive controls: α-naphthoflavone, sulfaphenazole, (+)-N-3-benzylnirvanol, quinidine, and ketoconazole, final concentration was 3 µM in MeOH (90 µL), stock solutions were prepared in DMSO (3 mM for all the inhibitors). Test compounds were evaluated using five conc. points at the range of 15-5,000 µM. Human liver microsomes (Cat No. 452117, Corning) were used at the concentration of 0.253 mg/mL (PB: 44,431 mL; vol. of microsomes: 569 mL), while NADPH (Cat No. 00616, Chem-impex international) was used at the concentration of 10 mM (MgCh: 20,0 mL, 33 mM). Main procedure: working solution (100_{×}) of the test compound and standard inhibitors were prepared, microsomes were pulled out of the -80° C. freezer to thaw on ice, labeled the date and put it back to the freezer immediately after use. Substrates (20 µL) and PB (20 µL) were added to corresponding wells and Blank wells, respectively. Test compound (2 µL) and positive control working solution were added to corresponding wells, then solvent (2 µL) was added to No Inhibitor wells and Blank wells. HLM working solution (158 µL) was added to all wells of the incubation plate. Plate was pre-warmed for about 10 min at 37° C. in a water bath, then NADPH (20 µL) was added to all incubation wells. CYPs were mixed and incubated for 10 min at 37° C. in a water bath. The reaction was terminated at the time point by adding a 400 µL cold stop solution (200 ng/mL tolbutamide and labetalol in ACN). Samples were centrifuged at 4000 rpm for 20 minutes to precipitate protein. Supernatant (200 µL) was transferred to 100 µL HPLC water and shaken for 10 min, then samples were subjected for LC/MS/MS analysis. XL fit was used to plot the percent of vehicle control vs the test compound concentrations, and for non-linear regression analysis of the data. IC₅₀ values were determined using 3- or 4-parameter logistic equations. IC₅₀ values were reported as “>50 µM” when % inhibition at the highest concentration (50 µM) is less than 50%.

Representative data for CYP P450 are described in Table 4.

TABLE 4

Metabolic (microsomal) stability (MLM and HLM) and small CYP P450 panel assessment..

| Compound ID | MMS, HLM, CLᵢₙₜ(mic), (µL/min/mg) | MMS, MLM, CLᵢₙₜ(mic), (pL/min/mg) | CYP P450 isoform, µM | | | | |
|---|---|---|---|---|---|---|---|
| | | | 1A2 | 2C9 | 2C19 | 2D6 | 3A4 |
| INSCoV-501A | 211.20 | 311.3 | >50 | 7.59 | 5.79 | 6.75 | 0.59 |
| INSCoV-5011 | 37.0 | 46.7 | >50 | 35.1 | 16.7 | 6.45 | 1.13 |
| INSCoV-517A | 23.6 | 22.4 | >50 | >50 | >50 | >50 | >50 |
| INSCoV-600J | 17.0 | 27.8 | >50 | >50 | 49.9 | 9.43 | 43.20 |
| INSCoV-600J(l) | 20.7 | 19.5 | >50 | >50 | >50 | 30.8 | 38 |
| INSCoV-600K | 45.5 | 58.9 | 25.1 | 17.7 | 15.8 | 2.13 | 1.17 |
| INSCoV-600K(l) | 35.1 | 45.1 | >50 | >50 | 18.3 | 1.89 | 1.62 |
| INSCoV-601G | 46.2 | 437.6 | >50 | 20.8 | 4.56 | 3.52 | 0.551 |
| INSCoV-601H | 24.9 | 46.2 | >50 | >50 | >50 | 27.5 | 32.9 |
| INSCoV-601I | 123.7 | 629.9 | >50 | 11.3 | 9.51 | 5.54 | 0.257 |
| INSCoV-601I(l) | 128.8 | 617.5 | >50 | 16.4 | 16.4 | 4.67 | 0.725 |
| INSCoV-601K | 63.3 | 56.6 | 28.2 | 11.6 | 22.8 | 5.2 | 0.466 |
| INSCoV-601N | 169.4 | 531.3 | 24.9 | 3.25 | 5.65 | 0.706 | 0.218 |
| INSCoV-601Q(lA) | 178,6 | 511,6 | 38.6 | 20.7 | 17.3 | 16.2 | 1.57 |
| INSCoV-601P(lA) | 66 | 102.2 | >50 | >50 | >50 | 6.12 | 3.62 |
| INSCoV-614(lB) | 29.6 | 38.9 | 24.3 | 15.6 | 9.50 | 6.50 | 1.45 |
| INSCoV-614A(2A) | <9.6 | <9.6 | >50 | >50 | >50 | >50 | >50 |

### Example B-4: Permeability Studies

### Caco-2 Permeability

Caco-2 cells purchased from ATCC were seeded onto polyethylene membranes (PET) in 96-well Corning Insert plates at 1 x 105 cells/cm². Medium was refreshed every 4 \~5 days until the 21^{st} to 28^{th} day for confluent cell monolayer formation. The transport buffer was HBSS with 10 mM HEPES at pH=7.40±0.05. Test compound (2.00 µM) and digoxin (10.0 µM) were tested at bi-directionally in duplicate, while nadolol and metoprolol were tested at 2.00 µM in A to B direction in duplicate as well. Final DMSO concentration was adjusted to less than 1%. The plate was incubated for 2 h in the CO₂ incubator at 37±1° C., with 5% CO₂ at saturated humidity without shaking. All the samples were then mixed with acetonitrile containing internal standard and centrifuged at 3200 xg for 10 min. For nadolol and metoprolol, 100 µL supernatant solution was diluted with 300 µL distilled water for LC-MS/MS analysis. For digoxin and test compounds, 100 µL supernatant solution was diluted with 100 µL distilled water for LC-MS/MS analysis. Concentrations of test and control compounds in starting solution, donor solution, and receiver solution were quantified by LC-MS/MS methodologies, using peak area ratio of analyte/internal standard. After transport assay, lucifer yellow rejection assay was applied to determine the Caco-2 cell monolayer integrity. The apparent permeability coefficient Papp (cm/s) was calculated using the equation:

$${P}^{a p p} = \left(d C r / d t\right) \times V r / \left(A \times {C}_{0}\right)$$

where d/Cr/6t is the cumulative concentration of compound in the receiver chamber as a function of time (µM/s); Vᵣ is the solution volume in the receiver chamber (0.075 mL on the apical side, 0.25 mL on the basolateral side); A is the surface area for the transport, i.e. 0.0804 cm² for the area of the monolayer; C₀ is the initial concentration in the donor chamber (µM).

The efflux ratio was calculated using the equation:

$$E f f l u x R a t i o = {P}^{a p p} \left(B A\right) / {P}^{a p p} \left(A B\right)$$

where V_{d} is the volume in the donor chambers (0.075 mL on the apical side, 0.25 mL on the basolateral side); C_{d} and Cᵣ are the final concentrations of the transport compound in donor and receiver chambers, respectively.

Data analysis was performed by analogy to procedure described for CYP inhibition assay and is displayed in Table 5.

### PAMPA Permeability Assay

2.6 g KH₂PO₄ and 18.5 g K₃PHO₄×3H₂O were dissolved in 1000 mL of ultra-pure water, mixed thoroughly. The pH was adjusted to 7.40 ± 0.05, using either 1 M sodium hydroxide or 1 M hydrochloric acid. 0.2 mM working solution was prepared by diluting 10 mM stock solution with DMSO. 10 µM donor solution (5% DMSO) was prepared by diluting 20 µL of working solution with 380 µL PBS. 150 µL of 10 µM donor solutions to each well of the donor plate, whose PVDF membrane was precoated with 5 µL of 1% lecithin/dodecane mixture. Duplicates were prepared. 300 µL of PBS was added to each well of the PTFE acceptor plate. The donor plate and acceptor plate were combined together and incubated for 4 h at room temperature with shaking at 300 rpm. Preparation of T0 sample: 20 µL donor solution was transferred to the new well followed by the addition of 250 µL PBS (DF: 13.5), 130 µL ACN (containing internal standard) as T₀ sample. Preparation of acceptor sample: the plate was removed from the incubator. 270 µL solution was transferred from each acceptor well and mixed with 130 µL ACN (containing internal standard) as acceptor sample. Preparation of donor sample: 20 µL solution was transferred from each donor well and mixed with 250 µL PBS (DF: 13.5), 130 µL ACN (containing internal standard) as donor sample. Acceptor samples and donor samples were all analyzed by LC-MS/MS. The equation used to determine permeability rates (Pe) was displayed as follows:

$${P}_{e} = C \times \left(- ln \left(1 - \frac{{\left[d r u g\right]}_{a c c e p t o r}}{{\left[d r u g\right]}_{e q u i l i b r i u m}}\right)\right) \times {10}^{- 7} , w h e r e C = \left(\frac{{V}_{D} \times {V}_{A}}{\left({V}_{D} + {V}_{A}\right) \times A r e a \times t i m e}\right)$$

$${\left[d r u g\right]}_{e q u i l i b r i u m} = \left({\left[d r u g\right]}_{d o n o r} \times {V}_{D} + {\left[d r u g\right]}_{a c c e p t o r} \times {V}_{A}\right) / \left({V}_{D} + {V}_{A}\right) ,$$

$${V}_{D} = 0.15 m L ; {V}_{A} = 0.30 m L ; A r e a = 0.28 c {m}^{2} ; t i m e = 14400 s ,$$

$${\left[d r u g\right]}_{a c c e p t o r} = {\left({A}_{a} / {A}_{i} \times D F\right)}_{a c c e p t o r} ; {\left[d r u g\right]}_{d o n o r} = {\left({A}_{a} / {A}_{i} \times D F\right)}_{d o n o r} ,$$

where: Aₐ/A: Peak area ratio of analyte and internal standard; DF: Dilution factor.

Representative permeability data is displayed in Table 5.

TABLE 5

Permeability and solubility assessment data.

| Compound ID | Caco-2, Mean Pₐₚₚ, (10-⁶ cm/s), A to B | Caco-2, Efflux ratio | PAMPA, Mean Pe (nm/s) | Kinetic solubility in water, µM |
|---|---|---|---|---|
| INSCoV-501A | 0.15 | 26.90 | 9.42 | 2.88 |
| INSCoV-5011 | <0.0373 | >193 | 0.74 | <1.56 |
| INSCoV-517A | 0.305 | 36.2 | 12.8 | 5.31 |
| INSCoV-600J | 0.00210 | 1383 | 0.81 | 34.5 |
| INSCoV-600J(l) | <0.00186 | >527 | 0.873 | 169 |
| INSCoV-600K | <0.00502 | >452 | 1.26 | 7.25 |
| INSCoV-600K(l) | <0.00416 | >172 | 1.37 | 161 |
| INSCoV-601G | 0.04 | 17.1 | 9.05 | <1.56 |
| INSCoV-601H | 0.0837 | 17.9 | 8.03 | <1.56 |
| INSCoV-601I | 0.0424 | 6.62 | 29.1 | <1.56 |
| INSCoV-60 1I(l) | <0.00866 | >13.8 | 30.5 | <1.56 |
| INSCoV-601K | <0.0743 | >12.5 | 5.97 | <1.56 |
| INSCoV-601N | <0.0434 | NA | 155 | <1.56 |
| INSCoV-601Q(lA) | 0.0610 | 56.3 | 0.708 | 114 |
| INSCoV-601P(lA) | <0.480 | >0.489 | 6.26 | 142 |
| INSCoV-614(lB) | 0.464 | 48 | 1.88 | 164 |
| INSCoV-614A(2A) | 0.418 | 54.5 | 1.23 | 158 |

### Example B-5: Plasma Stability

Propantheline bromide was used as a reference compound in this assay. The pooled frozen plasma was thawed in a water bath at 37° C. prior to experiment. Plasma was centrifuged at 4000 rpm for 5 min and the clots were removed if any. The pH will be adjusted to 7.4 ± 0.1 if required.

Preparation of compounds: 1 mM intermediate solution was prepared by diluting 10 µL of the stock solution with 90 µL DMSO; 1 mM intermediate of positive control Propantheline was prepared by diluting 10 µL of the stock solution with 90 µL ultrapure water. For test compounds, 100 µM dosing solution was prepared by diluting 10 µL of the intermediate solution (1 mM) with 90 µL DMSO. For positive controls, 100 µM dosing solution was prepared by diluting 10 µL of the intermediate solution (1 mM) with 90 µL 45% MeOH/H₂O. 98 µL of blank plasma was spiked with 2 µL of dosing solution (100 µM) to achieve 2 µM of the final concentration in duplicate and samples were Incubated at 37° C. in a water bath. At each time point (0,10, 30, 60 and 120 min), 400 µL of stop solution (200 ng/mL tolbutamide and 200 ng/mL Labetalol in 100% ACN) was added to precipitate protein and mixed thoroughly. Centrifuged sample plates at 4,000 rpm for 10 min. An aliquot of supernatant (50 µL) was transferred from each well and mixed with 100 µL ultra-pure water. The samples were shaken at 800 rpm for about 10 min before submitting to LC-MS/MS analysis. The % remaining of test compound after incubation in plasma was calculated using following equation:

$$\% R e m a i n i n g = 100 \times \left(P A R a t a p p o i n t e d i n c u b a t i o n t i m e / P A R a t {T}_{0} t i m e\right)$$

where PAR is the peak area ratio of analyte versus internal standard (IS).

Representative plasma stability data is shown in Table 6.

TABLE 6

Plasma proteins binging (PPB) and stability in plasma assessment.

| Compound ID | PPB, mice, % (Unbound/ Bound/ Recovery) | PPB, human, % (Unbound/ Bound/ Recovery) | Plasma stability, mice, T_{½}, min | Plasma stability, human, T_{½}, min |
|---|---|---|---|---|
| INSCoV-600J(l) | 49.27 / 50.73 / 10.4 | 51.60/48.40/36.9 | 31.3 | 46.3 |
| INSCoV-600K(l) | 66.70 | 61.34 | 27.6 | 48.9 |
| INSCoV-601G | NA | NA | 3.8 | 46.3 |
| INSCoV-601H | NA | NA | 2.7 | 2.3 |
| INSCoV-601I | NA | NA | 3.2 | 4.8 |
| INSCoV-60 1I(l) | NA | NA | 3.3 | 4.2 |
| INSCoV-601K | 40.00/ 60.00/ 22.6 | 61.26/38.74/15.6 | 23.1 | 30.2 |
| INSCoV-601N | NA/NA/0.1 | NA/NA/0.2 | 21.1 | 32.6 |
| INSCoV-601P(lA) | NA/NA/2.8 | NA/NA/1.3 | 13.1 | 16.3 |
| INSCoV-601Q(lA) | NA/NA/3.1 | NA/NA/3.5 | 6.9 | 11.6 |
| INSCoV-614(lB) | 16.22/ 83.78/ 84.7 | 29.41/ 70.59/ 79.1 | >289.1 | >289.1 |
| INSCoV-614A(2A) | 26/74/88.8 | 29.78/70.22/83.4 | >289.1 | >289.1 |

### Example B-6: Pharmacokinetic Profile in Mice

Male CD-1 mice (n = 3 per group, age: 7-9 weeks) were treated with a solution of compound 5{55}-SR dissolved in 0.5% Tween80 in 10 mM PBS pH7.4 (homogenous hazy suspension, 5 mg/mL) at the administered dose of 20 mg/kg (subcutaneous). Blood samples (about 0.025 mL per time point) were collected at 0.083, 0.25, 0.5, 1, 2, 4, 8, and 24 h after administration. All blood samples were transferred into pre-chilled commercial EDTA-K2 tubes and placed on wet-ice until centrifugation. Serum samples were obtained following the standard procedures and the concentrations of the compound in the supernatant were analyzed by LC-MS/MS. Lung tissue homogenate was prepared by homogenizing tissue with 4 volumes (w:v) of homogenizing solution (15 mM PBS:MeOH = 2:1 ).

### Protein Precipitation (PPT) Using 96 Well Plate (Plasma)

An aliquot of 6 µL unknown sample, calibration standard, quality control, dilute quality control, single blank and double blank samples were added to the 96-well plate. Each sample (except the double blank) was quenched with 180 µL IS1 respectively (double blank sample was quenched with 180 µL ACN), and then the mixture was vortex-mixed for 10 min at 800 rpm and centrifuged for 15 min at 3220 g (4000 rpm), 4° C. 60 µL supernatant was transferred to another clean 96-well plate and centrifuged for 5 min at 3220 g, 4° C., then the supernatant was directly injected for LC-MS/MS analysis.

### Protein Precipitation (PPT) Using 1.5 mL Tube (Lung Homogenate)

An aliquot of 30 µL unknown sample, calibration standard, single blank and double blank samples were added to the 1.5 mL tube. Each sample (except the double blank) was quenched with 900 µL IS1 respectively (double blank sample was quenched with 900 µL ACN), and then the mixture was vortex-mixed well (at least 15 s) with vortexer and centrifuged for 15 min at 12000 g, 4° C. 60 µL supernatant was transferred to the 96-well plate and centrifuged for 5 min at 3220 g, 4° C., then the supernatants were directly injected for LC-MS/MS analysis

Table 7 displays an ADME profile for generic compounds INSCoV-600K(I) and INSCoV-614(1B).

TABLE 7

General summary for compounds INSCoV-600K(I) and INSCoV-614(1B)

| | INSCoV-600K(1) | INSCoV-614(1B) |
|---|---|---|
| SARS-CoV-2 M^{pro} IC_{50′} nM | 50 | 91 |
| PAMPA, Mean Pe (nm/s)^{a} | 1.37 | 1.88 |
| CYP 1A2 IC_{50′} µM | 25.1 | 24.3 |
| CYP 2C9 IC_{50′} µM | 17.7 | 15.6 |
| CYP 2C19 IC_{50′} µM | 15.8 | 9.50 |
| CYP 2D6 IC_{50′} µM | 2.13 | 6.50 |
| CYP 3A4 IC_{50′} µM | 1.17 | 1.45 |
| HLM, CLᵢₙₜ(mic), (µL/min/mg)^{b} | 35.1 | 29.6 |
| MLM, CLᵢₙₜ(mic), (µL/min/mg)^{c} | 45.1 | 38.9 |
| Plasma stability, T_{½}, min, mice | 27.6 | >289.1 |
| Plasma stability, T_{½}, min, human | 48.9 | >289.1 |

^{a} Permeability in PAMPA artificial membrane assay,
^{b} Intrinsic clearance measured in Human Liver Microsomes
^{c} Intrinsic clearance measured in Mice Liver Microsomes

Compound INSCoV-600K(l) was found to be instable in plasma and was found to be unacceptable for further PK studies. The insertion of a fluorine atom and the α-chloroacetamide warhead resulted in the significantly improved stability in plasma for the generic compound INSCoV-614(1B). The compound INSCoV-614(1B) was further assessed for PK properties in mice.

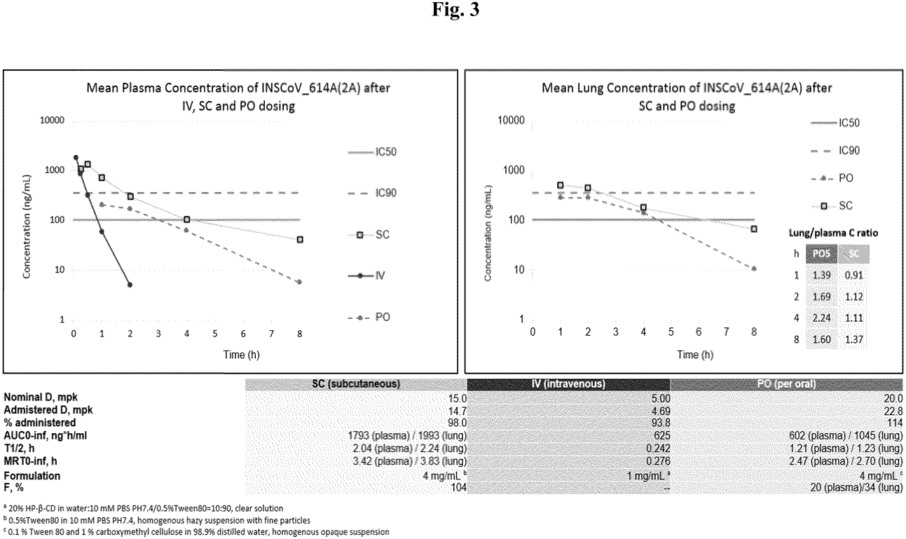

FIGS. 2 and 3 provide a PK summary for INSCoV-614(1B) and INSCoV-614A(2A), respectively.

### Example B-7: Crystal Structure of INSCoV-601I(1) Covalently Bound to Cys145 Residue of SARS-CoV-2 M^{pro}

It was observed that INSCoV-601I(l) has a unique binding mode that has not been reported previously. Thus, similar to the published covalent M^{pro} inhibitors supramolecular interactions are the following: α-atom of the chloroacetamide warhead is covalently attached to the conservative Cys145, isothiazole is located within the deep hydrophobic sub-pocket formed by His41, Cys44, Met49, Met165, and one pyrazine acceptor interacts with His163 via hydrogen bond, phenyl fragment and isothiazole moiety of the ligand interact with His41 via planar and shifted π-stacking, respectively. The novel binding was realized by a) triple H-bonding interface of the warhead carbonyl oxygen with Gly143 and Cys145 NH protons of the polypeptide backbone, b) amide proton of the ligand forms H-bond with the side-chain carbonyl atom of Asn142 (this interaction has been reported previously for several fragments outputted from the extensive fragment-based screening campaign), c) the second pyrazine acceptor and amide carbonyl atom forms H-bonds with water molecules, and d) 4-difluorocyclohexane is placed at the exit of the pocket. It should be especially noted that amide carbonyl atom interacts with Asn142 whereas in many reported inhibitors amide bond in this spatial surrounding forms H-bond with Glu166.

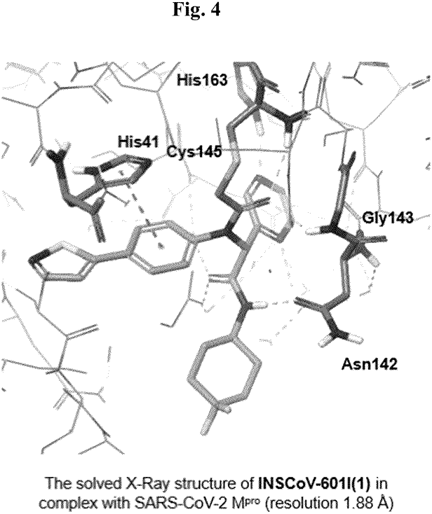

The crystal structure of INSCoV-601I(l) covalently bound to Cys145 residue of SARS-CoV-2 M^{Pro} is shown in FIG. 4. This demonstrates that the compounds disclosed herein covalently modify Cys145.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A compound having the structure of Formula (X), or a pharmaceutically acceptable salt or solvate thereof: 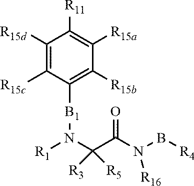
wherein,
B₁ and B are each independently a bond, C₁-C₄ alkylene, C₁-C₄ heteroalkylene, or C₃-C₆ cyclene linker, wherein the alkylene, heteroalkylene or cyclene is optionally substituted;
Ri is halo acetyl, glyoxyl, heterocyclo acyl, cyanide acetyl, vinylsulfonyl, vinylsulfinyl, or acrylo acyl;
R₃ is an optionally substituted heteroaryl;
R₄ is an C₁-C₆ alkyl, aryl, heteroaryl, cycloalkyl or heterocycloalkyl, each of which is optionally substituted;
R₅ is H, C₁-C₆ alkyl, or C₁-C₃ haloalkyl
R₁₁ is amino, halogen, —CN, —OH, C₁-C₆ alkyl, C₁-C₆ haloalkyl, C₁-C₆ alkoxy, cycloalkyl, heterocycloalkyl, aryl, or heteroaryl, wherein each of the alkyl, alkoxy, aryl, cycloalkyl, heterocycloalkyl, or heteroaryl is optionally substituted;
R₁₅ₐ, R_{15b}, R_{15c}, and R_{15d} are each independently H, amino, halogen, —CN, —OH, —OCF₃, C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl is optionally substituted with one, two, or three R²⁰;
wherein optionally, R₁₅ₐ and R₁₁, taken in combination with the carbon atom to which they attach, form a 5-6 membered substituted or unsubstituted ring; or
wherein optionally, R₁₅ₐ and R_{15b}, taken in combination with the carbon atom to which they attach, form a 5-6 membered substituted or unsubstituted ring;
R₁₆ is H, C₁-C₆ alkyl, or C₁-C₃ haloalkyl; and
R₂₀ is oxo, halogen, —CN, —NH₂, —NH(C₁—₆ alkyl), —N(C₁—₆ alkyl)₂, —OH, —CO₂H, —CO₂—C₁—₆ alkyl, —C(═O)NH₂, —C(═O)NH(C₁—₆ alkyl), —C(═O)N(C₁—₆ alkyl)₂, —S(═O)₂NH₂, —S(═O)₂NH(C₁—₆ alkyl), —S(═O)₂N(C₁—₆ alkyl)₂, C₁-C₆ alkyl, C₁-C₆ haloalkyl, C₃₋₈ cycloalkyl, C₁-C₆ heteroalkyl, C₁-C₆ alkoxy, C₁-₆ fluoroalkoxy, C₂-₇ heterocycloalkyl, aryl, heteroaryl, aryloxy, alkylthio, arylthio, alkylsulfoxide, arylsulfoxide, cycloalkylsulfone, alkylsulfone, and arylsulfone.

2. The compound of claim 1, or a pharmaceutically acceptable salt or solvate thereof, wherein the compound has a structure of Formula (XA) or Formula (XB):

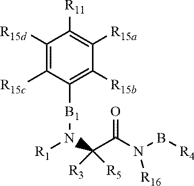

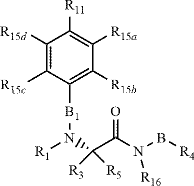

3. (canceled)

4. The compound of claim 1, or a pharmaceutically acceptable salt or solvate thereof, wherein the compound has a structure of Formula (XI): 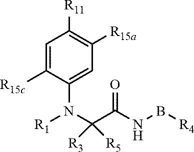
wherein,
B is a bond, C₁-C₄ alkylene, or C₃-C₆ cyclene linker;
R₁ is halo acetyl, glyoxyl, heterocyclo acyl or acrylo acyl;
R₃ is a heteroaryl optionally substituted with one, two, or three R₁₈;
R₄ is an aryl, heteroaryl, cycloalkyl or heterocycloalkyl, each of which is optionally substituted with one, two, three, or four R¹⁹;
R₅ is H, C₁-C₆ alkyl, or C₁-C₃ haloalkyl;
R₁₁ is amino, halogen, —CN, —OH, —OCF₃, C₁-C₆ alkyl, C₁-C₆ haloalkyl, C₁-C₆ alkoxy, cycloalkyl, heterocycloalkyl, aryl, or heteroaryl, wherein each of the alkyl, aryl, cycloalkyl, heterocycloalkyl, or heteroaryl is optionally substituted with one, two, or three R₁₇;
R₁₅ₐ and R_{15c} are each independently H, amino, halogen, —CN, —OH, —OCF₃, C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl is optionally substituted with one, two, or three R²⁰;
each R₁₇, R₁₈ , R₁₉, and R₂₀ is independently selected from oxo, halogen, —CN, —NH₂, —NH(C₁—₆ alkyl), —N(C₁—₆ alkyl)₂, —OH, —CO₂H, —CO₂—C₁—₆ alkyl, —C(═O)NH₂, —C(═O)NH(C₁—₆ alkyl), —C(═O)N(C₁—₆ alkyl)₂, —S(═O)₂NH₂, —S(═O)₂NH(C₁—₆ alkyl), —S(═O)₂N(C₁—₆ alkyl)₂, C₁-C₆ alkyl, C₁-C₆ haloalkyl, C₃₋₈ cycloalkyl, C₁-C₆ heteroalkyl, C₁-C₆ alkoxy, C₁-₆ fluoroalkoxy, C₂-₇ heterocycloalkyl, aryl, heteroaryl, aryloxy, alkylthio, arylthio, alkylsulfoxide, arylsulfoxide, cycloalkylsulfone, alkylsulfone, and arylsulfone.

5. The compound of claim 1, or a pharmaceutically acceptable salt or solvate thereof, wherein the compound has a structure of Formula (XI): 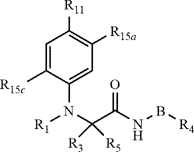
wherein,
B is a bond, C₁-C₄ alkylene, or C₃-C₆ cyclene linker;
Ri is halo acetyl, glyoxyl, heterocyclo acyl or acrylo acyl;
R₃ is a heteroaryl optionally substituted with one, two, or three R₁₈;
R₄ is a substituted cycloalkyl or an optionally substituted heterocycloalkyl, wherein when substituted the each of which is substituted with one, two, three, or four R¹⁹;
R₅ is H, C₁-C₆ alkyl, or C₁-C₃ haloalkyl;
R₁₁ is amino, halogen, —CN, —OH, —OCF₃, C₁-C₆ alkyl, C₁-C₆ haloalkyl, C₁-C₆ alkoxy, cycloalkyl, heterocycloalkyl, aryl, or heteroaryl, wherein each of the alkyl, aryl, cycloalkyl, heterocycloalkyl, or heteroaryl is optionally substituted with one, two, or three R₁₇;
R₁₅ₐ and R_{15c} are each independently H, amino, halogen, —CN, —OH, —OCF₃, C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl is optionally substituted with one, two, or three R²⁰;
each R₁₇, R₁₈ , R₁₉, and R₂₀ is independently selected from oxo, halogen, —CN, —NH₂, —NH(C₁—₆ alkyl), —N(C₁—₆ alkyl)₂, —OH, —CO₂H, —CO₂—C₁—₆ alkyl, —C(═O)NH₂, —C(═O)NH(C₁—₆ alkyl), —C(═O)N(C₁—₆ alkyl)₂, —S(═O)₂NH₂, —S(═O)₂NH(C₁—₆ alkyl), —S(═O)₂N(C₁—₆ alkyl)₂, C₁-C₆ alkyl, C₁-C₆ haloalkyl, C₃₋₈ cycloalkyl, C₁-C₆ heteroalkyl, C₁-C₆ alkoxy, C₁-₆ fluoroalkoxy, C₂-₇ heterocycloalkyl, aryl, heteroaryl, aryloxy, alkylthio, arylthio, alkylsulfoxide, arylsulfoxide, alkylsulfone, and arylsulfone.

6-9. (canceled)

10. The compound of claim 1 or a pharmaceutically acceptable salt or solvate thereof, wherein B and B₁ is bond.

11. The compound of claim 1 or a pharmaceutically acceptable salt or solvate thereof, wherein R₃ is a 6-membered heteroaryl containing 1 to 3 N atoms.

12. The compound of claim 11, or a pharmaceutically acceptable salt or solvate thereof, wherein the 6-membered heteroaryl is pyridine, pyrimidine, pyrazine, or pyridazine.

13-19. (canceled)

20. The compound of claim 1 or a pharmaceutically acceptable salt or solvate thereof, wherein R₅ is H.

21-26. (canceled)

27. The compound of claim 1 or a pharmaceutically acceptable salt or solvate thereof, wherein R₁₁ is amino, halogen, —CN, —OH, —OCF₃, C₁-C₆ alkyl, C₁-C₆ haloalkyl, or C₁-C₆ alkoxy, optionally substituted with one, two, or three R₁₇.

28-32. (canceled)

33. The compound of claim 1 or a pharmaceutically acceptable salt or solvate thereof, wherein R₄ is heterocycloalkyl optionally substituted with one, two, or three R₁₉.

34. The compound of claim 33, or a pharmaceutically acceptable salt or solvate thereof, wherein each R₁₉ is independently halogen, oxo, —CN, —NH₂, —NH(C₁—₆ alkyl), —N(C₁—₆ alkyl)₂, —OH, —CO₂H, —CO₂—C₁—₆ alkyl, —C(═O)NH₂, —C(═O)NH(C₁—₆ alkyl), —C(═O)N(C₁—₆ alkyl)₂, —S(═O)₂NH₂, —S(═O)₂NH(C₁—₆ alkyl), —S(═O)₂N(C₁—₆ alkyl)₂, C₁-C₆ alkyl, C₁-C₆ haloalkyl, C₃₋₈ cycloalkyl, C₁-C₆ heteroalkyl, C₁-C₆ alkoxy, C₁-₆ fluoroalkoxy, C₂-₇ heterocycloalkyl, aryl, heteroaryl, aryloxy, alkylthio, arylthio, alkylsulfoxide, arylsulfoxide, alkylsulfone, and arylsulfone.

35. (canceled)

36. The compound of claim 1 or a pharmaceutically acceptable salt or solvate thereof, wherein R₄ is cycloalkyl, optionally substituted with one two or three R₁₉.

37. The compound of claim 36, or a pharmaceutically acceptable salt or solvate thereof, wherein each R₁₉ is independently halogen, oxo, —CN, —NH₂, —NH(C₁—₆ alkyl), —N(C₁—₆ alkyl)₂, —OH, —CO₂H, —CO₂—C₁—₆ alkyl, —C(═O)NH₂, —C(═O)NH(C₁—₆ alkyl), —C(═O)N(C₁—₆ alkyl)₂, —S(═O)₂NH₂, —S(═O)₂NH(C₁—₆ alkyl), —S(═O)₂N(C₁—₆ alkyl)₂, C₁-C₆ alkyl, C₁-C₆ haloalkyl, C₃₋₈ cycloalkyl, C₁-C₆ heteroalkyl, C₁-C₆ alkoxy, C₁-₆ fluoroalkoxy, C₂-₇ heterocycloalkyl, aryl, heteroaryl, aryloxy, alkylthio, arylthio, alkylsulfoxide, arylsulfoxide, alkylsulfone, and arylsulfone.

38. (canceled)

39. (canceled)

40. The compound of claim 1 , or a pharmaceutically acceptable salt or solvate thereof, wherein R₄ is selected from 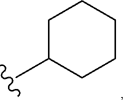 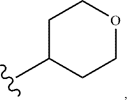 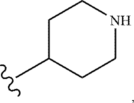 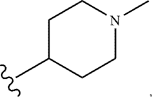 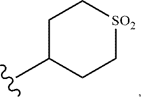 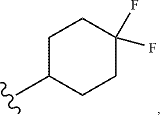 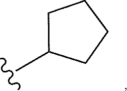 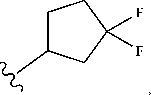 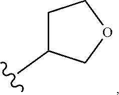 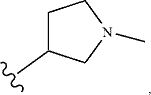 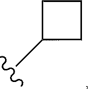 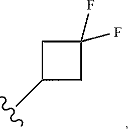 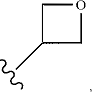 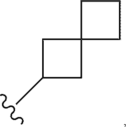 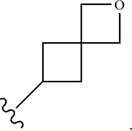 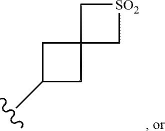 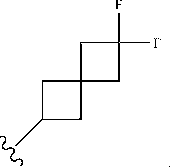
.

41. (canceled)

42. The compound of claim 1 or a pharmaceutically acceptable salt or solvate thereof, wherein R₁ is halo acetyl, heterocyclo acyl or acrylo acyl.

43. (canceled)

44. The compound of claim 1 or a pharmaceutically acceptable salt or solvate thereof, wherein R₁ is selected from 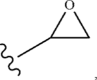 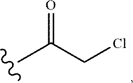 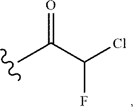 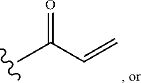 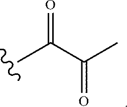
.

45. (canceled)

46. (canceled)

47. A compound that is selected from Table 1, or a pharmaceutically acceptable salt or solvate thereof.

48. The compound of claim 1 or a pharmaceutically acceptable salt or solvate thereof, wherein the compound is selected from: 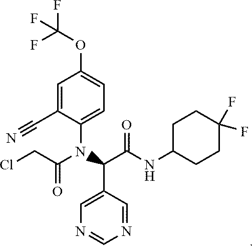 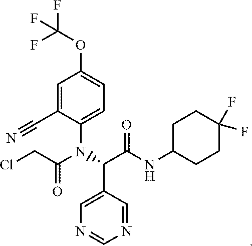 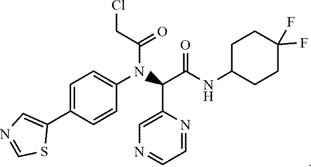 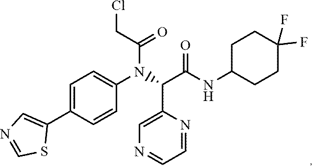 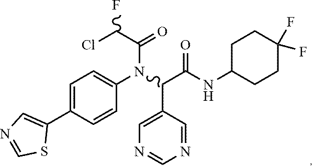 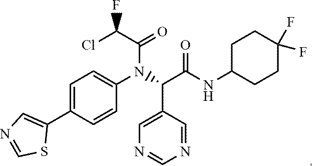 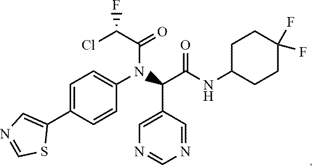 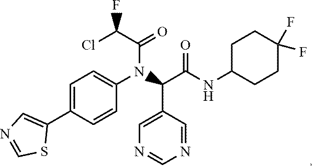 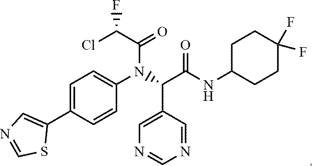 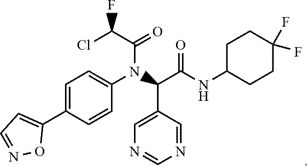 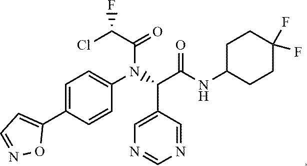 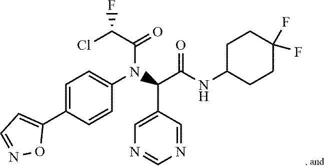 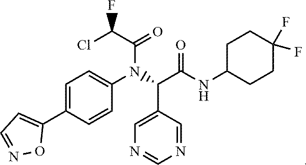
.

49-66. (canceled)

67. A pharmaceutical composition comprising a compound of claim 1 and a pharmaceutically acceptable excipient.

68. A method of treating or preventing a SARS—CoV—2 infection in a patient in need thereof, comprising administering to the patient a compound of claim 1.

69. (canceled)

70. (canceled)

71. An in vivo method of inhibiting a protease of SARS—CoV—2, comprising contacting the protease with a compound of claim 1.

72-75. (canceled)
